(19) United States
(12) Patent Application Publication
(10) Pub. No.: US 2024/0159756 A1
(43) Pub. Date: May 16, 2024

(54) PROTEOGENOMIC ANALYSIS OF NON-SMALL CELL LUNG CANCER

(71) Applicant: FenoMark Diagnostics AB, Stockholm (SE)

(72) Inventors: Lukas Orre, Spånga (SE); Taner Arslan, Bromma (SE); Ioannis Siavelis, Solna (SE); Janne Lehtiö, Bandhagen (SE)

(21) Appl. No.: 18/552,330

(22) PCT Filed: Mar. 29, 2022
(86) PCT No.: PCT/EP2022/058334

§ 371 Date: Sep. 25, 2023

(30) Foreign Application Priority Data
Mar. 29, 2021 (GB) 2104422.7

(51) Int. Cl. G01N 33/574; A61K 45/06; G01N 33/68; G16H 20/10; G16H 50/20

(52) U.S. Cl. CPC G01N 33/57423; A61K 45/06; G01N 33/6848; G16H 20/10; G16H 50/20

(57) ABSTRACT

The present invention provides various methods for determining the prognosis of Non-Small Cell Lung Cancer (NSCLC) in an individual, generally comprising the steps of: (a) providing a test sample from the individual; (b) determining a biomarker signature by measuring in the test sample the presence and/or amount of biomarkers characterizing specific NSCLC molecular sub types; and (c) classifying the NSCLC in the individual, wherein the prognosis of NSCLC in the individual is determined based on the classification. Also provided are methods of treatment based on said classification.

The present invention relates to methods for determining the prognosis of Non-Small Cell Lung Cancer (NSCLC) in an individual, as well as methods of treatment based on said prognosis.

Lung cancer is the most common type of cancer worldwide with 2.1 million new cases each year. The majority of cases are diagnosed when the cancer has already metastasized and surgical resection is no longer an option, resulting in a dismal overall 5-year survival rate for NSCLC of 24% and only 6% in stage 4 disease (seer.cancer.gov). Rapid development of targeted therapies and immunotherapy present a major opportunity, but the impact on survival so far is blunted by lack of biomarkers for therapy selection and limited knowledge of how therapies should be combined.

Exploratory omics-analysis of clinical cancer cohorts have demonstrated the value of a systems level analysis of cancer [1,2]. Most previous cancer landscape studies have placed emphasis on genetic alterations for stratification of patients into different subtypes. There is still a need to provide improved methods of determining the prognosis of NSCLC.

Against that background, the present inventors have defined, for the first time, a number of distinct subtypes of NSCLC based on the NSCLC proteome landscape. Surprisingly, those subtypes can be used to more-accurately determine the prognosis of NSCLC, and the present invention therefore provides new approaches for classifying and clinically managing the cancer.

In a first aspect, the invention provides a method for determining the prognosis of Non-Small Cell Lung Cancer (NSCLC) in an individual, the method comprising the steps of:
(1-a) providing a test sample from the individual;
(1-b) determining a biomarker signature, by measuring in the test sample the presence and/or amount of the biomarkers defined in Table 1 and/or Table 2 and/or Table 3 and/or Table 4 and/or Table 5 and/or Table 6;
(1-c) classifying the NSCLC in the individual on the basis of Step (1-b); wherein the prognosis of NSCLC in the individual is determined on the basis of the classification in Step (1-c).

Thus, the invention provides a method for determining the prognosis of NSCLC based on particular biomarkers identified by the present inventors. As explained in more detail in the accompanying Examples, the inventors performed an in-depth analysis of the NSCLC proteome landscape, covering nearly 14,000 biomarkers and all major NSCLC histological subtypes. That analysis identified that the particular biomarkers defined herein could be used to classify NSCLC and more-accurately determine the prognosis of the cancer.

By “determining the prognosis” we include determining the chance of survival of the individual with NSCLC over a defined period. It can also include the chance of the NSCLC recurring over a defined period. In the context of this invention, determining the prognosis of NSCLC relies on the classification of NSCLC into one of six prognostic sub-types 1 to 6.

By “Non-Small Cell Lung Cancer (NSCLC)” we include any type of lung cancer that is not Small Cell Lung Cancer (SCLC). For example, the NSCLC may be adenocarcinoma; squamous cell carcinoma; adenosquamous carcinoma; large cell carcinoma; or large cell neuroendocrine cancer.

By “test sample” (or sample to be tested) we include a sample to be tested in the invention, such as a sample taken or derived from an individual to be tested, wherein the sample comprises endogenous proteins and/or nucleic acid molecules. Preferably the sample to be tested is provided from an individual that is a mammal. In some embodiments, the individual may be a primate (for example, a human; a monkey; an ape); a rodent (for example, a mouse, a rat, a hamster, a guinea pig, a gerbil, a rabbit); a canine (for example, a dog); a feline (for example, a cat); an equine (for example, a horse); a bovine (for example, a cow); or a porcine (for example, a pig). Most preferably, the mammal is human.

The sample to be tested in the methods of the invention may comprise or consist of: a cell; tissue; fluid sample (or derivative thereof); and may preferably comprise or consist of blood (fractionated or unfractionated), plasma, plasma cells, serum, tissue cells, pleural fluid, pleural cells or equally preferred, protein or peptide or nucleic acid derived from a cell or tissue sample. It will be appreciated that the test and any control samples should be from the same species.

In one particularly preferred embodiment, the sample is a lung tissue sample. In an alternative or additional embodiment, the sample is a sample comprising or consisting of lung cells, for example epithelial cells or alveolar cells or pleural cells. In a preferred embodiment, the sample comprises one or more lung cancer cells.

The methods of this invention are suitable for testing a sample from any individual who has, or is suspected of having, NSCLC. For example, the individual may be from one of the following groups:
Individuals with previously diagnosed NSCLC (of any type or stage);
Individuals with suspected NSCLC;
Individuals with symptoms suggestive of or consistent with NSCLC (e.g. persistent coughing, coughing up blood, chest pain or pain when breathing, shortness of breath, fatigue, unintentional or unexplained weight loss, wheezing, hoarseness);
Individuals who have previously had lung cancer.

By “biomarker” we include naturally-occurring biological molecules (or components or fragments thereof) that provide information that is useful in the classification of NSCLC, that can in turn provide information on the prognosis of NSCLC. In the context of Tables 1-6 and Tables A-G, the biomarker may be a protein or polypeptide. The biomarker may also be a nucleic acid molecule, for example an mRNA or cDNA molecule.

It will be appreciated that mRNA (or cDNA) analysis may also be used as an effective approximation of the molecular phenotype. For example, previous studies have shown that in a few cancer types, molecular subtyping based on gene expression, assayed by transcriptomics, creates robust and clinically highly relevant patient stratification. It has been previously demonstrated that gene expression analysis can be used to stratify breast cancer samples with the potential to improve clinical prognostication [3].

By “biomarker signature” we mean the combination of biomarkers that are measured in the sample that are useful in the classification of NSCLC.

By “classifying the NSCLC in the individual”, we include assigning NSCLC in an individual into a particular group. These groups (or subtypes) are defined based on the biomarker signature. The NSCLC within these groups may have similar physical properties or pathologies, they may be expected to behave similarly, or the individuals with these NSCLC groups may be expected to have similar prognoses. In a preferred embodiment, individuals with NSCLC in the same group or subtype have a similar or the same prognosis. As discussed herein and demonstrated in the accompanying Examples, the present inventors have shown that classifying NSCLC in this way advantageously allows a more-accurate prediction of the expected timescale of the disease.

In some embodiments, this may include classifying the NSCLC based on the biomarker signature into one or more of the following subtypes:
Prognosis Subtype 1 (associated with the biomarker signature of Table 1);
Prognosis Subtype 2 (associated with the biomarker signature of Table 2);
Prognosis Subtype 3 (associated with the biomarker signature of Table 3);
Prognosis Subtype 4 (associated with the biomarker signature of Table 4);
Prognosis Subtype 5 (associated with the biomarker signature of Table 5);
Prognosis Subtype 6 (associated with the biomarker signature of Table 6).

The Prognosis Subtypes 1-6 associated with the invention are associated with detection of the presence and/or amount of the biomarkers associated with them. It will be evident that this may be indicative of shared features within the molecular phenotype of NSCLC having the same subtype. These common features may include, but are not limited to, one or more of the following:
the histological type of NSCLC;
the identity of drivers including driver mutations;
the tumour mutation burden (TMB);
the level of immune cell infiltration;
the level of antigen processing and presentation machinery (APM)
the neoantigen (NB) burden;
the type and level of immune-checkpoint proteins (such as, but not limited to, PD-L1, FGL1 and B7-H4, PD-1/PDCD1);
the type and level of cancer and driver related proteins (CDRPs);
the type of immune infiltration (T-cells, B-cells etc.);
the presence and abundance of particular histological features such as tertiary lymphoid structures (TLS).

For example, in preferred embodiments:
Subtype 1-4 may be associated with the NSCLC being adenocarcinoma (AC);
Subtype 5 may be associated with the NSCLC being large-cell neuroendocrine lung cancer (LCNEC);
Subtype 6 may be associated with the NSCLC being squamous cell lung carcinoma (SqCC);
Subtype 2: may be associated with the NSCLC being immune-infiltrated, a high tumour mutation burden, active antigen presentation, high CXCL9 level, and high PD-L1 level;
Subtype 4: may be associated with over-active mTOR signalling.
Subtype 1: may be associated with EGFR mutation and over-active EGFR signalling;
Subtype 3: may be associated with immune-infiltration, high B-cell infiltration, high tertiary lymphoid structure (TLS) counts;
Subtype 4-6: may be associated with high neoantigen burden (NB);
Subtype 4: may be associated with high TMB, high FGL1 level;
Subtype 6: may be associated with high B7-H4 level.

Therefore, the methods of the invention are capable of determining the Dominant Molecular Cancer Phenotype (DMCP), by which we mean the most distinct features of the tumour. Advantageously, this level of information is crucial for understanding how cancer cells acquire hallmark capabilities such as oncogenic growth, evasion of cell death signalling and immune evasion, and in turn for determining the prognosis with improved accuracy. Determining the DMCP, and consequently the prognosis, is independent of any histological based typing or staging of NSCLC.

The classification in Step (i-c) may be achieved using one or more of the following techniques: comparison of the presence and/or amount of the biomarkers to those in positive and/or negative control samples; comparison of the presence and/or amount of the biomarkers to pre-determined reference values; and/or algorithm-based techniques. Examples of algorithm-based techniques include but are not limited to the following:
Linear Models (for example Ordinary Least Squares, Ridge Classification, Lasso, Elastic-Net, Logistic Regression, Generalized Linear Classification, Stochastic Gradient Descent, Perceptron)
Linear and Quadratic Discriminant Analysis
Support Vector Machines (SVM) (for example SVM with linear kernel, SVM with polynomial (degree) kernel, SVM with Radial Basis Function Kernel)
Nearest Neighbours
Gaussian Process
Naïve Bayes
Decision Trees (for example Random Forest)
Ensemble Methods (for example Bagging, Boosting, Random Forests, Extremely Randomized Trees, AdaBoost, Gradient Tree Boosting, XGBoost, LightGBM)
Neural-Networks Classifiers (for example Multi-layer Perceptron, Artificial Neural-Networks, Deep-Learning)
Top Scoring Pairs (TSP) (for example TSP, k-TSP)

It will be clear to the skilled person that by “measuring in the test sample the presence and/or amount of the biomarkers defined in Table 1 and/or Table 2 and/or Table 3 and/or Table 4 and/or Table 5 and/or Table 6” we include the situation where less than all of the biomarkers defined in each of Tables 1 to 6 are measured in Step (1-b).

For instance, in some embodiments, Step (1-b) comprises measuring in the test sample the presence and/or amount of:
39 or more of the biomarkers defined in Table 1; and/or
11 or more of the biomarkers defined in Table 2; and/or
2 or more of the biomarkers defined in Table 3; and/or
8 or more of the biomarkers defined in Table 4; and/or
137 or more of the biomarkers defined in Table 5; and/or
36 or more of the biomarkers defined in Table 6.

In this embodiment, Step (1-b) may comprise measuring in the test sample the presence and/or amount of around 30% of the biomarkers defined in Table 1 and/or Table 2 and/or Table 3 and/or Table 4 and/or Table 5 and/or Table 6. In other embodiments, Step (1-b) comprises measuring the presence and/or amount of around 35%, 40%, or 45% of the biomarkers defined in Table 1 and/or Table 2 and/or Table 3 and/or Table 4 and/or Table 5 and/or Table 6.

In some embodiments, Step (1-b) comprises measuring in the test sample the presence and/or amount of:
66 or more of the biomarkers defined in Table 1; and/or
19 or more of the biomarkers defined in Table 2; and/or
3 or more of the biomarkers defined in Table 3; and/or
14 or more of the biomarkers defined in Table 4; and/or
229 or more of the biomarkers defined in Table 5; and/or
61 or more of the biomarkers defined in Table 6.

In this embodiment, Step (1-b) may comprise measuring in the test sample the presence and/or amount of around 50% of the biomarkers defined in Table 1 and/or Table 2 and/or Table 3 and/or Table 4 and/or Table 5 and/or Table 6. In other embodiments, Step (1-b) comprises measuring the presence and/or amount of around 55%, 60%, 65%, 70%, or 75% of the biomarkers defined in Table 1 and/or Table 2 and/or Table 3 and/or Table 4 and/or Table 5 and/or Table 6.

For instance, in some particularly preferred embodiments, Step (1-b) comprises measuring in the test sample the presence and/or amount of:
105 or more of the biomarkers defined in Table 1; and/or
30 or more of the biomarkers defined in Table 2; and/or
4 or more of the biomarkers defined in Table 3; and/or
22 or more of the biomarkers defined in Table 4; and/or
367 or more of the biomarkers defined in Table 5; and/or
97 or more of the biomarkers defined in Table 6.

In other embodiments, Step (1-b) comprises measuring the presence and/or amount of around 80% of the biomarkers defined in Table 1 and/or Table 2 and/or Table 3 and/or Table 4 and/or Table 5 and/or Table 6. In other embodiments, Step (1-b) comprises measuring the presence and/or amount of around 85%, 90%, 95%, 96%, 97%, 98%, 99% or 100% of the biomarkers defined in Table 1 and/or Table 2 and/or Table 3 and/or Table 4 and/or Table 5 and/or Table 6.

It will be clear to the skilled person that the method may comprise or consist of measuring a combination of different numbers (or percentages) of biomarkers from each of Tables 1-6. For instance, the method may comprise or consist of measuring 50% of the biomarkers in each of Tables 1-6. In other embodiments, the method may comprise measuring 80% of the biomarkers of one of the Tables 1-6, along with 50% of the biomarkers from one of the other Tables 1-6.

It will be appreciated that any combination of the biomarkers within each of Tables 1, 2, 3, 4, 5, and 6 may be measured in this embodiment. In addition, the method can also involve measuring different combinations of biomarkers from each of Tables 1-6. In some embodiments, Step (1-b) comprises measuring in the test sample the presence and/or amount of all of the biomarkers defined in Table 1 and/or Table 2 and/or Table 3 and/or Table 4 and/or Table 5 and/or Table 6. For example, in some embodiments, Step (1-b) comprises or consists of measuring the presence and/or amount of all of the biomarkers defined in Table 1, Table 2, Table 3, Table 4, Table 5, and Table 6.

In some embodiments, Step (1-b) comprises measuring in the test sample the presence and/or amount of some or all of the biomarkers defined in two or more, or three or more, or four or more, or five or more, or all of Tables 1-6. In this embodiment, measuring biomarkers from a combination of Tables 1-6 allows greater levels of discrimination between different sub-types to be achieved when performing a single iteration of the method on a single sample. This will also allow the analysis to be carried out with improved resolution, leading to better accuracy in the classification.

In some other embodiments, the method comprises comparing the biomarker signature in Step (1-b) with the corresponding biomarker signature of a control sample. The control sample may be a negative control or a positive control.

Therefore, in some embodiments the method of the first aspect further comprises the steps of:
(1-d) Providing one or more negative control sample(s);
(1-e) Determining a biomarker signature of the control sample(s), by measuring in the control sample(s) the presence and/or amount of the biomarkers defined in Table 1 and/or Table 2 and/or Table 3 and/or Table 4 and/or Table 5 and/or Table 6;
   wherein the classification in Step (1-c) is based on determining whether the presence and/or amount in the test sample of the biomarkers measured in Step (1-b) is different from the presence and/or amount in the negative control sample of the biomarkers measured in Step (1-e).

By “is different from the presence and/or amount in the negative control sample” we include the situation where the biomarker is detected in the test sample, but is not detected in the negative control sample(s), and vice versa. We also include the situation where the biomarker in question is upregulated or downregulated in the test sample compared to the same biomarker in the control sample. By “upregulated or downregulated” we include where the amount of the biomarker in the test sample differs from the amount of the biomarker in the control sample by at least ±5%, ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, ±24%, ±25%, ±26%, ±27%, ±28%, ±29%, ±30%, ±31%, ±32%, ±33%, ±34%, ±35%, ±36%, ±37%, ±38%, ±39%, ±40%, ±41%, ±42%, ±43%, ±44%, ±45%, ±41%, ±42%, ±43%, ±44%, ±55%, ±60%, ±65%, ±66%, ±67%, ±68%, ±69%, ±70%, ±71%, ±72%, ±73%, ±74%, ±75%, ±76%, ±77%, ±78%, ±79%, ±80%, ±81%, ±82%, ±83%, ±84%, ±85%, ±86%, ±87%, ±88%, ±89%, ±90%, ±91%, ±92%, ±93%, ±94%, ±95%, ±96%, ±97%, ±98%, ±99%, ±100%, ±125%, ±150%, ±175%, ±200%, ±225%, ±250%, ±275%, ±300%, ±350%, ±400%, ±500% or at least ±1000% from the one or more negative control sample(s).

Alternatively or additionally, the presence or amount in the test sample differs from the mean presence or amount in the control samples by at least >1 standard deviation from the mean presence or amount in the control samples, for example, ≥1.5, ≥2, ≥3, ≥4, ≥5, ≥6, ≥7, ≥28, ≥9, ≥10, ≥11, ≥12, ≥13, ≥14 or ≥15 standard deviations from the mean presence or amount in the control samples. Any suitable means may be used for determining standard deviation, however, in one embodiment, standard deviation is determined using the direct method (i.e., the square root of [the sum the squares of the samples minus the mean, divided by the number of samples]). In additional or alternative embodiments, other statistical methods that are well known in the art can be used to determine whether there is a difference between the presence or amount of a biomarker in the test sample compared to a control sample. Such methods may include, but are not limited to the following: Student t-test, Mann-Whitney U test, one-way analysis of variance (ANOVA), Kruskal-Wallis test, Limma test.

By “negative control sample” we include one or more of the following: a sample derived from normal lung tissue from the individual (e.g. healthy tissue adjacent to the NSCLC tissue taken during a biopsy); or from a healthy individual; or a pool of healthy individuals. By “healthy individual” we include individuals not afflicted with NSCLC or other types of lung cancer or other types of lung disease or condition. In the case where the negative control is derived from a pool of healthy individuals, the amount of the biomarker may be an average value of the amount of the biomarker measured in each of samples from the healthy individuals.

In additional or alternative embodiments, the method of the first aspect further comprises the steps of:
(1-f) Providing one or more positive control sample(s);
(1-g) Determining a biomarker signature of the positive control sample(s), by measuring in the control sample(s) the presence and/or amount of the biomarkers defined in Table 1 and/or Table 2 and/or Table 3 and/or Table 4 and/or Table 5 and/or Table 6;
   wherein the classification in Step (1-c) is based on determining whether the presence and/or amount in the test sample of the biomarkers measured in Step (1-b) corresponds to the presence and/or amount in the positive control sample of the biomarkers measured in Step (1-g).

Therefore, in some embodiments the method of the first aspect may further comprise Steps (1-d) and (1-e) and/or Steps (1-f) and (1-g).

By “corresponds to the presence and/or amount in the positive control sample” we include the situation where the biomarker is detected in both the test sample and the control sample. We also include that the presence and/or amount is identical to that of the positive control sample(s), or closer to that of one or more positive control sample(s) than to one or more negative control sample(s). Preferably, the amount of the biomarker in the test sample is within ±50% of that of the one or more control sample(s), for example, is within ±45%, ±40%, ±35%, ±30%, ±25%, ±20%, ±15%, ±10%, ±9%, ±8%, ±7%, ±6%, ±5%, ±4%, ±3%, ±2%, ±1%, ±0.5% of the amount of the biomarker in one or more positive control sample(s).

In an alternative or additional embodiment, the difference in the presence and/or amount in the test sample is ≥5 standard deviation from the mean presence or amount in the positive control sample(s), for example, ≥4.5, ≥4, ≥3.5, ≥3, ≥2.5, ≥2, ≥1.5, ≥1.4, ≥1.3, ≥1.2, ≥1.1, ≥1, ≥0.9, ≥0.8, ≥0.7, ≥0.6, ≥0.5, ≥0.4, ≥0.3, ≥0.2, ≥0.1 or 0 standard deviations from the from the mean presence or amount in the control sample(s).

By “positive control sample” we include samples derived from an individual with confirmed NSCLC or a pool of NSCLC samples. In the case where the positive control is a pool of NSCLC samples, the amount of the biomarker may be an average value of the amount of the biomarker measured in each of the NSCLC samples.

Therefore, in some embodiments the classification of Step (1-c) may be achieved by comparing the presence and/or amount of biomarkers in the test sample to those in the one or more positive and/or negative control sample(s).

For instance, the test sample may be classified as being in Prognosis Subtype 1 if greater than 50% of the biomarkers in the test sample measured from Table 1 are different from or correspond to the presence and/or amount of the corresponding biomarkers measured from Table 1 in the negative and/or positive control sample(s). In other embodiments, the classification into Prognosis Subtype 1 may be made if greater than 60%, 65%, 70%, 75%, 80%, 85%, 90%, 95%, 96%, 97%, 98%, or 99% of the biomarkers in the test sample measured from Table 1 are different from or correspond to the presence and/or amount of the corresponding biomarkers measured from Table 1 in the negative and/or positive control sample(s).

In some embodiments, 100% of the biomarkers measured from Table 1 are different from or correspond to the presence and/or amount of the corresponding biomarkers from Table 1 in the negative and/or positive control sample(s). The skilled person would also understand that the test sample may be classified as Prognosis Subtypes 2-6 upon measurement of the appropriate proportions of biomarkers from Tables 2-6, respectively, as defined in the preceding paragraph.

In a second aspect, the invention provides a method for determining the prognosis of Non-Small Cell Lung Cancer (NSCLC) in an individual, the method comprising the steps of:
(2-a) providing a test sample from the individual;
(2-b) determining in the test sample the presence and/or amount of the biomarkers in Table A and/or one or more of Tables B-G;
(2-c) applying a classification algorithm to the information obtained in step (2-b) in order to classify the NSCLC in the individual;
(2-d) classifying the NSCLC in the individual on the basis of Step (2-c), wherein the NSCLC is classified according to the biomarkers defined in Table 1 (as Prognosis Subtype 1) and/or Table 2 (as Prognosis Subtype 2) and/or Table 3 (as Prognosis Subtype 3) and/or Table 4 (as Prognosis Subtype 4) and/or Table 5 (as Prognosis Subtype 5) and/or Table 6 (as Prognosis Subtype 6);
   wherein the prognosis of NSCLC in the individual is determined on the basis of the classification in step (2-d).

By “determining the prognosis” we include determining the chance of survival of the individual with NSCLC over a defined period. It can also include the chance of the NSCLC recurring over a defined period. In the context of this invention, the prognosis of NSCLC relies on the classification of NSCLC into one of six prognostic sub-types 1 to 6.

By “Non-Small Cell Lung Cancer (NSCLC)” we include any type of lung cancer that is not Small Cell Lung Cancer (SCLC). For example, the NSCLC may be adenocarcinoma; squamous cell carcinoma; adenosquamous carcinoma; large cell carcinoma; or large cell neuroendocrine cancer.

By “test sample” (or sample to be tested) we include a sample to be tested in the invention, such as a sample taken or derived from an individual to be tested, wherein the sample comprises endogenous proteins and/or nucleic acid molecules. Preferably the sample to be tested is provided from an individual that is a mammal. In some embodiments, the individual may be a primate (for example, a human; a monkey; an ape); a rodent (for example, a mouse, a rat, a hamster, a guinea pig, a gerbil, a rabbit); a canine (for example, a dog); a feline (for example, a cat); an equine (for example, a horse); a bovine (for example, a cow); or a porcine (for example, a pig). Most preferably, the mammal is human.

The sample to be tested in the methods of the invention may comprise or consist of: a cell; tissue; fluid sample (or derivative thereof); and may preferably comprise or consist of blood (fractionated or unfractionated), plasma, plasma cells, serum, tissue cells, pleural fluid, pleural cells or equally preferred, protein or polypeptide or nucleic acid derived from a cell or tissue sample. It will be appreciated that the test and any control samples should be from the same species.

In one particularly preferred embodiment, the sample is a lung tissue sample. In an alternative or additional embodiment, the sample is a sample comprising or consisting of lung cells, for example epithelial cells or alveolar cells or pleural cells. In a preferred embodiment, the sample comprises one or more lung cancer cells.

The methods of this invention are suitable for testing a sample from any individual who has, or is suspected of having, NSCLC. For example, the individual may be from one of the following groups:
Individuals with previously diagnosed NSCLC (of any type or stage);
Individuals with suspected NSCLC;
Individuals with symptoms suggestive of or consistent with NSCLC (e.g. persistent coughing, coughing up blood, chest pain or pain when breathing, shortness of breath, fatigue, unintentional or unexplained weight loss, wheezing, hoarseness);
Individuals who have previously had lung cancer.

By “biomarker” we include naturally-occurring biological molecules (or components or fragments thereof) that provides information that is useful in the classification of NSCLC, that can in turn provide information on the prognosis of NSCLC. In the context of Tables 1-6 and Tables A-G, the biomarker may be the protein or polypeptide. The biomarker may be a nucleic acid molecule, for example an mRNA or cDNA molecule.

By “biomarker signature” we mean the combination of biomarkers that are measured in the sample that are useful in the classification of NSCLC.

By “classifying the NSCLC in the individual”, we include classifying the NSCLC based on the biomarker signature into one or more of the following subtypes:
Prognosis Subtype 1 (associated with the biomarker signature of Table 1 (or Table A(i));
Prognosis Subtype 2 (associated with the biomarker signature of Table 2 (or Table A(ii));
Prognosis Subtype 3 (associated with the biomarker signature of Table 3 (or Table A(iii));
Prognosis Subtype 4 (associated with the biomarker signature of Table 4 (or Table A(iv));
Prognosis Subtype 5 (associated with the biomarker signature of Table 5 (or Table A(v));
Prognosis Subtype 6 (associated with the biomarker signature of Table 6 (or Table A(vi)).

As discussed herein and demonstrated in the accompanying Examples, the present inventors have shown that classifying NSCLC in this way advantageously allows a more-accurate prediction of the expected timescale of the disease.

The Prognosis Subtypes 1-6 associated with the invention are associated with detection of the presence and/or amount of common biomarkers. It will be evident that this may be indicative of shared features within the molecular phenotype of NSCLC within the same subtype. These common features may include, but are not limited to, one or more of the following:
the histological type of NSCLC;
the identity of drivers including driver mutations;
the tumour mutation burden (TMB);
the level of immune cell infiltration;
the level of antigen processing and presentation machinery (APM);
the neoantigen (NB) burden;
the type and level of immune-checkpoint proteins (such as, but not limited to, PD-L1, FGL1 and B7-H4, PD-1/PDCD1);
the type and level of cancer and driver related proteins (CDRPs);
the type of immune infiltration (T-cells, B-cells etc.);
the presence and abundance of particular histological features such as tertiary lymphoid structures (TLS).

For example, in preferred embodiments:
Subtype 1-4 may be associated with the NSCLC being adenocarcinoma (AC);
Subtype 5 may be associated with the NSCLC being large-cell neuroendocrine lung cancer (LCNEC);
Subtype 6 may be associated with the NSCLC being squamous cell lung carcinoma (SgCC);
Subtype 2: may be associated with the NSCLC being immune-infiltrated, a high tumour mutation burden, active antigen presentation, and high PD-L1 level;
Subtype 4: may be associated with over-active mTOR signalling;
Subtype 1: may be associated with EGFR mutation and over-active EGFR signalling;
Subtype 3: may be associated with immune-infiltration, high B-cell infiltration, high tertiary lymphoid structure (TLS) counts;
Subtype 4-6: may be associated with high neoantigen burden (NB);
Subtype 4: may be associated with high TMB, high FGL1 level;
Subtype 6: may be associated with high B7-H4 level.

Therefore, the methods of the invention are capable of determining the Dominant Molecular Cancer Phenotype (DMCP), by which we mean the most distinct features of the tumour. This level of information is crucial for understanding how cancer cells acquire hallmark capabilities such as oncogenic growth, evasion of cell death signalling and immune evasion, and in turn for determining the prognosis. Determining the DMCP, and consequently the prognosis, is independent of any histological based typing or staging of NSCLC.

It will be clear to the skilled person that by “measuring in the test sample the presence and/or amount of the biomarkers defined in Table A” we include the situation where less than all of the biomarkers defined in Table A, and each of Tables A(i)-(vii) are be measured in Step (2-b).

By “classification algorithm” we include any algorithm that is capable of taking the data from the presence and/or amount of the biomarkers measured in Step (2-b) and using it to sort the individual into an NSCLC subtype, preferably wherein the NSCLC subtype is a prognosis subtype known herein as Prognosis Subtypes 1-6. The skilled person will be aware of common classification algorithms used in the art. Common examples are, but are not limited to, the following:
Linear Models (for example Ordinary Least Squares, Ridge Classification, Lasso, Elastic-Net, Logistic Regression, Generalized Linear Classification, Stochastic Gradient Descent, Perceptron)
Linear and Quadratic Discriminant Analysis
Support Vector Machines (SVM) (for example SVM with linear kernel, SVM with polynomial (degree) kernel, SVM with Radial Basis Function Kernel)
Nearest Neighbours
Gaussian Process
Naïve Bayes
Decision Trees (for example Random Forest)
Ensemble Methods (for example Bagging, Boosting, Random Forests, Extremely Randomized Trees, AdaBoost, Gradient Tree Boosting, XGBoost, LightGBM)
Neural-Networks Classifiers (for example Multi-layer Perceptron, Artificial Neural-Networks, Deep-Learning)
Top Scoring Pairs (TSP) (for example TSP, k-TSP).

All of these examples are suitable for performing the classification in Step (2-d). In some preferred embodiments, the classification algorithm is selected from:
Support Vector Machine-protein (“SVM-protein”);
K-Top Scoring Pairs (“k-TSP”); or
Support Vector Machine-Peptide (“SVM-peptide”).

A Support Vector Machine (SVM) is a supervised learning model that can be used for classification and regression analysis. Given a set of training examples, each marked as belonging to one or the other of two categories, an SVM training algorithm builds a model that assigns new examples to one category or the other. An SVM model is a representation of the examples as points in space, mapped so that the examples of the separate categories are divided by a clear gap that is as wide as possible. New examples are then mapped into that same space and predicted to belong to a category based on the side of the gap on which they fall.

An SVM constructs a hyperplane or set of hyperplanes in a high or infinite dimensional space, which can be used for classification, regression or other tasks. Intuitively, a good separation is achieved by the hyperplane that has the largest distance to the nearest training data points of any class (so-called functional margin), since in general the larger the margin the lower the generalization error of the classifier.

Therefore, in some embodiments the SVM is trained prior to performing the methods of the invention using profiles of biomarkers from individuals known to have NSCLC of a particular prognosis subtype, for example Prognosis Subtypes 1-6. This allows the SVM to learn which profiles are associated with the prognosis subtypes, and to learn which features and parameters are most important to the model, to allow accurate classification when test samples are applied. In some cases, the SVM can be validated using a separate data set, or a cross-validation can be performed using the training data set, for example using a Monte-Carlo cross validation method. SVM methods can be used to classify samples based on levels of protein biomarkers, peptide biomarkers, and nucleic acids (e.g. mRNA) coding for said proteins or peptides.

K-Top Scoring Pairs (k-TSP) is a classification method that is based on a set of paired measurements. Essentially, each of the two possible orderings of a pair of measurements (e.g. levels of biomarkers) is associated with one of two classes. K-TSP is the aggregation of a collection of such two-feature decision rules. K-TSP can be trained and validated in a similar way to the SVMs described above, and can also be trained using pre-defined reference values for each biomarker, leading to development of a classification algorithm capable of classifying test samples into prognostic subtypes. K-TSP methods can also be used to classify samples based on levels of protein biomarkers, peptide biomarkers, and nucleic acids (e.g. mRNA) coding for said proteins or peptides.

In some embodiments, performing training on one of the above classification algorithms may lead to identification of a combination of biomarkers that can serve as a biomarker signature that allows classification of NSCLC in an individual. It will be appreciated that each of the above algorithms may identify slightly different biomarker signatures that work best when test samples are classified using that particular algorithm.

Therefore, in some embodiments the classification algorithm is a Support Vector Machine-protein (“SVM-protein”), and Step (2-b) comprises measuring the presence and/or amount of 145 or more of the biomarkers defined in Table B, and/or 60 or more of the biomarkers defined in Table C. In this embodiment, Step (2-b) may comprise measuring in the test sample the presence and/or amount of around 30% of the biomarkers defined in Table B and/or Table C. In other embodiments, Step (2-b) comprises measuring the presence and/or amount of around 35%, 40% or 45% of the biomarkers defined in Table B and/or Table C.

In some embodiments the classification algorithm is a Support Vector Machine-protein (“SVM-protein”), and Step (2-b) comprises measuring the presence and/or amount of 243 or more of the biomarkers defined in Table B, and/or 100 or more of the biomarkers defined in Table C. In this embodiment, Step (2-b) may comprise measuring in the test sample the presence and/or amount of around 50% of the biomarkers defined in Table B and/or Table C. In other embodiments, Step (2-b) comprises measuring the presence and/or amount of around 55%, 60%, 65%, 70%, or 75% of the biomarkers defined in Table B and/or Table C.

In other embodiments, the classification algorithm is a Support Vector Machine-protein (“SVM-protein”), and Step (2-b) comprises measuring the presence and/or amount of 388 or more of the biomarkers defined in Table B, and/or 160 or more of the biomarkers defined in Table C. In some embodiments all of the biomarkers of Table B and/or Table C are measured.

In other embodiments, Step (2-b) comprises measuring the presence and/or amount of around 80% of the biomarkers defined in Table B and/or Table C. In other embodiments, Step (2-b) comprises measuring the presence and/or amount of around 85%, 90%, 95%, 96%, 97%, 98%, 99% or 100% of the biomarkers defined in Table B and/or Table C.

In some embodiments, the classification algorithm is K-Top Scoring Pairs (“k-TSP”), and Step (2-b) comprises measuring the presence and/or amount of pairs of biomarkers from within Table A(i) and/or Table A(ii) and/or Table (iii) and/or Table (iv) and/or Table (v) and/or Table (vi), and optionally Table A(vii), to facilitate the classification based on paired measurements. It will be understood that, in some embodiments, the biomarkers of each pair are found within different tables defined herein, i.e. they are associated with different prognostic subtypes. In some other embodiments, the biomarkers of each pair are found within the same table defined herein, i.e. they are associated with the same prognostic subtype. Multiple pairs of biomarkers may be measured in order to perform the classification of Step (2-d) when k-TSP is the classification algorithm.

In some embodiments, preferred pairs of biomarkers for use with the k-TSP method are defined in Tables D and E herein. Therefore, in some embodiments, the classification algorithm is k-TSP and Step (2-b) comprises measuring the presence and/or amount of 489 or more of the biomarker pairs defined in Table D, and/or 67 or more of the biomarker pairs defined in Table E. In this embodiment, Step (2-b) may comprise measuring in the test sample the presence and/or amount of around 30% of the biomarker pairs defined in Table D and/or Table E. In other embodiments, Step (2-b) comprises measuring the presence and/or amount of around 35%, 40% or 45% of the biomarker pairs defined in Table D and/or Table E.

In some embodiments, the classification algorithm is k-TSP and Step (2-b) comprises measuring the presence and/or amount of 815 or more of the biomarker pairs defined in Table D, and/or 112 or more of the biomarker pairs defined in Table E. In this embodiment, Step (2-b) may comprise measuring in the test sample the presence and/or amount of around 50% of the biomarker pairs defined in Table D and/or Table E. In other embodiments, Step (2-b) comprises measuring the presence and/or amount of around 55%, 60%, 65%, 70%, or 75% of the biomarker pairs defined in Table D and/or Table E.

Therefore, in some embodiments, the classification algorithm is k-TSP and Step (2-b) comprises measuring the presence and/or amount of 1304 or more of the biomarker pairs defined in Table D, and/or 180 or more of the biomarker pairs defined in Table E. In some embodiments, all of the biomarker pairs of Table D and/or Table E are measured.

In other embodiments, Step (2-b) comprises measuring the presence and/or amount of around 80% of the biomarker pairs defined in Table D and/or Table E. In other embodiments, Step (2-b) comprises measuring the presence and/or amount of around 85%, 90%, 95%, 96%, 97%, 98%, 99% or 100% of the biomarker pairs defined in Table D and/or Table E.

In some embodiments, the classification algorithm is Support Vector Machine-peptide (“SVM-peptide”) and Step (2-b) comprises measuring the presence and/or amount of 174 or more of the biomarkers defined in Table F, and/or 60 or more of the biomarkers defined in Table G. In this embodiment, Step (2-b) may comprise measuring in the test sample the presence and/or amount of around 30% of the biomarkers defined in Table F and/or Table G. In other embodiments, Step (2-b) comprises measuring the presence and/or amount of around 35%, 40% or 45% of the biomarkers defined in Table F and/or Table G.

In some embodiments, the classification algorithm is Support Vector Machine-peptide (“SVM-peptide”) and Step (2-b) comprises measuring the presence and/or amount of 290 or more of the biomarkers defined in Table F, and/or 100 or more of the biomarkers defined in Table G. In this embodiment, Step (2-b) may comprise measuring in the test sample the presence and/or amount of around 50% of the biomarkers defined in Table F and/or Table G. In other embodiments, Step (2-b) comprises measuring the presence and/or amount of around 55%, 60%, 65%, 70%, or 75% of the biomarkers defined in Table F and/or Table G.

In some embodiments, the classification algorithm is Support Vector Machine-peptide (“SVM-peptide”) and Step (2-b) comprises measuring the presence and/or amount of 464 or more of the biomarkers defined in Table F, and/or 160 or more of the biomarkers defined in Table G. In some embodiments, all of the biomarkers of Table F and/or Table G are measured.

In other embodiments, Step (2-b) comprises measuring the presence and/or amount of around 80% of the biomarkers defined in Table F and/or Table G. In other embodiments, Step (2-b) comprises measuring the presence and/or amount of around 85%, 90%, 95%, 96%, 97%, 98%, 99% or 100% of the biomarkers defined in Table F and/or Table G.

In some embodiments, the classification algorithm is Support Vector Machine-peptide (“SVM-peptide”) and Step (2-b) comprises measuring the presence and/or amount of polypeptide biomarkers derived from or mapping to the protein biomarkers of Table A and/or Table F and/or Table G.

The biomarkers referred to herein were initially identified by screening for biomarkers that were statistically significant (abs(log 2FC)>0.5, DEqMS p.adj<0.01) in level between any of the subtypes. A priority subset of these markers (1755 In total) was generated by screening for biomarkers with abs(log 2FC)>1. This priority subset is included as Table A referred to herein.

The biomarkers of Tables 1-6 (and Tables A(i)-(vi)) are subsets of the biomarkers of Table A. The biomarkers of Table A(vii) are all of those biomarkers from the priority subset of Table A that are not found within any of Tables 1-6, of which there are 1118 biomarkers in total.

The subsets of biomarkers of Tables 1-6 (relating to the prognostic subtypes 1-6) were defined as biomarkers that were more abundant than in any of the other of the five subtypes (log 2FC>0.5) with statistical significance (DEqMS p.adj.<0.01).

The biomarkers of Tables B-G were identified using specific classifiers, and contain biomarkers selected by preferred features of these classifiers. The biomarkers of Table C are the priority subset of the biomarkers of Table B, and these biomarkers were identified during optimisation of the SVM-protein classifier. The biomarkers of Table E are the priority subset of the biomarkers of Table D, and these biomarkers were identified during optimisation of the k-TSP classifier. The biomarkers of Table G are the priority subset of the biomarkers of Table F, and these biomarkers were identified during optimisation of the SVM-peptide classifier. In each case the biomarkers are preferred for their respective classifier, however are not limited to being measured in methods using these classification algorithms specifically. Priority subsets are the most powerful in the respective classifiers.

It will be clear to the skilled person that by “measuring in the test sample the presence and/or amount of the biomarkers defined in Table A” we include the situation where less than all of the biomarkers defined in Table A, and each of Tables A(i)-(vii) are be measured in Step (2-b).

Therefore, in some embodiments, Step (2-b) comprises measuring in the test sample the presence and/or amount of 526 or more of the biomarkers of Table A.

In this embodiment, Step (2-b) may comprise measuring in the test sample the presence and/or amount of around 30% or more of the biomarkers defined in Table A. In other embodiments, Step (2-b) comprises measuring the presence and/or amount of around 35%, 40% or 45% of the biomarkers defined in Table A.

In some embodiments, Step (2-b) comprises measuring in the test sample the presence and/or amount of 877 or more of the biomarkers of Table A.

In this embodiment, Step (2-b) may comprise measuring in the test sample the presence and/or amount of around 50% or more of the biomarkers defined in Table A. In other embodiments, Step (2-b) comprises measuring the presence and/or amount of around 55%, 60%, 65%, 70%, or 75% of the biomarkers defined in Table A.

For instance, in some particularly preferred embodiments, Step (2-b) comprises measuring in the test sample the presence and/or amount of 1404 or more of the biomarkers defined in Table A.

In this embodiment, Step (2-b) comprises measuring the presence and/or amount of around 80% of the biomarkers defined in Table A. In other embodiments, Step (2-b) comprises measuring the presence and/or amount of around 85%, 90%, 95%, 96%, 97%, 98%, 99% or 100% of the biomarkers defined in Table A. Therefore, in some embodiments, Step (2-b) comprises determining the presence and/or amount of all of the biomarkers defined in Table A.

In some embodiments, Step (2-b) comprises determining the presence and/or amount of a subset of the biomarkers of Table A, which correspond to the biomarkers of Tables 1-6 and therefore the prognostic subtypes 1-6. In this embodiment, Step (2-b) comprises measuring the biomarkers of Table A(i) and/or Table A(ii) and/or Table A(Iii) and/or Table A(iv) and/or Table A(v) and/or Table (vi). It will be evident to the skilled person that this includes the situation where some, but not all, of the biomarkers of each of Tables A(i-vi) are measured.

In some preferred embodiments, Step (2-b) comprises determining the presence and/or amount of:
39 or more of the biomarkers defined in Table A(i); and/or
11 or more of the biomarkers defined in Table A(ii); and/or
2 or more of the biomarkers defined in Table A(iii); and/or
8 or more of the biomarkers defined in Table A(iv); and/or
137 or more of the biomarkers defined in Table A(v); and/or
36 or more of the biomarkers defined in Table A(vi).

In this embodiment, Step (2-b) may comprise measuring in the test sample the presence and/or amount of around 30% of the biomarkers defined in Table A(i) and/or Table A(ii) and/or Table A(iii) and/or Table A(iv) and/or Table A(v) and/or Table A(vi). In other embodiments, Step (2-b) comprises measuring the presence and/or amount of around 35%, 40% or 45% of the biomarkers defined in Table A(i) and/or Table A(ii) and/or Table A(iii) and/or Table A(iv) and/or Table A(v) and/or Table A(vi).

In some preferred embodiments, Step (2-b) comprises determining the presence and/or amount of:
66 or more of the biomarkers defined in Table A(i); and/or
19 or more of the biomarkers defined in Table A(ii); and/or
3 or more of the biomarkers defined in Table A(iii); and/or
14 or more of the biomarkers defined in Table A(iv); and/or
229 or more of the biomarkers defined in Table A(v); and/or
61 or more of the biomarkers defined in Table A(vi).

In this embodiment, Step (2-b) may comprise measuring in the test sample the presence and/or amount of around 50% of the biomarkers defined in Table A(i) and/or Table A(ii) and/or Table A(iii) and/or Table A(iv) and/or Table A(v) and/or Table A(vi). In other embodiments, Step (2-b) comprises measuring the presence and/or amount of around 55%, 60%, 65%, 70%, or 75% of the biomarkers defined in Table A(i) and/or Table A(ii) and/or Table A(iii) and/or Table A(iv) and/or Table A(v) and/or Table A(vi).

For Instance, in some particularly preferred embodiments, Step (2-b) comprises measuring in the test sample the presence and/or amount of:
105 or more of the biomarkers defined in Table A(i); and/or
30 or more of the biomarkers defined in Table A(ii); and/or
4 or more of the biomarkers defined in Table A(iii); and/or
22 or more of the biomarkers defined in Table A(iv); and/or
367 or more of the biomarkers defined in Table A(v); and/or
97 or more of the biomarkers defined in Table A(vi).

In other embodiments, Step (2-b) comprises measuring the presence and/or amount of around 80% of the biomarkers defined in Table A(i) and/or Table A(ii) and/or Table A(iii) and/or Table A(iv) and/or Table A(v) and/or Table A(vi). In other embodiments, Step (2-b) comprises measuring the presence and/or amount of around 85%, 90%, 95%, 96%, 97%, 98%, 99% or 100% of the biomarkers defined in Table A(i) and/or Table A(ii) and/or Table A(iii) and/or Table A(iv) and/or Table A(v) and/or Table A(vi).

In some embodiments, Step (2-b) comprises determining the presence and/or amount of all of the biomarkers of Table A(i). In additional or alternative embodiments, Step (2-b) comprises determining the presence and/or amount of all of the biomarkers of Table A(ii). In additional or alternative embodiments, Step (2-b) comprises determining the presence and/or amount of all of the biomarkers of Table A(iii). In additional or alternative embodiments, Step (2-b) comprises determining the presence and/or amount of all of the biomarkers of Table A(iv). In additional or alternative embodiments, Step (2-b) comprises determining the presence and/or amount of all of the biomarkers of Table A(v). In additional or alternative embodiments, Step (2-b) comprises determining the presence and/or amount of all of the biomarkers of Table A(vi).

Therefore, in some embodiments, Step (2-b) comprises determining the presence and/or amount of all of the biomarkers defined in each of Table A(i) and Table A(ii) and Table A(iii) and Table A(iv) and Table A(v) and Table A(vi).

It will be clear to the skilled person that the method may comprise or consist of measuring a combination of different numbers (or percentages) of biomarkers from each of Tables A(i-vi). For instance, the method may comprise or consist of measuring 50% of the biomarkers in each of Tables A(i-vi). In other embodiments, the method may comprise measuring 80% of the biomarkers of one of the Tables A(i-vi), along with 50% of the biomarkers from one of the other Tables A(i-vi).

It will be appreciated that any combination of the biomarkers within each of Tables A(i-vi) may be measured in this embodiment. In addition, the method can also involve measuring different combinations of biomarkers from each of Tables A(i-vi).

In some additional embodiments, the method of the second aspect further comprises determining the presence and/or amount of one or more biomarkers defined in Table A(vii). These biomarkers may be measured in addition to the biomarkers of one or more of Tables A(i-vi) described herein. The biomarkers of Tables A(vii) In some preferred embodiments, the presence and/or amount of at least 10% of the biomarkers defined in Table A(vii) are measured, for example at least 20%, 30%, 40%, 50%, 60%, 70%, 80%, 85%, 90%, 95% or 100% of the biomarkers of Table A(vii) are measured. Therefore, in some embodiments all of the biomarkers of Table A(vii) are measured. In some preferred embodiments, at least 335 of the biomarkers of Table A(vii) are measured.

In a third aspect, the invention provides a method for determining the prognosis of Non-Small Cell Lung Cancer (NSCLC) in an individual, the method comprising the steps of:
(3-a) providing a test sample from the individual;
(3-b) determining in the test sample the presence and/or amount of the biomarkers defined in Table A and/or one or more of Tables B-G;
(3-c) applying a classification algorithm to the information obtained in step (3-b) in order to classify the NSCLC in the individual, wherein the classification algorithm is selected from:
   Support Vector Machine-protein (“SVM-protein”) and the biomarkers defined in Table B or C; or
   K-Top Scoring Pairs (“k-TSP”) and the biomarkers defined in Table D or E; or
   Support Vector Machine-peptide (“SVM-peptide”) and the biomarkers defined in Table F or G;
(3-d) classifying the NSCLC in the individual on the basis of Step (3-c);
   wherein the prognosis of NSCLC in the individual is determined on the basis of the classification in step (3-d).

By “determining the prognosis” we include determining the chance of survival of the individual with NSCLC over a defined period, both with and without treatment. It can also include the chance of the NSCLC recurring over a defined period. In the context of this invention, the prognosis of NSCLC relies on the classification of NSCLC into one of six prognostic sub-types 1 to 6.

By “Non-Small Cell Lung Cancer (NSCLC)” we Include any type of lung cancer that Is not Small Cell Lung Cancer (SCLC). For example, the NSCLC may be adenocarcinoma; squamous cell carcinoma; adenosquamous carcinoma; large cell carcinoma; or large cell neuroendocrine cancer.

By “test sample” (or sample to be tested) we Include a sample to be tested in the Invention, such as a sample taken or derived from an individual to be tested, wherein the sample comprises endogenous proteins and/or nucleic acid molecules. Preferably the sample to be tested is provided from an Individual that is a mammal. In some embodiments, the Individual may be a primate (for example, a human; a monkey; an ape); a rodent (for example, a mouse, a rat, a hamster, a guinea pig, a gerbil, a rabbit); a canine (for example, a dog); a feline (for example, a cat); an equine (for example, a horse); a bovine (for example, a cow); or a porcine (for example, a pig). Most preferably, the mammal is human.

The sample to be tested in the methods of the Invention may comprise or consist of: a cell; tissue; fluid sample (or derivative thereof); and may preferably comprise or consist of blood (fractionated or unfractionated), plasma, plasma cells, serum, tissue cells, pleural fluid, pleural cells or equally preferred, protein or peptide or nucleic acid derived from a cell or tissue sample. It will be appreciated that the test and any control samples should be from the same species.

In one particularly preferred embodiment, the sample is a lung tissue sample. In an alternative or additional embodiment, the sample is a sample comprising or consisting of lung cells, for example epithelial cells or alveolar cells or pleural cells. In a preferred embodiment, the sample comprises one or more lung cancer cells.

The methods of this invention are suitable for testing a sample from any Individual who has, or is suspected of having, NSCLC. For example, the Individual may be from one of the following groups:
Individuals with previously diagnosed NSCLC (of any type or stage);
Individuals with suspected NSCLC;
Individuals with symptoms suggestive of or consistent with NSCLC (e.g. persistent coughing, coughing up blood, chest pain or pain when breathing, shortness of breath, fatigue, unintentional or unexplained weight loss, wheezing, hoarseness);
Individuals who have previously had lung cancer.

By “biomarker” we include naturally-occurring biological molecules (or components or fragments thereof) that provides information that is useful in the classification of NSCLC, that can in turn provide information on the prognosis of NSCLC. In the context of Tables 1-6 and Table A, the biomarker may be the protein or polypeptide. The biomarker may be a nucleic acid molecule, for example an mRNA or cDNA molecule.

By “biomarker signature” we mean the combination of biomarkers that are measured in the sample that are useful in the classification of NSCLC.

By “classifying the NSCLC in the individual”, we include assigning NSCLC in an individual into a particular group. These groups (or subtypes) are defined based on the biomarker signature. The NSCLC within these groups may have similar physical properties or pathologies, they may be expected to behave similarly, or the individuals with these NSCLC groups may be expected to have similar prognoses. In a preferred embodiment, Individuals with NSCLC in the same group or subtype have a similar or the same prognosis. As discussed herein and demonstrated in the accompanying Examples, the present Inventors have shown that classifying NSCLC in this way advantageously allows a more-accurate prediction of the expected timescale of the disease.

In this aspect of the Invention, the classification algorithm is a Support Vector Machine-protein (SVM-protein), K-Top Scoring Pairs (k-TSP) or Support Vector Machine-peptide (SVM-peptide), which are further defined herein in relation to the second aspect. In some embodiments, the classification algorithm of Step (3-c) is k-TSP and the pairs of biomarkers defined in Tables D and E are measured and compared. In some embodiments, the classification algorithm of Step (3-c) is k-TSP and the biomarkers are polypeptides derived from or mapping to the pairs of biomarkers defined in Table D and/or Table E.

In some embodiments of the third aspect, classification of the NSCLC based on the biomarker signature is into one or more of the following subtypes:
Prognosis Subtype 1 (associated with the biomarker signature of Table 1 (or Table A(i));
Prognosis Subtype 2 (associated with the biomarker signature of Table 2 (or Table A(ii));
Prognosis Subtype 3 (associated with the biomarker signature of Table 3 (or Table A(iii));
Prognosis Subtype 4 (associated with the biomarker signature of Table 4 (or Table A(iv));
Prognosis Subtype 5 (associated with the biomarker signature of Table 5 (or Table A(v));
Prognosis Subtype 6 (associated with the biomarker signature of Table 6 (or Table A(vi)).

The Prognosis Subtypes 1-6 associated with the invention are associated with detection of the presence and/or amount of common biomarkers. It will be evident that this may be indicative of shared features within the molecular phenotype of NSCLC within the same subtype.

These common features may include, but are not limited to, one or more of the following:
the histological type of NSCLC;
the Identity of drivers Including driver mutations;
the tumour mutation burden (TMB);
the level of immune cell infiltration;
the level of antigen processing and presentation machinery (APM);
the neoantigen (NB) burden;
the type and level of immune-checkpoint proteins (such as, but not limited to, PD-L1, FGL1 and B7-H4, PD-1/PDCD1);
the type and level of cancer and driver related proteins (CDRPs);
the type of immune infiltration (T-cells, B-cells etc.);
the presence and abundance of particular histological features such as tertiary lymphoid structures (TLS).

For example, in preferred embodiments:
Subtype 1-4 may be associated with the NSCLC being adenocarcinoma (AC);
Subtype 5 may be associated with the NSCLC being large-cell neuroendocrine lung cancer (LCNEC);
Subtype 6 may be associated with the NSCLC being squamous cell lung carcinoma (SgCC);
Subtype 2: may be associated with the NSCLC being immune-infiltrated, a high tumour mutation burden, active antigen presentation, and high PD-L1 level;
Subtype 4: may be associated with over-active mTOR signalling;
Subtype 1: may be associated with EGFR mutation and over-active EGFR signalling;
Subtype 3: may be associated with Immune-infiltration, high B-cell infiltration, high tertiary lymphoid structure (TLS) counts;
Subtype 4-6: may be associated with high neoantigen burden (NB);
Subtype 4: may be associated with high TMB, high FGL1 level;
Subtype 6: may be associated with high B7-H4 level.

Therefore, the methods of the invention are capable of determining the Dominant Molecular Cancer Phenotype (DMCP), by which we mean the most distinct features of the tumour. This level of information is crucial for understanding how cancer cells acquire hallmark capabilities such as oncogenic growth, evasion of cell death signalling and immune evasion, and in turn for determining the prognosis. Determining the DMCP, and consequently the prognosis, is Independent of any histological based typing or staging of NSCLC.

It will be clear to the skilled person that by “measuring in the test sample the presence and/or amount of the biomarkers defined in Table A and/or one or more of Tables B-G” we include the situation where less than all of the biomarkers defined in Tables A-G, and each of Tables A(i)-(vii), are measured in Step (3-b).

As discussed above, the biomarkers measured in Step (3-b) may be the biomarkers of Tables B-G, in some preferred embodiments. In some embodiments, one or more biomarkers from any one, two, three, four, five, or six of Tables B, C, D, E, F and/or G may be measured.

Therefore, in some embodiments, the classification algorithm of Step (3-c) is a Support Vector Machine-protein (“SVM-protein”), and Step (3-b) comprises measuring the presence and/or amount of 145 or more of the biomarkers defined in Table B, and/or 60 or more of the biomarkers defined in Table C. In this embodiment, Step (3-b) may comprise measuring in the test sample the presence and/or amount of around 30% of the biomarkers defined in Table B and/or Table C. In other embodiments, Step (3-b) comprises measuring the presence and/or amount of around 35%, 40% or 45% of the biomarkers defined in Table B and/or Table C.

In some embodiments the classification algorithm is a Support Vector Machine-protein (“SVM-protein”), and Step (3-b) comprises measuring the presence and/or amount of 243 or more of the biomarkers defined in Table B, and/or 100 or more of the biomarkers defined in Table C. In this embodiment, Step (3-b) may comprise measuring in the test sample the presence and/or amount of around 50% of the biomarkers defined in Table B and/or Table C. In other embodiments, Step (3-b) comprises measuring the presence and/or amount of around 55%, 60%, 65%, 70%, or 75% of the biomarkers defined in Table B and/or Table C.

In other embodiments, the classification algorithm is a Support Vector Machine-protein (“SVM-protein”), and Step (3-b) comprises measuring the presence and/or amount of 388 or more of the biomarkers defined in Table B, and/or 160 or more of the biomarkers defined in Table C. In some embodiments all of the biomarkers of Table B and/or Table C are measured.

In other embodiments, Step (3-b) comprises measuring the presence and/or amount of around 80% of the biomarkers defined in Table B and/or Table C. In other embodiments, Step (3-b) comprises measuring the presence and/or amount of around 85%, 90%, 95%, 96%, 97%, 98%, 99% or 100% of the biomarkers defined in Table B and/or Table C.

In some embodiments, the classification algorithm of Step (3-c) is K-Top Scoring Pairs Step (3-b) comprises measuring the presence and/or amount of 489 or more of the biomarker pairs defined in Table D, and/or 67 or more of the biomarker pairs defined in Table E. In this embodiment, Step (3-b) may comprise measuring in the test sample the presence and/or amount of around 30% of the biomarker pairs defined in Table D and/or Table E. In other embodiments, Step (3-b) comprises measuring the presence and/or amount of around 35%, 40% or 45% of the biomarker pairs defined in Table D and/or Table E.

In some embodiments, the classification algorithm is k-TSP and Step (3-b) comprises measuring the presence and/or amount of 815 or more of the biomarker pairs defined in Table D, and/or 112 or more of the biomarker pairs defined in Table E. In this embodiment, Step (3-b) may comprise measuring in the test sample the presence and/or amount of around 50% of the biomarker pairs defined in Table D and/or Table E. In other embodiments, Step (3-b) comprises measuring the presence and/or amount of around 55%, 60%, 65%, 70%, or 75% of the biomarker pairs defined in Table D and/or Table E.

Therefore, in some embodiments, the classification algorithm is k-TSP and Step (3-b) comprises measuring the presence and/or amount of 1304 or more of the biomarker pairs defined in Table D, and/or 180 or more of the biomarker pairs defined in Table E. In some embodiments, all of the biomarker pairs of Table D and/or Table E are measured.

In other embodiments, Step (3-b) comprises measuring the presence and/or amount of around 80% of the biomarker pairs defined in Table D and/or Table E. In other embodiments, Step (3-b) comprises measuring the presence and/or amount of around 85%, 90%, 95%, 96%, 97%, 98%, 99% or 100% of the biomarker pairs defined in Table D and/or Table E.

In some embodiments, the classification algorithm is Support Vector Machine-peptide (“SVM-peptide”) and Step (3-b) comprises measuring the presence and/or amount of 174 or more of the biomarkers defined in Table F, and/or 60 or more of the biomarkers defined in Table G. In this embodiment, Step (3-b) may comprise measuring in the test sample the presence and/or amount of around 30% of the biomarkers defined in Table F and/or Table G. In other embodiments, Step (3-b) comprises measuring the presence and/or amount of around 35%, 40% or 45% of the biomarkers defined in Table F and/or Table G.

In some embodiments, the classification algorithm is Support Vector Machine-peptide (“SVM-peptide”) and Step (3-b) comprises measuring the presence and/or amount of 290 or more of the biomarkers defined in Table F, and/or 100 or more of the biomarkers defined in Table G. In this embodiment, Step (3-b) may comprise measuring in the test sample the presence and/or amount of around 50% of the biomarkers defined in Table F and/or Table G. In other embodiments, Step (3-b) comprises measuring the presence and/or amount of around 55%, 60%, 65%, 70%, or 75% of the biomarkers defined in Table F and/or Table G.

In some embodiments, the classification algorithm of Step (3-c) is Support Vector Machine-peptide (“SVM-peptide”) and Step (3-b) comprises measuring the presence and/or amount of 464 or more of the biomarkers defined in Table F, and/or 160 or more of the biomarkers defined in Table G. In some embodiments, all of the biomarkers of Table F and/or Table G are measured.

In other embodiments, Step (3-b) comprises measuring the presence and/or amount of around 80% of the biomarkers defined in Table F and/or Table G. In other embodiments, Step (3-b) comprises measuring the presence and/or amount of around 85%, 90%, 95%, 96%, 97%, 98%, 99% or 100% of the biomarkers defined in Table F and/or Table G.

In some embodiments, the classification algorithm of Step (3-c) is Support Vector Machine-peptide (“SVM-peptide”) and Step (3-b) comprises measuring the presence and/or amount of polypeptide biomarkers derived from or mapping to the protein biomarkers of Table A and/or Table F and/or Table G.

Therefore, in some embodiments, Step (3-b) comprises measuring in the test sample the presence and/or amount of 526 or more of the biomarkers of Table A.

In this embodiment, Step (3-b) may comprise measuring in the test sample the presence and/or amount of around 30% or more of the biomarkers defined in Table A. In other embodiments, Step (3-b) comprises measuring the presence and/or amount of around 35%, 40% or 45% of the biomarkers defined in Table A.

In some embodiments, Step (3-b) comprises measuring in the test sample the presence and/or amount of 877 or more of the biomarkers of Table A.

In this embodiment, Step (3-b) may comprise measuring in the test sample the presence and/or amount of around 50% or more of the biomarkers defined in Table A. In other embodiments, Step (3-b) comprises measuring the presence and/or amount of around 55%, 60%, 65%, 70%, or 75% of the biomarkers defined in Table A.

For instance, in some particularly preferred embodiments, Step (3-b) comprises measuring in the test sample the presence and/or amount of 1404 or more of the biomarkers defined in Table A.

In this embodiment, Step (3-b) comprises measuring the presence and/or amount of around 80% of the biomarkers defined in Table A. In other embodiments, Step (3-b) comprises measuring the presence and/or amount of around 85%, 90%, 95%, 96%, 97%, 98%, 99% or 100% of the biomarkers defined in Table A. Therefore, in some embodiments, Step (3-b) comprises determining the presence and/or amount of all of the biomarkers defined in Table A.

In some embodiments, Step (3-b) comprises determining the presence and/or amount of a subset of the biomarkers of Table A, which correspond to the biomarkers of Tables 1-6 and therefore the prognostic subtypes 1-6. In this embodiment, Step (3-b) comprises measuring the biomarkers of Table A(i) and/or Table A(ii) and/or Table A(iii) and/or Table A(iv) and/or Table A(v) and/or Table (vi). It will be evident to the skilled person that this includes the situation where some, but not all, of the biomarkers of each of Tables A(i-vi) are measured.

In some preferred embodiments, Step (3-b) comprises determining the presence and/or amount of:
39 or more of the biomarkers defined in Table A(i); and/or
11 or more of the biomarkers defined in Table A(ii); and/or
2 or more of the biomarkers defined in Table A(iii); and/or
8 or more of the biomarkers defined in Table A(iv); and/or
137 or more of the biomarkers defined in Table A(v); and/or
36 or more of the biomarkers defined in Table A(vi).

In this embodiment, Step (3-b) may comprise measuring in the test sample the presence and/or amount of around 30% of the biomarkers defined in Table A(i) and/or Table A(ii) and/or Table A(iii) and/or Table A(iv) and/or Table A(v) and/or Table A(vi).

In other embodiments, Step (2-b) comprises measuring the presence and/or amount of around 35%, 40% or 45% of the biomarkers defined in Table A(i) and/or Table A(ii) and/or Table A(iii) and/or Table A(iv) and/or Table A(v) and/or Table A(vi).

In some preferred embodiments, Step (3-b) comprises determining the presence and/or amount of:
66 or more of the biomarkers defined in Table A(i); and/or
19 or more of the biomarkers defined in Table A(ii); and/or
3 or more of the biomarkers defined in Table A(iii); and/or
14 or more of the biomarkers defined in Table A(iv); and/or
229 or more of the biomarkers defined in Table A(v); and/or
61 or more of the biomarkers defined in Table A(vi).

In this embodiment, Step (3-b) may comprise measuring in the test sample the presence and/or amount of around 50% of the biomarkers defined in Table A(i) and/or Table A(ii) and/or Table A(iii) and/or Table A(iv) and/or Table A(v) and/or Table A(vi). In other embodiments, Step (2-b) comprises measuring the presence and/or amount of around 55%, 60%, 65%, 70%, or 75% of the biomarkers defined in Table A(i) and/or Table A(ii) and/or Table A(iii) and/or Table A(iv) and/or Table A(v) and/or Table A(vi).

For instance, in some particularly preferred embodiments, Step (3-b) comprises measuring in the test sample the presence and/or amount of:
105 or more of the biomarkers defined in Table A(i); and/or
30 or more of the biomarkers defined in Table A(ii); and/or
4 or more of the biomarkers defined in Table A(iii); and/or
22 or more of the biomarkers defined in Table A(iv); and/or
367 or more of the biomarkers defined in Table A(v); and/or
97 or more of the biomarkers defined in Table A(vi).

In other embodiments, Step (3-b) comprises measuring the presence and/or amount of around 80% of the biomarkers defined in Table A(i) and/or Table A(ii) and/or Table A(Iii) and/or Table A(iv) and/or Table A(v) and/or Table A(vi). In other embodiments, Step (3-b) comprises measuring the presence and/or amount of around 85%, 90%, 95%, 96%, 97%, 98%, 99% or 100% of the biomarkers defined in Table A(i) and/or Table A(ii) and/or Table A(iii) and/or Table A(iv) and/or Table A(v) and/or Table A(vi).

In some embodiments, Step (3-b) comprises determining the presence and/or amount of all of the biomarkers of Table A(i). In additional or alternative embodiments, Step (3-b) comprises determining the presence and/or amount of all of the biomarkers of Table A(ii). In additional or alternative embodiments, Step (3-b) comprises determining the presence and/or amount of all of the biomarkers of Table A(iii). In additional or alternative embodiments, Step (3-b) comprises determining the presence and/or amount of all of the biomarkers of Table A(iv). In additional or alternative embodiments, Step (3-b) comprises determining the presence and/or amount of all of the biomarkers of Table A(v). In additional or alternative embodiments, Step (3-b) comprises determining the presence and/or amount of all of the biomarkers of Table A(vi).

Therefore, in some embodiments, Step (3-b) comprises determining the presence and/or amount of all of the biomarkers defined in each of Table A(i) and Table A(ii) and Table A(iii) and Table A(iv) and Table A(v) and Table A(vi).

It will be clear to the skilled person that the method may comprise or consist of measuring a combination of different numbers (or percentages) of biomarkers from each of Tables A(i-vi). For instance, the method may comprise or consist of measuring 50% of the biomarkers in each of Tables A(i-vi). In other embodiments, the method may comprise measuring 80% of the biomarkers of one of the Tables A(i-vi), along with 50% of the biomarkers from one of the other Tables A(i-vi).

It will be appreciated that any combination of the biomarkers within each of Tables A(i-vi) may be measured in this embodiment. In addition, the method can also involve measuring different combinations of biomarkers from each of Tables A(i-vi).

In some additional embodiments, the method of the third aspect further comprises determining the presence and/or amount of one or more biomarkers defined in Table A(vii). These biomarkers may be measured in addition to the biomarkers of one or more of Tables A(i-vi) described herein.

In some preferred embodiments, the presence and/or amount of at least 10% of the biomarkers defined in Table A(vii) are measured, for example at least 20%, 30%, 40%, 50%, 60%, 70%, 80%, 85%, 90%, 95% or 100% of the biomarkers of Table A(vii) are measured. Therefore, in some embodiments all of the biomarkers of Table A(vii) are measured. In some preferred embodiments, at least 335 of the biomarkers of Table A(vii) are measured.

In Step (3-c), the classification Into the prognostic subtypes 1-6 is based on the measured biomarkers of Tables B and C (where the classification algorithm is SVM-protein), Tables D and E (where the classification algorithm is k-TSP), and/or Tables F and G (where the classification algorithm is SVM-peptide) It will be clear to the skilled person that the methods of the first, second and third aspects all allow, for the first time, classification of NSCLC into six prognostic subtypes based on a novel set of biomarkers.

The following embodiments are relevant to each of the first, second and third aspects described above: The methods of each of the first, second and third aspects involve determining the presence and/or amount (wherein “amount” is intended to have the same meaning as “level”) of various biomarkers defined herein. In some preferred embodiments, the expression of protein or polypeptide biomarkers is measured. In some embodiments, measurement of the protein biomarker signatures is advantageous as it may be considered more representative of the proteome status of the cell, and therefore can be used to more accurately subtype test samples. Therefore, in some embodiments, the biomarkers measured are protein or polypeptide biomarkers.

In some embodiments when protein or polypeptide biomarkers are detected, the measurement is carried out using a mass-spectrometry or an affinity-based method.

In a preferred embodiment, determining the presence and/or amount of the biomarkers is achieved using mass spectrometry (MS). Mass spectrometric methods are generally known in the art. The MS methods compatible with the methods of the invention include, but are not limited to, the following:
Bottom-up and Top-down proteomics methods
MS-methods using ionization techniques including electrospray, Matrix-Assisted Laser Desorption/Ionisation (MALDI) or other methods;
MS-methods using mass separation and detection techniques including but not limited to Orbitrap™, Time-of-Flight analyser (TOF), Fourier Transform (Fr)-MS, Linear ion trap (LT), Quadropole (Q), Triple quadropole (QQQ) and Ion-mobility separation alone or in combination;
MS-methods using Data-Dependent Acquisition (DDA), Data-Independent Acquisition (DIA), Sequential Windowed Acquisition of All Theoretical Fragment Ions (SWATH), Parallel Reaction Monitoring (PRM), Multiple Reaction Monitoring (MRM), Selected Reaction Monitoring (SRM).
MS-methods using label-based quantification by isobaric or isotopic labelling such as ICAT, ITRAQ, Tandem Mass Tag (TMT), trimethyl labelling, SILAC or comparable;
MS-methods using spike-in based labelling where peptides or other molecules are added to the sample as references for quantification;
MS-methods using label-free quantification by peak area or height or intensity, by spectral counts or other comparable methods.

In some embodiments, the test samples and control samples are treated prior to mass spectrometric analysis to extract the proteins therein for analysis. Techniques for doing so are well known in the art. Extracted proteins may then be digested (e.g. by treatment with trypsin) to produce protein fragments (polypeptides/peptides). Therefore, in some embodiments, the biomarker detected may be a polypeptide biomarker derived from the protein biomarkers described herein.

In some embodiments, the resulting peptides derived from the test or control sample described above are labelled to aid quantification of protein. Quantification of protein may also be achieved using label-free techniques. In some embodiments, the label may be an Isotope coded affinity tag, isobaric labelling, or metal coded tag. In some embodiments the peptides or proteins are labelled using a Tandem Mass Tag (TMT). There are six varieties of TMT available: TMTzero, TMTduplex, TMTsixplex, TMT 10-plex, TMTpro 16plex, and TMTpro Zero. The tags contain four regions, namely a mass reporter region (M), a cleavable linker region (F), a mass normalization region (N) and a protein reactive group (R). The chemical structures of all the tags are identical but each contains isotopes substituted at various positions, such that the mass reporter and mass normalization regions have different molecular masses in each tag.

In some embodiments, the proteins or peptides derived from the test or control sample(s) may be separated (e.g. by size, hydrophobicity, charge and/or Isoelectric point) prior to being detected and identified by mass spectrometry. Separation can occur either before or after protein digestion and labelling. In some embodiments, this prior separation step may Involve one or more of the following techniques: Isoelectric focusing; high resolution Isoelectric focusing (HiRIEF); liquid chromatography; or High Performance Liquid Chromatography (HPLC). In some preferred embodiments, the samples are separated first by HiRIEF followed by liquid chromatography (e.g. HPLC), following which they are fed directly into the mass spectrometer via electrospray ionization.

After separation, the labelled peptides can be introduced into the mass spectrometer for signal generation. For a signal to be generated, the sample must be gaseous, which can be achieved using electrospray ionisation or MALDI, in some embodiments. In some embodiments, tandem mass spectrometry (MS/MS) is used. Tandem MS allows data from an initial spectrum (which provides Information on the peptide mass) to be combined with another spectrum produced by fragmenting the peptide in a collision cell. The resulting data can then be used to analyse the mass of the peptide fragment to a high degree of accuracy, and this can be compared to peptide masses calculated in silico using expected masses from digestion of proteins found within a database (e.g. the Ensembl database).

MS-based identification and quantification is accomplished by determination of the mass and charge of ions in the sample. This is a two-step process where in the first step the mass and charge of the intact peptide is determined by the MS instrument (MS1). In the second step the intact peptide is fragmented, and the masses and charges of resulting peptide fragments are determined by the MS instrument (MS2). Based on the generated information, i.e. the intact peptide mass and charge and the masses and charges of the peptide fragments, the identity of the peptide and the corresponding protein is determined by matching of the information to a search database.

Examples of methods used to Identify the proteins in the test and control samples are Data Independent Acquisition (DIA) and Data Dependent Acquisition (DDA). DDA and DIA differ in the way peptide ions are collected for generation of MS1 and MS2 spectra:

DDA
Selection of peptides for fragmentation and identification is based on the peak detection in MS1. Intense peaks are normally prioritized over less Intense peaks that may be missed in the analysis. In complex samples such as clinical samples this can result in large differences in the identified proteins between samples. This can result in limited overlap between samples and difficulties in the downstream comparison between samples.
DDA can provide greater analytical depth (more identified proteins) in each sample compared to DIA, even if the overlap between samples is lower.

DIA
Selection of peptides for fragmentation is performed according to a pre-determined schedule, and not dependent on the data generated in MS1. This approach can be more robust and can produce more comparable data between samples. This can be especially important for development of assays since scheduling can be optimized to identify and quantify as many of the predefined markers as possible.

Using a mass-spectrometry based analysis has several benefits, that include but are not limited to: no need to use affinity reagents; a greater analytical depth; limited background signal; limited unspecific signal; cost efficiency; improved specificity; multiplexing capacity; and analysis speed.

In other embodiments, determining the presence and/or amount of the biomarkers is achieved using an affinity-based method. Such methods are generally known in the art, and can Include, but are not limited to, the following:
Methods based on affinity binders including antibodies, affibodies, aptamers or similar;
Antibody arrays or microarrays;
Antibody bead arrays;
Proximity extension assay;
Western blot;
Multiplex Western blotting;
Reverse lysate protein arrays;
Enzyme-Linked Immunosorbent Assay (ELISA);
Multiplexed ELISA, nanostring or mass cytometry;
Multiplexed IHC.

In some embodiments, the affinity-based method is an array.

In some embodiments, determining the presence and/or amount of the biomarkers defined in Tables 1-6 and A-G may be performed using one or more first binding agents capable of binding to a biomarker (i.e. a protein or polypeptide). It will be appreciated by persons skilled in the art that the first binding agent may comprise or consist of a single species with specificity for one of the protein biomarkers or a plurality of different species, each with specificity for a different protein biomarker.

Suitable binding agents (also referred to as binding molecules) can be selected from a library, based on their ability to bind a given target molecule, as discussed below.

In one preferred embodiment, at least one type of the binding agents, and more typically all of the types, may comprise or consist of an antibody or antigen-binding fragment of the same, or a variant thereof.

Methods for the production and use of antibodies are well known in the art, for example see Antibodies: A Laboratory Manual, 1988, Harlow & Lane, Cold Spring Harbor Press, ISBN-13: 978-0879693145, Using Antibodies: A Laboratory Manual, 1998, Harlow & Lane, Cold Spring Harbor Press, ISBN-13: 978-0879695446 and Making and Using Antibodies: A Practical Handbook, 2006, Howard & Kaser, CRC Press, ISBN-13: 978-0849335280 (the disclosures of which are incorporated herein by reference).

Thus, a fragment may contain one or more of the variable heavy (V_{H}) or variable light (Vi) domains. For example, the term antibody fragment includes Fab-like molecules (Better et al (1988) Science 240, 1041); Fv molecules (Skerra et al (1988) Science 240, 1038); single-chain Fv (scFv) molecules where the V_{H} and V_{L} partner domains are linked via a flexible oligopeptide (Bird et al (1988) Science 242, 423; Huston et a (1988) Proc. Natl. Acad. Sci. USA 85, 5879) and single domain antibodies (dAbs) comprising isolated V domains (Ward eta (1989) Nature 341, 544).

For example, the binding agent(s) may be whole antibodies or scFv molecules.

The term “antibody variant” includes any synthetic antibodies, recombinant antibodies or antibody hybrids, such as but not limited to, a single-chain antibody molecule produced by phage-display of Immunoglobulin light and/or heavy chain variable and/or constant regions, or other immuno-interactive molecule capable of binding to an antigen in an immunoassay format that is known to those skilled in the art.

A general review of the techniques involved in the synthesis of antibody fragments which retain their specific binding sites is to be found in Winter & Milstein (1991) Nature 349, 293-299.

Molecular libraries such as antibody libraries (Clackson et al, 1991, Nature 352, 624-628; Marks et al, 1991, J Mol Biol 222(3): 581-97), peptide libraries (Smith, 1985, Science 228(4705): 1315-7), expressed cDNA libraries (Santi et al (2000) J Mol Biol 296(2): 497-508), libraries on other scaffolds than the antibody framework such as affibodies (Gunneriusson et al, 1999, App Environ Microbiol 65(9): 4134-40) or libraries based on aptamers (Kenan et al, 1999, Methods Mol Biol 118, 217-31) may be used as a source from which binding molecules that are specific for a given motif are selected for use in the methods of the invention.

Conveniently, the binding agent(s) may be immobilised on a surface (e.g., on a multiwell plate or array).

In one embodiment of the methods of the invention, determining the presence and/or amount of the biomarkers defined in Tables 1-6 and A-G is performed using an assay comprising a second binding agent capable of binding to the one or more biomarkers, the second binding agent comprising a detectable moiety. For example, an immobilised (first) binding agent may initially be used to ‘trap’ the protein biomarker on to the surface of a microarray, and then a second binding agent may be used to detect the ‘trapped’ protein.

The second binding agent may be as described above in relation to the (first) binding agent, such as an antibody or antigen-binding fragment thereof.

It will be appreciated by skilled person that the one or more biomarkers (e.g., proteins) in the test sample may be labelled with a detectable moiety. Likewise, the one or more biomarkers in the control sample(s) may be labelled with a detectable moiety.

Alternatively, or in addition, the first and/or second binding agents may be labelled with a detectable moiety.

By a “detectable moiety” we include the meaning that the moiety is one which may be detected and the relative amount and/or location of the moiety (for example, the location on an array) determined.

Suitable detectable moieties are well known in the art. For example, the detectable moiety may be selected from the group consisting of: a fluorescent moiety; a luminescent moiety; a chemiluminescent moiety; a radioactive moiety; an enzymatic moiety.

In one preferred embodiment, the detectable moiety is biotin.

In one embodiment, the biotinylated biomarkers are detected using streptavidin labelled with a detectable moiety selected from the group consisting of: a fluorescent moiety; a luminescent moiety; a chemiluminescent moiety; a radioactive moiety; an enzymatic moiety.

Thus, the detectable moiety may be a fluorescent and/or luminescent and/or chemiluminescent moiety which, when exposed to specific conditions, may be detected. For example, a fluorescent moiety may need to be exposed to radiation (i.e., light) at a specific wavelength and Intensity to cause excitation of the fluorescent moiety, thereby enabling it to emit detectable fluorescence at a specific wavelength that may be detected.

Alternatively, the detectable moiety may be an enzyme which is capable of converting a (preferably undetectable) substrate into a detectable product that can be visualised and/or detected. Examples of suitable enzymes are discussed in more detail below in relation to, for example, ELISA assays.

In a further alternative, the detectable moiety may be a radioactive atom which is useful in imaging. Suitable radioactive atoms Include ^{99m}Tc and ¹²³I for scintigraphic studies. Other readily detectable moieties include, for example, spin labels for magnetic resonance imaging (MRI) such as ¹²³I again, ¹³¹I, ¹¹¹In, ¹⁹F, ¹³C, ¹⁵N, ¹⁷O, gadolinium, manganese or iron. Clearly, the agent to be detected (such as, for example, the one or more biomarkers in the test sample and/or control sample described herein and/or an antibody molecule for use in detecting a selected protein) must have sufficient of the appropriate atomic isotopes in order for the detectable moiety to be readily detectable.

Preferred assays for detecting proteins or polypeptides include enzyme linked immunosorbent assays (ELISA), radioimmunoassay (RIA), immunoradiometric assays (IRMA) and Immunoenzymatic assays (IEMA), including sandwich assays using monoclonal and/or polyclonal antibodies. Exemplary sandwich assays are described by David et al in U.S. Pat. Nos. 4,376,110 and 4,486,530, hereby incorporated by reference. Antibody staining of cells on slides may be used in methods well known in cytology laboratory diagnostic tests, as well known to those skilled in the art.

Conveniently, the assay may be an ELISA (Enzyme Linked Immunosorbent Assay) which typically Involves the use of enzymes giving a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemiluminescent systems based on enzymes such as luciferase can also be used.

ELISA methods are well known in the art, for example see The ELISA Guidebook (Methods in Molecular Biology), 2000, Crowther, Humana Press, ISBN-13: 978-0896037281 (the disclosures of which are incorporated by reference).

In one embodiment, the detectable moiety is fluorescent moiety (for example an Alexa Fluor dye, e.g. Alexa647).

In one preferred embodiment, the detection may be performed using an array.

Arrays per se are well known in the art. Typically, they are formed of a linear or two-dimensional structure having spaced apart (i.e. discrete) regions (“spots”), each having a finite area, formed on the surface of a solid support. An array can also be a bead structure where each bead can be identified by a molecular code or colour code or identified in a continuous flow. Analysis can also be performed sequentially where the sample is passed over a series of spots each adsorbing the class of molecules from the solution. The solid support is typically glass or a polymer, the most commonly used polymers being cellulose, polyacrylamide, nylon, polystyrene, polyvinyl chloride or polypropylene. The solid supports may be in the form of tubes, beads, discs, silicon chips, microplates, polyvinylidene difluoride (PVDF) membrane, nitrocellulose membrane, nylon membrane, other porous membrane, non-porous membrane (e.g. plastic, polymer, perspex, silicon, amongst others), a plurality of polymeric pins, or a plurality of microtitre wells, or any other surface suitable for immobilising proteins, polynucleotides and other suitable molecules and/or conducting an immunoassay. The binding processes are well known in the art and generally consist of cross-linking covalently binding or physically adsorbing a protein molecule, polynucleotide or the like to the solid support. By using well-known techniques, such as contact or non-contact printing, masking or photolithography, the location of each spot can be defined. For reviews see Jenkins, R. E., Pennington, S. R. (2001, Proteomics, 2, 13-29) and Lal et al (2002, Drug Discov Today 15; 7(18 Suppl):S143-9).

Typically, the array is a microarray. By “microarray” we include the meaning of an array of regions having a density of discrete regions of at least about 100/cm², and preferably at least about 1000/cm². The regions in a microarray have typical dimensions, e.g., diameters, in the range of between about 10-250 μm, and are separated from other regions in the array by about the same distance. The array may also be a macroarray or a nanoarray.

Once suitable binding molecules (discussed above) have been identified and isolated, the skilled person can manufacture an array using methods well known in the art of molecular biology.

In some embodiments, determining the presence and/or amount of the protein or polypeptide biomarkers is achieved by one or more of the following methods:
Protein sequencing (e.g. by Nanopore or another high throughput sequencing technique);
Edman degradation;
Labelling and imaging based methods (one example of such a method (as discussed in Swaminathan et al., Nat Biotechnol., 2018, 36: 1076-1082) involves selective fluorescent labelling of cysteine and lysine residues in peptide samples, immobilization of labelled peptides on a glass surface, and Imaging by total internal reflection microscopy (TIRF) to monitor reduction in fluorescence following consecutive rounds of Edman degradation) In alternative embodiments, the expression of a nucleic acid molecule encoding the biomarkers disclosed herein is measured. The nucleic acid molecule may be an mRNA or cDNA molecule. In some preferred embodiments, the nucleic acid molecule is an mRNA molecule.

In some embodiments, measurement of mRNA is advantageous as mRNA is readily available and can be simply amplified using the Polymerase Chain Reaction (PCR). In addition, measurement of mRNA may be useful for particular sample types that are more difficult to extract proteins from for analysis.

In some embodiments when nucleic acid biomarkers are detected, measurement of the nucleic acid is carried out using a transcriptomics-based technique. These include techniques generally known in the art for detecting nucleic acids (e.g. mRNA) in a sample. They may include, but are not limited to, the following:
Chromogenic in-situ hybridization (CISH);
Fluorescence in-situ hybridization (FISH);
RNA-sequencing;
RNA microarrays;
Quantitative RT-PCR;
Digital RT-PCR;
Northern blot;
Digital colour-coded nucleic acid barcode (nCounter®) technology;
RT-PCR-ELISA.

For example, measuring the expression of the one or more biomarker(s) may be performed using one or more binding moieties, each individually capable of binding selectively to a nucleic acid molecule encoding one of the biomarkers identified in Tables 1-6 or Tables A-G.

Conveniently, the one or more binding moieties each comprise or consist of a nucleic acid molecule, such as DNA, RNA, peptide nucleic acid (PNA), locked nucleic acid (LNA), glycol nucleic acid (GNA), threose nucleic acid (TNA), or a phosphorodiamidate morpholino oligomer (PMO).

It will be appreciated that the nucleic acid-based binding moieties may comprise a detectable moiety.

Thus, the detectable moiety may be selected from the group consisting of: a fluorescent moiety; a luminescent moiety; a chemiluminescent moiety; a radioactive moiety (for example, a radioactive atom); or an enzymatic moiety.

Alternatively or additionally, the detectable moiety may comprise or consist of a radioactive atom, for example selected from the group consisting of technetium-99m, iodine-123, iodine-125, Iodine-131, indium-iii, fluorine-19, carbon-13, nitrogen-15, oxygen-17, phosphorus-32, sulphur-35, deuterium, tritium, rhenium-186, rhenium-188 and yttrium-90.

Alternatively or additionally, the detectable moiety of the binding moiety may be a fluorescent moiety.

In one embodiment, expression of the one or more biomarker(s) is determined using an RNA or DNA microarray.

In some embodiments, determining the prognosis of NSCLC in an individual involves determining the chance of survival of the individual with NSCLC over a defined period.

It can also include the chance of the NSCLC recurring over a defined period.

In some preferred embodiments, it includes determining the probable survival time of an Individual, e.g. by defining the number of months or years the individual may be expected to survive, for example determining the probability of survival over a 2 year or 5 year period. One advantage of the present invention is that classifying the NSCLC based on the biomarker signatures described herein allows the subtyping of NSCLC Into defined groups with more defined prognoses, as once the subtype is determined, prognosis can be estimated based on prior knowledge of the typical clinical outcome for each subtype.

In some embodiments, the probability of survival in the short term can be estimated following classification using the methods of the invention. By “short term” we include survival for up to around 1 year from diagnosis. By “up to around 1 year” we include survival for any time from diagnosis to approximately 1.5 years from diagnosis.

In alternative or additional embodiments, the probability of survival in the medium term can be estimated following classification using the methods of the Invention. By “medium term” we include survival for up to around 2-4 years from diagnosis. By “up to around 2-4 years” we include survival for any time from approximately 1.5 years to approximately 4.5 years from diagnosis.

In alternative or additional embodiments, the probability of survival in the long term can be estimated following classification using the methods of the invention. By “long term” we Include survival for around 5 years or more from diagnosis. By “around 5 years or more” we include survival for any time from approximately 4.5 years and beyond from diagnosis.

The skilled person will appreciate that survival is dependent on multiple factors, for example stage at diagnosis, age, sex, demographic, socioeconomic status, lifestyle, and underlying conditions and comorbidities, and that the generally accepted definitions of short, medium and long term survival times above may differ in different groups based on these factors.

Therefore, in some embodiments it will be beneficial to express the short, medium or long term survival of a patient compared to median survival for NSCLC of a certain stage in a certain demographic, for example. In other embodiments, it may be beneficial to express the short, medium or long term survival of a patient compared to the median survival for NSCLC for patients with certain co-morbidities or underlying conditions.

The skilled person will appreciate that NSCLC survival probabilities have previously been categorised by NSCLC type and/or stage at diagnosis. For instance, the SEER database provides information on percentages of patients surviving for a number of years for adenocarcinoma, large cell carcinoma and squamous cell carcinoma at various stages at diagnosis (localised (which corresponds to Stage 1 in the AJCC TNM staging model discussed below), regional (which corresponds to Stages 2/3, and distant (which corresponds to Stage 4)):
https://seer.cancer.gov/explorer/application.html?site=47&data_type=4&graph_type=6&compareBy=subtype&chk_subtype_612=612&chk_subtype_613=613&chk_subtype_610=610&sex=1&race=1&age_range=1&stage=104&advopt_precision=1&advopt_display=2.

Alternatively, in some embodiments of the present invention, the Prognostic Subtypes 1-6 defined herein are associated with a particular survival probability. Therefore, in some embodiments, the probability of 2 year survival for an Individual with NSCLC classified as Prognosis Subtype 1 is in the range of 0.90-1.00. In some embodiments the 2 year survival probability is 0.99. In other embodiments, the probability of 2 year survival for an individual with NSCLC classified as Prognosis Subtype 2 is in the range of 0.85-0.95. In some embodiments, the 2 year survival probability is 0.87. In other embodiments, the probability of 2 year survival for an individual with NSCLC classified as Prognosis Subtype 3 is in the range of 0.85-0.95. In some embodiments, the 2 year survival probability is 0.88. In other embodiments, the probability of 2 year survival for an individual with NSCLC classified as Prognosis Subtype 4 is in the range of 0.75-0.85. In some embodiments, the 2 year survival probability is 0.82. In other embodiments, the probability of 2 year survival for an individual with NSCLC classified as Prognosis Subtype 5 is in the range of 0.50-0.60. In some embodiments, the 2 year survival probability is 0.54. In other embodiments, the probability of 2 year survival for an individual with NSCLC classified as Prognosis Subtype 6 is in the range of 0.70-0.80. In some embodiments, the 2 year survival probability is 0.74.

In some embodiments, the probability of 5 year survival for an individual with NSCLC classified as Prognosis Subtype 1 is in the range of 0.85-0.95. In some embodiments, the 5 year survival probability is 0.89. In other embodiments, the probability of 5 year survival for an individual with NSCLC classified as Prognosis Subtype 2 is in the range of 0.60-0.70. In some embodiments, the 5 year survival probability is 0.66. In other embodiments, the probability of 5 year survival for an individual with NSCLC classified as Prognosis Subtype 3 is in the range of 0.70-0.80. In some embodiments, the 5 year survival probability is 0.75. In other embodiments, the probability of 5 year survival for an individual with NSCLC classified as Prognosis Subtype 4 is in the range of 0.60-0.70. In some embodiments, the 5 year survival probability is 0.66. In other embodiments, the probability of 5 year survival for an individual with NSCLC classified as Prognosis Subtype 5 is in the range of 0.35-0.45. In some embodiments, the 5 year survival probability is 0.37. In other embodiments, the probability of 5 year survival for an individual with NSCLC classified as Prognosis Subtype 6 is in the range of 0.55-0.65. In some embodiments, the 5 year survival probability is 0.58.

In some alternative or additional embodiments, determining the prognosis of NSCLC in an individual involves determining the number of months or years that a certain proportion of Individuals with NSCLC of a particular subtype would be expected to survive from diagnosis. For example, this may be expressed as the number of months that 50%, 55%, 60%, 65%, 70%, 75%, 80%, 85%, 90%, 99%, or 100% of individuals in that subtype would be expected to survive from diagnosis. Preferably, this may be expressed as the number of months that 75% of individuals would be expected to survive from diagnosis.

In all of the following embodiments, survival expectations are expressed in months from diagnosis. Therefore, in some embodiments, an individual with NSCLC classified as Prognosis Subtype 1 may be expected to survive for 85-95 months. In some embodiments an Individual with NSCLC classified as Prognosis Subtype 1 may be expected to survive for 88 months. In other embodiments, an individual with NSCLC classified as Prognosis Subtype 2 may be expected to survive for 45-55 months. In some embodiments, an Individual with NSCLC classified as Prognosis Subtype 2 may be expected to survive for 49 months. In other embodiments, an individual with NSCLC classified as Prognosis Subtype 3 may be expected to survive for 55-65 months. In some embodiments, an individual with NSCLC classified as Prognosis Subtype 3 may be expected to survive for 61 months. In other embodiments, an individual with NSCLC classified as Prognosis Subtype 4 may be expected to survive for 30-40 months. In some embodiments, an individual with NSCLC classified as Prognosis Subtype 4 may be expected to survive for 35 months. In other embodiments, an Individual with NSCLC classified as Prognosis Subtype 5 may be expected to survive for 10-20 months. In some embodiments, an individual with NSCLC classified as Prognosis Subtype 5 may be expected to survive for 15 months. In other embodiments, an Individual with NSCLC classified as Prognosis Subtype 6 may be expected to survive for 15-25 months. In some embodiments, an Individual with NSCLC classified as Prognosis Subtype 6 may be expected to survive for 21 months.

In some embodiments, the test sample comprises one or more lung cancer cell(s). By “lung cancer cell” we Include any cell that is derived from a lung cell and also has the characteristics of a cancer cell (e.g. increased rate of cell division compared to non-cancerous cells, abnormal cellular features, propensity to form tumours). These cells may be cancer cells derived from any of the cells of the lung, e.g. alveolar cells (e.g. type I and II pneumocytes) and airway epithelial cells.

In some embodiments, the test sample is selected from: a biopsy (such as a core needle biopsy; fine needle biopsy; bronchoscopy sample); a tissue sample; an organ sample; a bodily fluid sample (such as pleural fluid). In some embodiments, the biopsy can be analysed using the methods of the present invention either with or without purification of cancer cells from the biopsy sample. The test sample can be taken specifically for the purpose of performing the methods of the present invention, or, in alternative embodiments, the methods of the invention can be carried out on historical samples that have been appropriately stored. In this alternative embodiment, the methods of the present invention can be used to retrospectively classify lung cancer samples.

It will be appreciated that the methods of the invention can be used to classify NSCLC in an individual into the prognostic subtypes described herein independently of the widely accepted classification of NSCLC into stages. Staging of NSCLC is used to describe how advanced the cancer is (which is in turn used to provide a prognosis) and is based on: (1) the size and extent of the main tumour; (ii) the spread to nearby lymph nodes; and (Iii) metastasis to different sites.

By “staging” we include determining the stage of a NSCLC, for example, determining whether the NSCLC is stage 0, stage I, stage II, stage III or stage IV (e.g., stage I, stage II, stage I-II, stage III-IV or stage I-IV), and/or determining whether the NSCLC is stage 0, stage IA, stage IB, stage IIA, stage IIB, stage IIIA, stage IIIB or stage IV, and/or determining whether the NSCLC is stage 0, stage IA1, stage IA2, stage IA3, stage IB, stage IIA, stage IIB, stage IIIA, stage IIIB, stage IIIC, stage IVA, or stage IVB. It is understood that stages 0, I and II are “early stage” NSCLC, and stages III and IV are “late stage” NSCLC. The methods of the present invention may be used to classify early stage NSCLC (i.e. Stage 0, I or II) in an individual. In other embodiments, the methods of the present invention may be used to classify late stage NSCLC (i.e. Stage III or Stage IV) In an individual. In some preferred embodiments, the NSCLC is early stage NSCLC.

Staging may correspond to the stages determined by the American Joint Committee on Cancer (AJCC) TNM system (e.g., see:
https://www.cancer.org/cancer/non-small-cell-lung-cancer/detection-diagnosis-staging/staging.html.

Therefore, the methods of the invention may be used to classify NSCLC in any of the above stages into the prognostic subtypes described herein. This is advantageous as the present invention provides prognostic information independently of the NSCLC stage, and also provides information on the molecular phenotype of tumours (which is not revealed by traditional staging which relies on the physical features of the tumour and pathology) at the level of expression of various protein or nucleic acid biomarkers, and can therefore provide a more accurate indicator of the cancer driving and immune regulation pathways involved. The methods of the invention therefore provide a systems view of the tumour state, combining the impact of genomic aberrations as well as epigenetic, transcriptional and post-transcriptional regulation.

In some embodiments, the methods of the invention further comprise, after determining the prognosis of NSCLC in the individual, selecting a treatment for the individual based on the prognosis. As discussed above, more accurately determining the prognosis based on the molecular phenotype allows the selection of a treatment appropriate to that prognosis, e.g. in terms of the type of treatment, its duration, and frequency. Such selections will be apparent to those skilled in the art, once a prognosis has been made. In some embodiments, this treatment is administered to the Individual.

Therefore, a further aspect of the invention provides a method for treating NSCLC in an individual, the method comprising the steps of:
determining the prognosis of NSCLC In the individual by the method defined herein by the first, second or third aspects; and
selecting a treatment for the individual, on the basis of the prognosis of NSCLC in the Individual, and administering the selected treatment to the individual.

The types of treatment available for NSCLC are well known in the art, and can include, but are not limited to, the following: chemotherapy, Immunotherapy, adoptive cell therapies, gene therapies, cancer vaccines, and oncolytic virus therapies.

NSCLC can be analysed to determine whether there are driver mutations present that drive the neoplastic transformation. If the NSCLC has an identifiable driver mutation, it can be treated using targeted therapies in the first instance (e.g. therapeutic small molecules and monoclonal antibodies targeting mTOR, EGFR, ALK, ROS, MET, and KRAS). This can be supplemented with any of the other treatment types discussed herein.

Driver mutation negative (typically EGFR-, ALK-, ROS-, BRAF-) NSCLC can be treated using immunotherapies (therapeutic small molecules and monoclonal antibodies targeting PDL1, PD1 or CTLA4, cytokines, adoptive cell therapies, gene therapies, cancer vaccines, oncolytic virus therapies) with or without chemotherapies.

In some alternative or additional embodiments, the methods of the invention further comprise, after determining the prognosis of NSCLC in the Individual, selecting a treatment for the individual based on the classification of the NSCLC determined by the methods disclosed herein. As discussed above, the methods of the Invention may facilitate classification of NSCLC into six prognostic subtypes (referred to as Prognostic Subtypes 1-6). On the basis of this classification, appropriate treatments can be selected based on the features that may be common to particular prognostic subtypes. In some embodiments, this treatment is administered to the individual.

Therefore, a further aspect of the invention provides a method for treating NSCLC in an individual, the method comprising the steps of:
determining the prognosis of NSCLC in the individual by the method defined in any of the first, second or third aspects; and
selecting a treatment for the individual, on the basis of the classification of the NSCLC in Step (1-c), Step (2-d) or Step (3-d) or the first, second or third aspects, and administering the selected treatment to the individual.

As mentioned above, treatments available for NSCLC are well known in the art, and can include, but are not limited to, the following: chemotherapy, immunotherapy, adoptive cell therapies, gene therapies, cancer vaccines, and oncolytic virus therapies.

In some embodiments, the treatment can be selected based on targeting driver mutations (e.g. EGFR, ALK, mTOR, ROS, MET, KRAS) identified as a common feature of a certain prognostic subtype.

In some embodiments, the selection of the treatment may additionally be based on the prognosis of the NSCLC in the individual, as determined by the method of the first, second or third aspects described herein. In this embodiment, the selection of the treatment is appropriate both for the common features of the prognostic subtype as described herein, and also for the prognosis of the patient.

As discussed herein, the NSCLC can be classified as Prognosis Subtype 1 and/or Prognosis Subtype 2 and/or Prognosis Subtype 3 and/or Prognosis Subtype 4 and/or Prognosis Subtype 5 and/or Prognosis Subtype 6.

Therefore, in some embodiments, the selection of the treatment based on the classification of the NSCLC is based on the classification into the prognostic subtypes 1-6. In some embodiments, the treatment based on the classification may include the following:
the NSCLC is classified as Prognosis Subtype 1 and the treatment is an EGFR targeting therapy; or
the NSCLC is classified as Prognosis Subtype 4 and the treatment is an mTOR targeting therapy.

By “targeting therapy” we Include a therapy designed to target species (for example proteins or enzymes) involved, either directly or indirectly, in the proliferation of NSCLC. In some embodiments, this may include inhibiting or reducing the action or activity of a protein involved in proliferation of the NSCLC. In other embodiments, this may include promoting or increasing the action or activity of a protein involved in Inhibiting proliferation of the NSCLC. Examples of targeting therapies for cancer are well-known in the art.

EGFR targeting therapies include, but are not limited to, the following: Erlotinib; Afatinib; Gefitinib; Osimertinib; Dacomitinib; and Necitumumab. mTOR targeting therapies Include, but are not limited to, the following: rapamycin and derivatives and analogues of rapamycin.

A further related aspect of the invention provides for use of the protein biomarkers defined in Table 1 and/or Table 2 and/or Table 3 and/or Table 4 and/or Table 5 and/or Table 6 for determining the prognosis of NSCLC in an individual.

A further related aspect of the invention provides for use of the protein biomarkers defined in Table B and/or Table C and/or Table D and/or Table E and/or Table F and/or Table G, for classifying and/or determining the prognosis of NSCLC In individual.

A further related aspect of the invention provides a computer program for operating the methods the invention. The computer program may be a programmed SVM-protein, k-TSP or SVM-peptide classification algorithm. The computer program may be recorded on a suitable computer readable carrier known to skilled persons. Suitable computer-readable-carriers may include compact discs (including CD-ROMs, DVDs, Blu-ray and the like), floppy discs, flash memory drives, ROM or hard disc drives. The computer program may be installed on a computer suitable for executing the computer program.

TABLES All biomarkers/genes referred to in the following tables refer to HGNC official symbols that can be retrieved from commonly used databases (e.g. Ensembl or the NCBI Gene Portal).

As is evident from the present disclosure, the selection of all biomarkers defined in Tables 1-6, Tables A, A(i)-(vii) and B-G is based on the experimental data and described in accompanying Example 1 and 2. Lehtio et al. (2021, Nature Cancer 2, 1224-1242) corresponds to Example 1 and is hereby incorporated by reference. The contents of the Tables is summarised below:
Table A contains 1755 markers that are significantly different between the NSCLC Prognosis Subtypes (abs(log 2FC)>1, DEqMS p.adj<0.01) as defined in FIG. 7a of Example 1. The methods used for generating the underlying data and for Identification of these markers is described in the Results and Methods sections of Example 1.
Table A(i) and Table 1 are NSCLC Prognosis Subtype 1 markers, defined as 132 non-overlapping markers from FIG. 6b (129 markers) and FIG. 7b (right part, 12 markers). The methods used for generating the underlying data and for Identification of these markers is described in the Results and Methods sections of Example 1.
Table A(ii) and Table 2 are NSCLC Prognosis Subtype 2 markers, defined as 38 non-overlapping markers from FIG. 6b (32 markers) and FIG. 7b (right part, 14 markers). The methods used for generating the underlying data and for Identification of these markers is described in the Results and Methods sections of Example 1.
Table A(Iii) and Table 3 are NSCLC Prognosis Subtype 3 markers, defined as 6 markers from FIG. 6b. The methods used for generating the underlying data and for Identification of these markers is described in the Results and Methods sections of Example 1.
Table A(iv) and Table 4 are NSCLC Prognosis Subtype 4 markers, defined as 28 non-overlapping markers from FIG. 6b (21 markers) and FIG. 7b (right part, 13 markers). The methods used for generating the underlying data and for identification of these markers is described in the Results and Methods sections of Example 1.
Table A(V) and Table 5 are NSCLC Prognosis Subtype 5 markers, defined as 459 markers from FIG. 6b. The methods used for generating the underlying data and for Identification of these markers is described in the Results and Methods sections of Example 1.
Table A(vi) and Table 6 are NSCLC Prognosis Subtype 6 markers, defined as 122 markers from FIG. 6b. The methods used for generating the underlying data and for identification of these markers is described in the Results and Methods sections of Example 1.
Table A(vii) is defined as the marker subset of Table A, 1118 markers, that is not covered by Tables A(i) to A(vi).
Table B contains 486 markers for SVM based classification of NSCLC Prognosis Subtype defined in FIG. 67c. The methods used for generating the underlying data and for Identification of these markers is described in the Results and Methods sections of Example 1.
Table C contains 200 priority markers for SVM based classification of NSCLC Prognosis Subtype. Table C is a subset of Table B defined in FIG. 67c, top-left quadrant. The methods used for generating the underlying data and for Identification of these markers is described in the Results and Methods sections of Example 1.
Table D contains 1630 marker-pairs for k-TSP based classification of NSCLC Prognosis Subtype defined in FIG. 67d. The methods used for generating the underlying data and for Identification of these markers is described in the Results and Methods sections of Example 1.
Table E contains 225 priority marker-pairs for k-TSP based classification of NSCLC Prognosis Subtype. Table E is a subset of Table D defined in FIG. 67d, top-left quadrant. The methods used for generating the underlying data and for identification of these markers is described in the Results and Methods sections of Example 1.
Table F contains 581 markers for SVM-peptide based classification of NSCLC Prognosis Subtype defined in FIG. 81f. The methods used for generating the underlying data and for identification of these markers is described in the Results and Methods sections of Example 2.
Table G contains 200 priority markers for SVM-peptide based classification of NSCLC Prognosis Subtype. Table G is a subset of Table F defined in FIG. 81f, top-left quadrant. The methods used for generating the underlying data and for identification of these markers is described in the Results and Methods sections of Example 2.

TABLE 1
Prognosis Subtype 1 (132 biomarkers)
Biomarker

| | | |
|---|---|---|
| AGR3 | PLLP | GPD1L |
| CRYM | ITGB6 | PALM3 |
| SCGB1A1 | PTPN13 | MYO5C |
| HPGD | ATP13A4 | NR3C2 |
| CAV3 | SPTLC3 | FUCA1 |
| DDAH1 | ECHDC2 | SULT1C2 |
| BMP3 | DNALI1 | RMDN2 |
| KIAA0408 | NEGR1 | TMOD1 |
| GJB1 | C1QTNF7 | MIPOL1 |
| SCN4B | MGLL | EPHX2 |
| SLC15A2 | SCARA5 | HMCN1 |
| VSIG2 | SUSD2 | ITPR3 |
| RAB27B | PYROXD2 | ADAMTSL5 |
| CYP4B1 | TRPC6 | FMN1 |
| SCN7A | ZNF385B | NECTIN3 |
| FMO5 | SNX25 | C11orf54 |
| VEPH1 | SLPI | C11orf96 |
| CAPS | TFPI | PITPNM3 |
| ADGRF5 | EPB41L1 | SLC16A7 |
| C16orf89 | CTSH | TMEM163 |
| CXCL17 | MISP3 | BCAM |
| MLPH | PLA2G4F | SPR |
| ESYT3 | MACC1 | PTPRU |
| SELENBP1 | PPP1R14A | CD34 |
| KCNK5 | ACAD8 | SORBS2 |
| COL10A1 | ADH1B | ANXA4 |
| FAAH | SORBS1 | RRAD |
| MAMDC2 | MYH11 | ACTN2 |
| EVPL | FHL1 | ITGA9 |
| NAPSA | CRYZ | TLR3 |
| TPPP3 | CHRDL1 | CYTH3 |
| CPAMD8 | ABCA8 | SMCO3 |
| ROBO2 | APCS | PID1 |
| ABCC3 | ARHGEF37 | TNXB |
| PPL | CAB39L | PGM5 |
| C1orf116 | GSTT1 | LTBP4 |
| NOG | SLC4A4 | CHDH |
| MFAP4 | DMD | PTPRG |
| AK1 | ABCC6 | CAMK2D |
| FBLN5 | CYB5A | GALNT10 |
| RGN | PYGB | ITGA8 |
| NOSTRIN | MMP28 | WNT7B |
| C2orf54 | HNF1B | SPEF1 |
| KLHDC7A | LIPH | CYP4X1 |

TABLE 2
Prognosis Subtype 2 (38 biomarkers)
Biomarker

| | |
|---|---|
| AREG | WARS |
| NRK | AMOTL2 |
| MROH6 | SPATS2L |
| ERRFI1 | BAIAP2L1 |
| CD274 | ACTG1 |
| MET | STAT1 |
| FOSL1 | SHCBP1 |
| STRIP2 | CASP8 |
| IDO1 | RASAL2 |
| DCBLD2 | HSD11B1 |
| TSC22D2 | UBE2L6 |
| GBP5 | FAM3C |
| UPP1 | CRYBG1 |
| STK17A | ZNF114 |
| NAMPT | CXCL9 |
| GZMB | FHOD3 |
| GBP1 | TMPRSS11E |
| GLS | MELTF |
| GPAT3 | CALHM6 |

TABLE 3
Prognosis Subtype 3 (6 biomarkers)
Biomarker

TNFRSF13C
BANK1
CD27
GPR183
IGDCC4
LIME1

TABLE 4
Prognosis Subtype 4 (28 biomarkers)
Biomarker

UGT3A1
CPS1
HGD
SLC7A2
FGL1
ATP6V0A4
FURIN
TC2N
HMGB3
PLA2G4A
OAZ2
SLC29A2
AKR1C4
SYT7
PDE4D
ENO3
FASN
GCNT2
MAP7
IDI1
SHTN1
B4GALNT2
KLK13
AKR1C2
ODC1
MSI1
BMP6
DNAJC12

TABLE 5
Prognosis Subtype 5 (459 biomarkers)
Biomarker

| | | |
|---|---|---|
| HOXB9 | RCC2 | SOX13 |
| ZIC5 | GDAP1 | BCL2L11 |
| LHX2 | MSH6 | ARID2 |
| SLC7A14 | MACROD2 | SPINDOC |
| DGKB | HAT1 | RNF138 |
| SEZ6 | MCM2 | GMNN |
| ELAVL3 | UHRF2 | LCORL |
| SRL | E2F3 | EED |
| MAST1 | ING4 | ZNF836 |
| ACTL6B | CDC7 | PINLYP |
| HPCA | USP1 | NCAPH2 |
| ADPRHL1 | RAD54L | CENPQ |
| ELAVL4 | PAPD7 | BCL7A |
| TCEAL2 | DCLK2 | PHF13 |
| TUBB2B | TCEANC2 | PPP1R3E |
| SH3GL2 | ZBTB39 | BRIP1 |
| INSM1 | JADE3 | SPIN1 |
| KIF5C | ZNF250 | BARD1 |
| ISL1 | KIF18B | CENPL |
| SCG3 | ATXN7L2 | PPM1D |
| DCX | MCM6 | PARP1 |
| SYN1 | REV3L | FIZ1 |
| AK5 | CDK4 | RSBN1 |
| SEZ6L | SYP | ZMYM2 |
| BTBD17 | CHAF1B | MAZ |
| PROX1 | JAKMIP1 | ZRANB2 |
| ATP8A2 | EHF | DEK |
| PPM1E | KIF22 | STRBP |
| NCAM1 | MSH2 | TTLL5 |
| ACBD7 | KIF24 | PHIP |
| TAGLN3 | MAP6 | ZNF136 |
| MYT1 | SMARCC1 | ARID1A |
| ZNF711 | KIAA1958 | CDKN1C |
| ST18 | MCM7 | AKT3 |
| SYN2 | CLGN | RIC8B |
| SCGN | CXCR4 | CDK2 |
| MTMR7 | TOP3A | ZNF646 |
| SPIN4 | KIF18A | DCAF16 |
| ZMAT4 | SKP2 | MBD3 |
| ZNF232 | TOP2A | TOPBP1 |
| ELOVL2 | MCM3 | MEX3B |
| NOL4 | MCM4 | NDC80 |
| KLHL14 | PHC1 | ZBTB33 |
| MYB | NASP | FANCL |
| E2F1 | RRM1 | ADNP2 |
| PSIP1 | SUGP2 | ZNF32 |
| CDCA7L | MCM9 | PSRC1 |
| TFAP2B | 44077 | THAP12 |
| RIMKLA | CENPU | CACNB3 |
| CDKN2A | FAM102B | MANEAL |
| E2F2 | PHF6 | MBTD1 |
| STMN2 | PIAS2 | TRIM28 |
| CDH2 | ZNF14 | SUPT16H |
| DPF1 | DHX40 | RAB39B |
| STXBP5L | POLA2 | PBX1 |
| FRMD3 | NUSAP1 | STK19 |
| CRMP1 | UBR7 | GINS4 |
| SOBP | MYBL2 | MPP2 |
| FAM111B | ZKSCAN8 | HNRNPA0 |
| SIX1 | ZNF420 | ZBED5 |
| TCEAL5 | ALKBH2 | ING1 |
| CADPS | ATAD2 | ZSWIM9 |
| ACYP1 | CCDC14 | DAXX |
| TRIM9 | MCM5 | RSF1 |
| NPTXR | HDAC2 | SMC2 |
| REEP1 | OXCT1 | SMAD4 |
| ZBTB18 | WEE1 | CBX1 |
| MSI1 | RNFT2 | SPC25 |
| DDC | TMEM108 | MSI2 |
| RCOR2 | KMT5C | SAMD1 |
| SMOC1 | CCP110 | UBTF |
| DLL1 | SSRP1 | NXT1 |
| SPATA33 | CBX2 | UNG |
| RFX3 | ITGB3BP | POLE |
| PLEKHG4B | SSX2IP | PDXP |
| PHYHIPL | SLX4IP | USP11 |
| SRSF12 | SRSF4 | NPAT |
| ASF1B | PARP2 | SMARCE1 |
| SV2A | PACSIN1 | MBD4 |
| NPTX1 | CDK2AP1 | SARM1 |
| CBFA2T2 | NFIB | MEN1 |
| KIFC1 | KCTD1 | CHAF1A |
| UHRF1 | MYEF2 | ZNF740 |
| BEND5 | SMARCA4 | RPS6KA5 |
| E2F7 | TFDP1 | KDM2B |
| STMN1 | DNMT3B | RAB39A |
| KIAA1586 | PRMT6 | PKNOX1 |
| SIX2 | SUZ12 | PHC2 |
| TYMS | DNMT1 | PPIG |
| FSD1L | RBP1 | H2AFY2 |
| GNAZ | ZNF48 | MND1 |
| PTPRN2 | PRR36 | CTPS1 |
| EYA2 | GNAO1 | BRCA1 |
| MEX3A | MDC1 | TLK2 |
| WNT11 | NT5DC2 | NCAPG |
| FSD1 | HELLS | STAG2 |
| PTBP2 | HACE1 | KIF2C |
| CKB | EYA3 | PSMC3IP |
| SNAP25 | ECT2 | BCOR |
| POLA1 | MEST | KAT7 |
| PRIM1 | ZFP82 | FAM122B |
| ZNF219 | TIMELESS | DSN1 |
| CENPF | BEND3 | RBBP4 |
| MARCKSL1 | HMGB2 | ZNF532 |
| CCNE2 | ATAD5 | FEN1 |
| NRCAM | TPX2 | TBPL1 |
| KIF1A | CASZ1 | RIF1 |
| SCML2 | ESCO2 | C19orf47 |
| E2F8 | PRPF4B | KDM4C |
| PIMREG | CHD7 | CEP97 |
| CENPV | PATZ1 | IPO8 |
| CADM2 | RALYL | MIS18BP1 |
| MTF2 | ZNF768 | PMF1 |
| BCL2 | GAB2 | QSER1 |
| OR4K3 | GPANK1 | ZFP37 |
| SS18L1 | NCAPG2 | RBL1 |
| GIPC2 | KMT2A | ZNF445 |
| PCSK2 | VEZF1 | ZMYM4 |
| NSD2 | ASF1A | ELF2 |
| ZNF664 | NCAPD3 | MTA1 |
| ZNF84 | LIG1 | PHF8 |
| WDR76 | SLC36A4 | TEAD1 |
| CKAP2 | ZBTB12 | CHD8 |
| L3MBTL4 | FGD1 | ZNF516 |
| CTNNA2 | CDCA8 | FAM76B |
| CASP8AP2 | ATF3 | CDK11B |
| TRIM36 | SAAL1 | PRDM15 |
| SMAD9 | NAPB | ACTL6A |
| ZNF292 | CENPI | SMARCB1 |
| HDGFL2 | BRI3BP | USP37 |
| TXNDC16 | UBE2S | CHD6 |
| AMPH | NUF2 | EHMT1 |
| EZH2 | USP48 | HAUS1 |
| SGO1 | FRG1 | EMSY |
| CBX5 | TIPIN | CENPP |
| TEX30 | SKA1 | ZNF672 |
| RELN | SPIN3 | WDR70 |
| PRIM2 | WASF1 | MUTYH |
| PHF10 | RAB9B | RLF |
| BRD3 | CDCA2 | UBE2T |
| RMI1 | ZRANB3 | SHPRH |
| HIRIP3 | FOXRED2 | SMC1A |
| SMARCD1 | ZNF525 | YEATS4 |
| MAD2L2 | ZNF362 | TMPO |
| ZNF428 | TSHZ1 | RAB3A |
| FAM221A | PHF20 | LIN7A |
| DNMT3A | KIF20A | PNMA1 |
| DPYSL4 | PBRM1 | USP22 |
| DACH1 | TMEM206 | ATF1 |
| RPRD1A | CDK19 | ZBTB14 |
| CHN1 | GINS1 | TSN |
| GKAP1 | E2F6 | ZNF579 |
| AVIL | PDS5B | GLMN |

TABLE 6
Prognosis Subtype 6 (122 biomarkers)
Biomarker

| | | |
|---|---|---|
| SPRR1B | GJB5 | COL4A6 |
| DSG3 | TGM1 | WNT5A |
| KRT6A | IL1RN | DSC2 |
| PKP1 | TNS4 | FGFR3 |
| TRIM29 | S100A7 | DSP |
| KRT5 | ADH7 | NDRG4 |
| DSC3 | TMEM40 | SFN |
| CLCA2 | RGMA | CD109 |
| KRT15 | CLDN1 | BCL11A |
| S100A2 | PCDH19 | LAMC2 |
| KRT6B | FAM83B | FRMD6 |
| KRT16 | NECTIN1 | FAM83H |
| KRT17 | SERPINB2 | KLHL13 |
| NTRK2 | FST | SLC1A3 |
| ANXA8 | KRT72 | SGK1 |
| SERPINB5 | JAG1 | ITGB8 |
| SERPINB13 | GBP7 | MICALL1 |
| KRT13 | ACKR3 | ABCC1 |
| VSNL1 | RAPGEFL1 | FSCN1 |
| ANXA8L1 | PPP1R14C | TRPV4 |
| FAT2 | GJA1 | TNFAIP6 |
| GBP6 | VTCN1 | PARD6G |
| CERS3 | GPC1 | CLSTN1 |
| COL7A1 | ULBP2 | OLFML2A |
| KRT75 | UPK1B | SLC1A4 |
| A2ML1 | SNAI2 | C3orf58 |
| COL17A1 | BNC1 | BICD2 |
| TP63 | DENND2C | ST6GALNAC2 |
| SERPINB3 | MMP3 | TMEM189 |
| CALML3 | DUSP9 | FZD6 |
| KRT14 | DUSP14 | ACAP2 |
| LYPD3 | MMP10 | EGFR |
| TENM2 | KRT3 | TMEM132A |
| KRT6C | FERMT1 | ABCA13 |
| CAPNS2 | PLCH2 | SDC1 |
| SERPINB4 | FGFR2 | ZNF385A |
| PTPRZ1 | CYP2S1 | KLF5 |
| CLCA4 | JUP | SLC6A9 |
| IRF6 | SLC2A1 | IGSF3 |
| CSTA | TMPRSS11D | TMTC3 |
| ABCC5 | ITGB4 | |

TABLE Ai
(132 biomarkers):
Biomarker

| | | |
|---|---|---|
| AGR3 | PLLP | GPD1L |
| CRYM | ITGB6 | PALM3 |
| SCGB1A1 | PTPN13 | MYO5C |
| HPGD | ATP13A4 | NR3C2 |
| CAV3 | SPTLC3 | FUCA1 |
| DDAH1 | ECHDC2 | SULT1C2 |
| BMP3 | DNALI1 | RMDN2 |
| KIAA0408 | NEGR1 | TMOD1 |
| GJB1 | C1QTNF7 | MIPOL1 |
| SCN4B | MGLL | EPHX2 |
| SLC15A2 | SCARA5 | HMCN1 |
| VSIG2 | SUSD2 | ITPR3 |
| RAB27B | PYROXD2 | ADAMTSL5 |
| CYP4B1 | TRPC6 | FMN1 |
| SCN7A | ZNF385B | NECTIN3 |
| FMO5 | SNX25 | C11orf54 |
| VEPH1 | SLPI | C11orf96 |
| CAPS | TFPI | PITPNM3 |
| ADGRF5 | EPB41L1 | SLC16A7 |
| C16orf89 | CTSH | TMEM163 |
| CXCL17 | MISP3 | BCAM |
| MLPH | PLA2G4F | SPR |
| ESYT3 | MACC1 | PTPRU |
| SELENBP1 | PPP1R14A | CD34 |
| KCNK5 | ACAD8 | SORBS2 |
| COL10A1 | ADH1B | ANXA4 |
| FAAH | SORBS1 | RRAD |
| MAMDC2 | MYH11 | ACTN2 |
| EVPL | FHL1 | ITGA9 |
| NAPSA | CRYZ | TLR3 |
| TPPP3 | CHRDL1 | CYTH3 |
| CPAMD8 | ABCA8 | SMCO3 |
| ROBO2 | APCS | PID1 |
| ABCC3 | ARHGEF37 | TNXB |
| PPL | CAB39L | PGM5 |
| C1orf116 | GSTT1 | LTBP4 |
| NOG | SLC4A4 | CHDH |
| MFAP4 | DMD | PTPRG |
| AK1 | ABCC6 | CAMK2D |
| FBLN5 | CYB5A | GALNT10 |
| RGN | PYGB | ITGA8 |
| NOSTRIN | MMP28 | WNT7B |
| C2orf54 | HNF1B | SPEF1 |
| KLHDC7A | LIPH | CYP4X1 |

TABLE Aii
(38 biomarkers):

| | |
|---|---|
| AREG | WARS |
| NRK | AMOTL2 |
| MROH6 | SPATS2L |
| ERRFI1 | BAIAP2L1 |
| CD274 | ACTG1 |
| MET | STAT1 |
| FOSL1 | SHCBP1 |
| STRIP2 | CASP8 |
| IDO1 | RASAL2 |
| DCBLD2 | HSD11B1 |
| TSC22D2 | UBE2L6 |
| GBP5 | FAM3C |
| UPP1 | CRYBG1 |
| STK17A | ZNF114 |
| NAMPT | CXCL9 |
| GZMB | FHOD3 |
| GBP1 | TMPRSS11E |
| GLS | MELTF |
| GPAT3 | CALHM6 |

TABLE Aiii
(6 biomarkers):

TNFRSF13C
BANK1
CD27
GPR183
IGDCC4
LIME1

TABLE Aiv
(28 biomarkers)

UGT3A1
CPS1
HGD
SLC7A2
FGL1
ATP6V0A4
FURIN
TC2N
HMGB3
PLA2G4A
OAZ2
SLC29A2
AKR1C4
SYT7
PDE4D
ENO3
FASN
GCNT2
MAP7
IDI1
SHTN1
B4GALNT2
KLK13
AKR1C2
ODC1
MSI1
BMP6
DNAJC12

TABLE Av
(459 biomarkers):

| | | |
|---|---|---|
| HOXB9 | RCC2 | SOX13 |
| ZIC5 | GDAP1 | BCL2L11 |
| LHX2 | MSH6 | ARID2 |
| SLC7A14 | MACROD2 | SPINDOC |
| DGKB | HAT1 | RNF138 |
| SEZ6 | MCM2 | GMNN |
| ELAVL3 | UHRF2 | LCORL |
| SRL | E2F3 | EED |
| MAST1 | ING4 | ZNF836 |
| ACTL6B | CDC7 | PINLYP |
| HPCA | USP1 | NCAPH2 |
| ADPRHL1 | RAD54L | CENPQ |
| ELAVL4 | PAPD7 | BCL7A |
| TCEAL2 | DCLK2 | PHF13 |
| TUBB2B | TCEANC2 | PPP1R3E |
| SH3GL2 | ZBTB39 | BRIP1 |
| INSM1 | JADE3 | SPIN1 |
| KIF5C | ZNF250 | BARD1 |
| ISL1 | KIF18B | CENPL |
| SCG3 | ATXN7L2 | PPM1D |
| DCX | MCM6 | PARP1 |
| SYN1 | REV3L | FIZ1 |
| AK5 | CDK4 | RSBN1 |
| SEZ6L | SYP | ZMYM2 |
| BTBD17 | CHAF1B | MAZ |
| PROX1 | JAKMIP1 | ZRANB2 |
| ATP8A2 | EHF | DEK |
| PPM1E | KIF22 | STRBP |
| NCAM1 | MSH2 | TTLL5 |
| ACBD7 | KIF24 | PHIP |
| TAGLN3 | MAP6 | ZNF136 |
| MYT1 | SMARCC1 | ARID1A |
| ZNF711 | KIAA1958 | CDKN1C |
| ST18 | MCM7 | AKT3 |
| SYN2 | CLGN | RIC8B |
| SCGN | CXCR4 | CDK2 |
| MTMR7 | TOP3A | ZNF646 |
| SPIN4 | KIF18A | DCAF16 |
| ZMAT4 | SKP2 | MBD3 |
| ZNF232 | TOP2A | TOPBP1 |
| ELOVL2 | MCM3 | MEX3B |
| NOL4 | MCM4 | NDC80 |
| KLHL14 | PHC1 | ZBTB33 |
| MYB | NASP | FANCL |
| E2F1 | RRM1 | ADNP2 |
| PSIP1 | SUGP2 | ZNF32 |
| CDCA7L | MCM9 | PSRC1 |
| TFAP2B | 44077 | THAP12 |
| RIMKLA | CENPU | CACNB3 |
| CDKN2A | FAM102B | MANEAL |
| E2F2 | PHF6 | MBTD1 |
| STMN2 | PIAS2 | TRIM28 |
| CDH2 | ZNF14 | SUPT16H |
| DPF1 | DHX40 | RAB39B |
| STXBP5L | POLA2 | PBX1 |
| FRMD3 | NUSAP1 | STK19 |
| CRMP1 | UBR7 | GINS4 |
| SOBP | MYBL2 | MPP2 |
| FAM111B | ZKSCAN8 | HNRNPA0 |
| SIX1 | ZNF420 | ZBED5 |
| TCEAL5 | ALKBH2 | ING1 |
| CADPS | ATAD2 | ZSWIM9 |
| ACYP1 | CCDC14 | DAXX |
| TRIM9 | MCM5 | RSF1 |
| NPTXR | HDAC2 | SMC2 |
| REEP1 | OXCT1 | SMAD4 |
| ZBTB18 | WEE1 | CBX1 |
| MSI1 | RNFT2 | SPC25 |
| DDC | TMEM108 | MSI2 |
| RCOR2 | KMT5C | SAMD1 |
| SMOC1 | CCP110 | UBTF |
| DLL1 | SSRP1 | NXT1 |
| SPATA33 | CBX2 | UNG |
| RFX3 | ITGB3BP | POLE |
| PLEKHG4B | SSX2IP | PDXP |
| PHYHIPL | SLX4IP | USP11 |
| SRSF12 | SRSF4 | NPAT |
| ASF1B | PARP2 | SMARCE1 |
| SV2A | PACSIN1 | MBD4 |
| NPTX1 | CDK2AP1 | SARM1 |
| CBFA2T2 | NFIB | MEN1 |
| KIFC1 | KCTD1 | CHAF1A |
| UHRF1 | MYEF2 | ZNF740 |
| BEND5 | SMARCA4 | RPS6KA5 |
| E2F7 | TFDP1 | KDM2B |
| STMN1 | DNMT3B | RAB39A |
| KIAA1586 | PRMT6 | PKNOX1 |
| SIX2 | SUZ12 | PHC2 |
| TYMS | DNMT1 | PPIG |
| FSD1L | RBP1 | H2AFY2 |
| GNAZ | ZNF48 | MND1 |
| PTPRN2 | PRR36 | CTPS1 |
| EYA2 | GNAO1 | BRCA1 |
| MEX3A | MDC1 | TLK2 |
| WNT11 | NT5DC2 | NCAPG |
| FSD1 | HELLS | STAG2 |
| PTBP2 | HACE1 | KIF2C |
| CKB | EYA3 | PSMC3IP |
| SNAP25 | ECT2 | BCOR |
| POLA1 | MEST | KAT7 |
| PRIM1 | ZFP82 | FAM122B |
| ZNF219 | TIMELESS | DSN1 |
| CENPF | BEND3 | RBBP4 |
| MARCKSL1 | HMGB2 | ZNF532 |
| CCNE2 | ATAD5 | FEN1 |
| NRCAM | TPX2 | TBPL1 |
| KIF1A | CASZ1 | RIF1 |
| SCML2 | ESCO2 | C19orf47 |
| E2F8 | PRPF4B | KDM4C |
| PIMREG | CHD7 | CEP97 |
| CENPV | PATZ1 | IPO8 |
| CADM2 | RALYL | MIS18BP1 |
| MTF2 | ZNF768 | PMF1 |
| BCL2 | GAB2 | QSER1 |
| OR4K3 | GPANK1 | ZFP37 |
| SS18L1 | NCAPG2 | RBL1 |
| GIPC2 | KMT2A | ZNF445 |
| PCSK2 | VEZF1 | ZMYM4 |
| NSD2 | ASF1A | ELF2 |
| ZNF664 | NCAPD3 | MTA1 |
| ZNF84 | LIG1 | PHF8 |
| WDR76 | SLC36A4 | TEAD1 |
| CKAP2 | ZBTB12 | CHD8 |
| L3MBTL4 | FGD1 | ZNF516 |
| CTNNA2 | CDCA8 | FAM76B |
| CASP8AP2 | ATF3 | CDK11B |
| TRIM36 | SAAL1 | PRDM15 |
| SMAD9 | NAPB | ACTL6A |
| ZNF292 | CENPI | SMARCB1 |
| HDGFL2 | BRI3BP | USP37 |
| TXNDC16 | UBE2S | CHD6 |
| AMPH | NUF2 | EHMT1 |
| EZH2 | USP48 | HAUS1 |
| SGO1 | FRG1 | EMSY |
| CBX5 | TIPIN | CENPP |
| TEX30 | SKA1 | ZNF672 |
| RELN | SPIN3 | WDR70 |
| PRIM2 | WASF1 | MUTYH |
| PHF10 | RAB9B | RLF |
| BRD3 | CDCA2 | UBE2T |
| RMI1 | ZRANB3 | SHPRH |
| HIRIP3 | FOXRED2 | SMC1A |
| SMARCD1 | ZNF525 | YEATS4 |
| MAD2L2 | ZNF362 | TMPO |
| ZNF428 | TSHZ1 | RAB3A |
| FAM221A | PHF20 | LIN7A |
| DNMT3A | KIF20A | PNMA1 |
| DPYSL4 | PBRM1 | USP22 |
| DACH1 | TMEM206 | ATF1 |
| RPRD1A | CDK19 | ZBTB14 |
| CHN1 | GINS1 | TSN |
| GKAP1 | E2F6 | ZNF579 |
| AVIL | PDS5B | GLMN |

TABLE Avi
(122 biomarkers):

| | | |
|---|---|---|
| SPRR1B | GJB5 | COL4A6 |
| DSG3 | TGM1 | WNT5A |
| KRT6A | IL1RN | DSC2 |
| PKP1 | TNS4 | FGFR3 |
| TRIM29 | S100A7 | DSP |
| KRT5 | ADH7 | NDRG4 |
| DSC3 | TMEM40 | SFN |
| CLCA2 | RGMA | CD109 |
| KRT15 | CLDN1 | BCL11A |
| S100A2 | PCDH19 | LAMC2 |
| KRT6B | FAM83B | FRMD6 |
| KRT16 | NECTIN1 | FAM83H |
| KRT17 | SERPINB2 | KLHL13 |
| NTRK2 | FST | SLC1A3 |
| ANXA8 | KRT72 | SGK1 |
| SERPINB5 | JAG1 | ITGB8 |
| SERPINB13 | GBP7 | MICALL1 |
| KRT13 | ACKR3 | ABCC1 |
| VSNL1 | RAPGEFL1 | FSCN1 |
| ANXA8L1 | PPP1R14C | TRPV4 |
| FAT2 | GJA1 | TNFAIP6 |
| GBP6 | VTCN1 | PARD6G |
| CERS3 | GPC1 | CLSTN1 |
| COL7A1 | ULBP2 | OLFML2A |
| KRT75 | UPK1B | SLC1A4 |
| A2ML1 | SNAI2 | C3orf58 |
| COL17A1 | BNC1 | BICD2 |
| TP63 | DENND2C | ST6GALNAC2 |
| SERPINB3 | MMP3 | TMEM189 |
| CALML3 | DUSP9 | FZD6 |
| KRT14 | DUSP14 | ACAP2 |
| LYPD3 | MMP10 | EGFR |
| TENM2 | KRT3 | TMEM132A |
| KRT6C | FERMT1 | ABCA13 |
| CAPNS2 | PLCH2 | SDC1 |
| SERPINB4 | FGFR2 | ZNF385A |
| PTPRZ1 | CYP2S1 | KLF5 |
| CLCA4 | JUP | SLC6A9 |
| IRF6 | SLC2A1 | IGSF3 |
| CSTA | TMPRSS11D | TMTC3 |
| ABCC5 | ITGB4 | |

TABLE Avii
(1118 biomarkers):

| | | | | |
|---|---|---|---|---|
| CDT1 | F3 | FAM81B | AMD1 | KNSTRN |
| RNF168 | FAM84A | FAM92B | ANKRD22 | KPNA2 |
| SLC16A4 | FANCI | FANCA | ANKRD42 | KRT12 |
| SLC44A4 | FBXO27 | FANCB | ANXA2 | KRT71 |
| ABCC2 | FOXF2 | FANCD2 | APCDD1 | KRT76 |
| AGR2 | FOXM1 | FANCG | APOL3 | KRT8 |
| CDC20 | FRAS1 | FAXC | AQP5 | LAYN |
| COL8A2 | FXYD1 | FBXO5 | ARHGEF9 | LDHD |
| EPHA7 | GKN2 | FCER1A | ARMC3 | LEF1 |
| FAM180A | GLDC | FCER1G | ARMC4 | LILRB4 |
| FANCE | GP2 | FCGBP | ARRB1 | LIMS2 |
| FGFBP1 | GPM6A | FGF2 | ASPH | LMNA |
| GPR39 | GPRC5C | FOXC1 | ASS1 | LPAR1 |
| KCTD15 | GPT2 | FREM2 | ATP8A1 | LPCAT1 |
| SLC34A2 | GTSE1 | FUT1 | B4GALNT1 | MAD2L1 |
| SMIM22 | HINFP | GABRG3 | BAZ2B | MAGEA6 |
| WNK2 | HJURP | GDF15 | BCL2L14 | MANSC1 |
| CA3 | HLA-DQB2 | GGH | BCO1 | MAP1B |
| CEACAM21 | HOXB7 | GIMAP4 | BLM | MARC1 |
| CTSE | HSD17B6 | GIMAP7 | BORA | MASTL |
| FAM83F | HTATSF1 | GINS3 | BRD4 | MATN2 |
| GPD1 | IGF2BP2 | GLB1L2 | BRSK2 | MDFIC2 |
| IL2RA | IVL | GLT1D1 | BTBD11 | MFNG |
| IYD | KIAA1324 | GPAT2 | BTLA | MMP23B |
| NGFR | KIAA1549 | GREM2 | BUB1 | MTERF2 |
| OIP5 | KIF14 | GRPEL2 | BUD13 | MTFR2 |
| PCYT1B | KIF5A | GSTM5 | C11orf70 | MTHFD2 |
| PLA2G2A | KIT | GSTO2 | C18orf25 | MTTP |
| RACGAP1 | KITLG | GZMA | C1QTNF2 | MUS81 |
| SCD5 | KNL1 | HAPLN1 | C4orf19 | MVP |
| SKA3 | KRT23 | HHLA2 | C7orf57 | MYLK |
| SSPN | KRT7 | HIST1H1A | C9orf40 | MZB1 |
| STEAP4 | KYNU | HIST1H2BA | CA2 | NAALADL2 |
| VSTM2L | LAD1 | HKDC1 | CACNA1C | NARF |
| YBX2 | LGI4 | HLA-DOB | CALCA | NDUFA4L2 |
| ADIRF | LTBP2 | HMGN4 | CAMK4 | NFASC |
| ARG2 | MARK1 | HMMR | CAPN8 | NFE2L2 |
| AURKB | MDH1B | HNMT | CAPN9 | NFIA |
| BIRC5 | MEGF6 | HOXB6 | CAPSL | NFIX |
| CDC25C | MELK | HPCAL4 | CBR1 | NME5 |
| CDK1 | METTL7B | HPGDS | CCDC151 | NME9 |
| CKAP2L | MGST1 | HPSE2 | CCDC181 | NR0B1 |
| DEPDC1B | MMP11 | ID1 | CCL20 | NR1D2 |
| EDARADD | MMP7 | IGKV1-8 | CCL28 | NRIP2 |
| FAM83A | MS4A1 | IL33 | CCNA2 | NRXN3 |
| GINS2 | MS4A15 | INCENP | CCND2 | NT5E |
| HMGA2 | MUC13 | IQGAP3 | CCNE1 | NT5M |
| HSPB6 | MUC3A | ITGA6 | CD2 | OCLN |
| KIF11 | MUC5B | ITGA7 | CD22 | OPHN1 |
| LDB3 | NAF1 | JAKMIP2 | CD3E | ORC1 |
| MGP | NCAPD2 | JAM3 | CD3G | ORC6 |
| MKI67 | NOS2 | JPH2 | CD48 | OXER1 |
| MYCL | NPR3 | KCNJ15 | CD63 | P2RY8 |
| OGN | NQO1 | KCNN4 | CD69 | PACRG |
| PHLDA2 | NRG1 | KIF15 | CD79B | PADI3 |
| PYGM | NUDT10 | KLB | CDH17 | PAF1 |
| RAB3B | OLFML1 | KLC3 | CDKN2B | PAQR4 |
| RHEX | OSMR | KLK11 | CDS1 | PARD6B |
| SFTPB | PAX7 | KLRD1 | CEL | PBLD |
| SGMS2 | PDZK1IP1 | KNTC1 | CENPN | PCDH1 |
| SH3RF2 | PLA2G12B | KRT19 | CFAP52 | PCDH7 |
| SIGLEC6 | PLAC9 | LAMA3 | CFAP53 | PCDHB14 |
| SLC38A1 | PLAT | LAMB3 | CGREF1 | PDE10A |
| SOX2 | PLAU | LEO1 | CHD2 | PDE1C |
| SP6 | PNMA3 | LIMCH1 | CHTF8 | PHF24 |
| ST6GALNAC1 | PPP1R26 | LPIN2 | CKS1B | PHLDA3 |
| STMN3 | PRC1 | LPL | CLCN2 | PHOSPHO2 |
| TNFRSF19 | PRDM16 | LRP4 | CLDN10 | PI15 |
| TRIP13 | PRKG1 | LRRC17 | CLDN2 | PI16 |
| VANGL2 | PROM1 | LRRC23 | CLEC10A | PKMYT1 |
| ZBTB7C | PROSER3 | LRRC75A | CLEC5A | PLA2G16 |
| ZNF697 | PTN | LRRN4 | CLSTN2 | PLA2G2D |
| ADAM23 | RAB3C | LTB | CNTN1 | PLAUR |
| ADGRD1 | RAD54B | MAGEA3 | COBL | PLCB4 |
| ALKAL2 | RAP1GAP | MAGEA4 | COL2A1 | PLCH1 |
| AOC3 | RARG | MAOA | COL6A5 | PLEKHA6 |
| AP3B2 | REEP6 | MAOB | COL6A6 | PLEKHG5 |
| ARHGEF39 | RHBDL2 | MAP3K13 | CORIN | PLIN2 |
| ARHGEF4 | RHOD | MDGA1 | CPA4 | PLIN4 |
| BCAS1 | RUNDC3A | MIS18A | CPNE7 | PLOD2 |
| BCL2L15 | S100P | MMP1 | CPNE8 | POLD3 |
| BEND7 | SDR16C5 | MMP13 | CRB3 | PRSS8 |
| BRSK1 | SFTPD | MMS22L | CREB3L4 | PRTFDC1 |
| CCNB1 | SGCD | MPZL2 | CRLF1 | PSAT1 |
| CCR6 | SGCG | MRGPRF | CROCC2 | PTAFR |
| CD207 | SH3GL3 | MTMR8 | CSRP3 | PTER |
| CDC6 | SLC6A4 | MTUS1 | CTR9 | PTGS2 |
| CEP55 | SLFN13 | MUC15 | CTSF | PTPRO |
| CHGB | SMC4 | NAALAD2 | CTSO | PVALB |
| CIP2A | SNCG | NDNF | CTTNBP2 | PYGL |
| CLDN18 | SNX22 | NOS1 | CWC22 | RAB19 |
| CLIC6 | SOX6 | NOVA1 | CYB5R2 | RAB38 |
| COL14A1 | SOX9 | NPR1 | CYBRD1 | RAB3IP |
| COL8A1 | SPATA18 | NPTX2 | CYP24A1 | RASEF |
| CPA3 | SRPX | NTN4 | CYP27B1 | RASL11A |
| CYP2W1 | STARD5 | NTS | CYP4F11 | RASSF4 |
| DAPL1 | STX1A | OAT | DACT2 | RAX |
| DBF4 | SYTL2 | OLR1 | DACT3 | RBKS |
| DCDC2 | TMEM45B | OVOL2 | DCC | RDH10 |
| DEPDC1 | TMPRSS4 | PAH | DCN | RET |
| DES | TOX3 | PCNA | DDX11 | RETN |
| DLL3 | TP53 | PDK4 | DIRAS2 | RFC5 |
| DLX5 | TSPAN8 | PDLIM2 | DLGAP1 | RFLNA |
| DTL | TSPYL5 | PGC | DNA2 | RHCG |
| ELAVL2 | TTC39A | PHGDH | DNAH7 | RNF128 |
| ELOVL4 | TTK | PITX1 | DNAI2 | ROPN1L |
| FBP1 | UBE2C | PLK1 | DNAJC6 | RP1 |
| FBP2 | UCHL1 | POLE2 | DOT1L | RRM2 |
| FHL5 | UNC5CL | PON2 | DPY19L1 | RSPH1 |
| FMO1 | VSIG1 | PON3 | DQX1 | RSPH4A |
| FOLR1 | VWA5B2 | POU2F2 | DSCC1 | RSRC2 |
| FOXA1 | WDR62 | POU3F2 | DUSP6 | RTF1 |
| FOXE1 | WNT4 | PPP1R14D | EFCAB11 | RTN1 |
| FXYD6 | XDH | PPP1R9A | EFR3B | S100A1 |
| GCH1 | ZBTB26 | PRAME | EGF | S100A7A |
| GRB14 | ZC3H8 | PRND | EGFL7 | S100A9 |
| GRIK3 | ZCCHC3 | PRSS57 | ENDOD1 | S100B |
| GSDMC | ZNF367 | PTMA | ENKUR | SAMD8 |
| HASPIN | ZNF93 | RAB17 | EPHA1 | SAMD9L |
| IGF2BP3 | AADAC | RAD51AP1 | EPHA2 | SCIN |
| IL37 | ABI3BP | RERG | EPHA3 | SCN2B |
| INA | ACE2 | RFC4 | EPHX1 | SCUBE2 |
| KIF23 | ACSM3 | RHAG | EPS8L1 | SCUBE3 |
| KIF4A | ADCY5 | RHOF | ERO1A | SEC11C |
| KLRG2 | ADGRF1 | RIN1 | ERVMER34-1 | SEMA3B |
| KRT24 | ADGRG3 | RPH3AL | FADS2 | SEMA3F |
| LAMA1 | ADH6 | RSPH3 | FAH | SEPT1 |
| LINGO1 | AGA | RSPH9 | FAM174B | SFRP4 |
| LMOD1 | AK8 | S100A10 | FAM200A | SFRP5 |
| MAGIX | AKR1C1 | S100A14 | FAM3D | SGSM1 |
| MAPK8IP1 | ANKRD65 | S100A6 | FAM83C | SH2D1A |
| MEOX2 | ANXA9 | S100A8 | FAM89A | SH2D4A |
| MFSD4A | APEX2 | SAPCD2 | FAM9C | SH3KBP1 |
| MUC1 | APOBEC3B | SASS6 | FAS | SIDT1 |
| MYC | AQP4 | SBSPON | FAT4 | SLAMF1 |
| NCAPH | ARHGEF38 | SCEL | FCRL2 | SLC16A3 |
| NELL1 | ARSE | SELP | FHOD1 | SLC16A5 |
| NKX2-1 | ART3 | SEMA6C | FNIP2 | SLC20A2 |
| NUDT11 | ASPM | SFTPA1 | FOLR2 | SLC22A23 |
| OTX1 | ASRGL1 | SGO2 | FOSL2 | SLC2A12 |
| PADI1 | ATP11A | SGPP2 | FOXA2 | SLC38A2 |
| PAX6 | B3GNT6 | SH3BGR | FSTL4 | SLC43A3 |
| PBK | BHMT2 | SH3BGRL2 | FUT2 | SLC44A3 |
| PCSK1 | BMP7 | SHC3 | G6PD | SLC50A1 |
| PDGFA | BMPER | SHISA2 | GALE | SLC7A5 |
| PGAP1 | BRD2 | SHISA4 | GALNT16 | SMOC2 |
| PIGR | C1orf112 | SIAE | GALNT3 | SMPD3 |
| PITX2 | Clorf87 | SIGIRR | GALNT5 | SNX21 |
| PLEKHG6 | CA12 | SKAP1 | GAS1 | SOAT1 |
| POU2F3 | CABYR | SLC10A4 | GBP2 | SOX21 |
| SATB2 | CARD17 | SLC16A1 | GBP4 | SPAG5 |
| SCGB2A1 | CASP14 | SLC39A4 | GCNT3 | SPAG6 |
| SCGB3A2 | CASP5 | SLC4A11 | GDA | SPEF2 |
| SLC22A3 | CAV1 | SLC6A17 | GDF10 | SPEG |
| SLC38A3 | CAVIN1 | SLC7A1 | GFI1 | SPP2 |
| SLC6A12 | CAVIN2 | SPATA6 | GFI1B | SPRR3 |
| SNAP91 | CCDC138 | SPC24 | GFPT2 | SSC5D |
| SNCAIP | CCNB2 | SPINK5 | GIMAP6 | SSH3 |
| SPON1 | CD177 | SPOCK3 | GLI3 | SSTR2 |
| SYNPO2 | CD1C | SRXN1 | GLOD5 | ST3GAL5 |
| TBX15 | CD247 | STARD10 | GLTPD2 | ST6GALNAC3 |
| TFAP2C | CD79A | STIL | GPC3 | STAG3 |
| UGT1A7 | CD8A | SYPL2 | GPM6B | STEAP1 |
| VEGFD | CDA | SYT1 | GRAMD2A | STEAP2 |
| WNK3 | CDH13 | SYT13 | GRIP1 | STK32A |
| ACSL5 | CDH23 | TACSTD2 | GTF2F1 | STOX1 |
| ADH1C | CECR2 | TBC1D30 | GZMH | STXBP6 |
| ADSSL1 | CELSR3 | TCL1A | GZMK | SUSD5 |
| AFAP1L2 | CENPK | TDO2 | HAL | TACC3 |
| AHNAK2 | CENPM | TDRD10 | HAS3 | TBC1D8B |
| AIM2 | CENPO | TEKT1 | HEPACAM2 | TCEANC |
| ANLN | CENPT | TEKT2 | HHIP | TCF3 |
| ARHGEF19 | CERS4 | TESC | HIBADH | TDG |
| ART4 | CES2 | TET3 | HIGD1B | TEKT3 |
| ATAD3B | CGN | TFCP2L1 | HIST1H1B | TFAP2A |
| AURKA | CHST9 | TIMP3 | HMCN2 | TFRC |
| B4GALNT4 | CIB1 | TMEM243 | HNF1A | THSD7A |
| BAAT | CLDN3 | TMEM246 | HNF4G | TICRR |
| BRWD1 | CLIC5 | TMEM35A | HS6ST2 | TKT |
| BTC | CMTM3 | TMEM79 | HSD17B2 | TLL1 |
| BUB1B | COCH | TNFRSF10A | HSPA12A | TMC4 |
| C19orf57 | COL4A5 | TNFSF11 | HSPA4L | TMEM159 |
| C22orf39 | COLEC12 | TNS1 | HSPB1 | TMEM231 |
| CA13 | CPA6 | TP73 | HSPB7 | TMEM63A |
| CA8 | CPT1B | TPH1 | ICAM1 | TMEM92 |
| CACNA2D2 | CR2 | TPM2 | ICOS | TNFRSF10B |
| CAMK2A | CRABP2 | TRAC | IGF1R | TNFRSF10C |
| CARD14 | CT55 | TRBV20-1 | IGF2BP1 | TNFSF10 |
| CASQ2 | CTSV | TSGA10 | IGFBP2 | TONSL |
| CD1A | CXCL13 | TTC25 | IGFBP5 | TPPP |
| CDC45 | CXCL14 | TUBD1 | IGFBPL1 | TRAF1 |
| CDCA5 | CXCL16 | UBXN10 | IGHV1-58 | TRIM2 |
| CDH3 | CXorf21 | UCK2 | IGHV3-21 | TRIM24 |
| CDKN2D | CYB5D1 | USPL1 | IGHV3-48 | TRIM72 |
| CDON | CYP3A5 | WDHD1 | IGHV3OR16-13 | TSHZ2 |
| CEACAM6 | CYP4F8 | WDR17 | IGKV1-16 | TTC22 |
| CEBPG | DCDC2B | WFDC2 | IGKV3-20 | UGDH |
| CELSR2 | DHRS9 | WNT10A | IGLC3 | UGT1A6 |
| CENPH | DMRTA1 | ZNF606 | IGLV1-40 | UNC45B |
| CEP72 | DNAH11 | ZNF69 | IGLV2-18 | VAMP5 |
| CHEK1 | DNAI1 | ZNF704 | IGSF9 | VIL1 |
| CKMT2 | DNAJB13 | ZWILCH | IL17D | VLDLR |
| CLSPN | DPP10 | ABAT | IL18 | VRK1 |
| CMA1 | DSG2 | ABLIM2 | INPP5J | VWA5A |
| CNKSR2 | EEF1A2 | ABO | IQSEC2 | WDR66 |
| CNN1 | EFHD1 | ACKR1 | IRAK2 | WISP2 |
| COL22A1 | EFNB3 | ACOX2 | ITGA2 | XPO5 |
| CPE | EHD2 | ACSF2 | ITGA3 | XXYLT1 |
| CRABP1 | EIF1AY | ACSM4 | ITPKA | YTHDC1 |
| CRACR2B | EMILIN3 | ACSM5 | IWS1 | ZBTB10 |
| CXCL12 | ENTPD2 | ACTB | JADE1 | ZDBF2 |
| CYP4F3 | ENTPD3 | ADD2 | KANK2 | ZKSCAN1 |
| DKK2 | EPCAM | ADH1A | KANK4 | ZMYND10 |
| DLGAP5 | EPDR1 | ADIPOR2 | KCTD14 | ZNF185 |
| DNASE1L3 | EPN3 | ADK | KCTD16 | ZNF391 |
| DPP4 | ERCC6L | AEBP2 | KHDRBS2 | ZNF503 |
| DUSP4 | ERICH3 | AHSP | KHDRBS3 | ZNF628 |
| EFHC2 | ERN2 | AKAP14 | KIAA1211L | ZNF703 |
| ELN | ERO1B | AKR1B15 | KIAA1841 | ZNF780A |
| EMCN | EVA1C | AKR1C3 | KIAA2026 | ZNF829 |
| EREG | EVL | ALCAM | KIF20B | ZNF91 |
| ERICH5 | FABP5 | ALDH1L1 | KLHDC7B | ZWINT |
| ESPL1 | FAM107A | ALDH3A1 | KLK12 | |
| EXO1 | FAM3B | ALPK3 | KLRK1 | |

TABLE B
SVM biomarkers (486 biomarkers)
Biomarker

| | | | |
|---|---|---|---|
| AGR3 | STMN2 | TACSTD2 | AURKA |
| PID1 | PLPP2 | FCGR3A | RNF168 |
| GBP5 | TNFSF10 | KIFC1 | SERPINB13 |
| CDC20 | LYPD3 | ADGRG1 | NECTIN1 |
| CALHM6 | TESC | KDM7A | PLCH1 |
| CRYBG1 | ACOX2 | ST6GALNAC2 | FCHO1 |
| SKA3 | GRTP1 | SGCD | CASZ1 |
| CLDN4 | CRACR2B | EVL | FAM210B |
| RAB27B | CDC7 | FHL1 | GAREM1 |
| APCS | GCHFR | DSG2 | CYTH3 |
| SLC34A2 | LPIN2 | MTF2 | ACAD8 |
| COL7A1 | ERRFI1 | IRF6 | ABI3BP |
| PKP1 | FMO5 | E2F6 | DCLK2 |
| MET | SERPINB5 | RERG | TACC3 |
| AREG | GBP2 | TSPAN6 | MPP7 |
| AFAP1L2 | TC2N | BAIAP2L1 | KIF22 |
| DHRS9 | FURIN | JARID2 | FADS1 |
| KIAA1324 | PRIM1 | CYTIP | SLCO2B1 |
| KIF1A | AURKB | ACSL5 | EVPL |
| PROX1 | CD247 | DDX21 | GIMAP7 |
| TUBB2B | APOL3 | TNS4 | UNC13B |
| FERMT1 | WNK2 | RAPGEF5 | SELENBP1 |
| CHRDL1 | ASF1B | KIAA0408 | EFNA1 |
| SHCBP1 | ZNF428 | CKAP2 | HDGFL3 |
| GBP1 | OAT | DSC2 | CENPF |
| SDC4 | TAX1BP1 | KIF23 | LAX1 |
| MZB1 | DSP | TMEM132A | PGAP1 |
| TLR3 | CALML3 | RGCC | LYAR |
| HMGB3 | CBFA2T2 | ITGA2 | FLVCR1 |
| SKA1 | ARHGEF19 | SPR | ECHDC2 |
| GPAT3 | ITGA8 | KRT8 | SPATS2L |
| FMO1 | KIT | AGA | ASAH1 |
| AGR2 | TMOD1 | PIK3AP1 | SORT1 |
| CLIC6 | GBP6 | WARS | TYROBP |
| KIF5C | KRT5 | SLC20A2 | ACYP1 |
| SCD5 | RACGAP1 | KMT5C | TBPL1 |
| PLA2G4A | DDAH1 | FSD1L | SNX25 |
| ANXA8L1 | DCBLD2 | LILRB4 | CCNB1 |
| HGD | HEPH | FBLN5 | CKAP2L |
| FAM83F | PHLDA2 | IRF4 | NASP |
| KRT3 | IL1RN | UCK2 | EHD2 |
| IVL | CD79A | FOXP3 | TP73 |
| CRYM | CEP55 | SEC11C | STAT1 |
| CDCA8 | GZMA | APLP2 | MELK |
| ZNF219 | ANLN | MYO5C | OSMR |
| EYA2 | CD274 | FAM46C | PRSS8 |
| DSG3 | ABCC3 | ADA | SMPDL3B |
| WNT5A | MEGF6 | SEMA4A | TTK |
| MYH11 | KRT6A | HSPB6 | FAM83B |
| CIP2A | COMT | DAAM2 | CYP27A1 |
| IGKV3-20 | BANK1 | C16orf89 | TMC4 |
| SYTL2 | IGHG1 | TBC1D30 | SFN |
| FAT2 | MAMDC2 | SGMS2 | PBK |
| SMIM22 | CDS1 | RFX3 | ANK3 |
| SOX2 | SLC1A4 | FAM174B | MCM2 |
| NAMPT | MANSC1 | IQGAP3 | BTLA |
| GBP4 | PDE4D | KRT75 | BCL7A |
| FAAH | SC5D | MARK1 | GIMAP6 |
| SIX1 | ASPH | GLS | FAS |
| KRT15 | FAM111B | BUB1B | KCTD15 |
| CLCA2 | MKI67 | MGLL | CD2 |
| CRMP1 | CLDN3 | FYB1 | EPHA2 |
| AK1 | BCL11A | C11orf54 | ELF3 |
| MEOX2 | DTL | CLSPN | DMD |
| KRT17 | KCNK5 | SIGIRR | STMN1 |
| EDARADD | MTCL1 | TPX2 | FMN1 |
| PCDH7 | ATP1B1 | BDH2 | CAB39L |
| COL14A1 | SPON1 | FCER1G | SEPT1 |
| MEX3A | ERO1B | STK17A | HPGDS |
| IDO1 | THEM6 | RAP1GAP | SAMD8 |
| CAPNS2 | KIF14 | RRAD | HNF1B |
| TRIM29 | GIMAP4 | KIF15 | ABCC1 |
| CNN1 | LEO1 | JAG1 | MAGI3 |
| GPR183 | CXCL16 | MARC1 | ADGRF5 |
| TOP2A | ITGB8 | LTC4S | CLSTN1 |
| PTPN13 | UPP1 | CD19 | CHEK1 |
| CD27 | FBP1 | UHRF1 | ASL |
| GPRC5C | LAMB3 | TIMELESS | RLF |
| FST | TMEM189 | TXNDC16 | SAMD9 |
| TRAC | CKB | ARHGEF38 | GPD1L |
| SCN4B | KRT16 | CHAF1B | ATP11B |
| ECT2 | SLC1A3 | SLC43A3 | VAMP5 |
| HELLS | DDX60 | EPN3 | FGF2 |
| RARG | GCH1 | CELSR2 | ABLIM2 |
| BIRC5 | ADH1B | HSPA4L | GULP1 |
| SFTPB | JAM3 | CTSS | AIF1 |
| CDK1 | PTPRZ1 | ALCAM | DPY19L1 |
| CD38 | GGH | PAF1 | TP63 |
| GINS1 | PIR | SKAP1 | SPAG5 |
| HSD11B1 | VSNL1 | SH2D4A | ARHGEF28 |
| SLC7A2 | TNFRSF10A | HMMR | MILR1 |
| CENPV | BHLHA15 | SLC11A2 | NARF |
| GZMB | GTSE1 | EZH2 | MCM7 |
| CENPU | IL18 | POLA1 | DDR1 |
| STEAP2 | S100A2 | C1orf112 | EPB41L5 |
| COL17A1 | EGFR | LIME1 | KMO |
| LAD1 | KRT6B | PPP1R16B | WDR76 |
| MLPH | GNAZ | RHOU | CD109 |
| C1orf116 | TJP3 | STS | NUP210 |
| CYP2S1 | CDH3 | TAP1 | AKAP12 |
| SORBS1 | VWA5A | GSTM5 | SMARCC1 |
| CENPI | MMP28 | SAMD9L | RBKS |
| CWC22 | SYT7 | SLAMF7 | GJA1 |
| PYGM | TCEANC2 | NEGR1 | HSPA12A |
| EPCAM | NCAPG | ALOX5 | CYB5A |
| KRT6C | GPR39 | CRELD1 | TMEM119 |
| BMP3 | GKAP1 | NFIA | PSIP1 |
| VANGL2 | TMEM45B | LIPH | PIAS2 |
| DNAJC12 | SH2D1A | MAP7 | HLA-DMA |
| SCN7A | DSC3 | PPL | CD5 |
| NAPSA | LDHD | STARD10 | CAMK4 |
| IGF2BP2 | OCLN | SLC2A1 | KIF4A |
| PPP1R14C | RTN1 | NKX2-1 | JADE1 |
| KRT14 | SLC22A23 | CRIM1 | ARFGEF3 |
| MFAP4 | CTR9 | ERCC6L | CDCA2 |
| CDC45 | FASN | FAM221A | CTSO |
| CSTA | ALDH1L2 | GPT2 | ATP11A |
| PCDHB2 | MAOA | MS4A6A | IL3RA |
| FBP2 | CYBA | ATAT1 | IGF1R |
| LAMC2 | ZCCHC3 | KIF11 | PITPNM3 |
| TPM1 | PPP1R9A | CLDN1 | IL3RA |
| PTPRO | ARVCF | RAB3IP | IGF1R |
| | | | PITPNM3 |

TABLE C
SVM biomarkers (200 biomarkers)
Biomarker

| | | | |
|---|---|---|---|
| AGR3 | IGKV3-20 | SORBS1 | CBFA2T2 |
| PID1 | SYTL2 | CENPI | ARHGEF19 |
| GBP5 | FAT2 | CWC22 | ITGA8 |
| CDC20 | SMIM22 | PYGM | KIT |
| CALHM6 | SOX2 | EPCAM | TMOD1 |
| CRYBG1 | NAMPT | KRT6C | GBP6 |
| SKA3 | GBP4 | BMP3 | KRT5 |
| CLDN4 | FAAH | VANGL2 | RACGAP1 |
| RAB27B | SIX1 | DNAJC12 | DDAH1 |
| APCS | KRT15 | SCN7A | DCBLD2 |
| SLC34A2 | CLCA2 | NAPSA | HEPH |
| COL7A1 | CRMP1 | IGF2BP2 | PHLDA2 |
| PKP1 | AK1 | PPP1R14C | IL1RN |
| MET | MEOX2 | KRT14 | CD79A |
| AREG | KRT17 | MFAP4 | CEP55 |
| AFAP1L2 | EDARADD | CDC45 | GZMA |
| DHRS9 | PCDH7 | CSTA | ANLN |
| KIAA1324 | COL14A1 | PCDHB2 | CD274 |
| KIF1A | MEX3A | FBP2 | ABCC3 |
| PROX1 | IDO1 | LAMC2 | MEGF6 |
| TUBB2B | CAPNS2 | TPM1 | KRT6A |
| FERMT1 | TRIM29 | PTPRO | COMT |
| CHRDL1 | CNN1 | STMN2 | BANK1 |
| SHCBP1 | GPR183 | PLPP2 | IGHG1 |
| GBP1 | TOP2A | TNFSF10 | MAMDC2 |
| SDC4 | PTPN13 | LYPD3 | CDS1 |
| MZB1 | CD27 | TESC | SLC1A4 |
| TLR3 | GPRC5C | ACOX2 | MANSC1 |
| HMGB3 | FST | GRTP1 | PDE4D |
| SKA1 | TRAC | CRACR2B | SC5D |
| GPAT3 | SCN4B | CDC7 | ASPH |
| FMO1 | ECT2 | GCHFR | FAM111B |
| AGR2 | HELLS | LPIN2 | MKI67 |
| CLIC6 | RARG | ERRFI1 | CLDN3 |
| KIF5C | BIRC5 | FMO5 | BCL11A |
| SCD5 | SFTPB | SERPINB5 | DTL |
| PLA2G4A | CDK1 | GBP2 | KCNK5 |
| ANXA8L1 | CD38 | TC2N | MTCL1 |
| HGD | GINS1 | FURIN | ATP1B1 |
| FAM83F | HSD11B1 | PRIM1 | SPON1 |
| KRT3 | SLC7A2 | AURKB | ERO1B |
| IVL | CENPV | CD247 | THEM6 |
| CRYM | GZMB | APOL3 | KIF14 |
| CDCA8 | CENPU | WNK2 | GIMAP4 |
| ZNF219 | STEAP2 | ASF1B | LEO1 |
| EYA2 | COL17A1 | ZNF428 | CXCL16 |
| DSG3 | LAD1 | OAT | ITGB8 |
| WNT5A | MLPH | TAX1BP1 | UPP1 |
| MYH11 | C1orf116 | DSP | FBP1 |
| CIP2A | CYP2S1 | CALML3 | LAMB3 |

TABLE D
k-TSP biomarker pairs (1630 biomarker pairs)
Biomarker pairs

| | | | | |
|---|---|---|---|---|
| MKI67-CRYM | LIG1-ABCC3 | AGR2-UPP1 | DSC3-CASP8 | CKB-CTSA |
| DDX21-AGR3 | PRPF19-LPP | PIR-GBP4 | PKP1-FCER1G | RCC2-LGALS3BP |
| CDK1-PRKG1 | MCM3-FAH | IDH2-SAMHD1 | KRT5-VAMP5 | STAG2-SEC23IP |
| TAP1-MYH11 | UHRF1-SSH3 | PIR-DOCK10 | DSG3-RGS3 | PHIP-FAM114A1 |
| GZMA-SORBS1 | MCM6-LPP | TESC-DOK2 | KRT15-TSC22D2 | SMARCB1-AGR2 |
| LCP2-CYBRD1 | CBX5-DUSP3 | ALCAM-RALB | COL7A1-FAM84B | TBPL1-CIB1 |
| WARS-SELENBP1 | FEN1-H6PD | ARFGEF3-RGS3 | ITGA6-FCHO2 | PBRM1-LPIN2 |
| EIF4A1-DPYSL2 | NASP-PCYOX1 | GLCE-NFATC2 | SLC1A5-SH3KBP1 | ZNF512-AGA |
| CHORDC1-GPD1L | MSH6-PARP4 | FN3K-PSMB10 | COL7A1-NFATC2 | ARID2-GALNS |
| RNF213-FN3K | PHF6-SFXN3 | SH3BGRL2-SPATS2L | PTGFRN-ASL | PHIP-SEC23IP |
| KPNA2-SH3BGRL2 | SMARCA5-H6PD | SLC12A2-APOL3 | CALML3-NFATC2 | USP48-JTB |
| GBP2-TPM1 | STMN1-CAPN2 | SLC12A2-VAMP5 | CALML3-NUB1 | MSH2-AGA |
| GBP1-DDAH1 | MSH6-MGLL | TM7SF2-CRYBG1 | PKP1-ADGRE5 | TBPL1-OPLAH |
| PARP14-SORBS3 | MCM5-FAH | SORBS2-SIGLEC1 | KRT16-AVL9 | SPIN1-EPS8L2 |
| STAT1-CYB5R3 | SMC2-RMDN2 | ATP1B1-RALB | SHTN1-EVL | JAM3-AP1M2 |
| TAP1-MFAP4 | HAT1-SFXN3 | CLIC6-CD40 | ESRP1-DOCK2 | ZNF326-TM9SF4 |
| PLEK-MYH11 | PHF6-GALE | KIAA1324-GBP4 | DDX21-IL16 | ZNF326-YIPF6 |
| LCP2-TNXB | HDAC2-TBC1D9B | SH3BGRL2-MAP7D1 | HMGB3-SAMHD1 | TBPL1-COX20 |
| RNF213-SORBS3 | MCM3-RAB27B | NEU1-MET | CLUH-RASAL3 | PDS5B-AGA |
| DDX21-MYO1D | UHRF1-RAB27B | HGD-FAS | MSH6-CD48 | PDXP-CIB1 |
| GBP2-FBLN5 | SMC2-CIB1 | KIAA1324-MET | TRMT6-SH2D1A | MSH2-CD46 |
| EIF4A1-CSRP1 | MCM6-HNMT | AGA-PPP1R18 | MAP7-ABI3 | EHMT1-EPS8L2 |
| FCGR3A-LMOD1 | MCM7-GPD1L | SH3BGRL2-ARHGAP18 | CDK1-DOK2 | UBR7-SIAE |
| SMC2-TLN2 | CDK2-RAB27B | PIR-CRYBG1 | RFC4-PPP1R18 | TUBB2B-IDH1 |
| CALHM6-SGCD | MCM2-NME3 | SORBS2-NFATC2 | HSPD1-LCP1 | MEN1-PRSS8 |
| DDX21-LTBP2 | MDC1-PARP4 | THEM6-ITGAL | CDH1-PLEKHO2 | ZNF512-SEC23IP |
| CHORDC1-MYO1D | MSH6-GALE | PRUNE1-LAIR1 | PSMB5-SH3KBP1 | PBRM1-SHC1 |
| STAT1-CSRP1 | MCM5-TNS1 | SHTN1-CD40 | TTC39C-CD3D | ZNF326-EPS8L2 |
| FCGR1A-LMOD1 | HAT1-PPL | SH3BGRL2-DOK2 | BCCIP-SASH3 | ASF1A-FAM114A1 |
| IDO1-TLN2 | FEN1-LPP | PSMB5-UPP1 | ESRP1-EVL | NASP-ASAH1 |
| PLEK-MFAP4 | ZNF326-FAH | SLC12A2-SIGLEC1 | MAP7-GIMAP7 | ZNF512-SIAE |
| FCGR3A-MFAP4 | SMC2-RBKS | HGD-RGS3 | SHTN1-PPP1R18 | TYMS-SSH3 |
| DDX21-MYH11 | SMARCB1-H6PD | FN3K-IL18 | IPO4-DOCK2 | EYA3-AP1M2 |
| CDK1-SORBS2 | MCM3-PPL | ABCB6-ITGAL | BCCIP-DOK2 | ZNF428-PLA2G4A |
| CRYBG1-SORBS1 | MDC1-RMDN2 | SLC12A2-GIMAP7 | NSUN2-CASP1 | ACYP1-AGA |
| GZMA-TNXB | HAT1-DUSP3 | ATP1B1-UPP1 | CDH1-SH3KBP1 | ZNF326-CTSA |
| NAA20-NFIX | MSH6-CIB1 | FN3K-LAIR1 | CDK1-ARHGAP25 | RBBP4-ASAH1 |
| TAP1-TNS1 | PSIP1-LPP | HMGB3-GBP1 | PIR-SH2D1A | PDXP-COX20 |
| NAMPT-CSRP1 | MSH2-SFXN3 | ADI1-GBP5 | LRPPRC-HCLS1 | PDS5B-SSH3 |
| RNF213-GPD1L | NCBP1-DUSP3 | SH3BGRL2-ITGAL | MPP7-ABI3 | UHRF1-SIAE |
| GBP2-MFAP4 | MCM2-GALE | CLIC6-FCGR1A | ATL2-GIMAP7 | ZNF512-COX20 |
| GZMA-GPD1L | NCAPD2-ENDOD1 | AGR2-IL18 | STAU2-PPP1R18 | ASF1A-AGA |
| LILRB4-SORBS1 | SMARCE1-PPL | PIR-AFAP1L2 | RFC4-WAS | TRIM28-IDH1 |
| RNF213-PCCA | CBX5-PPL | FASN-SAMHD1 | ATL2-DOK2 | USP48-OPLAH |
| FCGR3A-PRKG1 | PRPF19-TNS1 | CLIC6-CRYBG1 | DDX21-PLEKHO2 | PDS5B-GALE |
| RNF213-CSRP2 | STMN1-PCYOX1 | AGA-CASP4 | NSUN2-IL16 | ACYP1-AGR2 |
| FCGR1A-DDAH1 | MCM4-TNS1 | GLCE-FAS | MAP7-FOLR2 | PDXP-EPS8L2 |
| FCER1G-AGR3 | SMC2-PARP4 | PSMB6-GBP1 | CLDN3-LPXN | TYMS-MPP7 |
| RNF213-SLC25A4 | HCFC1-H6PD | FN3K-UPP1 | ESRP1-PPP1R18 | TYMS-ELMO3 |
| LCP2-KANK2 | MSH6-SSH3 | TACC2-SIGLEC1 | CDH1-IL16 | ASF1A-CIB1 |
| GBP1-SELENBP1 | PSIP1-TNS1 | NEU1-ITGAL | MSH2-DOCK2 | PDXP-SSH3 |
| STAT1-DPYSL2 | MSH2-HS1BP3 | PSMB5-GBP5 | CLDN3-PIP4K2A | FRG1-CIB1 |
| WARS-PEBP1 | MCM6-GALE | PRUNE1-VAMP5 | RFC4-SEPT1 | MEN1-SLC35A2 |
| IDO1-TNXB | MCM5-LPP | FN3K-LCP2 | ATL2-SASH3 | ASF1A-EPS8L2 |
| FCGR3A-MAOB | MCM2-PARP4 | SHTN1-TSC22D2 | MSH6-ANKRD44 | SARM1-OPLAH |
| PARP14-FN3K | MCM5-PPL | SLC12A2-DOCK10 | PSMB5-IL16 | STAG2-FAM114A1 |
| CDK1-MFAP4 | PRPF19-DUSP3 | KIF1A-VAMP5 | GGH-CASP1 | SMARCE1-SIAE |
| GZMA-LTBP2 | MCM7-RAB27B | IDH2-EFHD2 | PCNA-HCLS1 | ZNF326-SCAMP2 |
| DDX50-SORBS2 | MCM5-NME3 | HGD-NFATC2 | PUS7-SEPT1 | PDXP-AP1M2 |
| RNF213-TNS1 | PARP1-MVP | PIR-GBP5 | ESRP1-SH3KBP1 | ARID2-OPLAH |
| MAD2L1-TNXB | PHF6-SSH3 | FN3K-EVL | MAP7-SH2D1A | CKB-GFPT1 |
| CHORDC1-TPM1 | MCM3-NME3 | PSMB6-RALB | PIR-RASAL3 | HDAC2-EPS8L2 |
| GBP4-SGCD | HAT1-RAB27B | FN3K-PPP1R18 | BCCIP-GIMAP7 | PSIP1-PCBD1 |
| NAA15-FBLN5 | CBX5-FAH | BROX-APOL2 | TRMT6-SASH3 | PDS5B-TMEM87A |
| LCP2-PRKG1 | PHF6-FAH | SOX2-CD274 | CLUH-SH2D1A | PBRM1-ELMO3 |
| CDK1-TLN2 | TBPL1-ABCC3 | FN3K-CD40 | MAP7-NFATC2 | SPIN1-OPLAH |
| TSC22D2-SORBS2 | MSH6-HS1BP3 | ADI1-TSC22D2 | STAU2-IL16 | FRG1-AP1M2 |
| GBP1-PARVA | SMARCC1-ENDOD1 | TESC-CRYBG1 | CLDN3-SH3KBP1 | UHRF1-EPS8L2 |
| CALHM6-SH3BGRL2 | SMARCA5-DUSP3 | PSMB5-PPP1R18 | PIR-SEPT1 | PBRM1-TACC2 |
| PLEK-TPM1 | EHMT1-TMEM63A | KIAA1324-CD274 | TTC39C-RASAL3 | EYA3-OPLAH |
| OGFR-SLC25A4 | TBPL1-SSH3 | CLIC6-EVL | MAP7-RASAL3 | SMARCE1-GALE |
| STAT1-PEBP1 | PHF6-DUSP3 | SH3BGRL2-PPP1R18 | MPP7-SEPT1 | ARID2-ADAM9 |
| WARS-DPYSL2 | CBX5-GALE | AGA-PSMB10 | PIR-GIMAP7 | RCC2-ASAH1 |
| RNF213-PRKG1 | MCM7-SPR | AGR2-RALB | KPNA2-SASH3 | NASP-LGALS3BP |
| PLEK-AOC3 | PKP1-HNMT | SH3BGRL2-CASP4 | CDK1-LPXN | ACYP1-GALE |
| GBP2-MYH11 | DSG3-OCLN | TESC-VAMP5 | IDI1-SH3KBP1 | ARID2-AP1M2 |
| GBP1-TPM1 | KRT6A-AGR3 | NUDT16L1-NFATC2 | TTC39C-SH2D1A | TBPL1-TMEM87A |
| OGFR-CYBRD1 | VSNL1-CRYM | STARD10-VAMP5 | CLUH-SASH3 | HDAC2-SEC23IP |
| GBP5-MFAP4 | CSTA-ACAD8 | FN3K-SH3KBP1 | EPCAM-IL16 | ASF1A-COX20 |
| KPNA2-PCCA | KRT5-DDAH1 | SLC12A2-AFAP1L2 | MAP7-CD48 | SMARCE1-YIPF6 |
| RNF213-SH3BGRL2 | TRIM29-GPD1L | AGA-TSC22D2 | CDK1-SASH3 | ASF1B-TMEM87A |
| EIF4A1-PEBP1 | DSC3-SLC9A3R2 | TM7SF2-GBP5 | RFC4-DOCK2 | PDXP-AGA |
| OGFR-MFAP4 | DSP-SELENBP1 | SH3BGRL2-CRYBG1 | CDK1-ANKRD44 | TBPL1-ADAM9 |
| NAA15-TNS1 | ABCF3-SH3BGRL2 | THEM6-VAMP5 | PIR-ARHGAP25 | SMARCD1-OPLAH |
| GBP2-TNS1 | DDX21-GALE | TM7SF2-CD274 | MSH2-RASAL3 | ASF1B-OPLAH |
| CRYBG1-ECHDC2 | NECTIN1-CIB1 | NEU1-GBP4 | EPCAM-PLEKHO2 | SMARCD1-MPP7 |
| CDK1-TNXB | ATP1B3-MECP2 | FN3K-ITGAL | MSH6-DOCK10 | ASF1A-AP1M2 |
| CD274-SORBS1 | EIF4G1-DHRS7 | SLC12A2-GBP4 | EPCAM-GIMAP4 | CKB-ASAH1 |
| GZMA-KANK2 | JUP-CAPN2 | AGR2-GBP2 | MAP7-SEPT1 | HMGB2-TKT |
| DDX21-AOC3 | SLC1A5-DDAH1 | THEM6-MAP7D1 | STARD10-LPXN | FRG1-TMEM87A |
| KIF1BP-SH3BGRL2 | PKP1-AGR3 | AGA-SPATS2L | IDI1-PLEKHO2 | DSG3-CLDN3 |
| FCGR3A-LTBP2 | TRIM29-SH3BGRL2 | CLIC6-VAMP5 | MAP7-PHYKPL | KRT16-AP1M2 |
| CASP8-SORBS2 | NOP2-ACAD8 | TM7SF2-GBP4 | SHTN1-DOCK2 | KRT15-YIPF6 |
| CDK1-SORBS1 | DSC3-OCLN | THEM6-SPATS2L | CDH1-PPP1R18 | VSNL1-CYB561 |
| CRYBG1-SGCD | COL7A1-ABCC3 | THEM6-TSC22D2 | RFC4-DAB2 | KRT5-STARD10 |
| GZMA-LMOD1 | PSAT1-FN3K | NUDT16L1-AFAP1L2 | DDX21-CASP1 | SERPINB5-PYCR1 |
| RNF213-LTBP2 | DSG3-CRYM | KIAA1324-DOCK10 | ATL2-RASAL3 | DSC3-FN3K |
| MAD2L1-SORBS1 | KRT6A-GPD1L | ADI1-LCP2 | CDK1-GIMAP7 | ITGB4-SHTN1 |
| CHORDC1-FN3K | KRT5-HNMT | NEDD4L-ITGAL | MSH2-SEPT1 | PKP1-PCBD1 |
| CDK1-DAG1 | VSNL1-LIMCH1 | TUBB2B-UPP1 | PBK-SASH3 | NECTIN1-JTB |
| PLEK-TNS1 | NECTIN1-SLC9A3R2 | STAG2-PARP4 | PBK-ANKRD44 | KRT6B-CIB1 |
| NAMPT-SELENBP1 | DSG3-ACAD8 | GDAP1-MET | PBK-ARHGAP25 | KRT6A-ATP1B1 |
| GBP1-PCYOX1 | KRT6A-HNMT | MSH6-TRIP6 | MCM4-MYOF | TRIM29-PDXDC1 |
| DDX21-FBLN5 | COL7A1-TMEM63A | ZNF512-PARP14 | UHRF1-BIN1 | ITGA6-SH3BGRL2 |
| NAA15-MFAP4 | KRT5-SELENBP1 | CKB-ASPH | MCM5-DUSP3 | DGKA-LRBA |
| TSC22D2-SORBS1 | DSC3-ACSL5 | ZNF326-IFI35 | HAT1-LRP1 | KRT15-CLDN3 |
| DDX21-MAOB | SLC16A1-OCLN | MSH2-RNF213 | CBX5-LPXN | TRIM29-STARD10 |
| GZMA-PRKG1 | MCM2-SH3BGRL2 | SMARCE1-CASP4 | MSH2-SEC24D | DSG3-FN3K |
| MAD2L1-SORBS2 | RFC4-BDH2 | ACTL6A-RALB | SMARCB1-PLEKHO2 | VSNL1-SH3BGRL2 |
| DDX21-TPM1 | TRIM29-OCLN | NCBP1-NCEH1 | SMC3-SCAMP2 | KRT5-HMGB3 |
| TNPO3-TMOD1 | KRT6A-CRYM | RCC2-NAMPT | RCC2-SQOR | PKP1-EPCAM |
| NAA25-SH3BGRL2 | ABCF3-ACSL5 | UBA2-SQOR | NASP-PLEC | S100A2-CYB561 |
| CD274-SORBS2 | NECTIN1-SH3BGRL2 | H1FX-ERO1A | STMN1-TPP1 | SERPINB5-PCBD1 |
| NAMPT-PEBP1 | DSG3-TMEM63A | STMN1-PLIN3 | MCM3-ENTPD1 | CD109-LPIN2 |
| RNF213-AGR3 | IRF6-ECHDC2 | CENPV-PARP4 | MCM2-CTBS | TP63-EPB41L5 |
| TSC22D2-LMOD1 | CSTA-GPD1L | ACYP1-IFI35 | UHRF1-SFXN3 | CASP1-LPCAT1 |
| OGFR-LTBP2 | SLC1A4-MGLL | SPIN1-PARP14 | STAG2-CTBS | TRIM29-PYCR1 |
| TYMP-CSRP1 | SERPINB5-ECHDC2 | ZNF512-MET | MSH6-LRP1 | DSC3-SHTN1 |
| CALHM6-CYBRD1 | PKP1-CRYM | STAG2-RNF213 | ACYP1-PLEKHO2 | NECTIN1-LRBA |
| GBP5-KANK2 | KPNA2-MGLL | ASF1A-NMI | CBX5-SCAMP2 | KRT15-FN3K |
| CRYBG1-SORBS2 | PRMT1-DHRS7 | RPRD1A-SOAT1 | SMARCE1-BIN1 | EGFR-JTB |
| LCP2-LMOD1 | DSP-ERGIC1 | MSH2-RALB | RPRD1A-ARHGAP18 | ITGB4-OCLN |
| KPNA2-CSRP2 | KRT17-CYB5A | SMARCE1-LCP2 | SMC2-RAB43 | CASP1-FN3K |
| CD274-MFAP4 | DSG3-SLC9A3R2 | KAT7-RAB43 | MCM3-PARP4 | CD109-CYB561 |
| CDK1-LMOD1 | TRIM29-ACAD8 | MSH6-PARP14 | STAG2-BIN1 | KRT6A-STARD10 |
| CASP8-TNXB | KPNA2-CIB1 | CBX1-IFI35 | MCM3-LRP1 | JUP-HMGB3 |
| DDX50-HSPA12A | SERPINB5-ACAD8 | UBA2-NAMPT | NASP-SQOR | KRT5-AGR2 |
| CRYBG1-CRYM | DSG3-AGR3 | TRIM28-CALU | RCC2-PLEC | VSNL1-OCLN |
| MKI67-SORBS2 | TRIM29-HNMT | STRBP-TSC22D2 | NCBP2-ARHGAP18 | DSC3-ENPP4 |
| SPCS1-AGR3 | COL7A1-SNX30 | ACYP1-PARP4 | CBX5-PLEKHO2 | KRT6A-CLDN3 |
| SPCS3-ATP1B1 | NOP2-BDH2 | CENPV-CASP8 | MCM3-SEC24D | CASP1-YIPF6 |
| FYB1-ANK3 | NECTIN1-MGLL | ZNF512-CRYBG1 | MCM2-LRP1 | DSG3-CYB561 |
| CD38-SDC4 | CSTA-SLC9A3R2 | MSH2-PARP14 | MSH6-ARHGAP18 | ITGB4-SH3BGRL2 |
| MZB1-CAMK2D | PSAT1-MPRIP | STAG2-CASP4 | HAT1-DAB2 | KRT17-HMGB3 |
| ISG20-TMEM245 | KRT6A-BDH2 | ASF1A-PTPN12 | SMARCE1-LRP1 | VSNL1-LLGL2 |
| HCLS1-DDAH1 | CDK1-CRYM | CKB-NCEH1 | STAG2-ARHGAP18 | SAMD9-OCLN |
| DOCK2-RAB27B | DSC3-RMDN2 | PHF6-RNF213 | SMARCC1-PARP4 | KRT5-ATP1B1 |
| FKBP11-SH3BGRL2 | DSG3-MGLL | PSIP1-NT5C2 | HAT1-EPB41L2 | ITGB4-LPCAT1 |
| PTPRC-C11orf54 | DDX21-DDAH1 | KDM1A-FAS | PHF6-PLEKHO2 | GPC1-OCLN |
| LIMD2-APLP2 | FXR1-GALE | PDS5B-NMI | UHRF1-SH3KBP1 | KRT5-EPCAM |
| CYBA-HSDL2 | SLC1A5-MGLL | MSH2-ARHGAP18 | MCM3-DUSP3 | IRF6-LRBA |
| CORO1A-PRDX5 | ATP1B3-APOOL | SMARCE1-RNF213 | MCM2-PLEKHO2 | KRT6B-SFTPB |
| LPXN-MYO6 | KRT17-PRDX5 | H1FX-NAMPT | ZNF326-LPXN | PKP1-FN3K |
| SH2D1A-ADGRF5 | KRT6A-ACAD8 | UBA2-ERO1A | MCM5-SCAMP2 | COL7A1-AGA |
| SEC11C-SH3BGRL2 | COL7A1-SH3BGRL2 | TRIM28-PLIN3 | SMARCC1-ARHGAP18 | CD109-HGD |
| PTPRC-DDAH1 | NECTIN1-APOOL | CBX1-NMI | PSIP1-SQOR | NECTIN1-RBM47 |
| CD38-ENDOD1 | RFC4-SLC9A3R2 | SMARCE1-PARP14 | NASP-PLIN3 | ITGA6-YIPF6 |
| MZB1-FBLN5 | ATP1B3-FN3K | MSH6-TSC22D2 | STMN1-MVP | S100A2-OCLN |
| SPCS3-PYGB | VSNL1-SIGIRR | STAG2-TRIP6 | HAT1-SEC23A | KRT6B-AP1M2 |
| DOCK2-AGL | ATP1B3-GALE | ZNF326-PARP14 | MCM2-LPXN | PKP1-CD46 |
| UBE2J1-ANK3 | COL7A1-OCLN | MSH2-NMI | MDC1-TOR4A | ITGA6-FN3K |
| LIMD2-SDC4 | TRIM29-APOOL | ZNF512-CASP4 | MCM5-PLEKHO2 | KRT6A-HMGB3 |
| PLCG2-RAB27B | SLC1A5-FN3K | NCBP1-ASPH | MSH2-SH3KBP1 | DSC3-PDXDC1 |
| IRF4-PTPRG | KRT17-ANXA5 | STRBP-PARP4 | ZNF326-H6PD | DSG3-YIPF6 |
| BIN2-ANK3 | SLC1A4-OCLN | ACYP1-CASP4 | PSIP1-LACTB | SERPINB5-BROX |
| CD38-RAB27B | SERPINB5-AGR3 | CENPV-PTPN12 | HAT1-PLEKHO2 | SH3BP1-CYB561 |
| EVL-EVPL | COL7A1-TMEM245 | ASF1A-CASP8 | MCM3-BIN1 | TUBB6-PYCR1 |
| UBE2J1-ADGRF5 | KRT6A-APOOL | MSH2-TRIP6 | HAT1-SEC24D | COL7A1-SHTN1 |
| HCLS1-ATP1B1 | GLRX3-HNMT | DMAP1-TSC22D2 | PRPF19-ZYX | PKP1-YIPF6 |
| SEC11C-RAB27B | JUP-CYB5A | STAG2-SH3KBP1 | SMARCE1-DAB2 | EGFR-CLDN3 |
| PLEK-GPD1L | VSNL1-MGLL | MSH6-FAM114A1 | MCM3-H6PD | CRYBG1-SIGIRR |
| LIMD2-ENDOD1 | MCM2-SLC9A3R2 | ZNF326-FCGR3A | MCM2-H6PD | SERPINB5-LPCAT1 |
| DOCK2-SH3BGRL2 | PRMT1-SELENBP1 | STRBP-MET | MCM3-PLEKHO2 | DSG3-RBM47 |
| UBE2J1-ABCC3 | VSNL1-RMDN2 | ACYP1-SH3KBP1 | MSH6-WDR91 | DSG3-PYCR1 |
| FKBP11-CRYM | SERPINB5-OCLN | PHIP-NMI | MSH6-ADGRE5 | COL7A1-YIPF6 |
| NCKAP1L-MGLL | DSP-DDAH1 | SMARCC1-LCP2 | PRPF19-LACTB | VSNL1-MARS2 |
| BIN2-RAB27B | DDX21-FN3K | SMARCB1-RALB | HAT1-CTBS | ITGB4-PDXDC1 |
| CYBA-MYO6 | COL7A1-MGLL | PSIP1-NCEH1 | CBX5-SH3KBP1 | IFI16-PCBD1 |
| CD14-CAMK2D | DSG3-LIMCH1 | ACYP1-MET | MSH6-BIN1 | TUBB6-BROX |
| CD38-AGL | VSNL1-RAP1GAP | GDAP1-ARHGAP18 | ZNF326-ARHGAP18 | KRT16-CIB1 |
| ISG20-SDC4 | SLC1A5-AGR3 | PHIP-SOAT1 | SMC2-ADGRE5 | DSG3-AP1M2 |
| EVI2B-TMEM245 | ATP1B3-DDAH1 | STMN1-ERO1A | HDAC2-DAB2 | IRF6-TDRKH |
| ARHGAP25-ADGRF5 | CDK1-TMEM63A | SMARCC1-CASP4 | NCBP2-SEC24D | GBP6-AP1M2 |
| CD38-ANK3 | CSE1L-ERGIC1 | PSIP1-MYOF | MDC1-PLXDC2 | DSC3-JTB |
| PLCG2-TMEM245 | DSG3-GPD1L | CHAMP1-NMI | ADNP-CTBS | PKP1-ATP1B1 |
| PLEK-HSDL2 | KRT6A-OCLN | SMARCE1-LYN | PSIP1-ZYX | CD109-CLDN3 |
| CYBA-CDH1 | PKP1-OCLN | CBX1-SH3KBP1 | PHF6-SCAMP2 | CRYBG1-LLGL2 |
| ISG20-ABCC3 | DSC3-CRYM | MSH6-NMI | HAT1-SH3KBP1 | S100A2-SH3BGRL2 |
| NCKAP1L-EVPL | NECTIN1-BDH2 | STAG2-NMI | RPRD1A-DAB2 | VSNL1-SORT1 |
| BIN2-TMEM245 | VSNL1-ABCC3 | CENPV-CRYBG1 | DSG3-GIMAP4 | CD109-B3GAT3 |
| NCKAP1L-RAB27B | PSAT1-DDAH1 | ASF1A-PARP4 | SERPINB5-HNMT | ITGB4-CIB1 |
| CYBA-GPD1L | DDX21-GPD1L | STRBP-PARP14 | PKP1-GCHFR | ITGB4-LRBA |
| WAS-CRYM | MKI67-RGN | ACYP1-TRIP6 | VSNL1-DOK2 | FSCN1-HMGB3 |
| FKBP11-SDC4 | SLC1A5-GPD1L | STAG2-RAB31 | NECTIN1-ADA2 | VSNL1-LPIN2 |
| CD48-PTPRG | CSTA-C11orf54 | PHIP-CASP8 | DSC3-CYP27A1 | PKP1-AGA |
| PLEK-AGR3 | PKP1-ACAD8 | SMARCE1-SOAT1 | TRIM29-CTSS | KRT16-AIFM2 |
| LIMD2-TMEM245 | TMTC3-CRYM | USP48-CD274 | TMTC3-BIN1 | CRYBG1-NKX2.1 |
| UBE2J1-APLP2 | ABCF3-BDH2 | BCL2-MET | DSP-ASAH1 | ITGB4-CLDN3 |
| SEC11C-ENDOD1 | GLRX3-DHRS7 | NCBP1-RALB | SFN-FBP1 | IFI16-BROX |
| CYBA-EVPL | KPNA2-OCLN | MSH6-CASP4 | KRT5-ARHGDIB | KRT6A-PDXDC1 |
| EVL-PPL | NOP2-CIB1 | STAG2-SOAT1 | FERMT1-IKZF1 | ITGB4-TDRKH |
| PLEK-CDH1 | PKP1-DDAH1 | SPIN1-CASP8 | IRF6-INPP5D | KRT15-PYCR1 |
| ISG20-ATP9A | MCM2-RMDN2 | CKB-NT5C2 | ITGB4-ALOX5 | TRIM29-EPCAM |
| PLEK-PPL | VSNL1-SLC9A3R2 | MSH6-RNF213 | DSG3-FERMT1 | KRT6B-FN3K |
| MZB1-HSDL2 | ATP1B3-C11orf54 | ZNF326-PARP4 | NECTIN1-IKZF1 | KRT16-MARS2 |
| UBE2J1-SIGIRR | ABI3BP-MKI67 | ZNF512-NMI | SERPINB5-BIN1 | GPC1-LPIN2 |
| DOCK2-SDC4 | MCEE-MET | GDAP1-PARP4 | ITGB4-ADA2 | COL7A1-FN3K |
| PTPRC-HSDL2 | PTGFRN-PPAT | ADNP-CRYBG1 | TRIM29-GALM | SERPINB5-EPCAM |
| NCKAP1L-CDH1 | LMCD1-DDX21 | PHIP-CD274 | KRT5-ASAH1 | PKP1-BROX |
| LIMD2-SH3BGRL2 | SGCD-BAIAP2L1 | TRIM28-TYMP | KRT16-GIMAP4 | CRYBG1-CYB561 |
| LSP1-SPR | KANK2-MGEA5 | GDAP1-IFI35 | KRT17-TGM2 | EGFR-AGA |
| PLEK-GALE | MYH11-RHEB | ACYP1-SPATS2L | COL7A1-DOCK8 | TRIM29-CLDN3 |
| MZB1-DDAH1 | MYLK-NASP | HDAC2-ARHGAP18 | DSP-HNMT | IFI16-ATP1B1 |
| SEC11C-SDC4 | TPM1-GBP5 | RPRD1A-RAB31 | TMTC3-APBB1IP | CD109-OCLN |
| SPCS1-FBLN5 | CYBRD1-CDK1 | CENPV-MET | TP63-CD3E | NECTIN1-AP1M2 |
| CYBA-AGR3 | MYL9-UPP1 | CKB-RALB | DSP-CTSS | KRT6B-AGR2 |
| HSPA13-TMEM245 | AKAP12-SDC4 | CBX1-FCGR3A | SERPINB5-LPXN | NECTIN1-UHRF1 |
| LPXN-ECHDC2 | SORBS1-CASP8 | PSIP1-IFI35 | DSG3-ABI3BP | EGFR-PDXP |
| SEMA4A-RAP1GAP | CNRIP1-TK1 | STMN1-NAMPT | FERMT1-INPP5D | SERPINB5-PSIP1 |
| SEC11C-CRYM | MYH10-ASPH | PBRM1-TSC22D2 | TRIM29-MECP2 | ABCC1-MYEF2 |
| ISG20-ANK3 | KANK2-DNAAF5 | ZNF326-DPYD | IRF6-ALOX5 | CYP2S1-CENPV |
| CD48-SIGIRR | LTBP2-GBP5 | PCIF1-CASP8 | KRT6A-LPXN | GPC1-KDM1A |
| UBE2J1-TM7SF2 | AKAP12-TMBIM6 | ADNP-SOAT1 | KRT17-LCP1 | LAD1-ARHGEF2 |
| SPCS3-MAL2 | MYO1D-DDX21 | DMAP1-CD274 | COL7A1-CD84 | IGF2BP2-ACYP1 |
| BIN2-SDC4 | CSRP2-IGF2BP2 | PHF6-SH3KBP1 | ITGB4-CYP27A1 | DSG3-SPIN1 |
| FYB1-TM7SF2 | TGFB1I1-MGEA5 | SMARCC1-RAB31 | VSNL1-DOCK8 | KRT6A-PDS5B |
| SPCS1-PYGB | SGCD-CRYBG1 | DMAP1-MET | KRT5-TGM2 | KRT5-TUBB2B |
| ARHGAP25-ARFGEF3 | SORBS1-BAIAP2L1 | TBPL1-CD274 | RFC4-ALOX5 | PKP1-CBX5 |
| CYBA-TMEM245 | MYH11-INF2 | CBX1-RALB | FAM83H-CD3E | TRIM29-STAG2 |
| ISG20-RGN | SORBS2-TK1 | PCIF1-TSC22D2 | DSC3-ADA2 | DSC3-ZNF512 |
| CD3E-PTPRG | CORO2B-MKI67 | TBPL1-CASP8 | SLC2A1-FBP1 | KRT17-RCC2 |
| CD38-TMEM245 | CYBRD1-IGF2BP2 | ZNF326-SH3KBP1 | RFC4-BIN1 | KRT6A-UHRF1 |
| DOCK2-CRYM | CSRP2-CDK1 | PSIP1-RALB | KRT15-FBP2 | EGFR-SMARCD1 |
| NCKAP1L-PPL | TLN2-CD274 | RPRD1A-FAM114A1 | DSG3-CYP27A1 | TRIM29-CENPV |
| PLCG2-SDC4 | TPM1-RHEB | PDS5B-SOAT1 | DSC3-ALOX5 | PKP1-MYEF2 |
| ISG20-RASAL3 | SGCD-SPATS2L | SMARCB1-NT5C2 | FAM83H-DOK2 | SERPINB5-ACYP1 |
| LPXN-CDH1 | METTL7A-ASPH | SERPINB5-UPP1 | FXR1-NCKAP1L | DSC3-RPRD1A |
| SPCS1-ATP1B1 | KANK2-IGF2BP2 | DSG3-MET | ITGB4-EVL | DGKA-PDS5B |
| ISG20-SIGIRR | MYH11-SDC4 | KRT6B-NFATC2 | DSP-CD74 | ABCC1-ARHGEF2 |
| FKBP11-ECHDC2 | CORO2B-CD274 | PKP1-ITGAL | CALML3-NCKAP1L | JUP-RBBP4 |
| FASN-CAVIN1 | CNRIP1-BAIAP2L1 | TRIM29-AVL9 | NECTIN1-BIN1 | HSPB1-H2AFY |
| FXR2-TNXB | CNN1-RHEB | KRT6A-GBP5 | SERPINB5-NCKAP1L | CD109-UBR7 |
| NOP2-SORBS1 | LMCD1-INF2 | KRT16-ABR | DSC3-INPP5D | LAMB3-PDXP |
| KPNA2-PRKG1 | TMEM119-BAIAP2L1 | COL7A1-RGS3 | IRF6-ABI3BP | KRT6A-TRIM29 |
| IPO4-KANK2 | AKAP12-TSC22D2 | DSC3-ASL | CSNK1A1-DOCK8 | KRT16-CENPV |
| HMGB3-DPYSL2 | PRKG1-MKI67 | VSNL1-TSC22D2 | FAM83H-SNTB1 | ITGB4-KDM1A |
| PRMT1-RSU1 | TNS1-DDX21 | KRT5-GLS | VSNL1-CD84 | ABCC1-MDC1 |
| DDX21-SORBS3 | AOC3-CDK1 | KRT17-GALM | JUP-FBP2 | FERMT1-CHAMP1 |
| CDK1-AGO1 | CNRIP1-SMC2 | SERPINB3-FCHO2 | IGF2BP2-ALOX5 | FERMT1-STAG2 |
| MKI67-PTPRG | ABI3BP-CDH3 | ITGB4-LCP2 | DSG3-LPXN | LAMB3-CENPV |
| OAT-MFAP4 | CNN1-GBP5 | ITGA6-ARHGAP18 | SERPINB5-MECP2 | KRT6A-ACYP1 |
| POLR2H-ITGA1 | AKAP12-PPAT | COL7A1-MET | FAM83H-ARHGEF6 | TRIM29-RPRD1A |
| ESRP1-SGCD | MCEE-MKI67 | PKP1-SPATS2L | JUP-FBP1 | IGF2BP2-PDS5B |
| NAA25-TLN2 | MYH11-GBP5 | SERPINB5-INF2 | KRT16-CYP27A1 | MEMO1-ARHGEF2 |
| MTHFD2-PDLIM2 | KANK2-MET | ITGA6-ASL | KRT5-CD74 | KRT6A-CBX5 |
| KPNA2-TNXB | CSRP2-MGEA5 | KRT17-ASPH | FXR1-SH3KBP1 | SERPINB5-HAT1 |
| HMGB3-CAVIN1 | MYLK-UPP1 | ITGB4-FCHO2 | SLC2A1-HNMT | ABCC1-CENPV |
| PUS1-SORBS1 | NDUFA4-PKP3 | DSG3-UPP1 | GPC1-CD48 | SDC1-PSIP1 |
| NAA20-TLN2 | METTL7A-NASP | KRT6B-CD274 | KRT5-FBP1 | EGFR-UHRF1 |
| HDAC2-PTPRG | EFEMP2-CD274 | KRT14-TSC22D2 | JUP-ASAH1 | NECTIN1-PDS5B |
| TARS-HDGFL3 | ABI3BP-TK1 | VSNL1-FAM84B | RFC4-INPP5D | CD109-TRIM33 |
| MTHFD2-PRKG1 | MAOB-SDC4 | DSC3-ITGAL | PKP1-LPXN | GPC1-CENPV |
| CLUH-TNXB | SGCD-DNAAF5 | KRT16-UPP1 | KRT15-CYP27A1 | LAD1-CBX5 |
| MTHFD2-TLN2 | AKAP12-MET | KRT6B-ASL | IRF6-ITGAL | BAG3-STAG2 |
| CACYBP-PARVA | LMOD1-MKI67 | COL7A1-CD274 | DSC3-GCHFR | KRT6A-TUBB2B |
| CDK1-CNRIP1 | SGCD-TK1 | KRT5-GALM | SLC2A1-CD74 | TRIM29-ZNF512 |
| DDX21-MFAP4 | LTBP2-TMBIM6 | DSC3-FCHO2 | FERMT1-CYP27A1 | TACSTD2-HAT1 |
| TARS-HNMT | MYL9-FAM3C | KRT17-GLS | KRT6A-GALM | TRIP6-STAG2 |
| CLUH-SYNPO | MCEE-CRYBG1 | NECTIN1-VAMP5 | SERPINB5-APBB1IP | IRF6-SMARCD1 |
| OAT-AOC3 | MYO1D-INF2 | TMTC3-SOAT1 | COL7A1-ABI3BP | ITGB4-ACYP1 |
| FXR2-CNRIP1 | MYH11-HDGFL3 | KRT15-AVL9 | RFC4-CYP27A1 | KRT17-RBBP4 |
| LARP4B-SORBS1 | LTBP2-DDX21 | KRT14-UPP1 | DSP-GALM | EGFR-MDC1 |
| POLR2H-ADD1 | CSRP2-SDC4 | TRIM29-CASP8 | GPC1-TPK1 | ABCC1-KDM1A |
| HMGB3-AHNAK | AKAP12-CD274 | KRT17-GBP5 | SLC2A1-ASAH1 | CYP2S1-TRIM33 |
| OAT-MYLK | PTGFRN-IGF2BP2 | SERPINB5-RNF213 | IRF6-GRAP2 | DGKA-ZNF512 |
| MTHFD2-TNXB | TNS1-FAM3C | FERMT1-ITGAL | COL7A1-DOK2 | DSG3-UBR7 |
| MKI67-PHYKPL | CORO2B-BAIAP2L1 | KRT5-FAM84B | IRF6-CD84 | COL7A1-PDXP |
| CHORDC1-MAOB | NDUFA4-IGF2BP2 | DSG3-CD274 | PKP1-FBP1 | DSC3-KAT7 |
| PRMT1-PARVA | DDX10-TK1 | KRT6B-TSC22D2 | FAM83H-INPP5D | TRIP6-PDS5B |
| OAT-CAMK2D | TGFB1I1-CDK1 | KRT16-MET | SFN-FBP2 | IGF2BP2-UHRF1 |
| NUP210-BDH2 | CNN1-DDX21 | DSC3-NFATC2 | ATP1B3-IL16 | NECTIN1-MDC1 |
| NOP2-CYBRD1 | AOC3-PKP3 | KRT6A-CD2AP | PKP1-GIMAP4 | FERMT1-PDS5B |
| CDK1-HYI | SGCD-IGF2BP2 | KRT15-INF2 | SLC2A1-GALM | KRT6A-MYEF2 |
| EIF4A1-APCS | MCEE-DNAJC2 | SERPINB5-CD2AP | NECTIN1-ALOX5 | PKP1-FRG1 |
| NAA25-AGO1 | SORBS1-CDH3 | ITGA6-ITGAL | DSP-FBP1 | COL7A1-MEN1 |
| IPO4-SORBS1 | COL14A1-ASPH | KRT14-ASL | NECTIN1-APBB1IP | SFN-TUBB2B |
| OAT-MECP2 | EFEMP2-SPATS2L | KRT15-UPP1 | CALML3-SH3KBP1 | CYP2S1-PBRM1 |
| NOP2-PDLIM2 | MYO1D-FAM3C | ITGB4-VAMP5 | SERPINB5-ALOX5 | COL7A1-CHN1 |
| NUP210-VWA5A | MCEE-CD274 | SERPINB3-DOK2 | DSG3-SEPT1 | TRIM29-MEN1 |
| NAA25-SYNPO | AOC3-DDX21 | KRT6B-UPP1 | SERPINB5-EVL | ITGB4-CENPV |
| CACYBP-HNMT | CNRIP1-MET | KRT16-FCHO2 | TP63-HMCES | CYP2S1-MDC1 |
| OAT-MYO1D | CSRP1-NAMPT | KRT15-SH3KBP1 | KRT5-FBP2 | IGF2BP2-ZNF512 |
| HDAC2-PDLIM2 | SGCD-MGEA5 | COL7A1-ASL | KRT15-FBP1 | COL7A1-ZNF512 |
| FXR2-HYI | MYO1D-RHEB | DSC3-VAMP5 | SERPINB5-SEPT1 | KRT17-TUBB2B |
| PRMT1-MYLK | HSPB6-CD274 | TRIM29-ASL | DSC3-NCKAP1L | CD109-PDXP |
| CDK1-TLR3 | PRELP-ASPH | CALML3-AVL9 | LSG1-MSR1 | TRIP6-ARHGEF2 |
| FXR2-TLN2 | MYH11-UPP1 | IRF6-NFATC2 | FAM83H-CYP27A1 | IRF6-ZNF512 |
| MKI67-CNRIP1 | CNN1-PKP3 | DSC3-ABR | COL7A1-ALOX5 | ABCC1-PDS5B |
| MKI67-TPK1 | MAOB-PPAT | ITGA6-SOAT1 | UHRF1-JTB | IGF2BP2-SMARCE1 |
| PRMT1-FHL1 | CSRP2-TSC22D2 | KRT5-UPP1 | UBR7-PLA2G4A | TRIM29-ARHGEF2 |
| SRSF9-TNS1 | EFEMP2-DNAAF5 | DSG3-FAM84B | STAG2-AGA | KRT6A-STAG2 |
| BCCIP-CNRIP1 | ABI3BP-MAD2L1 | VSNL1-ACBD5 | ACYP1-YIPF6 | ITGB4-ASF1A |
| NOP2-KANK2 | TPM1-DDX21 | DSC3-NUB1 | CKB-LGALS3BP | FERMT1-MDC1 |
| BCCIP-TNXB | ABI3BP-CD274 | DSG3-CASP8 | MSH2-TM9SF4 | TACSTD2-CBX5 |
| FXR2-TANGO2 | NFIX-MKI67 | COL7A1-TSC22D2 | CBX5-GLB1 | SDC1-NCBP1 |
| ESRP1-ITGA1 | CNN1-IGF2BP2 | SERPINB5-SH3KBP1 | PSIP1-GFPT1 | NECTIN1-RPRD1A |
| MKI67-TNXB | MCEE-KPNA2 | CALML3-FCHO2 | NASP-FASN | GPC1-PBRM1 |
| DDX21-ADD1 | TAGLN-STAT1 | KRT6A-AVL9 | ZNF326-AGR2 | KRT6A-RPRD1A |
| CDK1-PRKCA | SGCD-DNAJC2 | COL7A1-UPP1 | PDS5B-COX20 | TRIM29-CBX5 |
| TARS-TNS1 | TLN2-NCAPG | TRIM29-NUB1 | TUBB2B-ASAH1 | LAMB3-KDM1A |
| CLUH-CNRIP1 | FBLIM1-MKI67 | KRT6B-MET | SMARCB1-ALDH18A1 | S100A2-PDXP |
| LARP4B-PDLIM2 | MYH11-FAM3C | VSNL1-CASP8 | UBA2-CTSA | COL7A1-ARHGEF2 |
| CDK1-GHDC | ABI3BP-DNAJC2 | CALML3-ASL | ASF1A-TMEM87A | IGF2BP2-STAG2 |
| MCM4-SPR | FBLIM1-BAIAP2L1 | KRT14-AVL9 | UHRF1-YIPF6 | LAMB3-SPIN1 |
| MCM7-NME3 | PRKG1-CDK1 | KRT6B-FAM84B | PBRM1-OPLAH | DSG3-CENPV |
| MCM2-RAB27B | PRKG1-MAD2L1 | KRT6B-AVL9 | RPRD1A-AGA | LAD1-ZNF512 |
| UHRF1-PARP4 | TMEM119-MGEA5 | VSNL1-CD274 | H1FX-FASN | BAG3-ARHGEF2 |
| MSH6-SFXN3 | CNRIP1-MAD2L1 | SLC1A5-RNF213 | PDS5B-EPS8L2 | KRT5-PSIP1 |
| HAT1-FAH | SORBS1-MKI67 | KRT6B-CASP8 | ACYP1-SCAMP2 | KRT5-RBBP4 |
| MCM5-GALE | LMOD1-MGEA5 | VSNL1-NFATC2 | TYMS-LPIN2 | LAD1-PDXP |
| MSH2-SSH3 | CNN1-FAM3C | KRT17-CD274 | ASF1A-JTB | SERPINB5-MYEF2 |
| RCC2-HNMT | SGCD-CASP8 | KRT6A-CASP8 | HMGB2-IDH1 | COL7A1-PDS5B |
| SMC2-TBC1D9B | SORBS1-CRYBG1 | PKP1-SDSL | JAM3-PLA2G4A | TRIP6-PDXP |
| HDAC2-CIB1 | AFAP1L2-CD274 | SLC1A5-VAMP5 | UHRF1-FAM114A1 | LAD1-HAT1 |
| NASP-SUCLG2 | STARD10-SH3KBP1 | SDC1-FKBP5 | ASF1A-OPLAH | PKP1-SMARCD1 |
| STMN1-PRDX5 | IDI1-PPP1R18 | KRT5-TSC22D2 | USP48-CIB1 | KRT6A-UBR7 |
| RBBP4-PCYOX1 | FN3K-GBP5 | KRT17-AVL9 | STMN1-ASAH1 | TRIM29-SPIN1 |
| PARP1-ARHGAP1 | TESC-NFATC2 | KRT15-ASL | TYMS-SLC35A2 | COL7A1-CENPV |
| MCM4-GPD1L | SORBS2-MET | KRT5-MET | PHIP-JTB | IRF6-ASF1B |
| MCM3-SPR | HMGB3-WARS | IRF6-TSC22D2 | UHRF1-AGA | DSG3-SMARCD1 |
| UHRF1-MGLL | THEM6-CD40 | FERMT1-ASL | SMARCA5-GLB1 | PKP1-UBR7 |
| MCM2-SFXN3 | CLIC6-ARHGAP18 | KRT14-ITGAL | SMARCE1-TM9SF4 | KRT6A-SPIN1 |
| MCM6-TNS1 | SH3BGRL2-GIMAP7 | DSG3-TSC22D2 | H1FX-ASAH1 | TRIM29-EHMT1 |
| MSH6-RAB27B | ATP1B1-TAP1 | ITGB4-DOK2 | PDS5B-FAM114A1 | TRIM29-TRIM33 |
| MSH2-H6PD | PRUNE1-DOK2 | KRT6B-RGS3 | STAG2-SIAE | TACSTD2-PDS5B |
| HDAC2-SSH3 | KIAA1324-CRYBG1 | KRT15-MET | ACTL6A-AGR2 | LAMB3-USP48 |

TABLE E
k-TSP biomarker pairs (225 biomarkers)
Biomarker pairs

| | | |
|---|---|---|
| CORO1A-PRDX5 | DDX21-MFAP4 | GLCE-NFATC2 |
| TUBB2B-UPP1 | CDK1-ARHGAP25 | RFC4-PPP1R18 |
| MCM4-MYOF | VSNL1-SH3BGRL2 | SERPINB5-BIN1 |
| HMGB3-WARS | MSH6-SFXN3 | SERPINB5-PCBD1 |
| MKI67-CRYM | RCC2-NAMPT | DDX21-MYO1D |
| PSIP1-GFPT1 | ZNF512-MET | CNN1-GBP5 |
| STARD10-SH3KBP1 | KRT6A-GBP5 | KRT6B-CD274 |
| FASN-CAVIN1 | CHORDC1-GPD1L | ZNF326-IFI35 |
| SHTN1-EVL | TESC-DOK2 | NASP-FASN |
| CKB-ASPH | SERPINB5-INF2 | MTHFD2-TLN2 |
| PKP1-GCHFR | HDAC2-CIB1 | POLR2H-ITGA1 |
| KPNA2-PRKG1 | PKP1-AGR3 | STMN1-PLIN3 |
| RCC2-HNMT | MSH2-RNF213 | DSC3-INPP5D |
| MCM5-DUSP3 | HSPB1-H2AFY | KRT5-HMGB3 |
| TAP1-MYH11 | CLUH-TNXB | ITGB4-ACYP1 |
| HSPD1-LCP1 | NCBP1-NCEH1 | DSG3-CRYM |
| IPO4-KANK2 | SMARCE1-PARP14 | SFN-FBP1 |
| CKB-LGALS3BP | TRIM29-AVL9 | STMN1-ASAH1 |
| STMN1-TPP1 | KRT17-RCC2 | DSC3-ACSL5 |
| CBX5-GLB1 | MCM5-GALE | TRIM29-OCLN |
| EGFR-JTB | SGCD-CRYBG1 | DSG3-FAM84B |
| ESRP1-SGCD | UBA2-SQOR | IRF6-INPP5D |
| WARS-SELENBP1 | COL7A1-MET | TRIM29-APOOL |
| MCM4-SPR | DDX21-PLEKHO2 | KRT14-TSC22D2 |
| CLUH-RASAL3 | TRIM29-GALM | VSNL1-FAM84B |
| UBR7-PLA2G4A | SPCS3-ATP1B1 | SMARCB1-PLEKHO2 |
| SERPINB5-PSIP1 | MCM2-RAB27B | STAG2-PARP4 |
| KRT16-ABR | SMC2-TBC1D9B | DSC3-CYP27A1 |
| TGFB1I1-MGEA5 | CSRP2-CDK1 | CDK1-CNRIP1 |
| SMC3-SCAMP2 | KIAA1324-CRYBG1 | MCM2-CTBS |
| MSH2-TM9SF4 | KRT17-ASPH | STAG2-ARHGAP18 |
| PUS1-SORBS1 | STAG2-AGA | SMARCB1-ALDH18A1 |
| MCM7-NME3 | ABI3BP-MKI67 | DSC3-RPRD1A |
| NOP2-ACAD8 | STAG2-PARP4 | DSG3-SPIN1 |
| MSH6-SFXN3 | UHRF1-FAM114A1 | SMC2-TLN2 |
| HAT1-FAH | GBP2-TPM1 | BIN2-RAB27B |
| CLIC6-ARHGAP18 | FYB1-ANK3 | VSNL1-LIMCH1 |
| SORBS2-MET | CBX5-LPXN | DDX21-IL16 |
| DDX21-GALE | NECTIN1-IKZF1 | PSMB5-SH3KBP1 |
| EIF4G1-DHRS7 | EGFR-PDXP | ITGB4-CYP27A1 |
| MCEE-MET | ABCF3-SH3BGRL2 | ABCC1-MDC1 |
| ALCAM-RALB | PTGFRN-PPAT | SPCS3-PYGB |
| UHRF1-BIN1 | TRMT6-SH2D1A | RBBP4-PCYOX1 |
| VSNL1-DOK2 | ESRP1-DOCK2 | MYH11-INF2 |
| PKP1-MYEF2 | KANK2-IGF2BP2 | ITGB4-ALOX5 |
| NASP-SUCLG2 | H1FX-ERO1A | KRT5-ASAH1 |
| KRT16-GIMAP4 | SMARCE1-LRP1 | DSG3-FN3K |
| MKI67-PTPRG | MCM3-SEC24D | NECTIN1-LRBA |
| MAP7-ABI3 | PRMT1-RSU1 | S100A2-CYB561 |
| MSH6-CD48 | PIR-GBP4 | CD109-CYB561 |
| RCC2-SQOR | PDS5B-COX20 | CYBA-HSDL2 |
| LPXN-MYO6 | STMN1-PRDX5 | ARFGEF3-RGS3 |
| ACYP1-YIPF6 | ASF1A-JTB | TESC-NFATC2 |
| EIF4A1-DPYSL2 | DSC3-SHTN1 | DSC3-ASL |
| UHRF1-SSH3 | EIF4A1-DPYSL2 | TUBB2B-ASAH1 |
| MYLK-NASP | SORBS1-CASP8 | CYBRD1-CDK1 |
| JUP-RBBP4 | ATP1B1-TAP1 | SLC12A2-GIMAP7 |
| HMGB3-SAMHD1 | LCP2-TNXB | DOCK2-SH3BGRL2 |
| NASP-PLEC | ITGA6-ARHGAP18 | KRT5-TUBB2B |
| PKP1-EPCAM | TRIM29-RPRD1A | CD38-SDC4 |
| PARP14-SORBS3 | IDH2-SAMHD1 | KRT15-INF2 |
| PTPRC-C11orf54 | MSH6-TRIP6 | KRT5-GLS |
| ACTL6A-RALB | KRT17-TGM2 | CDH1-IL16 |
| PKP1-SPATS2L | DOCK2-AGL | NAMPT-CSRP1 |
| DSG3-FERMT1 | HCLS1-DDAH1 | RNF213-FN3K |
| KRT16-AP1M2 | SPCS1-AGR3 | PARP1-ARHGAP1 |
| TRIM29-STARD10 | HMGB3-DPYSL2 | PHF6-SFXN3 |
| KRT5-HNMT | PRMT1-PARVA | COL7A1-ABCC3 |
| SLC1A5-DDAH1 | DGKA-PDS5B | COL7A1-SH3BGRL2 |
| IDI1-PPP1R18 | ISG20-TMEM245 | SERPINB5-ECHDC2 |
| BCCIP-DOK2 | KANK2-DNAAF5 | COL7A1-SHTN1 |
| KRT15-CLDN3 | SORBS2-TK1 | CD109-UBR7 |
| NECTIN1-UHRF1 | CBX1-IFI35 | H1FX-FASN |
| GZMA-SORBS1 | DGKA-LRBA | CALHM6-SGCD |
| MZB1-FBLN5 | MYH10-ASPH | STAT1-CYB5R3 |

TABLE F
SVM-peptide biomarkers (581 biomarkers)

| Biomarker | Gene |
|---|---|
| SEIDLNLIK | ANXA8L1 |
| SPEVLLGSAR | CDK1 |
| SDPVTLNVLYGPDVPTISPSK | CEACAM6 |
| VLTPELYAELR | CKB |
| VGDSVDAEGPAGDSVDAEGR | CLIC6 |
| LGTQHPESNSAGNDVFAK | CLIC6 |
| AYPAIGTPIPFDK | COL10A1 |
| LYFEELSLER | CPS1 |
| VTVSEEEILPATR | CRYBG1 |
| QFYSVFDR | CTSE |
| LGTFEVEDQIEAAR | DPP4 |
| VVIVDPETNK | DSP |
| VTVLGQPK | IGLL5 |
| LLEGEDAHLTQYK | KRT17 |
| TEAESWYQTK | KRT5 |
| DAQPSFSAEDIAK | MCM3 |
| ELISDNQYR | MCM3 |
| IQETQAELPR | MCM6 |
| LANLPEEVIQK | MSH6 |
| LLYNSTDPTLR | MYO1G |
| PGPQSPGSPLEEER | NCF1 |
| IIEGEPNLK | PIGR |
| EELIADALPVLADR | PKP1 |
| IDPYVFDR | PLA2G4A |
| ELPGFLQSGK | S100P |
| QFVEQHTPQLLTLVPR | SFTPB |
| PDQGIVIPLQYPVEK | SH2D1A |
| QLEAELGAER | SPR |
| VLTDELK | TGFBI |
| INVYYNEATGNK | TUBB2B |
| IIAVDINK | ADH1B |
| IDAASPLEK | ADH1B |
| FGFLLPESR | CPA3 |
| VGDYGSLSGR | CTSZ |
| VQAELDQVVGR | CYP1B1 |
| GEGAGQPSTSAQGQPAAPAPQK | HMGA2 |
| ESLVVNYEDLAAR | MCM2 |
| LHPDDVAGIQALYGK | MMP19 |
| TLILDVPPGVEK | PTPRC |
| LLLDTFEYQGLVK | CENPV |
| LQEQEQLLK | GBP1 |
| FDASVDQASINPGK | HK3 |
| NWQDYGVR | MZB1 |
| LVLLGGEEEGPR | RASAL3 |
| ETEVIDPQDLLEGR | SDC4 |
| DEDEDIQSILR | LAD1 |
| DTDEADLVLAK | CBX5 |
| DETFNLPR | GBP1 |
| AIASIPTER | ITGA2 |
| GINSSNVENQLQATQAAR | KPNA2 |
| DVEAWFFSK | KRT15 |
| DYSQYYR | KRT17 |
| AVEPQLQEEER | PLTP |
| ADIGAPSGFK | WAS |
| VPSIELR | COL7A1 |
| DLEGSDIDTR | CSE1L |
| GGSLLAGGGGFGGGSLSGGGGSR | KRT15 |
| LEGDSEFLQPLYAK | SGCD |
| ENVVQSVTSVAEK | SNCG |
| GLGVGFGSGGGSSSSVK | KRT5 |
| TVQGPPTSDDIFER | OAT |
| SFFTDDDK | ARHGDIB |
| IEEFLEEK | CLIC6 |
| EELAEALK | MCM4 |
| INHGILYDEEK | CFHR1 |
| SQNPVQPIGPQTPK | MMP1 |
| LLALGDSGVGK | RAB27B |
| YGLNELK | ABCD3 |
| ETGYTELVK | ASPH |
| INQEELASGTPPAR | FYB1 |
| IQDLPPVDLSLVNK | KYNU |
| IDAAVFNPR | MMP12 |
| EPLPLAVK | ABCA3 |
| SHIEIIR | GSR |
| ADYDTLSLR | PKP3 |
| EVDIYTVK | PRMT1 |
| ASNLLLNYK | VRK1 |
| HLSPDGQYVPR | AGR2 |
| SINGILFPGGSVDLR | GGH |
| TSSQPGFLER | TMEM43 |
| AQGGDGVVPDTELEGR | LAMC2 |
| GENLVHQIQYR | CD163 |
| GQHLSDAFAQVNPLK | GSTT1 |
| AGILTTLNAR | MCM7 |
| DNFDIAEGVR | OAS2 |
| EFLETVK | H2AFY2 |
| IEPHHTAVLGEGDSVQVENK | PIR |
| SGFSSVSVSR | KRT6A |
| SLEQNIQLPAALLSR | MCM7 |
| AGEIVVFK | SEC11C |
| SENFEELLK | CRABP2 |
| SLDNFFAK | CDV3 |
| VYTVDLGR | PIGR |
| EAVGGLQTVR | TAP2 |
| VESLDVDSEAK | NASP |
| YQYLLTGR | TIMP3 |
| YLDNPNALTER | KANK2 |
| DPANFQYPAESVLAYK | NQO1 |
| PVDFTGYWK | RBP1 |
| IEEELGDEAR | ENO2 |
| TIDDLEETLASAK | TPM2 |
| LTDEQVALVR | BOP1 |
| DDQITLDEFK | VSNL1 |
| EDAVSAAFK | LUM |
| LSLLLNDISR | TRIP13 |
| DIYSSFGFPR | MMP1 |
| ATASEQPLAQEPPASGGSPATTK | LAD1 |
| LTDAQILTR | RFC2 |
| APETFDNITISR | SLC34A2 |
| IGYSWYK | CEACAM6 |
| NTQIQVLPEGGETPIFK | SCIN |
| QVTVLELFR | SLC2A3 |
| ANPQLGAYAPPPHVIGR | APOL2 |
| LFDVGGQR | GNAO1 |
| ILQISPEGPLQGR | DOCK10 |
| LLGASELPIVTPALR | KPNA2 |
| GEQWTPIEPK | CYBA |
| ALEAVPAPPASGPR | CRYBG1 |
| AFEESLSTLK | ECE1 |
| PNIPEAIR | ERLIN2 |
| LADLTGPIIPK | TMOD1 |
| QVLLGDQIPK | PSMB6 |
| ENYNQYDLNR | CPM |
| NANTFISPQQR | MGP |
| FSLQDPPNK | UGDH |
| ATLPVFDK | BCAT1 |
| PGAFIPGAPVQPVVLR | LPCAT1 |
| NIETIINTFHQYSVK | S100A9 |
| IEPNLPSYR | DPP4 |
| ALNSAANNVYQYGR | TMEM245 |
| LTWHAYPEDAENK | CBX5 |
| INAQLPLTDK | DCAKD |
| AALDEQFEPQK | HPGD |
| VSVGEFSAEGEGNSK | STAU2 |
| QGGFLGLSNIK | MUC1 |
| AGVVTPGITEDQLWR | SFXN3 |
| IDVAFVDR | TRIP13 |
| VFIEDVSR | ASS1 |
| ETLDILYAR | CLYBL |
| VPAIYGVDTR | CPS1 |
| LIGIDDVPDAR | DAB2 |
| PQIAEIIR | PUM3 |
| TGAIIPELR | NT5DC3 |
| LAVEALSSLDGDLAGR | CKB |
| LGVAGQWR | UCHL1 |
| EPWPNSDPPFSFK | ITGB4 |
| ATGGGLSSVGGGSSTIK | KRT6B |
| DSPIAGFLQK | MCEE |
| AFSYYGPLR | SRSF7 |
| STGPGASLGTGYDR | CLDN3 |
| ASPSEVVFLDDIGANLK | EPHX2 |
| GGVSLAALK | HIST1H1A |
| APSTYGGGLSVSSR | KRT16 |
| DGYDYDGYR | SRSF1 |
| SVTGTDVDIVFSK | TPPP3 |
| FAFSPLSEEEEEDEQK | TMEM87A |
| ELIPEATEK | CES1 |
| TDAQAPLPGGPR | SLC22A18 |
| AVIDDAFAR | MMP9 |
| YWVDYFK | NCEH1 |
| SQDDIIPPSR | COL14A1 |
| IDAVFYSK | MMP12 |
| NLSPDGQYVPR | AGR3 |
| GIDEGPEGLK | GPD1L |
| SYEAYVLNIVR | ATP1B1 |
| ASSLESGVPSR | IGKV1-5 |
| SGGIETIANEYSDR | HSPA4L |
| VALLLLEK | ANK2 |
| QATLLLER | GSDME |
| FDSDVGVYR | HLA-DQB1 |
| SPNQNVQQAAAGALR | PKP1 |
| AEELQPGFSK | RMDN3 |
| VLEIPLEPK | NFATC2 |
| ENPIEDDLIFR | TRIM2 |
| LVTPHGESEQIGVIPSK | DLG3 |
| EGEPFELR | PTGFRN |
| TEDGGEFEEGASENNAK | HTATSF1 |
| AVVVHAGEDDLGR | SOD3 |
| VSPESNEDISTTVVYR | GLS |
| EVILPER | GUSB |
| GGQGDPAVPAQQPADPSTPER | ZNF185 |
| AVGPHQFLGDQEAIQAAIK | BPGM |
| LDLAAYDQEGR | NUP210 |
| IAGFDER | PRKDC |
| DAYSGGAVNLYHVR | PSMB5 |
| DVPFGFQTVTSDVNK | SERPINB5 |
| NPDEEDNTFYQR | LXN |
| EVSDSLLTSSK | PLIN2 |
| LGAQALLGAAK | PYCR1 |
| VGIFPISYVEK | SORBS2 |
| EIDNFYPER | SSH3 |
| AGAVGAHLPASGLDIFGDLK | SEC11C |
| LQGPQTSAEVYR | GPD1L |
| AAGDVDIGDAAYYFER | TACSTD2 |
| SQLSEFWK | CTSE |
| VDGILSEDK | NAPSA |
| EGTINVHDVETQFNQYK | MUC1 |
| AYYDGVAK | BAIAP2L1 |
| AQPFVAAANIDDK | PDLIM3 |
| SEHPGLSIGDTAK | HMGB2 |
| QNQIAVDEIR | MPO |
| NFPLPLPNK | LCP2 |
| TQIIQDFLR | LIG3 |
| IPSGLPELK | ASPN |
| IVLGQEQDSYGGK | APCS |
| AEFEQIVLGK | UPP1 |
| LGFEDGSVLK | UCHL1 |
| LSGAADTQALLR | CD3D |
| TPTLYLDFK | PIR |
| ISIEGNIAAGK | DCK |
| IFFAGTETATK | MAOA |
| SLPGQNEDLVLTGYQVDK | CSTA |
| DSEDIYNLK | CMBL |
| GFGYGQGAGALVHAQ | CSRP2 |
| TAFVPTALR | SLC27A3 |
| LLSLLEK | SPR |
| VLPVGDEVVGIVGYTSK | NT5E |
| YEDFGPLFTAK | FUCA2 |
| IPAANILGHENK | IVD |
| IQILEGWK | NQO1 |
| LASIVEQVSVLQNQGR | ALDH18A1 |
| IVPIGQPSQR | TNS1 |
| IFYNQQNHYDGSTGK | ADIPOQ |
| SPTGEWLPR | SFTPB |
| IVYEGGIDPILR | EPX |
| SSLPPVLGTESDATVK | MYLK |
| DEPEDDGYFVPPK | TOP1 |
| LILIGETIK | PTGS1 |
| AGSPSPQPSGELPR | ARHGAP45 |
| STNPGISIGDVAK | HMGB3 |
| LGEYEDVSR | TBCB |
| DLFDPIIEDR | CKB |
| DIFPYSENK | PSIP1 |
| AAIPAALPSDK | CYP27A1 |
| AAVIGDVIR | DDX21 |
| GPAYGLSAEVK | CNN1 |
| YGELEPYVYFNK | SFXN2 |
| GTVQGQLQGPISK | ACYP1 |
| VNSVSSGLAEEDLETLLQSR | CLYBL |
| SGVDADSSYFK | DHRS7 |
| PLLPGQTPEAAK | NDRG2 |
| GNYLVDVDGNR | ABAT |
| VLELNASDER | RFC4 |
| SLTTAFFR | RAB27B |
| SFAGNLNTYK | PFKP |
| AGVSSQPVSLADR | KIAA1324 |
| ASPSPTDPVVPAVPIGPPPAGFR | PC |
| ATAGAYIASQTVK | PSMB5 |
| EVGVGFATR | FABP4 |
| APAGQEEPGTPPSSPLSAEQLDR | UNG |
| FALLGDFFR | CAMP |
| FFTINPEDGFIK | CDH11 |
| LESEEEGVPSTAIR | CDK1 |
| YQLAVTQR | AOC3 |
| LPSVEEAEVPK | LAD1 |
| LYDIVTDLR | D2HGDH |
| EIENTYANVAK | CLIC2 |
| GEGAIGSLDYTPEER | UBE2J1 |
| LPSDVVTAVR | OAT |
| IGTDVLSTR | CPA3 |
| INEGFDLLHSGK | ADH1B |
| EEEAIALAEK | RAB27A |
| GIVEESVTGVHR | AHCYL2 |
| AVLGTSNFK | TFRC |
| LEYGGLGR | CMBL |
| PFYNDFER | ALOX5 |
| DALSSVQESQVAQQAR | APOC3 |
| SDIIFFQR | IL18 |
| VVPSFLPVDQGGSLVGR | DSG2 |
| VAVGNQPADIGYK | MX1 |
| ELETVDFK | SERPINB5 |
| YVQELPLEADGALR | VWA5A |
| VNSLAPGPISGTEGLR | DECR2 |
| HNYELDEAVTLQR | HLA-DPB1 |
| DPLLQEPPAWFK | TMEM97 |
| ILDVNDNIPVVENK | DSG2 |
| ESEDFIVEQYK | MCM6 |
| TIVEEVQDGK | KRT17 |
| QQQDEAYLASLR | FAF2 |
| VGEYSLYIGR | APCS |
| SEDYVDIVQGNR | PLOD2 |
| LNIPTDVLK | ERAP2 |
| TTPPVLDSDGSFFLYSR | IGHG4 |
| ELDVVDPDGSVPVGLR | LMOD1 |
| EVQEFYK | CD9 |
| EFYGENIK | ALDH3A2 |
| NPEVPVNFAEFSK | HMGB3 |
| YLAEVATGDDK | SFN |
| IIAATIENAQPILQIDNAR | KRT16 |
| DLHPNTDPFK | CDH13 |
| FEAWLAEVEGR | FNBP1 |
| GNQLWEYDPVK | GALNT1 |
| PGQAPVLVIYGK | IGLV3-19 |
| LSTPPPLAEEEGLASR | ANK1 |
| VEQEEPISPGSTLPEVK | DOCK2 |
| LISDIIR | GPD1L |
| TDLAAVPASR | IVD |
| VAFTGSTEVGK | ALDH1A1 |
| FLSLDYIPQR | CASP8 |
| LLEEEIIAFSK | SEPT10 |
| VTILELFR | SLC2A1 |
| LQGDANNLHGFEVDSR | POLR2H |
| VADYIPQLAK | GLS |
| IPTFQGLK | NPL |
| YGDVFQIR | CYP1B1 |
| SVQETVLPIK | MECP2 |
| IGEGTYGVVYK | CDK2 |
| LEVGTETIIDK | HMGCS1 |
| VYFPEQIHDVVR | NCEH1 |
| EQGYDVIAYLANIGQK | ASS1 |
| QVIGTGSFFPK | FHL1 |
| ETAELSETLTR | PPP1R18 |
| IVGPEENLSQAEAR | PLOD2 |
| AFAEALLLK | HPGD |
| VPVATYTNSSQPFR | POLD2 |
| AESLNGNPLFSK | FECH |
| ELLSHNEEFGR | CYP1B1 |
| LQNPAITGPAVPYSR | NEDD4L |
| LEGDLSLADK | AHNAK2 |
| ESNTVFSFLGLK | SH3BGRL2 |
| SFTPDHVVYAR | CAVIN1 |
| EAAAAGLPGLAR | CPD |
| VVFDDTYDR | CA3 |
| FADTVQGEVR | CRYM |
| ALPSEELNR | EIF4G1 |
| QGSVTTFLAK | FBLN2 |
| ELVAENLSVR | TRIP10 |
| DFYNPVVPEAQK | CLDN3 |
| LLGSVQQDLER | PBXIP1 |
| EEGEVPASAFQK | CAVIN3 |
| AEPGAPPAGGGLGGR | DPY19L1 |
| LYLLAAPAAER | DOK2 |
| PIFVPLSNYR | NAPSA |
| EAQAALAEAQEDLESER | MYH14 |
| TSVLYQYTDGK | RAB27A |
| DNFTLIPEGVNGIEER | CRMP1 |
| ATQPETTEEVTDLK | SHTN1 |
| LPEGQVPEGAGLK | AHNAK2 |
| SLGGQAVQIR | SCIN |
| TIVTTLQDSIR | THBS1 |
| ASFTTFTVTK | CAV1 |
| GDFVLQNEEASAK | GBP4 |
| VEGFDLVQK | PLIN2 |
| GESPPLPLDNLGK | ALOX15B |
| DGPGETDAFGNSEGK | GLS |
| AVDSLLNFETVK | ABCB6 |
| YLVIQGDER | EGFR |
| GEDLFFNYGNR | MRC1 |
| EALGGQAEEFSGR | CYP2S1 |
| IPDYSDSFK | BPI |
| WYAGLQK | TSPO |
| VPVYETPAGWR | PGM5 |
| IEEAVNAFK | ASPH |
| SDLVNEEATGQFHVYPELPK | CEACAM6 |
| TAVNALWGK | HBD |
| EGVVHGVATVAEK | SNCA |
| IGEPLVLK | AGER |
| LQPFATEADVEEALR | MCM5 |
| VIEDNEYTAR | LYN |
| EEVVGLTETSSQPK | MS4A1 |
| LEGNPIVLGK | OGN |
| ATEVPLSWDSFNK | SCIN |
| SPDEVTLTSIVPTR | PACSIN3 |
| VVVSGLPPSGSWQDLK | SRSF1 |
| FYQASTSELYGK | GMDS |
| VDFADSVTK | MYO1F |
| YTSGFDELQR | SPINT1 |
| YNFIADVVEK | HTRA1 |
| LTLFNAK | OGN |
| PDGSPVFIAFR | ALCAM |
| IDNELVVR | CYB5R2 |
| SGAPPPSGSAVSTAPQPK | SRPK1 |
| LNAFGNAFLNR | EHD3 |
| ALFDYNPNEDK | MPP7 |
| LEGELEELK | MYH14 |
| TASLTSAASVDGNR | NDRG2 |
| VGLQAQPVPDEIVK | ANK3 |
| GQESAGIVTSDGSSVPTFK | PPAT |
| LFPGFEIETVK | ASNS |
| SGEWTEDEVLR | CAPS |
| EIPLASLR | COL8A1 |
| ATPENYLFQGR | HLA-DPB1 |
| GNWDEQFDK | SERPINB6 |
| LYEPVVIPVGK | NCF2 |
| ELYGEVIR | INPP5D |
| VEADIPGHGQEVLIR | MB |
| QYFPNDEDQVGAAK | P4HA1 |
| ESVTDHVNLITPLEK | APCS |
| LINQPLPDLK | MAP2 |
| FNVSYLK | CLC |
| SAWQTLNEFAR | SEC23IP |
| APAPAPPGTVTQVDVR | EPS8 |
| LNGFEVFAR | ITGAV |
| VYVGNLGNNGNK | SRSF3 |
| QTLPEDNEEPPALPPR | HCLS1 |
| LPPAQQDEIIDR | C1orf198 |
| VVDLLATDADIISR | ADSSL1 |
| ASQDPFPAAIILK | PBK |
| LSWSQLGGSPAEPIPGR | BCAM |
| YENELALR | KRT13 |
| ELSELVYTDVLDR | MZB1 |
| QDILDDSGYVSAYK | TPPP3 |
| NSDEADLVPAK | CBX1 |
| DSYDSYATHNE | CIRBP |
| SGYGFNEPEQSR | ZNF326 |
| EEIFGPVQPILK | ALDH1A3 |
| YYGYTGAFR | LTF |
| YTELPYGR | CTSS |
| EPEQPPALWR | LPCAT1 |
| IPYTTVLR | SSRP1 |
| IDLYDVR | CACNA2D2 |
| SDLVNEEATGQFR | CEACAM5 |
| LDSPAGTALSPSGHTK | PYCR1 |
| ESQVYLLGTGLR | LCP2 |
| APHYPGIGPVDESGIPTAIR | SORBS2 |
| STATDITGPIILQTYR | NCF1 |
| SIVVSPILIPENQR | CDH13 |
| AFSVFLFNTENK | IDI1 |
| TDGGTTDYAAPVK | IGHV3-15 |
| VPLGSVIK | MAOB |
| SSSTLPVPVFK | CXCL13 |
| AGGPTTPLSPTR | LMNB1 |
| ATFSPIVTVEPR | ANK3 |
| IDLTSLR | SLFN5 |
| LGLPQDQDEPGLHLSK | C1orf116 |
| FGGFDPQGALR | HLA-DQA1 |
| IWGEDLR | NAMPT |
| GPDFFTR | AZU1 |
| DGNGFVSAAELR | CALML3 |
| AADTDGDGQVNYEEFVR | CALML3 |
| QEILEEVVR | EVL |
| AEELLDGILDK | NPL |
| EQANAVSEAVVSSVNTVATK | SNCG |
| ADGVVEGIDVNGPK | UBXN7 |
| FQWVDGSR | PRG2 |
| DDLGDDLLQDFIEK | GIMAP8 |
| FLDNFDSSEK | CAPS |
| AGNSQGDFYIR | EFEMP2 |
| FDNPAAVSPTPTR | TMTC3 |
| ISYIPDEEVSSPSPPQR | IGF2BP2 |
| VDLPLIDSLIR | SAAL1 |
| EGPFGTLVYTIK | TIMP3 |
| YDEELEER | TAGLN |
| ALAQEILPQAPIAVR | ECHDC2 |
| LVQGSILK | PCNA |
| LLNYNPEDPPGSEVLR | PON3 |
| GLIDEITK | NMI |
| DYSLLPLLAAAPQVGEK | COIL |
| FTPPQPAEPWSFVK | CES1 |
| FSGSGSGTDFTLTISR | IGKV3-20 |
| ELQQALEGQLR | DEF6 |
| FSASGELGNGNIK | PCNA |
| TDSFEYVDPVDGTVTK | PGM5 |
| SADGSAPAGEGEGVTLQR | SLC7A5 |
| SAYGGPVGAGIR | KRT7 |
| ISPVEESEDVSNK | SDC4 |
| EEDFGLFQLAGQR | GLMN |
| APPAAPAAEEPGNPGGPPR | KIAA1522 |
| SYPGLTSYLVR | LCN2 |
| TGQQAEPLVVDLK | DAB2 |
| SLFFPDEAINK | GCLC |
| GDPEWSSETDALVGSR | TMEM179B |
| VTSYIINNLQPDTTYR | FNDC3A |
| AALEDTLAETEAR | KRT19 |
| AEDSLLAAEEAAAK | TPM1 |
| AYIQEFQEFSK | OLFML1 |
| EDPAYLHYYDPAGAEDPLGAIHLR | PLEK |
| EWIEGVTGR | CNN1 |
| IEEVVLEAR | AGL |
| VEAIDVEEAK | MCM4 |
| LLPYWNER | BPGM |
| ALLSYDGLNQR | EPDR1 |
| ILGSDGAFR | ITGA2 |
| PLQQLEVPLISR | PRSS8 |
| AQHLSPAPGLAQPAAPAQASAAIPAAGK | C1orf116 |
| AETVQAALEEAQR | LAMB2 |
| LGHPDTLNQGEFK | S100A9 |
| LVTFYEDR | CRYM |
| VDSIQFSNTSNR | PHIP |
| SLGVGFATR | FABP3 |
| LTAGVPDTPTR | ITGB4 |
| GPVSVGVDAR | CTSS |
| YGIDEYLELK | ALDH5A1 |
| SGDGVVTVDDLR | CAPS |
| SGNELPLAVASTADLIR | LAS1L |
| ELLLPDTER | IPO4 |
| VPADLGAEAGLQQLLGALR | SPR |
| PYYEIGER | CD46 |
| VTILEADNR | IL4I1 |
| LVGDVDFEGVR | MTHFD2 |
| LDHHPEWFNVYNK | PCBD1 |
| SLYNLGGSK | KRT5 |
| AEEVELYLEK | TMEM87A |
| VINYEEFK | TPPP3 |
| LLAQTTLR | STOM |
| LVTSIGDVVNHDPVVGDR | PYGB |
| LLTEFNK | SERPINB3 |
| DFLIPIAWYEDR | HGD |
| VSIIDAPDISSLK | GIMAP8 |
| DGQYLLTGGDR | LRBA |
| ANDGEGGDEEAGTEEAVPR | CDC42EP4 |
| EQEPELSFLK | PBXIP1 |
| NDFIGQSTIPLNSLK | PLCD1 |
| EAEVLLLQQR | PPL |
| VDILYNNIK | SUPT16H |
| EHGVVPQADNATPSER | DNAJC2 |
| AATVGYGILR | ASRGL1 |
| GEEVTPISAIR | EPB41L3 |
| DSLSDDGVDLK | LYN |
| EPFTIAQGK | DEK |
| VAGLETISTATGR | TM7SF2 |
| LNAYTGVVYLQR | FBLN2 |
| GLPDPALSTQPAPASR | IL16 |
| ELAGGLEDGEPQQK | ELAC2 |
| QVLVENFSNFTNR | TBXAS1 |
| ALAEEVEQVHR | APOL2 |
| LPEFADWAQEQGDAPAILFDK | HLA-DMA |
| ISSVPAINNR | PRELP |
| LFGPFTR | CYBA |
| LWDLQQLR | IFI35 |
| ESGVFEGIPTYR | SCARB1 |
| FLEQELETITIPDLR | PLTP |
| EVTGIITQGAR | MFGE8 |
| YGFIEGHVVIPR | CD44 |
| LGEAAVLEIVER | SMPDL3B |
| LELFTNR | STAG1 |
| FYSVNVDYSK | NDUFA4 |
| EAVTEILGIEPDR | TSN |
| VGAPATGSGTPGPFTK | CHIT1 |
| EPAADAAPGPSAAFR | GTF3C4 |
| GPALLVLGPDAIQLR | DOK3 |
| VTIDSSYDIAK | CHI3L1 |
| TFDEIASGFR | SLC2A1 |
| VNIIPVIGK | SEPT1 |
| QQDHFFQAAFLEER | PLEK |
| IDFEDVIAEPEGTHSFDGIWK | CAV1 |
| LAADDFR | KRT14 |
| LTIESTPFNVAEGK | CEACAM5 |
| ITYEEIPLPIR | CLN5 |
| VASEEEEVPLVVYLK | SYNPO |
| SVNDIVVLGPEQFYATR | PON3 |
| IANVFTNAFR | MPO |
| YALELQK | ALPL |
| TDGFDEFK | PML |
| LTTDFGNAEK | TFRC |
| VVGQTTPESFEK | AKAP12 |
| LEDAADVYR | NAA15 |
| DLSLLSHGGR | CRYZ |
| VTLDSLR | ITGA1 |
| DFNFLTLNNQPPPGNR | MX2 |
| VVVHQETEIADE | EPB41 |
| AAPEASSPPASPLQHLLPGK | FAM129B |
| AFANPEDALR | PTGES |
| VNIIPVIAK | SEPT10 |
| IQLSDYTK | DPP4 |
| DQTPDENDQVVVK | IGF2BP3 |
| ALDPASLPR | THEMIS2 |
| NYDIGAALDTIQYSK | SQSTM1 |
| LETFIQEHLR | CD151 |
| ALLEAPLK | LTBP2 |
| EGWPSSAYGVTK | CBR1 |
| IDSVSEGNAGPYR | LAIR1 |
| ESQVQELVELIEK | GIMAP7 |
| EWDLSEYSYK | ITGA3 |
| TAVLTAFANGR | ACTL6A |
| DSDVEVYNIIK | SHMT1 |
| VVVGAPQEIVAANQR | ITGAM |
| FGPYESYDSR | ZNF326 |
| GFGGQYGIQK | HCLS1 |
| TQEPTQQHFSVAQVFLNNYDAENK | PRTN3 |
| GTSQNDPNWVVR | THBS1 |
| TELPQFVSYFQQR | TTLL12 |
| VSPGLPSPNLENGAPAVGPVQPR | LIMD1 |
| VSGVDGYETEGIR | MCM6 |
| LLLEFTDTSYEEK | GSTM3 |
| SEQLEELFSQVGPVK | RBM28 |
| LSSGDPSTSPSLSQTTPSK | EYA3 |
| GDLLFLTNFR | SEC11C |
| YEDVVQGLQK | PPL |
| LGFYEWTSR | GLA |
| GLESTTLADK | CSRP1 |

TABLE G
SVM-peptide biomarkers (200 biomarkers)

| Biomarker | Gene |
|---|---|
| SEIDLNLIK | ANXA8L1 |
| SPEVLLGSAR | CDK1 |
| SDPVTLNVLYGPDVPTISPSK | CEACAM6 |
| VLTPELYAELR | CKB |
| VGDSVDAEGPAGDSVDAEGR | CLIC6 |
| LGTQHPESNSAGNDVFAK | CLIC6 |
| AYPAIGTPIPFDK | COL10A1 |
| LYFEELSLER | CPS1 |
| VTVSEEEILPATR | CRYBG1 |
| QFYSVFDR | CTSE |
| LGTFEVEDQIEAAR | DPP4 |
| VVIVDPETNK | DSP |
| VTVLGQPK | IGLL5 |
| LLEGEDAHLTQYK | KRT17 |
| TEAESWYQTK | KRT5 |
| DAQPSFSAEDIAK | MCM3 |
| ELISDNQYR | MCM3 |
| IQETQAELPR | MCM6 |
| LANLPEEVIQK | MSH6 |
| LLYNSTDPTLR | MYO1G |
| PGPQSPGSPLEEER | NCF1 |
| IIEGEPNLK | PIGR |
| EELIADALPVLADR | PKP1 |
| IDPYVFDR | PLA2G4A |
| ELPGFLQSGK | S100P |
| QFVEQHTPQLLTLVPR | SFTPB |
| PDQGIVIPLQYPVEK | SH2D1A |
| QLEAELGAER | SPR |
| VLTDELK | TGFBI |
| INVYYNEATGNK | TUBB2B |
| IIAVDINK | ADH1B |
| IDAASPLEK | ADH1B |
| FGFLLPESR | CPA3 |
| VGDYGSLSGR | CTSZ |
| VQAELDQVVGR | CYP1B1 |
| GEGAGQPSTSAQGQPAAPAPQK | HMGA2 |
| ESLVVNYEDLAAR | MCM2 |
| LHPDDVAGIQALYGK | MMP19 |
| TLILDVPPGVEK | PTPRC |
| LLLDTFEYQGLVK | CENPV |
| LQEQEQLLK | GBP1 |
| FDASVDQASINPGK | HK3 |
| NWQDYGVR | MZB1 |
| LVLLGGEEEGPR | RASAL3 |
| ETEVIDPQDLLEGR | SDC4 |
| DEDEDIQSILR | LAD1 |
| DTDEADLVLAK | CBX5 |
| DETFNLPR | GBP1 |
| AIASIPTER | ITGA2 |
| GINSSNVENQLQATQAAR | KPNA2 |
| DVEAWFFSK | KRT15 |
| DYSQYYR | KRT17 |
| AVEPQLQEEER | PLTP |
| ADIGAPSGFK | WAS |
| VPSIELR | COL7A1 |
| DLEGSDIDTR | CSE1L |
| GGSLLAGGGGFGGGSLSGGGGSR | KRT15 |
| LEGDSEFLQPLYAK | SGCD |
| ENVVQSVTSVAEK | SNCG |
| GLGVGFGSGGGSSSSVK | KRT5 |
| TVQGPPTSDDIFER | OAT |
| SFFTDDDK | ARHGDIB |
| IEEFLEEK | CLIC6 |
| EELAEALK | MCM4 |
| INHGILYDEEK | CFHR1 |
| SQNPVQPIGPQTPK | MMP1 |
| LLALGDSGVGK | RAB27B |
| YGLNELK | ABCD3 |
| ETGYTELVK | ASPH |
| INQEELASGTPPAR | FYB1 |
| IQDLPPVDLSLVNK | KYNU |
| IDAAVFNPR | MMP12 |
| EPLPLAVK | ABCA3 |
| SHIEIIR | GSR |
| ADYDTLSLR | PKP3 |
| EVDIYTVK | PRMT1 |
| ASNLLLNYK | VRK1 |
| HLSPDGQYVPR | AGR2 |
| SINGILFPGGSVDLR | GGH |
| TSSQPGFLER | TMEM43 |
| AQGGDGVVPDTELEGR | LAMC2 |
| GENLVHQIQYR | CD163 |
| GQHLSDAFAQVNPLK | GSTT1 |
| AGILTTLNAR | MCM7 |
| DNFDIAEGVR | OAS2 |
| EFLETVK | H2AFY2 |
| IEPHHTAVLGEGDSVQVENK | PIR |
| SGFSSVSVSR | KRT6A |
| SLEQNIQLPAALLSR | MCM7 |
| AGEIVVFK | SEC11C |
| SENFEELLK | CRABP2 |
| SLDNFFAK | CDV3 |
| VYTVDLGR | PIGR |
| EAVGGLQTVR | TAP2 |
| VESLDVDSEAK | NASP |
| YQYLLTGR | TIMP3 |
| YLDNPNALTER | KANK2 |
| DPANFQYPAESVLAYK | NQO1 |
| PVDFTGYWK | RBP1 |
| IEEELGDEAR | ENO2 |
| TIDDLEETLASAK | TPM2 |
| LTDEQVALVR | BOP1 |
| DDQITLDEFK | VSNL1 |
| EDAVSAAFK | LUM |
| LSLLLNDISR | TRIP13 |
| DIYSSFGFPR | MMP1 |
| ATASEQPLAQEPPASGGSPATTK | LAD1 |
| LTDAQILTR | RFC2 |
| APETFDNITISR | SLC34A2 |
| IGYSWYK | CEACAM6 |
| NTQIQVLPEGGETPIFK | SCIN |
| QVTVLELFR | SLC2A3 |
| ANPQLGAYAPPPHVIGR | APOL2 |
| LFDVGGQR | GNAO1 |
| ILQISPEGPLQGR | DOCK10 |
| LLGASELPIVTPALR | KPNA2 |
| GEQWTPIEPK | CYBA |
| ALEAVPAPPASGPR | CRYBG1 |
| AFEESLSTLK | ECE1 |
| PNIPEAIR | ERLIN2 |
| LADLTGPIIPK | TMOD1 |
| QVLLGDQIPK | PSMB6 |
| ENYNQYDLNR | CPM |
| NANTFISPQQR | MGP |
| FSLQDPPNK | UGDH |
| ATLPVFDK | BCAT1 |
| PGAFIPGAPVQPVVLR | LPCAT1 |
| NIETIINTFHQYSVK | S100A9 |
| IEPNLPSYR | DPP4 |
| ALNSAANNVYQYGR | TMEM245 |
| LTWHAYPEDAENK | CBX5 |
| INAQLPLTDK | DCAKD |
| AALDEQFEPQK | HPGD |
| VSVGEFSAEGEGNSK | STAU2 |
| QGGFLGLSNIK | MUC1 |
| AGVVTPGITEDQLWR | SFXN3 |
| IDVAFVDR | TRIP13 |
| VFIEDVSR | ASS1 |
| ETLDILYAR | CLYBL |
| VPAIYGVDTR | CPS1 |
| LIGIDDVPDAR | DAB2 |
| PQIAEIIR | PUM3 |
| TGAIIPELR | NT5DC3 |
| LAVEALSSLDGDLAGR | CKB |
| LGVAGQWR | UCHL1 |
| EPWPNSDPPFSFK | ITGB4 |
| ATGGGLSSVGGGSSTIK | KRT6B |
| DSPIAGFLQK | MCEE |
| AFSYYGPLR | SRSF7 |
| STGPGASLGTGYDR | CLDN3 |
| ASPSEVVFLDDIGANLK | EPHX2 |
| GGVSLAALK | HIST1H1A |
| APSTYGGGLSVSSR | KRT16 |
| DGYDYDGYR | SRSF1 |
| SVTGTDVDIVFSK | TPPP3 |
| FAFSPLSEEEEEDEQK | TMEM87A |
| ELIPEATEK | CES1 |
| TDAQAPLPGGPR | SLC22A18 |
| AVIDDAFAR | MMP9 |
| YWVDYFK | NCEH1 |
| SQDDIIPPSR | COL14A1 |
| IDAVFYSK | MMP12 |
| NLSPDGQYVPR | AGR3 |
| GIDEGPEGLK | GPD1L |
| SYEAYVLNIVR | ATP1B1 |
| ASSLESGVPSR | IGKV1-5 |
| SGGIETIANEYSDR | HSPA4L |
| VALLLLEK | ANK2 |
| QATLLLER | GSDME |
| FDSDVGVYR | HLA-DQB1 |
| SPNQNVQQAAAGALR | PKP1 |
| AEELQPGFSK | RMDN3 |
| VLEIPLEPK | NFATC2 |
| ENPIEDDLIFR | TRIM2 |
| LVTPHGESEQIGVIPSK | DLG3 |
| EGEPFELR | PTGFRN |
| TEDGGEFEEGASENNAK | HTATSF1 |
| AVVVHAGEDDLGR | SOD3 |
| VSPESNEDISTTVVYR | GLS |
| EVILPER | GUSB |
| GGQGDPAVPAQQPADPSTPER | ZNF185 |
| AVGPHQFLGDQEAIQAAIK | BPGM |
| LDLAAYDQEGR | NUP210 |
| IAGFDER | PRKDC |
| DAYSGGAVNLYHVR | PSMB5 |
| DVPFGFQTVTSDVNK | SERPINB5 |
| NPDEEDNTFYQR | LXN |
| EVSDSLLTSSK | PLIN2 |
| LGAQALLGAAK | PYCR1 |
| VGIFPISYVEK | SORBS2 |
| EIDNFYPER | SSH3 |
| AGAVGAHLPASGLDIFGDLK | SEC11C |
| LQGPQTSAEVYR | GPD1L |
| AAGDVDIGDAAYYFER | TACSTD2 |
| SQLSEFWK | CTSE |
| VDGILSEDK | NAPSA |
| EGTINVHDVETQFNQYK | MUC1 |
| AYYDGVAK | BAIAP2L1 |
| AQPFVAAANIDDK | PDLIM3 |
| SEHPGLSIGDTAK | HMGB2 |

Preferred, non-limiting examples which embody certain aspects of the invention will now be described, with reference to the following figures and examples:

### DESCRIPTION OF THE FIGURES

FIG. 1. MS-based identification of NSCLC proteome subtypes. a. Bar plots showing histology and stage distribution in the patient cohort. b. Overview of experimental setup for MS-based proteome profiling, analysis output and supporting data levels.

FIG. 2. MS-based identification of NSCLC proteome subtypes. Hierarchical tree showing the results from consensus clustering used to identify NSCLC proteome subtypes. Annotation bars below indicate clinical information of samples, mRNA subtypes, infiltration signatures, common mutations as well as protein levels of selected markers.

FIG. 3. Proteome based consensus clustering of NSCLC based on 9793 proteins identified and quantified across all 141 samples in the cohort. Annotations Include: Histology, mRNA subtypes^{1,2,12}, Stage, Age, Sex, Smoking, Tumour cell content (“Purity”), immune and Stromal Signatures as described in¹⁷, TMB calculated from panel sequencing data, selected putative functional mutations from panel sequencing analysis, PD-L1 from IHC, PD-L1 from MS, KI-67 from MS. Histological subtype markers from MS (NCAM1, KRT5, NAPSA).

FIG. 4. Enrichments and tests for the NSCLC Proteome Subtypes. Volcano plots showing the output from enrichment tests of histology (a), smoking status (b), sex (c), Stage (d)

FIG. 5. Enrichments and tests for the NSCLC Proteome Subtypes. Volcano plots showing the output from enrichment tests of NSCLC mRNA subtypes (b) and AC mRNA subtypes (c). a. Boxplot indicating age distribution across the NSCLC Proteome Subtypes. e. Boxplot indicating the tumour cell content (“purity”, calculated based on panel sequencing data) across the NSCLC Proteome Subtypes. d. Scatterplot indicating the expression of SqCC markers KRT5 and KRT6A across the SqCC samples in the cohort labelled by SqCC mRNA subtype (centre) and proteome subtype (border). Horizontal dotted lines in all volcano plots indicate p-value=0.01. Dotted lines in boxplots indicate cohort median. P-values were calculated using hypergeometric test for enrichment analysis and Kruskal-Wallis test for boxplots.

FIG. 6. NSCLC proteome subtype markers a. Output from DEqMS analysis to identify differentially expressed proteins between NSCLC proteome subtypes. Numbers in the plot indicate for each comparison the number of significantly different proteins (DEqMS adjusted p-value<0.01, abs(log 2FC)>0.5). b. Bar plot indicating the number of proteins with subtype specific expression (DEqMS adjusted p-value<0.01, log 2FC>0.5 against all other subtypes). Below are shown selected examples from each subtype.

FIG. 7. NSCLC proteome subtype markers a. Stringency subset of output from DEqMS analysis to identify differentially expressed proteins between NSCLC proteome subtypes. Numbers in the plot indicate for each comparison the number of significantly different proteins (DEqMS adjusted p-value<0.01, abs(log 2FC)>1). b. Overview of markers able to distinguish between the 4 adenocarcinoma (AC) enriched subtypes (Subtypes-4). For each subtype the figure indicates the numbers of significantly different proteins compared to the other three subtypes (DEqMS adjusted p-value<0.01, abs(log 2FC)>1). Also indicated in the right part of the figure are proteins that were able to separate the subtype from all other AC enriched subtypes.

FIG. 8. NSCLC proteome subtype network analysis. NSCLC proteome subtype network analysis with UMAP plot grey-scale coloured by modules (left), modules vs iii subtypes heatmap (centre) and cell types/signalling pathway enrichment analysis output for the 10 modules (right).

FIG. 9. Cancer and driver related proteins. a. Boxplot indicating the number of overexpressed oncogenes per sample by NSCLC proteome subtype. P-value was calculated using Kruskal-Wallis test and number of samples per subtype is indicated in red. b. Bubble plot Indicating cancer and driver related proteins (CDRPs) commonly overexpressed in the NSCLC cohort. c. Scatterplot indicating mRNA to protein Pearson correlation of CDRPs. The corresponding correlation density plot is displayed on top. d. Scatterplot showing promoter methylation to mRNA correlation vs mRNA to protein correlation for CDRPs. indicated on top and to the right are the corresponding density plots for the full gene-wise overlap (9044 genes).

FIG. 10. Network analysis of NSCLC proteome subtypes. Left part shows a UMAP plot of 5257 proteins (quantified in at least 70 samples and significantly different between subtypes based on DEqMS analysis) grey-scale coloured by modules (10). Right part shows average log 2 ratio levels of module proteins for each NSCLC proteome subtype with simple annotation of each module.

FIG. 11. Network analysis of NSCLC proteome subtypes. UMAP plots for each proteome subtype separately. Different shades of grey indicate subtype median protein level (log 2) for the 5257 proteins.

FIG. 12. Network analysis of NSCLC proteome subtypes. Module enrichment analysis performed against MSigDB Hallmarks gene sets. indicated in the figure for each module are significantly enriched gene sets (p.adj<0.05).

FIG. 13. Network analysis of NSCLC proteome subtypes. Module enrichment analysis performed against cell subtypes gene sets gene sets. indicated in the figure for each module are significantly enriched gene sets (p.adj<0.05).

FIG. 14. NSCLC cohort panel sequencing results. Figure indicating putative functional mutations with a frequency above 4% in the 140 sequenced cohort samples, ordered by proteome subtype as in the original hierarchical clustering.

FIG. 15. NSCLC cohort panel sequencing results. Volcano plots showing mutation enrichment analysis for the six NSCLC proteome subtypes. P-values were calculated by Hypergeometric test, and horizontal dotted lines indicate p-value=0.01.

FIG. 16. CDRP quantitative outliers in NSCLC. Overview of CDRP definition and overlap with NSCLC proteome data. TSG: Tumor Suppressor Gene, OG: Oncogene. Hallmark, Tier 1 and Tier 2 refer to evidence levels as defined in COSMIC.

FIG. 17. CDRP quantitative outliers in NSCLC. a. Density plot indicating duplicate ratios of proteins for six cohort samples that were analysed as technical duplicates. Indicated in the plot are the distributions for all quantifications (66031 proteins for six duplicates) and the subset of quantifications that were made for proteins with Identification based on a single unique peptide. Vertical lines show the 1^{st} and 99^{th} percentile for each group. Lines Indicate the threshold for outlier expression used throughout the rest of the study. b. Scatterplot showing outlier expression pattern of CDRPs in the NSCLC cohort. The plot is based on 102955 quantifications made for 832 CDRPs in the 141 cohort samples. c. Bar plot showing the number of overexpressed oncogenes per sample. Inset shows the protein levels of 19 oncogenes with outlier expression in a specific subtype 5 sample.

FIG. 18. Examples of oncogene outliers in the NSCLC cohort. Bar plots indicating protein patterns for the oncogenes MYB, RET, EGFR, ERBB2, KRAS and SGK1 respectively. indicated for KRAS is also the mutation status of KRAS.

FIG. 19. Examples of oncogene outliers in the NSCLC cohort. Scatter plots indicating the mRNA and protein levels of the oncogenes MYB, RET, EGFR, ERBB2, KRAS and SGK1 in the NSCLC cohort. indicated in each plot is the number of samples with quantitative information at both mRNA and protein level as well as the trendline and the associated Pearson Rho and p-value.

FIG. 20. mRNA to protein correlation of CDRP outliers, a. mRNA-protein correlation for genes divided based on annotation as either miRNA targets or not according to previously published data²³. b. mRNA-protein correlation for genes divided based on mRNA and protein stability as previously determined²⁵. c. mRNA-protein correlation for genes divided based on corresponding proteins annotation as member of a protein complex according to CORUM²⁴. P-values in boxplots were calculated by Welch's t-test (a and c) or ANOVA test (b).

FIG. 21. mRNA to protein correlation of CDRP outliers. a-c. Scatter plots Indicating the mRNA and protein levels of the oncogenes HMGA2, E2F1, MUC4 in the NSCLC cohort. indicated in each plot is the number of samples with quantitative Information at both mRNA and protein level as well as the trendline and the associated Pearson Rho and p-value.

FIG. 22. mRNA to protein correlation of CDRP outliers. a. Scatter plot indicating the mRNA and protein levels of the oncogenes and IRS4 in the NSCLC cohort. indicated in each plot is the number of samples with quantitative information at both mRNA and protein level as well as the trendline and the associated Pearson Rho and p-value. b. Bar plot Indicating protein level of IRS4 in the cohort with indicated corresponding MS-TMT set number for samples with outlier levels. c. Number of unique IRS4 peptides identified per TMT set in the MS-analysis of the NSCLC cohort.

FIG. 23. Promoter methylation to mRNA to protein correlation of CDRP outliers. Scatterplot showing promoter methylation to mRNA correlation vs mRNA to protein correlation for full gene-wise overlap (9044 genes). indicated on top and to the right are the corresponding density plots.

FIG. 24. Promoter methylation to mRNA to protein correlation of CDRP outliers. Same as in FIG. 23 but showing only CDRPs with quantification in at least 60 samples.

FIG. 25. mRNA to protein correlation of CDRP outliers. Scatter plots Indicating the mRNA and protein levels of LCK, LCP1, CARD11. indicated in each plot is the number of samples with quantitative Information at both mRNA and protein level as well as the trendline and the associated Pearson Rho and p-value.

FIG. 26. mRNA to protein correlation of CDRP outliers. a. Scatter plots indicating the mRNA and protein levels of IRS2 and HNF1A. indicated in each plot is the number of samples with quantitative information at both mRNA and protein level as well as the trendline and the associated Pearson Rho and p-value. b. Scatter plot indicating the protein levels of IRS2 and HNF1A. indicated in the plot is a the trendline and the associated Pearson Rho and p-value.

FIG. 27. immune landscape and neoantigen burden in NSCLC. Overview of infiltrating immune cell subpopulations for each NSCLC proteome subtype.

FIG. 28. immune landscape and neoantigen burden in NSCLC. Scatter plot showing antigen processing/presentation machinery (APM) scores vs Tumour mutation burden (TMB) for each sample. Dotted lines indicate subdivision of the samples Into four subgroups: TMB-Low/APM-High, TMB-High/APM-High, TMB-Low/APM-Low, TMB-High/APM-Low as described in methods. Right side panels show for each subgroup enrichment analysis of NSCLC proteome subtypes. Y-axis denote enrichment p-values calculated using hypergeometric: test.

FIG. 29. immune landscape and neoantigen burden in NSCLC. a. Boxplot Indicating protein levels of PD-L1 based on MS-data (left). Right figure shows the result of PD-L1 IHC analysis for a subset of the samples. b. IHC analysis of tertiary lymph node structures (TLSs) in selected subtype 2 and 3 samples. P-values in boxplots were calculated by Wilcoxon test (b) or Kruskal-Wallis test (a).

FIG. 30. immune cell marker expression in NSCLC proteome subtypes. Boxplots indicating the protein levels of T-cell markers CD3E, CD4 and CD8A by proteome subtype as quantified by MS. P-values in all boxplots were calculated by Kruskal-Wallis test.

FIG. 31. immune cell marker expression in NSCLC proteome subtypes. Scatterplots showing MS-based quantification vs stromal staining determined by IHC for CD3E (left), and CD8A (right). indicated in the plots are also the trendlines and the associated Pearson Rho and p-values.

FIG. 32. immune cell marker expression in NSCLC proteome subtypes. Boxplots indicating the protein levels of B-cell markers CD19 and CD20 by proteome subtype as quantified by MS. P-values in all boxplots were calculated by Kruskal-Wallis test.

FIG. 33. immune cell marker expression in NSCLC proteome subtypes. Boxplots indicating the protein levels of macrophage markers CD68, CD206 and CD163 by proteome subtype as quantified by MS. P-values in all boxplots were calculated by Kruskal-Wallis test.

FIG. 34. CD3, CD8 and PD-L1 determined by IHC. Images showing example stainings for the immune cell markers CD3 (left) and CD8 (center), and PD-L1 (right). High stromal staining of CD3 and CD8 as well as cancer cell staining of PD-L1 as exemplified from three Subtype 2 samples.

FIG. 35. CD3, CD8 and PD-L1 determined by IHC. Images showing example stainings for the immune cell markers CD3 (left) and CD8 (center), and PD-L1 (right). Examples of low/negative staining for all three proteins from proteome Subtype 1 and 5 samples.

FIG. 36. Antigen processing/presentation and Immunomodulators. Heatmap indicating protein levels of HLA proteins across the NSCLC cohort samples. Indicated by bar plot on the left side is the maximum number of PSMs used for quantification of each HLA protein.

FIG. 37. Antigen processing/presentation and Immunomodulators. Boxplots indicating the median MHC class I (left) and class II (right) protein levels by proteome subtype. Lower scatter plot indicates the median MHC class I and class II levels in each sample grey-scale coloured by proteome subtype, with a trendline in green and associated Pearson Rho and p-value. P-values in all boxplots were calculated by Kruskal-Wallis test.

FIG. 38. Antigen processing/presentation and Immunomodulators. Scatter plots Indicating the median MHC class II protein expression plotted against the macrophage marker proteins CD68, CD163 and MRC1(CD206). In each plot samples are grey-scale coloured by proteome subtype, with a trendline and associated Pearson Rho and p-value.

FIG. 39. Antigen processing/presentation and Immunomodulators. Heatmaps indicating protein levels across the NSCLC cohort samples for MHC loading proteins (left), three immune modulators (top-right) and JAK-STAT signalling pathway proteins (bottom-right). indicated by bar plot on the left side is the maximum number of PSMs used for quantification of each protein.

FIG. 40. Tumour mutation burden (TMB) analysis in NSCLC cohort. Boxplots indicating TMB by histology (a), proteome subtype (b), tumour stage (c), smoking status (d), p53 mutations (e) and EGFR mutations (f). P-values indicated in boxplots were calculated using Kruskal-Wallis test (a-c) or Wilcoxon test (d-f). Number of samples per group is indicated in boxplots.

FIG. 41. Tumour mutation burden (TMB) analysis in NSCLC cohort. Scatterplots indicating TMB vs immune signature (a), stroma signature (b), proliferation (KI-67 by MS, c) and PD-L1 protein level quantified by MS (d). In each plot samples are grey-scale coloured by proteome subtype, with a trendline and associated Pearson Rho and p-value.

FIG. 42. Tertiary lymphoid structures (TLSs) and B-cell infiltration in NSCLC proteome subtypes. a. Scatterplot indicating protein levels of PD-L1 vs the B-cell marker CD20 in the entire NSCLC cohort. b. Heatmap indicating mRNA expression levels of known TLS marker genes. Cohort samples are ordered as in main FIG. 1. c. Scatterplot indicating protein levels of PD-L1 vs the B-cell marker CD20 in cohort subset selected for whole section IHC evaluation. d. TLS count (10 high power fields per sample) by subtype. e-f. IHC images showing examples of tertiary lymphoid structures from two different Subtype 3 samples. P-values in boxplots were calculated using Wilcoxon test.

FIG. 43. Tertiary lymphoid structures (TLSs) and B-cell infiltration in NSCLC proteome subtypes. a. Boxplot Indicating percent solid growth pattern in AC samples analysed by whole section IHC. b. Boxplot indicating stromal signature in Subtype 2 and 3 samples analysed by whole section IHC. c-h. IHC images showing examples of different growth patterns in AC samples analysed by whole section IHC. P-values in boxplots were calculated using Wilcoxon test.

FIG. 44. immune landscape and neoantigen burden in NSCLC. a. Overview of CT-antigen evaluation in NSCLC. Bottom part shows boxplot indicating the number of CTAs expressed per sample by proteome subtype. b. Overview of proteogenomics analysis by 6RFT database searching. Lower part shows boxplot indicating the number of non-canonical peptides per sample by proteome subtype. P-values in boxplots were calculated by Kruskal-Wallis test.

FIG. 45. immune landscape and neoantigen burden in NSCLC. a. Boxplot showing global methylation by proteome subtype. b. Top—Boxplot indicating TMB for each NSCLC proteome subtype. Bottom—Neoantigen burden by proteome subtype where neoantigen burden is defined as a summary score based on TMB, CTAs and NCPs. P-values in boxplots (b) were calculated by Kruskal-Wallis test.

FIG. 46. Cancer-Testis (CT) antigen expression analysis in NSCLC proteome subtypes. Overview of the CT-antigen analysis. Candidate CT antigen IDs were retrieved from the CT database or the Tissue Atlas, where 230 were identified at the protein level in the NSCLC cohort. Filtering was then applied based on at least two unique peptides per protein, outlier expression pattern and Tissue Atlas annotation as expressed in single or some tissues.

FIG. 47. Cancer-Testis (CT) antigen expression analysis in NSCLC proteome subtypes. The remaining 70 CT-antigens used in the continued analysis showed overall low Identification overlap across the NSCLC cohort as well as highly variable protein expression, Indicating sample specific, non-general protein expression of CT-antigens as expected. d. Bar plot Indicating the number of CT-antigen outliers per sample.

FIG. 48. Proteogenomics analysis for detection of non-canonical peptides (NCPs) In the NSCLC cohort. Overview of the proteogenomics analysis. Six reading frame translation (6RFT) database search was performed as previously described^{9,10}, and search hits were filtered based on FDR<1%, SpectrumAI for automatic MS2 spectrum Inspection/validation of single-substitution peptide Identifications and outlier expression pattern. Resulting 670 NCPs showed low Identification overlap across cohort samples indicating sample specific expression. Thirteen percent of corresponding genetic loci were supported by more than one unique peptide.

FIG. 49. Proteogenomics analysis for detection of non-canonical peptides (NCPs) In the NSCLC cohort. a. Bar plot indicating the number of identified NCPs per sample. b. Scatterplot showing the number of NCPs per sample vs TMB (left). Right part shows the output from a regression analysis between the number NCPs and TMB, Tumour cell content (“purity”), p53 mutations and proliferation (KI67 quantified by MS).

FIG. 50. Proteogenomics analysis for detection of non-canonical peptides (NCPs) in the NSCLC cohort. Bar plots Indicating the number of identified NCPs per subtype by mapping region type.

FIG. 51. Cancer Testis (CT) antigens and Non-canonical peptides (NCPs) vs methylation. Scatter plot indicating the global (a and c) or promoter (b and d) methylation plotted against the number of CT antigens per sample (a and b) or the number of NCPs per sample (c and d). In each plot samples are grey-scale coloured by proteome subtype, with a trendline in black and associated Pearson Rho and p-value. In all figures dotted lines Indicate median values.

FIG. 52. Cancer Testis (CT) antigens and Non-canonical peptides (NCPs) vs methylation. Boxplot Indicating the promoter methylation by proteome subtype with P-value by Kruskal-Wallis test. The dotted line Indicates median values.

FIG. 53. immune Checkpoints in NSCLC proteome subtypes. Boxplots Indicating protein levels of inhibitory receptors (IRs) and their ligands. All values represent protein level quantifications (log 2) except for CTLA4 where mRNA levels (log 2) are displayed since it was not detected by the MS data. P-values were calculated using Kruskal-Wallis test. Horizontal lines in boxplots Indicate median expression, and, where present, the upper outlier expression threshold. Arrows indicate ligand receptor specificity with thick arrows indicating subtype specific checkpoint activation. Question marks indicate unknown receptors. Inset box shows a scatterplot Indicating the correlation between checkpoint proteins and overall immune infiltration signature (x-axis), vs the correlation between checkpoint proteins and CD8A as a marker of cytotoxic T-cells (y-axis).

FIG. 54. STK11 inactivation in Subtype 4 results in co-expression of FGL1 and CPS1, predicting sensitivity to docetaxel and mTOR inhibitors. a. FGL1 mRNA and protein level correlations in the NSCLC cohort for 9244 genes with overlapping information for mRNA and protein level and quantitative information from at least 70 samples at protein level. b. FGL1 mRNA expression plotted against the FGL1 protein level grey-scale coloured by STK11 mutation status. c. FGL1 and CPS1 protein levels in the NSCLC cohort grey-scale coloured by proteome subtype.

FIG. 55. STK11 inactivation in Subtype 4 results in co-expression of FGL1 and CPS1, predicting sensitivity to docetaxel and mTOR inhibitors. a. CPS1 and FGL1 mRNA expression in the TCGA pan cancer dataset grey-scale coloured by cancer type. Indicated, are the 90^{th} percentiles of mRNA expression for both genes. b. CPS1 and FGL1 mRNA expression in the TCGA lung AC dataset coloured by STK11 mutation status. indicated by black lines is the median mRNA expression of both genes.

FIG. 56. FGL1 and STK11 and CPS1 in NSCLC proteome landscape. a. Scatterplot showing ranked protein level Pearson-correlations in the NSCLC cohort. The plot includes 11536 proteins where quantitative data was available for at least 70 samples. b. Scatterplot showing ranked mRNA level Pearson-correlations in the NSCLC cohort for 14548 mRNAs. C. Scatterplot showing protein vs mRNA level Pearson-correlations in the NSCLC cohort for 9244 genes where mRNA data and quantitative protein data was available for at least 70 samples. Dotted lines indicate 5^{th} and 95^{th} percentiles of mRNA and protein level correlations. d. Scatterplot showing STK11 vs STRADA protein levels in NSCLC cohort coloured by proteome subtype, with a trendline in green and associated Pearson Rho and p-value.

FIG. 57. FGL1 STK11 and CPS1 in NSCLC proteome landscape. a. Scatterplot showing CPS1 mRNA levels vs protein levels in NSCLC cohort coloured by STK11 mutation status, with a trendline in green and associated Pearson Rho and p-value. b. Scatterplot showing CPS1 vs FGL1 mRNA levels in NSCLC cohort grey-scale coloured by proteome subtype, with a trendline and associated Pearson Rho and p-value.

FIG. 58. FGL1 STK11 and CPS1 in NSCLC proteome landscape. Scatterplots for evaluation of HNF1A regulation showing promotor methylation vs mRNA level (left), promotor methylation vs protein level (centre) and mRNA level vs protein level (right) in NSCLC cohort grey-scale coloured by proteome subtype, with a trendline and associated Pearson Rho and p-value.

FIG. 59. FGL1, STK11 and CPS1 in TCGA pan-Cancer and LUAD. a. Scatterplot showing protein level Pearson-correlations in the NSCLC cohort vs mRNA level correlation in the TCGA PanCancer dataset for 10447 genes where mRNA data and quantitative protein data was available for at least 70 samples. Lines Indicate 5^{th} and 95^{th} percentiles of mRNA and protein level correlations. b. Boxplots showing FGL1 (left) and CPS1 (right) mRNA levels by STK11 mutation status in the TCGA lung adenocarcinoma (LUAD) dataset. P-values were calculated using Wilcoxon test.

FIG. 60. FGL1, STK11 and CPS1 in TCGA pan-Cancer and LUAD. a. Scatterplot showing STK11 vs FGL1 mRNA levels in the TCGA PanCancer dataset grey-scale coloured by cancer type. b. Scatterplot showing STK11 vs CPS1 mRNA levels in the TCGA PanCancer dataset grey-scale coloured by cancer type.

FIG. 61. FGL1, STK11 and CPS1 in TCGA pan-Cancer and LUAD. a. Scatterplot showing STK11 vs FGL1 mRNA levels in the TCGA LUAD dataset coloured by STK11 mutation status with a trendline and associated Pearson Rho and p-value. b. Scatterplot showing FGL1 vs HNF1A mRNA levels in the TCGA LUAD dataset grey-scale coloured by STK11 mutation status with a trendline and associated Pearson Rho and p-value.

FIG. 62. STK11 inactivation in Subtype 4 results in co-expression of FGL1 and CPS1, predicting sensitivity to docetaxel and mTOR inhibitors. a. CPS1 and FGL1 mRNA expression in the GDSC dataset grey-scale coloured by cell line tissue origin. Indicated are the 90^{th} percentiles of mRNA expression for both genes. b. Volcano plot indicating differences in drug sensitivity between NSCLC cells with high mRNA expression of CPS1/FGL1 vs remaining NSCLC cells. indicated in the plot is docetaxel as well as several drugs targeting mTOR.

FIG. 63. FGL1, STK11 and CPS1 in the Genomics of Drug Sensitivity in Cancer (GDSC) NSCLC cell lines vs drug response. Scatterplot showing CPS1 vs FGL1 mRNA levels in GDSC NSCLC cell lines coloured by STK11 mutation status with a trendline and associated Pearson Rho and p-value. Red lines indicate 90^{th} percentiles of CPS1 and FGL1 mRNA expression.

FIG. 64. FGL1, STK11 and CPS1 in the Genomics of Drug Sensitivity in Cancer (GDSC) NSCLC cell lines vs drug response. Boxplot showing the output from a differential drug response analysis between FGL1/CPS1 high NSCLC cell lines and remaining cell lines. Y-axis indicates the IC50 log 2 FC by drug target group.

FIG. 65. FGL1, STK11 and CPS1 in the Genomics of Drug Sensitivity in Cancer (GDSC) NSCLC cell lines vs drug response. Boxplot showing the output from a differential drug response analysis between FGL1/CPS1 high NSCLC cell lines and remaining cell lines. Y-axis indicates the p-value by drug target group as calculated by t-test.

FIG. 66. Volcano plot showing the output from a differential drug response analysis between STK11 mutated NSCLC cell lines and STK11 wild-type cell lines. Y-axis Indicates the −log 10 p-value as calculated by t-test, and x-axis Indicates the IC50 log 2 FC.

FIG. 67. NSCLC classification pipelines validate NSCLC proteome subtypes and Indicate clinical utility a. Overview of NSCLC Proteome Subtype classification pipelines. b. Violin plot indicating the accuracy of the SVM classifier and the k-TSP classifier. C. SVM classifier feature Importance evaluated by the frequency each feature was used across the Monte Carlo cross validation Iterations. d. k-TSP classifier feature pair Importance evaluated by the frequency each feature pair was used across the Monte Carlo cross validation iterations.

FIG. 68. Support Vector Machine (SVM-Protein) based cohort classifier for NSCLC subtype classification. Overview of the SVM classification optimisation. Briefly, Monte-Carlo-Cross-Validation (MCCV) with 100 iterations was used to estimate classifier accuracy, select the most important features and to build the final classifier. For each iteration the cohort samples were split into a training set and a testing set, and RFE-SVM was applied to select the 200 most Important features. After training, the accuracy of the classifier was estimated using the test set samples. The overall accuracy was reported as the average accuracy of the 100 iterations, and the most frequently used 200 features were used to build the final model.

FIG. 69. Support Vector Machine (SVM-Protein) based cohort classifier for NSCLC subtype classification. a. Sankay plot showing the SVM classification output from the SVM testing (100 iterations) with 94% accuracy. b. Stacked bar plots showing the subtype outlierness (Top, indicated by consensus index from the original clustering) and the classification output form the 100 MCCV Iterations (bottom). indicated by arrows are seven samples that were frequently mis-classified by the SVM.

FIG. 70. DIA-MS analysis of the lung cancer cohort. DIA-MS analysis of the 141 samples resulted in the identification of 6717 proteins (FDR<1%) with a minimum of 2220 proteins per sample and a full overlap of 1202 proteins across all samples. Left part shows protein-wise and sample-wise correlation between DIA-MS based, and DDA-MS based quantifications.

FIG. 71. k-Top Scoring Pairs (k-TSP) based single sample classifier for NSCLC subtype classification. Overview of the k-TSP classification optimisation. Briefly, Monte-Carlo-Cross-Validation (MCCV) with 100 Iterations was used to estimate classifier accuracy, select the most important feature pairs and to build the final classifier. For each Iteration the cohort samples were split into a training set and a testing set, and 15 binary models were built, each based on 15 feature pairs (k), selected based on accuracy in test data (right plot). After training, the accuracy of the classifier was estimated using the test set samples. The overall accuracy was reported as the average accuracy of the 100 iterations. The 15 most frequently used feature pairs for each binary model were used to build the final model, resulting in 225 final feature pairs.

FIG. 72. k-Top Scoring Pairs (k-TSP) based single sample classifier for NSCLC subtype classification. a. Sankay plot showing the classification output from the k-TSP testing (100 iterations) with 87% accuracy. b. Stacked barplots showing the subtype outlierness (top, indicated by consensus index from the original clustering) and the classification output form the 100 MCCV iterations (bottom). indicated by arrows are samples that were frequently mis-classified by the k-TSP.

FIG. 73. SVM-protein based classification of public domain AC transcriptomics data. a. SVM-based classification of the GEO NSCLC cohort based on mRNA level data. Indicated below is sample annotation by histology, mRNA subtype and marker/signature levels. b. Kaplan-Meier plot showing overall survival in the GEO NSCLC cohort by classified subtype.

FIG. 74. DIA-MS analysis and k-TSP based classification of a late-stage NSCLC cohort. a. DIA-MS data coverage of the k-TSP feature pairs in the late stage NSCLC cohort in relation to biopsy type and histology. b. k-TSP classifier output for the 61 late stage samples where at least 50% of k-TSP feature pairs were identified, grey scale coloured by histological subgroup. c. Scatterplots indicating Keratin 5 and Keratin 6A (KRT5, KRT6A SqCC markers) levels in the classified subset of the late stage NSCLC cohort as quantified by DIA-MS. Left plot is grey scale-coded by classified subtype and right plot by histology. indicated by arrows in the plots are six cases with unexpected classification output.

FIG. 75. SVM-protein based classification of public domain AC transcriptomics data. Output from SVM-based classification of the TCGA AC cohort based on mRNA level data. indicated below is sample annotation by mRNA subtype, mutation patterns and marker/signature levels.

FIG. 76. SVM-protein based classification of public domain AC transcriptomics data. Kaplan-Meier plot showing overall survival in the TCGA AC cohort by classified subtype.

FIG. 77. SVM-protein based classification of public domain AC proteomics data. a. Venn diagrams showing overlap between current NSCLC cohort and the Gillette et al. AC cohort in all identified proteins (top) and proteins with full overlap in respective cohorts (bottom). indicated by a circle is the overlap with 250 most frequently used features from the SVM classifier optimisation. b. Output from SVM-based classification of the Gillette et al. AC cohort. indicated below is sample annotation by mRNA and protein subtype, mutation patterns and marker/signature levels. To the right is shown the results by classified subtype including p-vales from Kruskal-Wallis test (markers and signatures) or hypergeometric test (mutations).

FIG. 78. k-TSP based classification of public domain AC proteomics data. Output from k-TSP-based classification of the Xu et al. AC cohort. indicated below is sample annotation by mutation patterns and marker/signature levels. To the right is shown the results by classified subtype including p-vales from Kruskal-Wallis test (markers and signatures) or hypergeometric test (mutations).

FIG. 79. k-TSP based classification of late-stage NSCLC cohort. a. Barplot showing the histologies of the 84 samples included in the late-stage cohort. b. Scatterplot showing mRNA and peptide yields from the sample prep of biopsy samples using Allprep kit followed by digestion, coloured by biopsy type, with a trendline in green and associated Pearson Rho and p-value. c. Experimental setup for DIA-MS analysis of late stage cohort samples. d. DIA-MS analysis of the 84 samples resulted in the Identification of 5124 proteins (FDR<1%). Excluding one sample with only 55 proteins identified, DIA-MS resulted in a minimum of 902 proteins per sample and a full overlap of 332 proteins across all samples. e. Scatterplot showing peptide yield vs DIA-MS protein IDs, grey-scale coloured by biopsy type, with a trendline and associated Pearson Rho and p-value. f. Scatterplot showing k-TSP feature pair coverage vs DIA-MS protein IDs, with a trendline and associated Pearson Rho and p-value. Horizontal line indicate threshold for classification inclusion.

FIG. 80. k-TSP based classification vs Histology in late-stage NSCLC cohort. a. Scatterplot indicating Napsin A (AC marker) vs Keratin 5 (SqCC marker) levels in the classified subset of the late-stage NSCLC cohort as quantified by DIA-MS. Left plot is labelled by classified subtype and right plot by histology. indicated by arrows in the plots are cases with unexpected classification output. b. Same as in a. but for Keratin 5 (SqCC marker) vs NCAM1 (Neuronal lineage marker).

FIG. 81. Peptide-Centric Classification using Support Vector Machine (SVM-peptide).

### EXAMPLES

### Non-Small Cell Lung Cancer Proteome Subtypes Expose Targetable Oncogenic Drivers and Immune Evasion Mechanisms

### Summary

Lung cancer is the deadliest cancer type and despite major advancements in treatment, long term survival is still rare. To gain understanding of how the molecular phenotype level regulation impacts targetable cancer driver pathways and immune evasion, the inventors performed in-depth mass spectrometry (MS)-based proteogenomics analysis of 141 cancers representing all major histologies of non-small cell lung cancer (NSCLC). With close to 14000 proteins quantified, and almost 10000 across all samples inventors' analysis indicated six distinct proteome subtypes driven by histology, growth pattern, immune cell Infiltration, driver mutations, oncogenic pathways, and cell types. The analysis reveals striking differences between subtypes in immune system engagement Including a T-cell Infiltrated subtype, a subtype featuring B-cell rich tertiary lymphoid structures and several immune-cold subtypes associated with subtype-specific expression of immune checkpoint receptor ligands. Unexpectedly, Inventors' proteogenomics analysis revealed that high neoantigen burden was linked to global hypomethylation, and that complex neoantigens mapping to genomics regions Including endogenous retroviral elements and Introns were produced in Immune-cold subtypes. Further, the Inventors link immune evasion in one immune cold subtype to STK11 mutation through activation of an HNF1A-driven liver-specific transcriptional program resulting in expression of FGL1, a secreted ligand to the Inhibitory T-cell receptor LAG3. Finally, the Inventors develop an DIA MS-based NSCLC subtype classification method and demonstrate the applicability of the method for both early and late stage NSCLC biopsy samples in a clinical setting.

### Introduction

Lung cancer is the most common type of cancer worldwide with 2.1 million new cases each year. The majority of cases are diagnosed when the cancer has already metastasized and surgical resection is no longer an option, resulting in a dismal overall 5-year survival rate for non-small cell lung cancer (NSCLC) of 24% and only 6% in stage 4 disease (seer.cancer.gov). Rapid development of targeted therapies and Immunotherapy present a major opportunity, but the Impact on survival so far is blunted by lack of biomarkers for therapy selection and limited knowledge of how therapies should be combined. Exploratory omics-analyses of clinical cancer cohorts have demonstrated the value of a systems level analysis of cancer^{1,2}. Most of previous cancer landscape studies have placed emphasis on genetic alterations for stratification of patients Into different subtypes. In a few cancer types though, it has been thoroughly demonstrated that molecular subtyping based on gene expression, assayed by transcriptomics, creates robust and clinically highly relevant patient stratification. Already 20 years ago, Charles Perou and co-workers demonstrated that gene expression analysis could be used to stratify breast cancer patients with the potential to Improve clinical prognostication³. This report and subsequent similar studies demonstrate that mRNA-level analysis can be used as an approximation of the molecular phenotype, and that this Information enables better understanding of the underlying disease.

With the Improved analytical depth provided by modern mass spectrometry (MS)-methodology the Inventors added a layer to measure the actual druggable molecular phenotype directly, i.e. the proteome, which has the potential to provide a more accurate understanding of the disease for predictive medicine. The Inventors hypothesize that comprehensive proteome-level data provides a more complete systems view of the tumour state, capturing the impact of genomic aberrations as well as epigenetic, transcriptional and post-transcriptional regulation. An important feature of such analysis is that it provides a readout not only the cancer cells in the sample, but also the stromal component and infiltrating immune cells. Altogether, this provides a picture of the dominant molecular cancer phenotype, or simply the most distinct features of the tumour as an organ⁴. This level of information is crucial for understanding how cancer cells acquire hallmark capabilities such as oncogenic growth, evasion of cell death signalling and immune evasion, and most importantly how to target these hallmarks to improve cancer treatment. Integration of proteome level analysis in cancer landscape studies has only just recently started to be performed. For lung cancer, proteogenomics studies have been performed on squamous cell carcinoma (SqCC, n=108)⁵, and on adenocarcinoma (AC) in three studies (Gillette et al.⁶, n=110; Xu et al.⁷, n=103 and Chen et al.⁸, n=103). For the AC studies, much focus was put on cancer in never-smokers (46%, 77% and 83% of cohorts respectively) and consequently also on EGFR mutation driven AC due to enrichment of this mutation in never-smoker AC cases (EGFR mutations in 34%, 50% and 85% samples respectively).

Here the Inventors have performed in-depth analysis of the NSCLC proteome landscape, covering nearly 14000 proteins and all major NSCLC histological subtypes. Based on this data, the inventors defined six proteome subtypes of NSCLC and used the protein level information to demonstrate clinical implications of the proteome subtypes, such as prognostic or treatment predictive value. Inventors' in-depth analysis provides crucial new information for potential stratification of NSCLC patients in relation to Immuno-therapy as well as targeted therapy, underscoring the value of the herein defined NSCLC proteome subtypes. Finally, the inventors developed a MS-based classification method that can be used for both early and late stage NSCLC samples in a clinical setting.

### Results

### 1. Proteome Subtypes of NSCLC

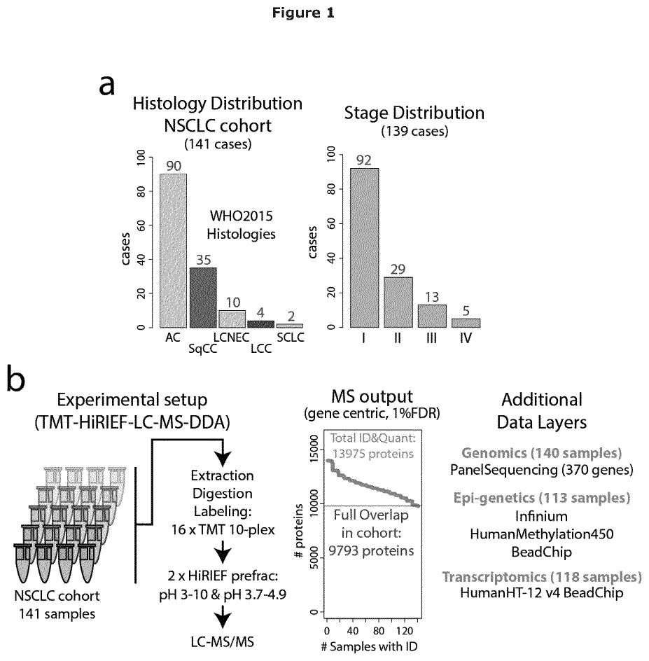

The most recent WHO classification scheme subdivides NSCLC into the histological subtypes AC, SqCC, large cell neuroendocrine carcinoma (LCNEC) and large cell lung cancer (LCC). In the current cohort of resected tissue samples (n=141), all these subtypes are Included, as well as two small cell lung cancer (SCLC) cases as reference samples (FIG. 1a).

The cohort primarily consists of early stage (I-II, 87%) cancer, as late stage (III-IV) NSCLC rarely Involves surgical removal of the tumour tissue. For a comprehensive phenotype-level analysis of NSCLC the inventors used their previously developed method for In-depth MS-based proteomics, HiRIEF-LC-MS^{9,10}, that the Inventors recently applied for proteome-level subtyping of breast cancer¹¹. The proteomics workflow using Isobaric labelling for relative quantification of proteins between samples (TMT-HiRIEF-LC-MS with data dependent acquisition, DDA) is shown in FIG. 1b. Overall, the MS analysis generated state-of-the-art analytical depth with 13975 identified proteins (gene-centric search, FDR<1%), and a full overlap across all samples of 9793 proteins (FIG. 1b). In addition to MS-data, mutation analysis for cancer associated genes was performed by panel sequencing (n=370 genes) and genome-wide methylation as well as mRNA-level data was available for the majority of samples¹²⁻¹⁴.

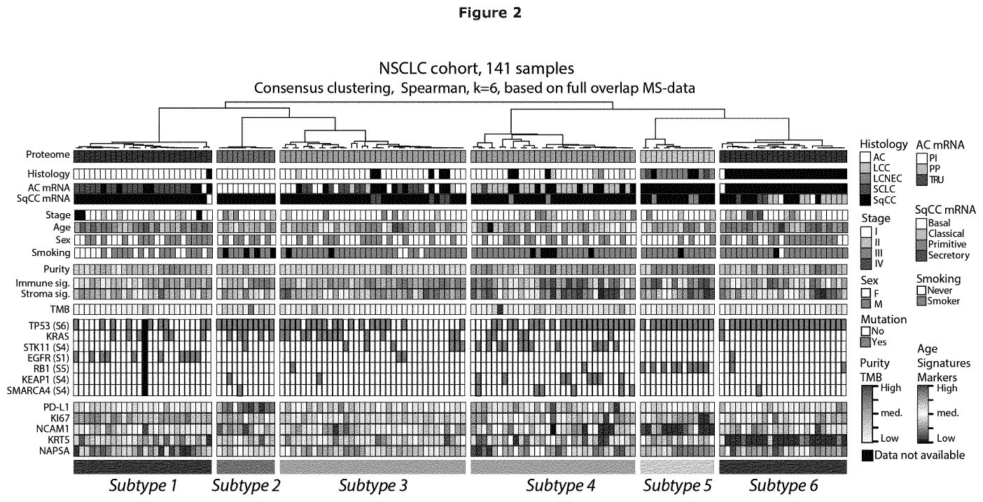

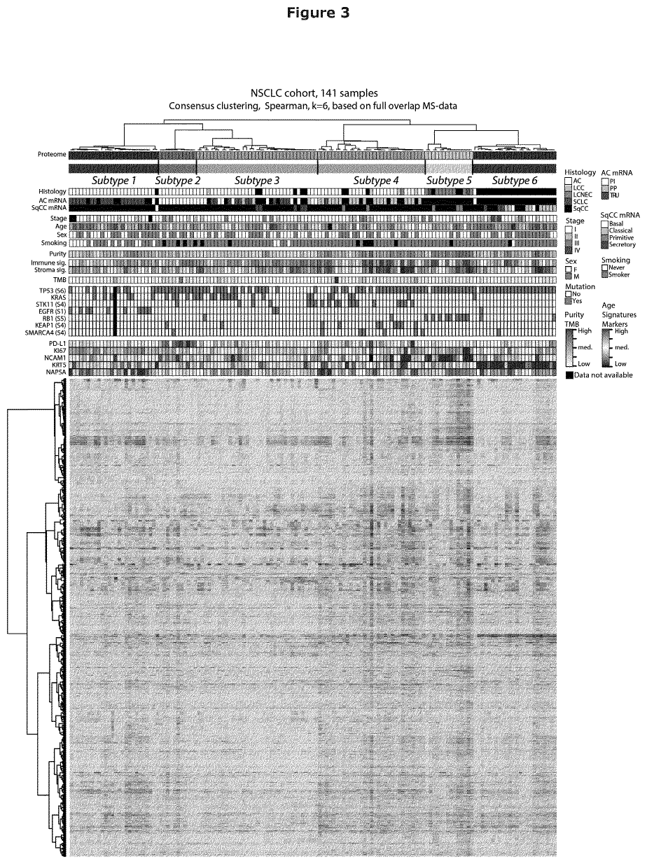

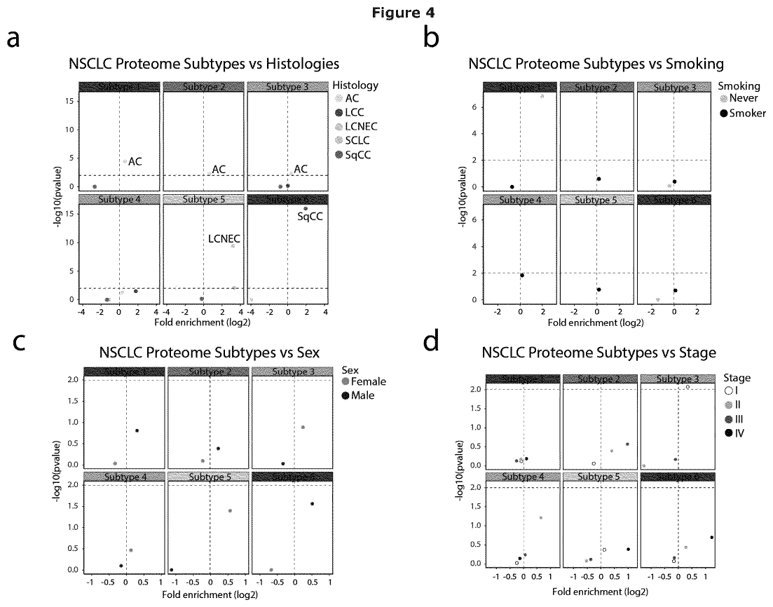

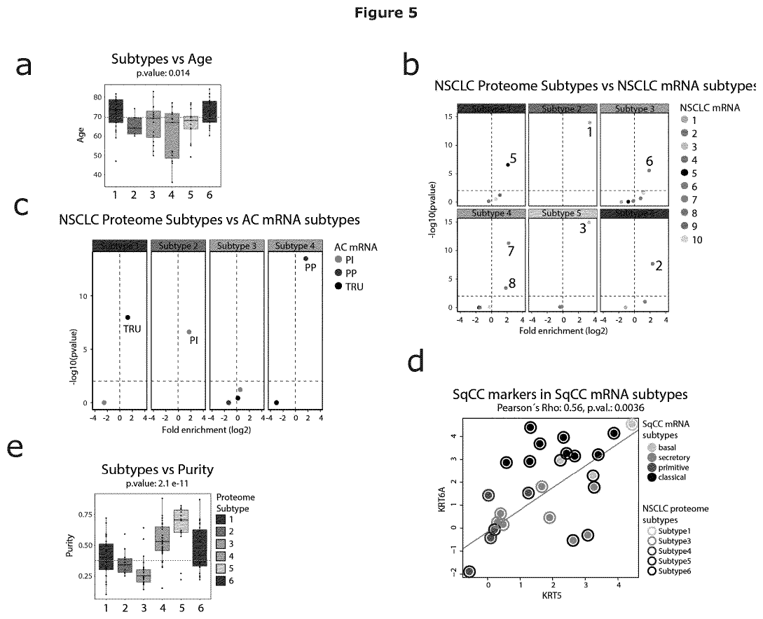

For proteome-level molecular subtyping of NSCLC, the Inventors applied Spearman consensus clustering using all proteins quantified across all 141 cohort samples (9793 proteins), resulting in six distinct clusters (FIG. 2, FIG. 3). Hereinafter, these six clusters will be referred to as (Proteome) Subtypes 1-6. Comparing with histological subtype Information, Subtype 1-4 samples were primarily AC (77-100%), Subtype 5 samples LCNEC (64%), and Subtype 6 samples SqCC (96%). Both SCLC samples grouped together with LCNEC samples as expected due to neuroendocrine lineage origin. Further, never-smokers were enriched in Subtype 1 while evaluation of sex, tumour stage and age distribution across the different subtypes did not reveal any specific enrichment patterns (FIG. 4, FIG. 5a). A previous subtyping of the current NSCLC cohort based on mRNA-level analysis¹² revealed ten different subtypes showing a partial overlap with the six proteome subtypes here identified (FIG. 2).

Notably, mRNA subtype 1 overlapped well with proteome Subtype 2, and mRNA subtype 3 with proteome Subtype 5 (FIG. 5b). Subtyping based on mRNA expression for AC specifically has been previously performed by the TCGA network². In this study three expression subtypes were identified; terminal respiratory unit (TRU); proximal-inflammatory (PI); and proximal proliferative (PP). Classification of the AC samples in the current cohort Into these three subtypes based on RNA-level data revealed that Subtype 1 consisted primarily of TRU samples, Subtype 2 of PI samples and Subtype 4 of PP samples (FIG. 2, FIG. 5c). Importantly, Subtype 3 did not show enrichment of any previous AC mRNA subtype. SqCC mRNA expression subtypes have also been described by the TCGA network previously¹. Interestingly, even though the majority of SqCC samples in inventors' analysis are found in Subtype 6, all of the “primitive” SqCC samples are found in Subtype 5 (3/5) or Subtype 4 (2/5), and 5/8 of the “secretory” SqCC In Subtype 3 (FIG. 2). SqCC samples clustering outside of Subtype 6 commonly also express lower levels of SqCC markers (KRT5 and KRT6A), indicating that these cancers may be more atypical SqCC (FIG. 5d). Recently, a proteomics-based subtyping was reported for SqCC, with 4880 proteins identified in at least 90% of samples⁵. This study also indicated that proteome level analysis contributes with Information not revealed by the mRNA level analysis.

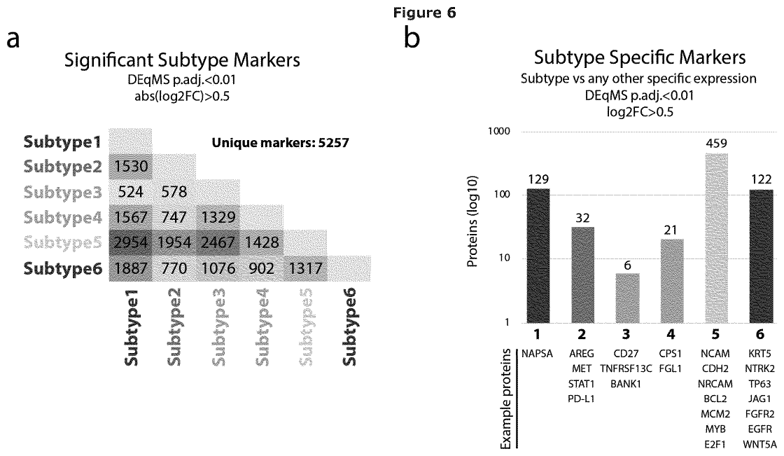

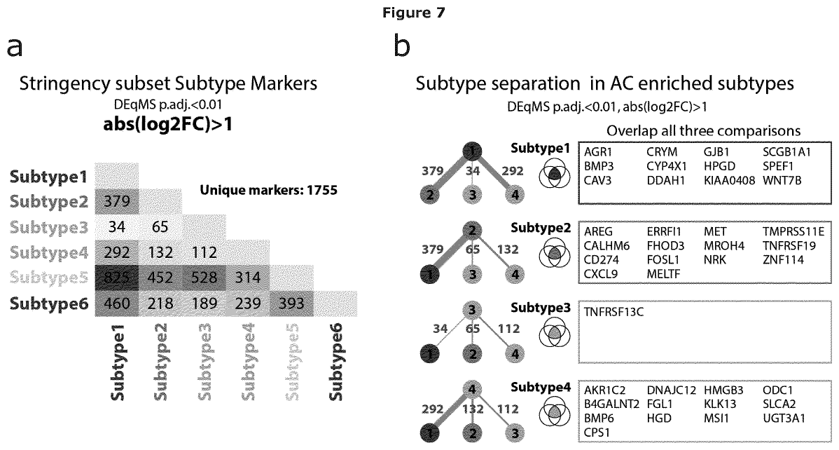

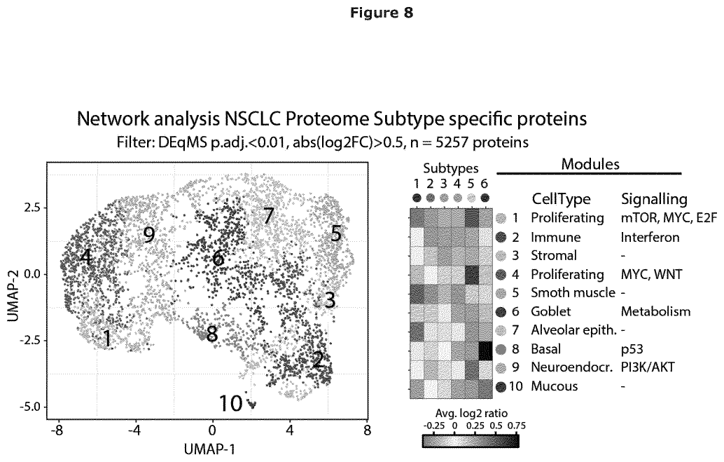

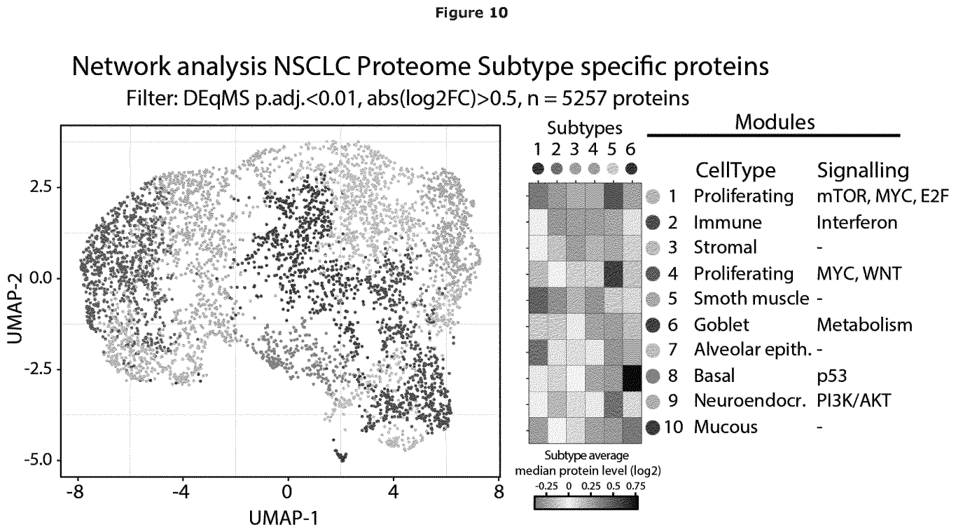

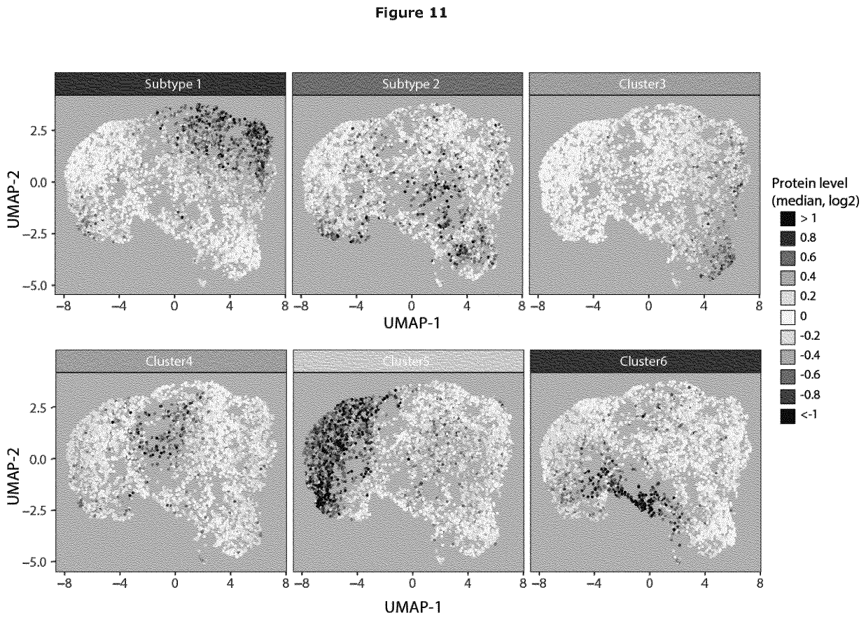

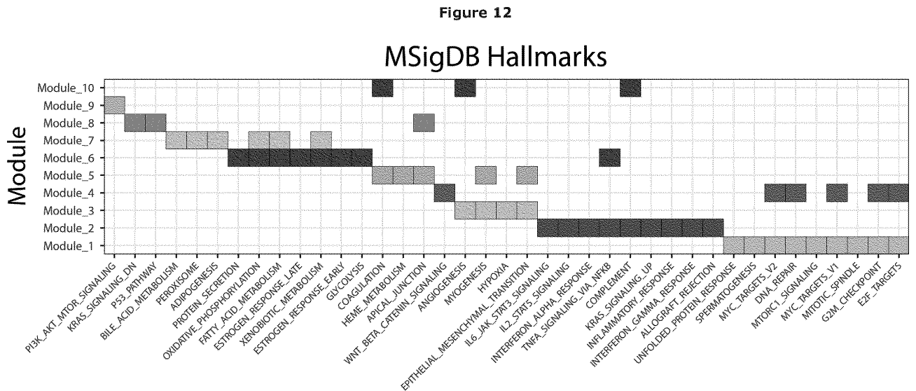

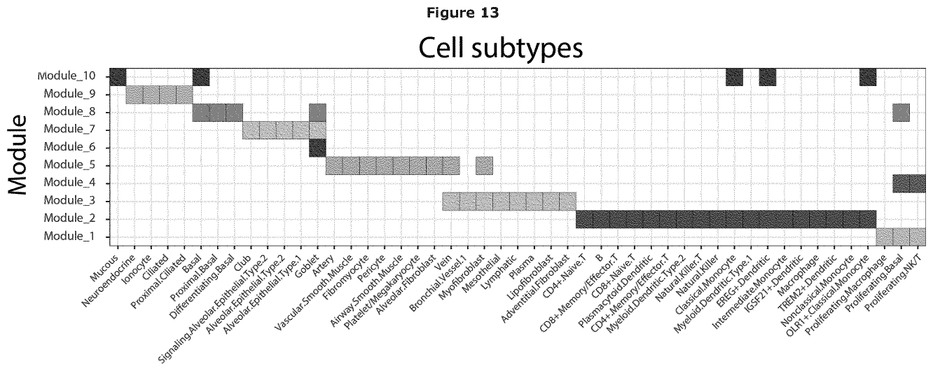

The inventors have previously shown that network analysis based on proteome level information is a powerful method to investigate biological pathways and processes associated with individual breast cancer subtypes¹¹. To generate a broad phenotypic characterization of the NSCLC proteome subtypes the inventors first identified differentially expressed proteins between subtypes using DEqMS¹⁵ (FIG. 6-7). Proteins with significantly different levels between subtypes (p.adj.<0.01 and |log 2 ratio|>0.5) were then used for network analysis by U-MAP¹⁶. Overall, the network analysis indicated that the proteome subtypes were separated based on cell types as well as cell signalling (FIG. 8, FIG. 10-13). immune infiltration was highest in Subtypes 2 and 3 and stromal component in Subtype 3, which was also supported by signature analysis using the previously described ESTIMATE method¹⁷ (FIG. 2). These results were in agreement with the cell composition evaluation, as Subtype 2 and 3 showed the lowest tumour cell content (“purity”, estimated based on panel sequencing data, FIG. 5e). Further, the network analysis indicated the highest proliferation in Subtype 5, and the lowest in Subtype 1, which was supported by KI67 levels as measured by MS (FIG. 2).

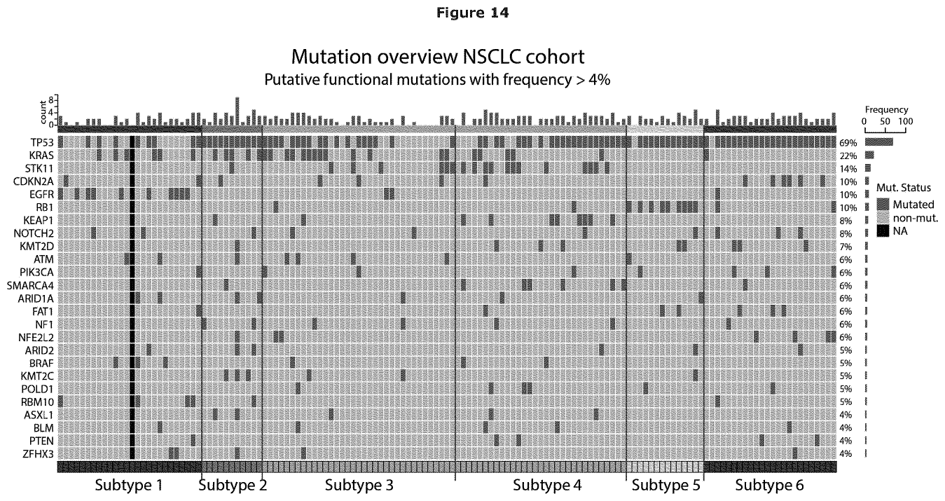

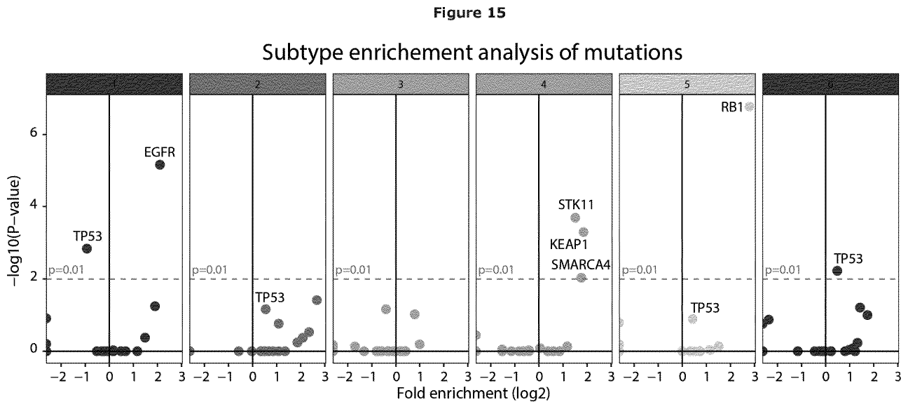

To annotate the cohort samples by mutation pattern in known cancer genes, panel sequencing was performed covering 370 genes. Overall this analysis confirmed previously reported mutation patterns in NSCLC and revealed enrichment of EGFR mutations in Subtype 1; STK11, KEAP1 and SMARCA4 in Subtype 4; RB1 mutations in Subtype 5 and TP53 mutations in Subtype 6 (FIG. 14-15). Further, the mutation patterns agree with the phenotype level network analysis as E2F1/MYC signalling and RB1 mutations were enriched in Subtype 5, metabolism and STK11 mutations in Subtype 4 and both p53 signalling and TP53 mutations in Subtype 6. Interestingly, all three SqCC samples in Subtype 5 harboured RB1 mutations, and the only LCNEC sample outside of Subtype 5 was mutated for both STK11 and KEAP1 and grouped with Subtype 4. This indicates that the NSCLC Proteome Subtypes capture dominant molecular cancer phenotypes related to driver signalling pathways notwithstanding the formal histological classification.

### 2. Cancer and Driver Related Proteins

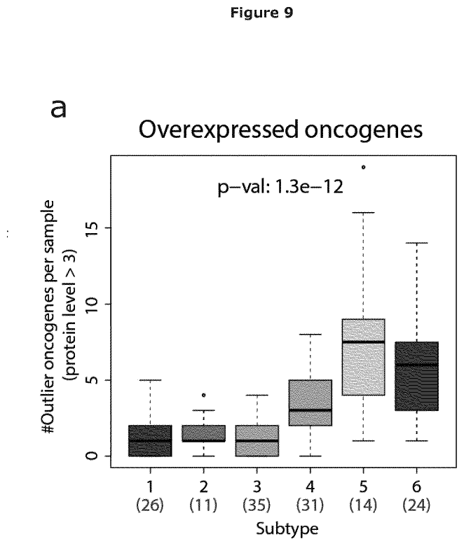

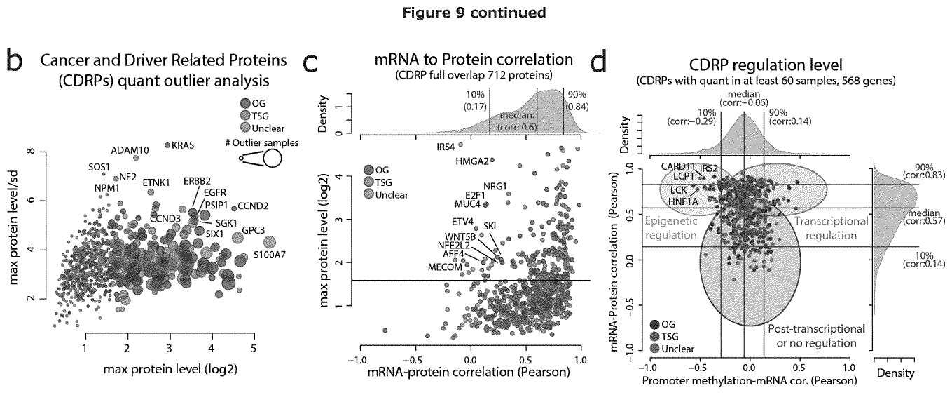

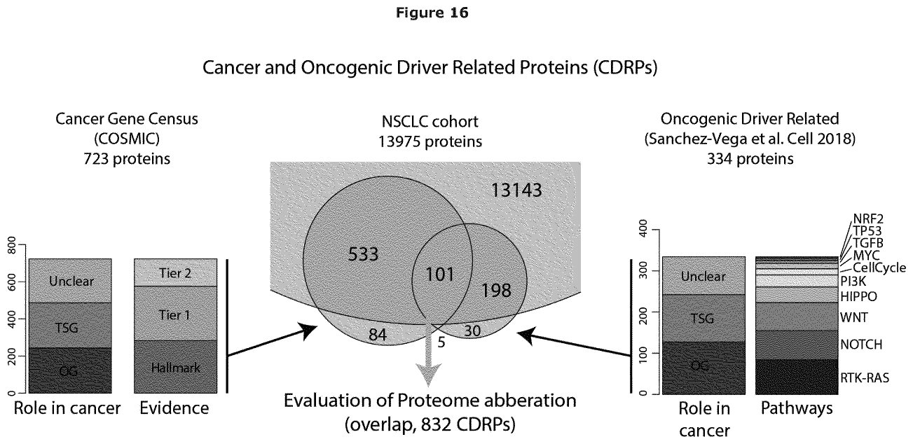

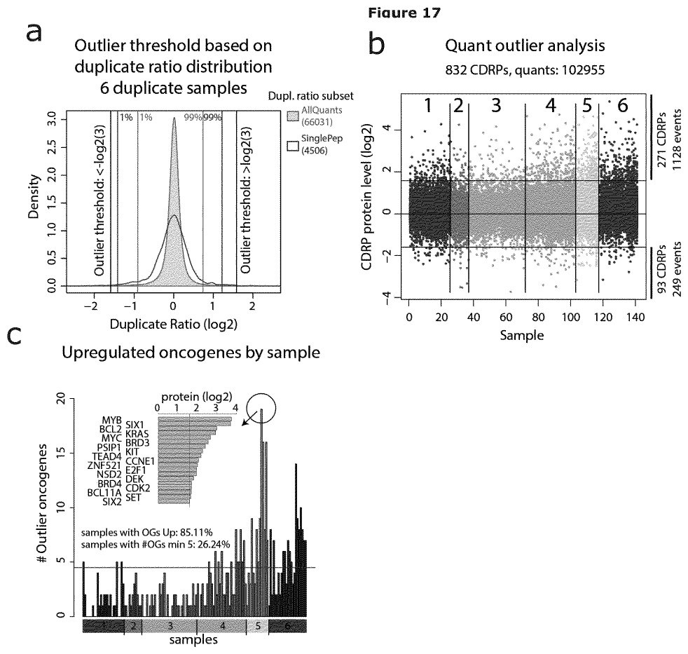

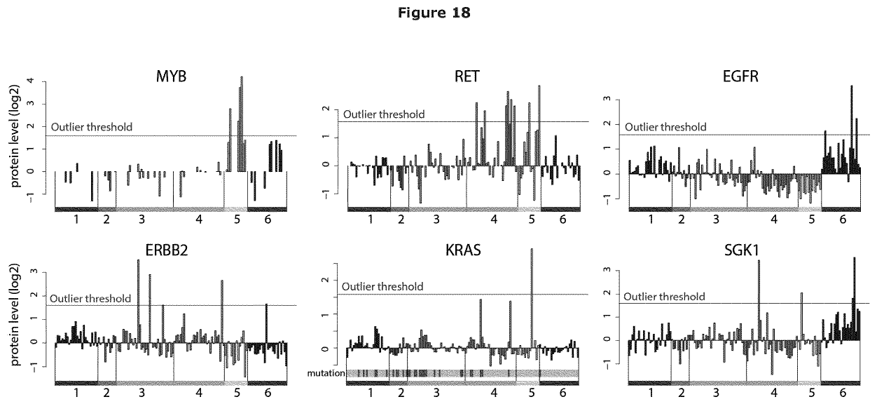

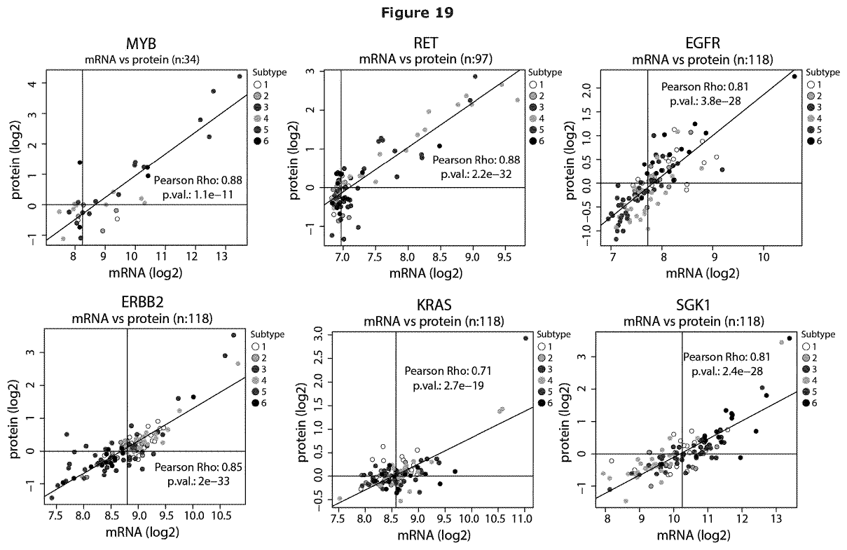

Large scale genomic studies on cancer have resulted in a long list of genes with association, or a direct causal link to cancer. To associate proteome level information to known cancer associated genes, the Inventors defined a list of proteins based on membership in 10 cancer-related signalling pathways as previously described¹⁸, and/or If causally linked to cancer according to the COSMIC cancer gene census effort¹⁹. This resulted in a list of 951 proteins out of which 832 were identified and quantified in the NSCLC cohort, referred to from this point on as “Cancer and Driver Related Proteins” (CDRPs, FIG. 16). Out of these CDRPs, 291 showed outlier levels (sample protein level>3-fold up or down compared to cohort median, FIG. 17a-b) In at least one sample, 85% of the samples showed outlier expression of at least one oncogene, and 26% of at least five (FIG. 17c). Subtype 5 showed the highest number of overexpressed oncogenes per sample (FIG. 9a), commonly including the transcriptional activator MYB. Of the AC enriched subtypes (Subtypes 1-4), Subtype 4 showed the highest number of overexpressed oncogenes per sample with common overexpression of the receptor tyrosine kinase RET (FIG. 18-19). Further, the analysis pointed out several oncogenic drivers previously shown to be overexpressed in NSCLC such as EGFR and ERBB2, but also overexpression of wild-type KRAS and of additional oncogenes not commonly implicated in NSCLC such as the oncogenic kinase SGK1 (FIG. 9b). SGK1 is linked to both Src²⁰ and mTOR²¹ signalling and has previously been shown regulated at the protein level by the ubiquitin-proteasome system. However, in inventors' cohort SGK1 protein level correlated closely to its mRNA level, suggesting transcriptional regulation, which was also true for the other here above-mentioned oncogenes (FIG. 18-19).

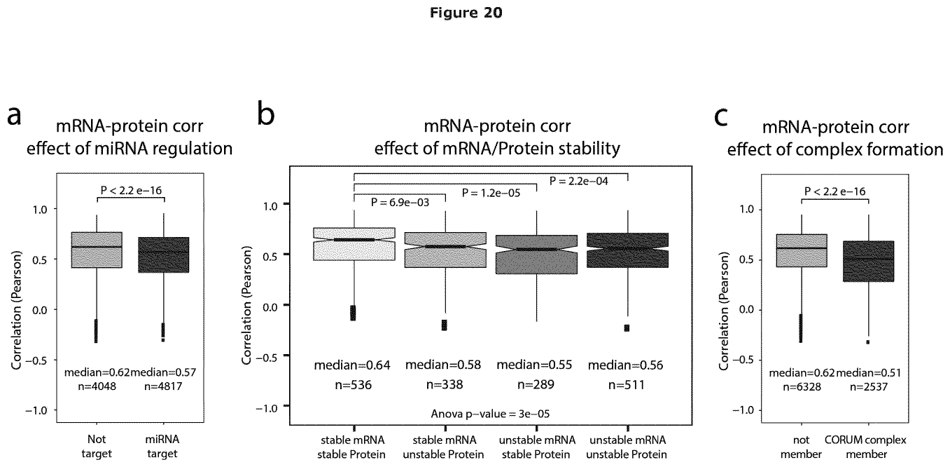

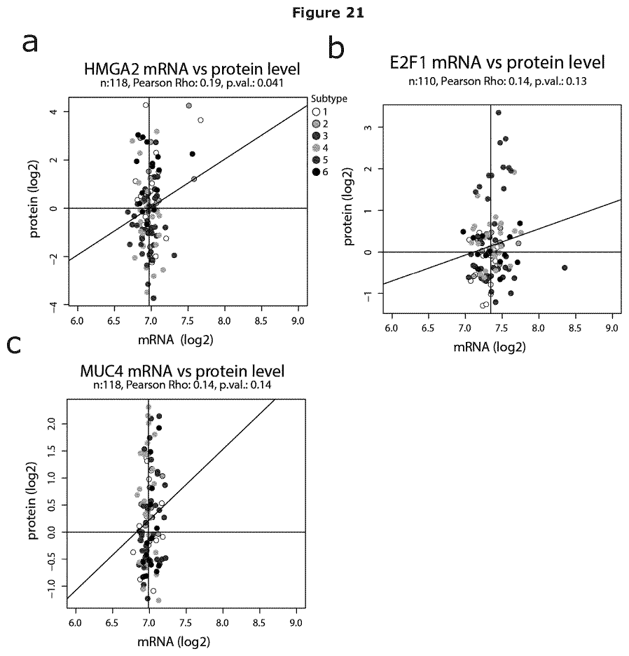

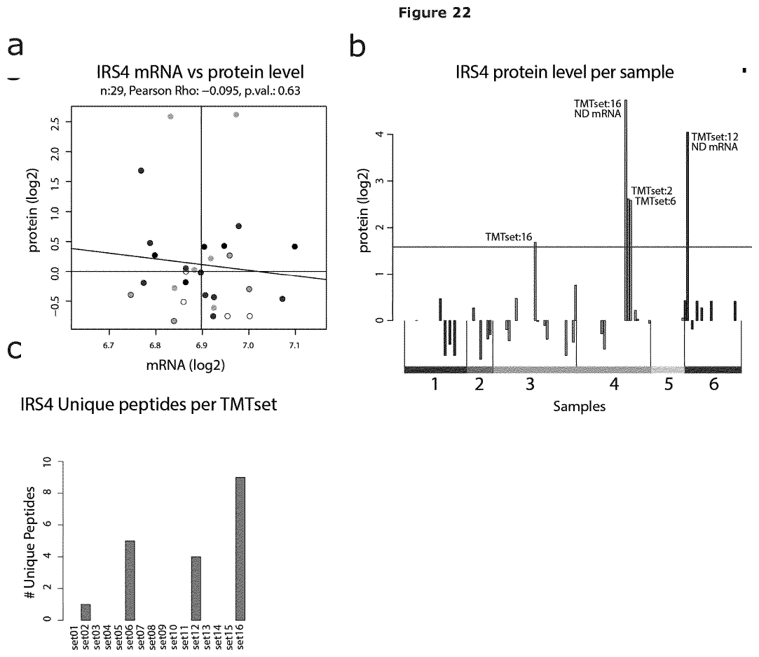

Overall, the mRNA-protein correlation for the majority of CDRPs with outlier expression was high, however, for a subset of CDRPs mRNA levels poorly explained the protein levels (FIG. 9c). As contributing causes for this, the Inventors noted significantly lower mRNA-protein correlation for known miRNA targets²³, known protein complex members²⁴ as well as mRNAs and proteins with low stability²⁵ (FIG. 20-22). As examples, the analysis pointed out low correlation for HMGA2, In line with previously described regulation by the let-7 microRNA²⁶, as well as E2F1 that has been shown regulated by the ubiquitin-proteasome system²⁷. Interestingly, E2F1 protein levels were specifically elevated in Subtype 5 samples, suggesting that E2F1 degradation was reduced specifically in this subtype (FIG. 20-22). Elevated E2F signalling in Subtype was also identified by the network analysis (FIG. 8). MUC4 is another example of regulated protein stability as this protein has been shown degraded via hypoxia-Induced autophagy²⁸. IRS4 is normally only expressed in embryonic tissues, adult brain and testis, but was found highly expressed and acting as an oncogenic driver in a subset of breast cancers. The data supports that IRS4 could be a driver in sporadic cases also in NSCLC but indicates post-transcriptional regulation (FIG. 20-22).

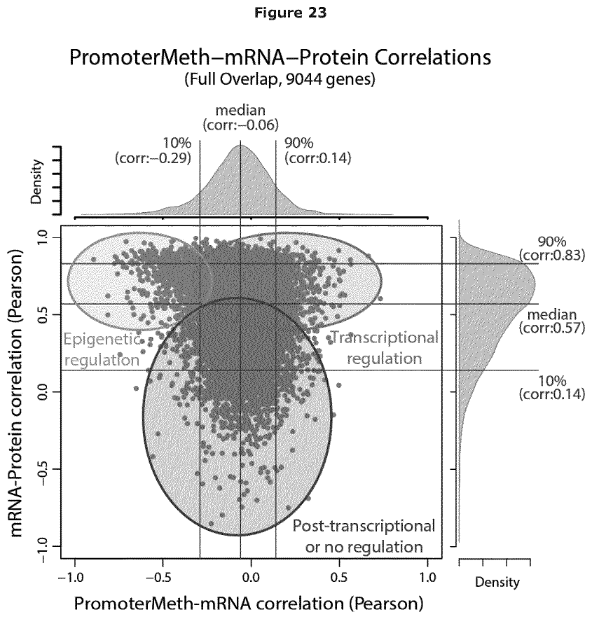

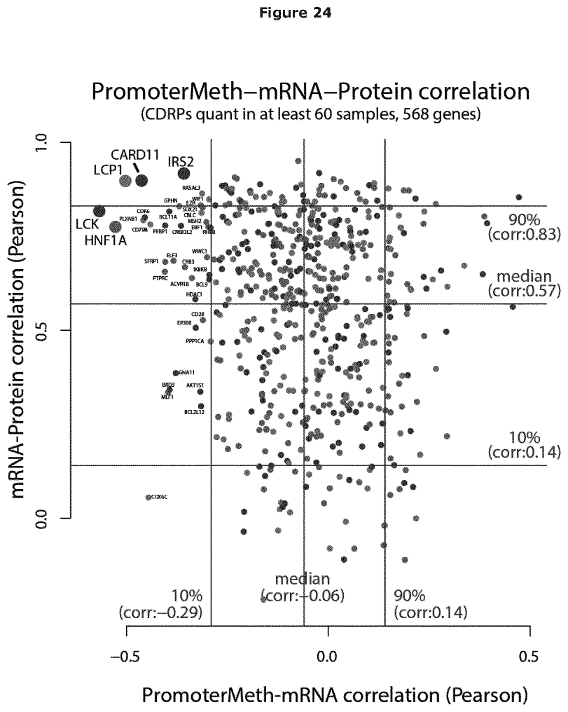

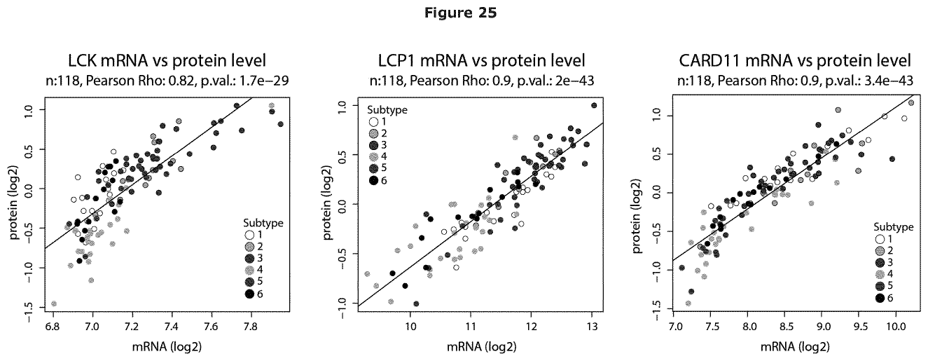

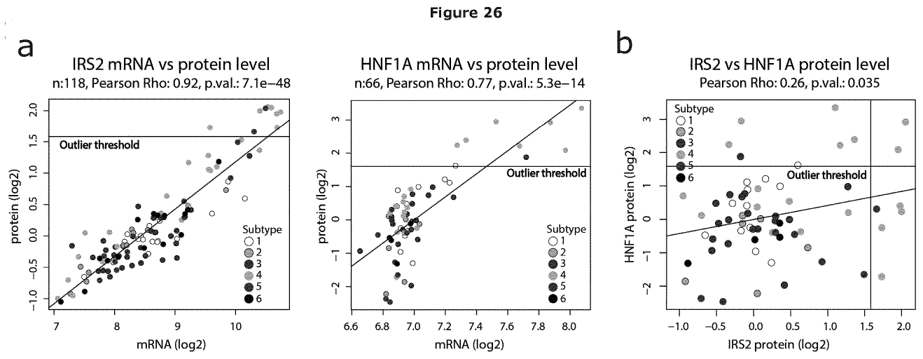

The analytical depth of inventors' MS-analysis, together with supporting genome-wide transcriptomics and methylation data allowed us to perform an overall analysis of gene regulation levels. Plotting the promoter methylation-mRNA correlation against mRNA-protein correlation indicated genes likely to be epigenetically regulated (negative methylation-mRNA and high mRNA-protein correlation), transcriptionally regulated (no/low methylation-mRNA and high mRNA-protein correlation) and post-transcriptionally regulated (no/low mRNA-protein correlation, also including non-regulated proteins with equal level across the cohort), FIG. 23-24). This analysis indicated several CDRPs likely to be epigenetically regulated such as LCK, HNF1A, LCP1, CARD11 and IRS2 (FIG. 9a). LCK, LCP1 and CARD11 all showed modestly higher mRNA and protein levels in subtypes that are more immune Infiltrated (Subtype 2 and 3, FIG. 25), consistent with blood cell and lymphoid tissue specific expression as indicated in the Human Protein Atlas (www.proteinatlas.org). IRS2 and HNF1A on the contrary showed outlier expression in a subset of Subtype 4 samples (FIG. 26a). Methylation of IRS2, a key insulin receptor substrate required for hormonal control of metabolism, was recently associated with high fasting Insulin levels, indicating epigenetic control of this gene³⁰. HNF1A is a liver specific transcription factor that is a master regulator of metabolism, and mutations in this gene is one of the most common causes of Maturity Onset Diabetes of the Young (MODY)³¹. Interestingly, overexpression of these two proteins occurred in different cases, suggesting that sample specific altered epigenetic control of genes involved in metabolism is a feature of Subtype 4 (FIG. 26b).

### 3. Immune Landscape and Neoantigen Burden in NSCLC

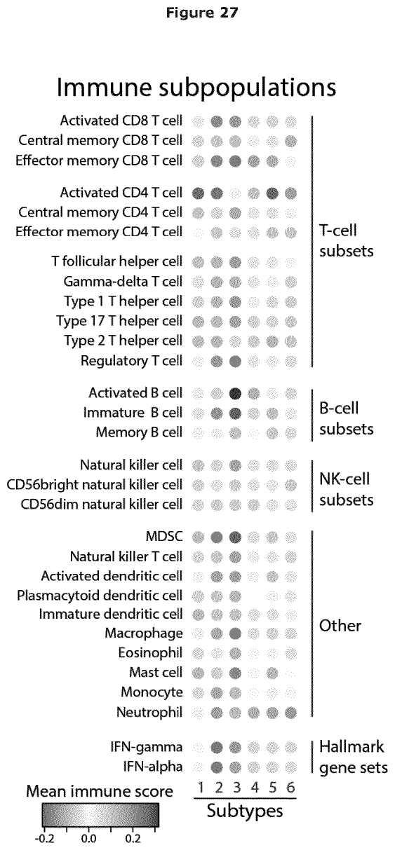

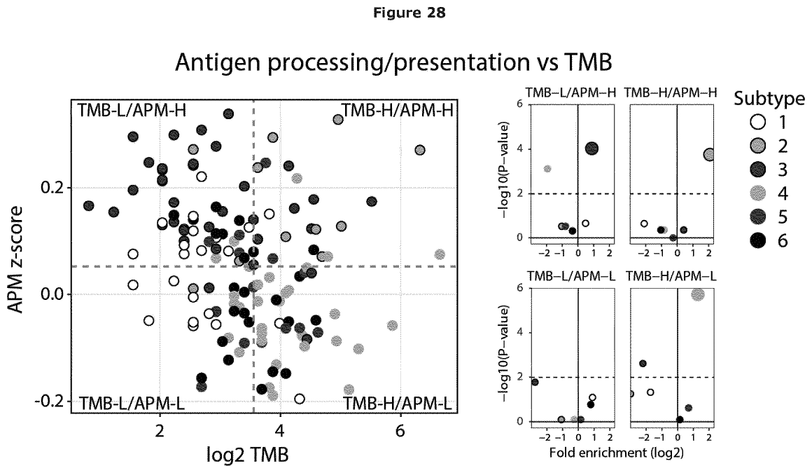

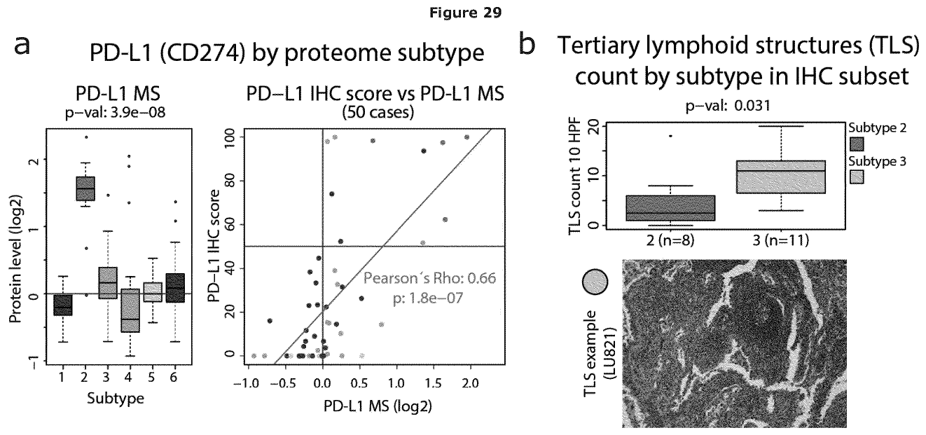

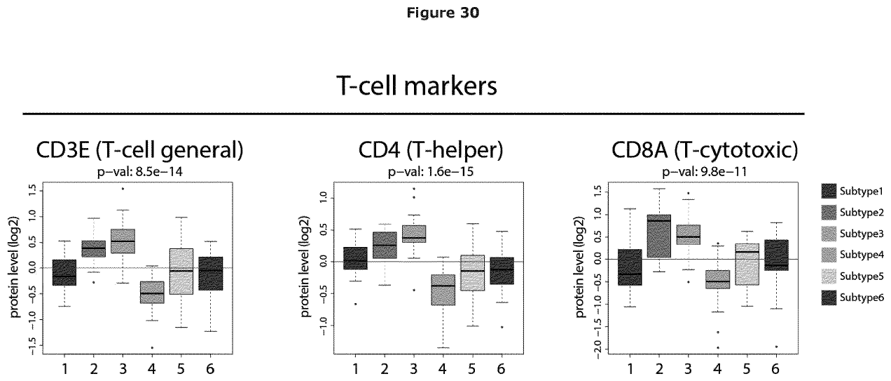

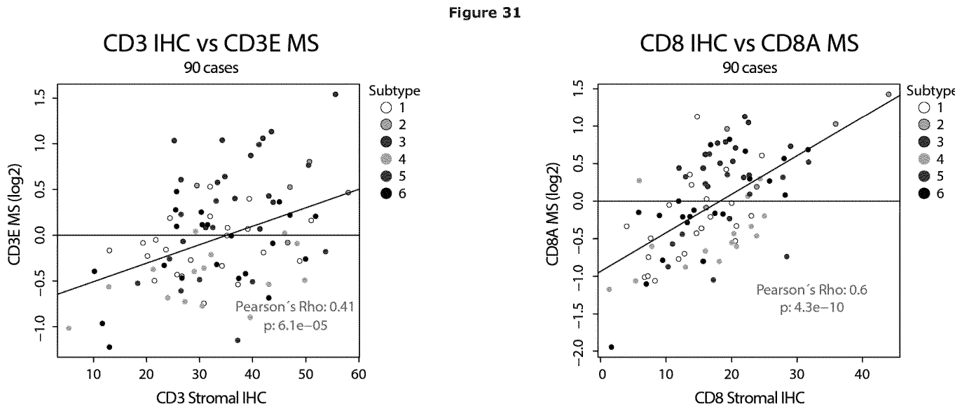

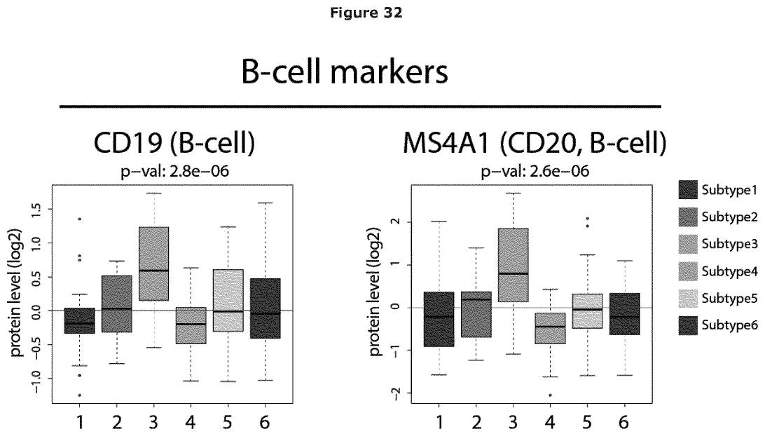

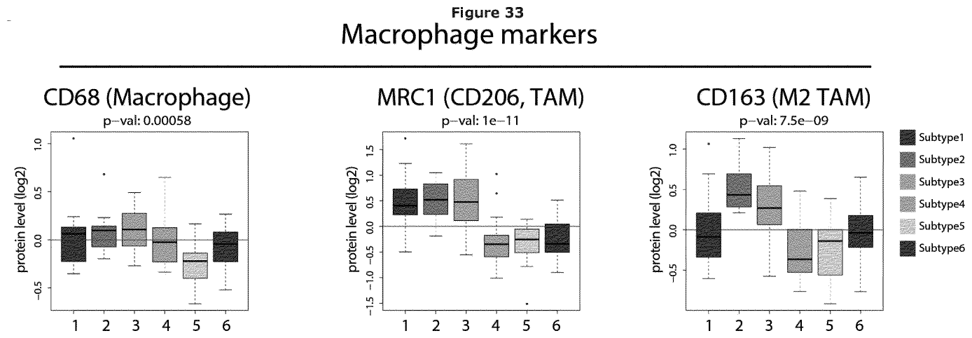

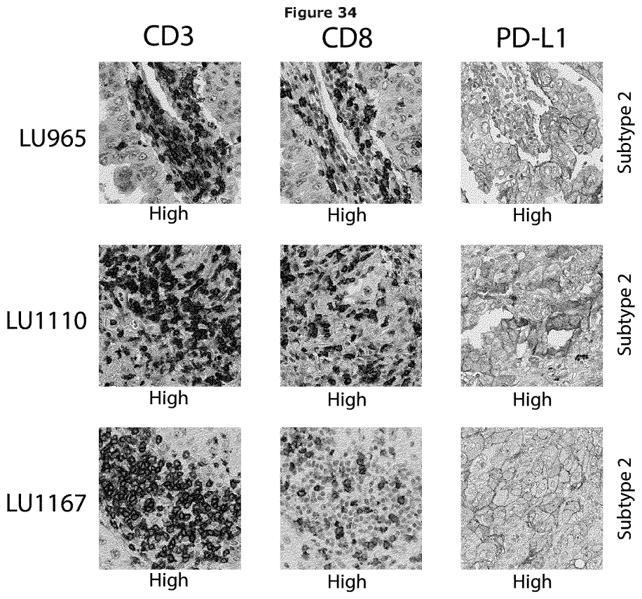

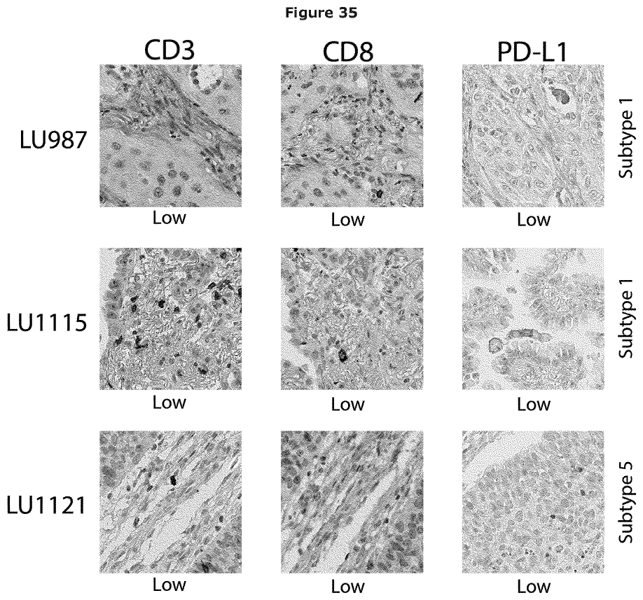

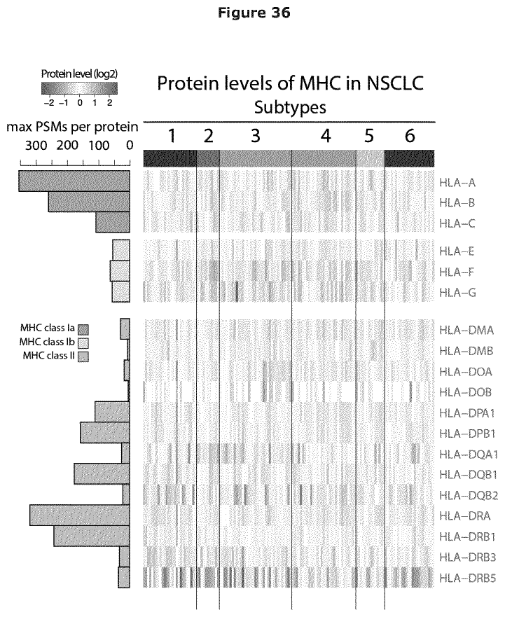

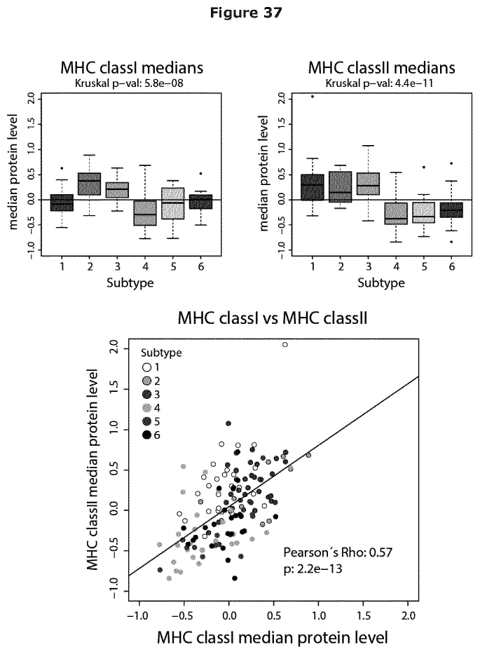

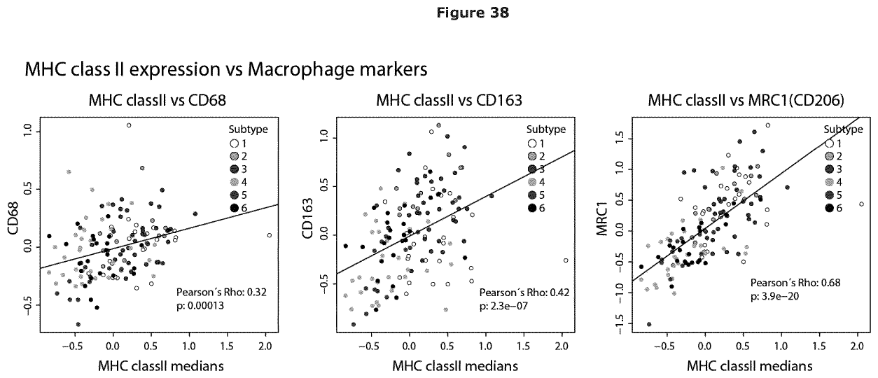

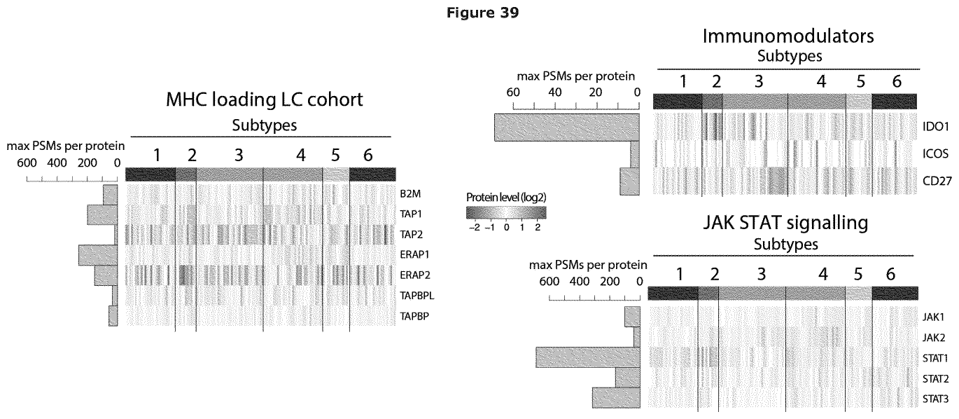

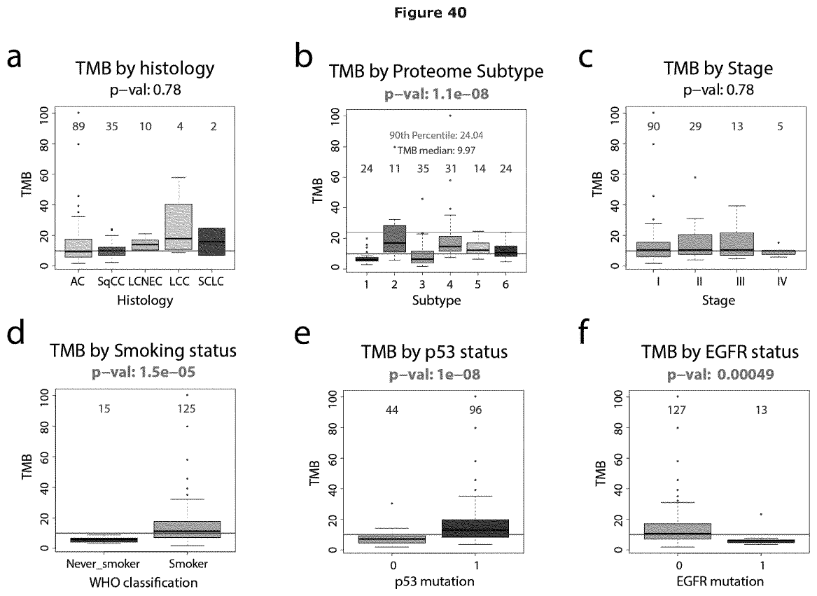

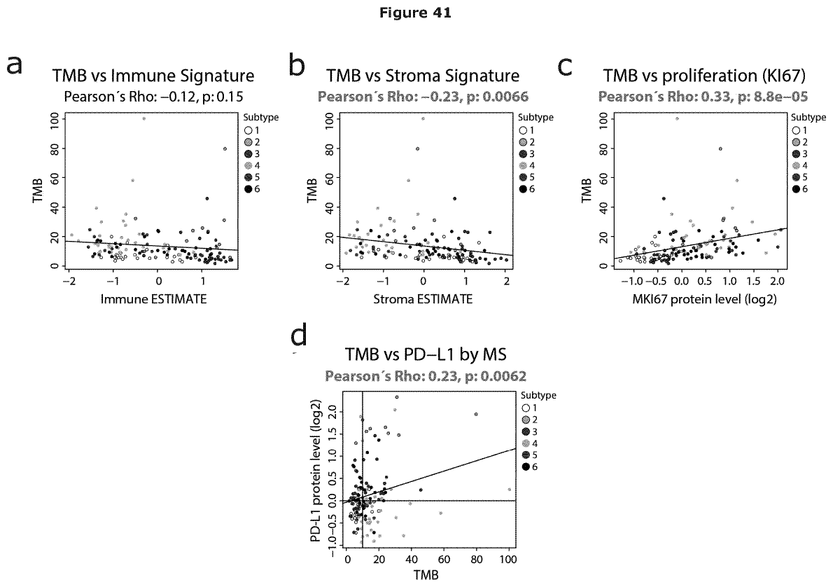

Longstanding intensive research on the Interplay between the immune system and cancer has led to recent major developments in the cancer immunotherapy field. The search for better predictive biomarkers and potential combination therapy strategies is an important area of research to improve and broaden the clinical use of immunotherapy. To get an overview of infiltrating immune cell subpopulations in the cohort samples, the Inventors evaluated the MS-data using previously described immune signatures³². This analysis confirmed the overall high immune infiltration in samples from Subtypes 2 and 3. In particular, there was high signal for T-cells and IFN signalling in Subtype 2, and for B-cells in Subtype 3, suggesting a differential Immune response in these two subtypes (FIG. 27, FIG. 30-33). CD3 and CD8A immunohistochemistry (IHC) was performed on a subset of cases indicating overall correlation between MS data and stromal staining (FIG. 30-34). In contrast, Subtype 4 showed very low signals for all immune cell subpopulations, Indicating an overall immune-cold subtype. Next, the inventors investigated antigen processing and presentation machinery (APM, FIG. 36-39) In relation to tumour mutation burden (TMB, FIG. 40-41) to evaluate the potential of neoantigen-dependent immune cell activation as recently performed for endometrial carcinoma³³. This analysis separated the different NSCLC Subtypes, specifically indicating that Subtype 2 samples were associated with both high TMB and APM, while Subtype 3 showed high APM but low TMB, and Subtype 4 high TMB but low APM (FIG. 28). Subtype 2 thus fulfils the requirements to elicit a strong immune activation as high TMB and APM would suggest production of neoantigens that are also presented. Interestingly, the subtype marker analysis revealed PD-L1 as one of the clearest marker proteins of Subtype 2 (FIG. 29a, FIG. 34), Indicating that PD-L1/PD-1 immune checkpoint is an important immune evasion mechanism in this group of tumours, suggesting targeting this checkpoint, would be efficient in these patients.

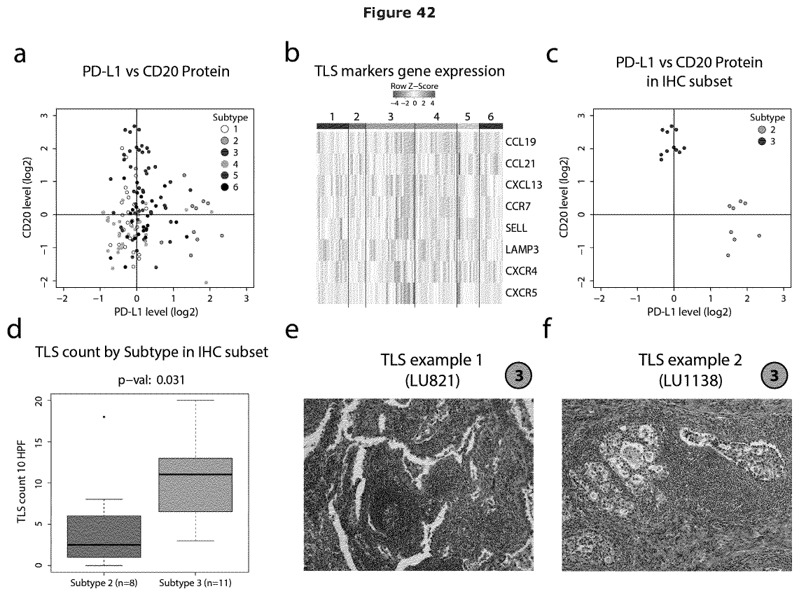

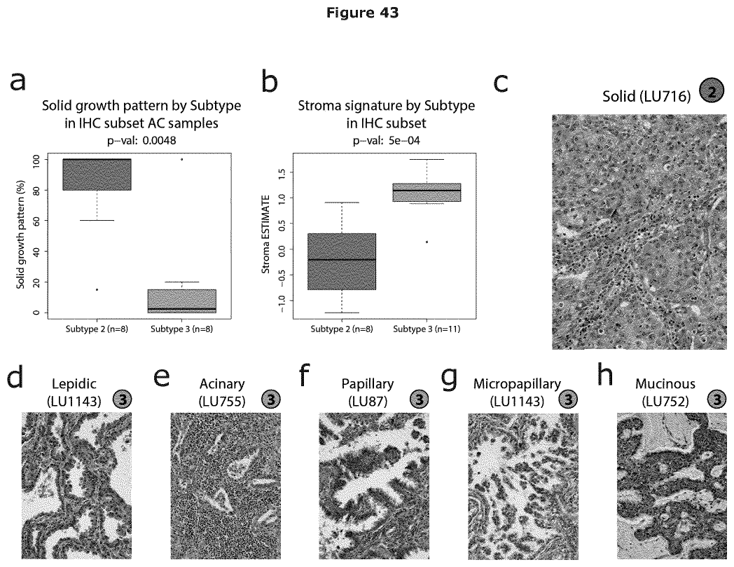

The immune landscape evaluation suggested high infiltration of B-cells in Subtype 3 samples, and in addition the inventors noted a dichotomy between the expression of B-cell markers and the expression of PD-L1 (FIG. 42). B-cell rich tertiary lymphoid structures (TLSs) have previously been shown associated with good prognosis' as well as response to immunotherapy³⁵. An evaluation of TLS markers based on mRNA level analysis as previously described³⁵ indicated high expression in a subset of Subtype 3 samples (FIG. 42). To investigate this further the Inventors evaluated tumour sections from a subset of the samples with either high PD-L1 (Subtype 2) or high levels of B-cell markers (Subtype 3, FIG. 42). This analysis supported the presence of TLSs in Subtype 3 (FIG. 29b, FIG. 42d-f), but also indicated differences in predominant growth patterns between AC samples in Subtype 2 and 3. While Subtype 2 samples almost exclusively showed a solid growth pattern with low stromal component, Subtype 3 samples showed variable degrees of lepidic, acinary, papillary, micropapillary, mucinous and solid growth patterns (FIG. 43). Overall these results emphasise that while both Subtype 2 and 3 samples are infiltrated by immune cells, the type of infiltrating immune cells as well as the AC growth pattern is strikingly different.

The use of TMB as an approximation of actual neoantigen burden is not necessarily accurate, since mutations are not the only source of neoantigens. Transcription and translation of genes normally silenced in tissues other than testis (so-called “cancer testis antigens”) as well as of DNA sequences not expected to produce proteins at all (so-called “non-canonical” or “alternative” or “aberrantly expressed”, from this point on referred to as non-canonical proteins/peptides or NCPs) could also elicit an immune reaction against the cancer cells. There is accumulating evidence that peptide neoantigens deriving from genomic regions annotated as non-coding are expressed in cancer^{11,36-38}. These complex neoantigens are suggested to be more immunogenic than single nucleotide variant (SNV)-mutation derived neoantigens, which are often too similar to the self-antigens^{39,40}.

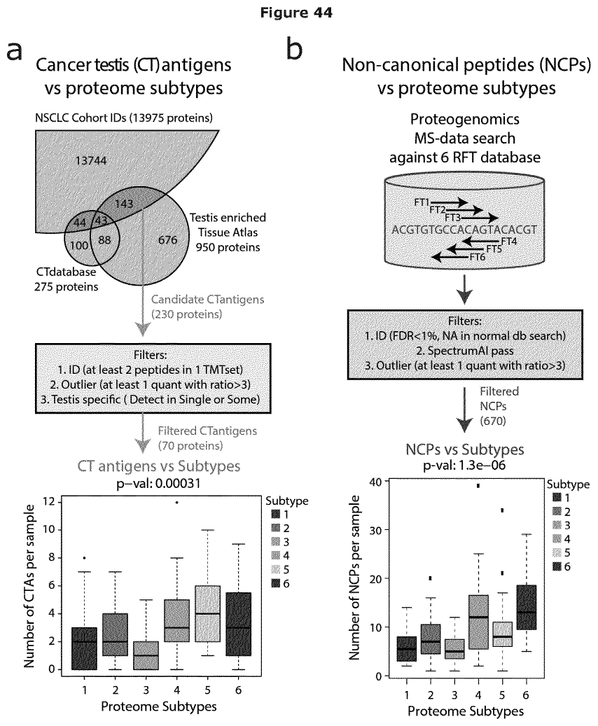

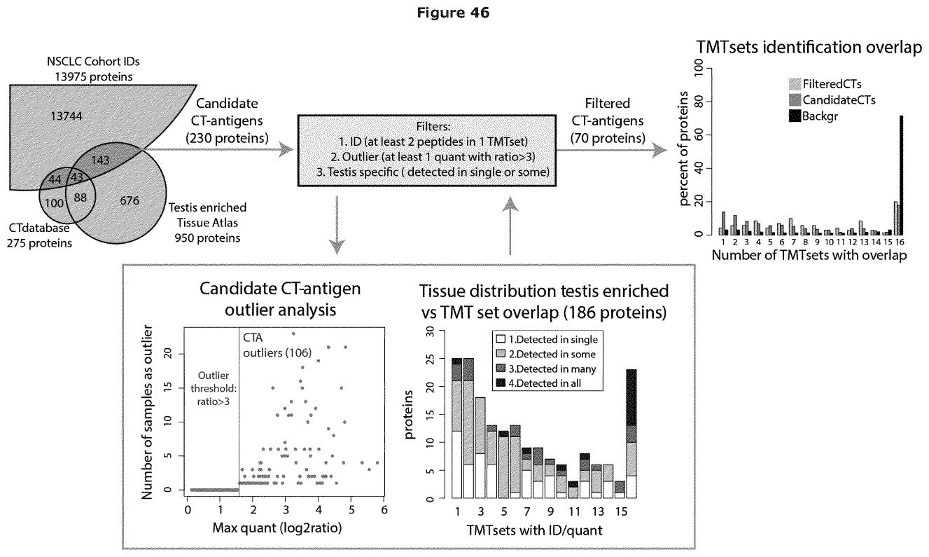

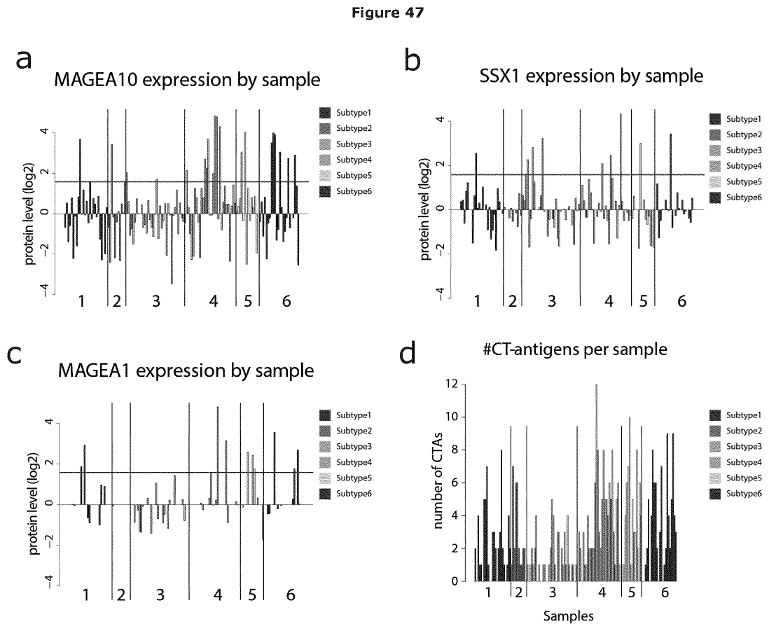

First, to evaluate the expression of cancer testis (CT) antigens in the current cohort the inventors defined CT antigens as genes present in the CTdatabase⁴¹ or genes annotated as testis-enriched according to the human protein atlas (www.proteinatlas.org) (FIG. 44a, FIG. 46-47). Out of these genes, 230 were identified at the protein level in the current cohort, and after filtering, 70 CT antigens identified with at least 2 unique peptides and outlier expression pattern (sample protein level>3-fold up compared to cohort median) were evaluated further. The expression of CT-antigens was found to be widespread across the cohort samples, with significant differences between the six proteome subtypes and, intriguingly, with more expression in the immune-cold subtypes (Subtype 4-6, FIG. 44a).

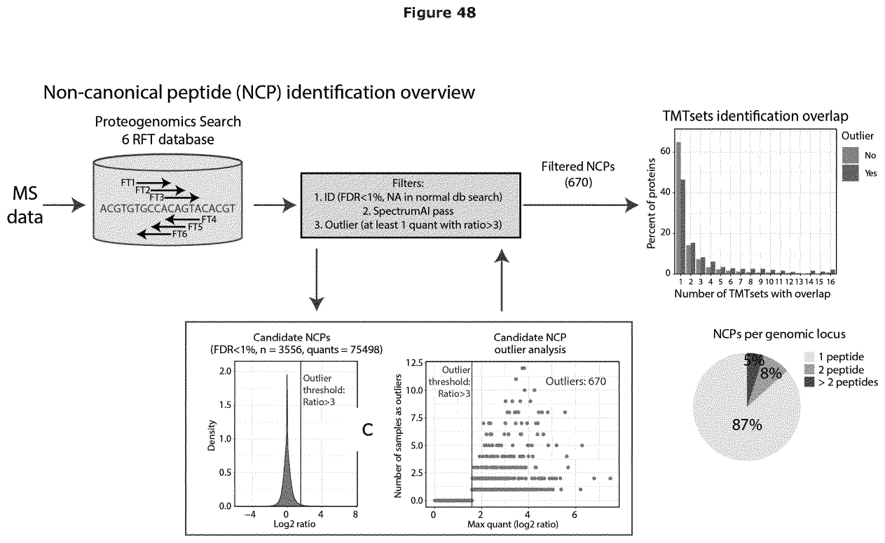

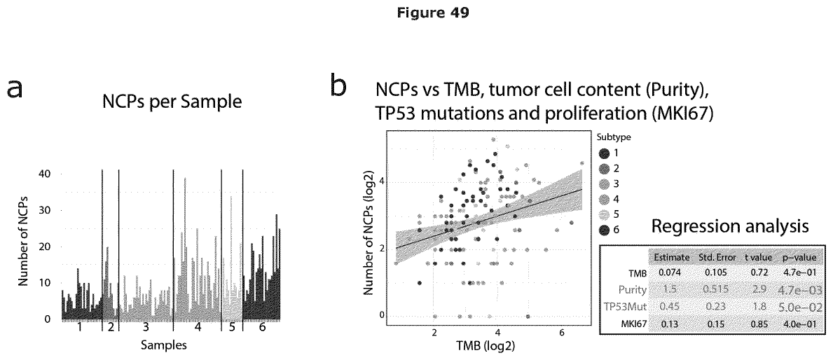

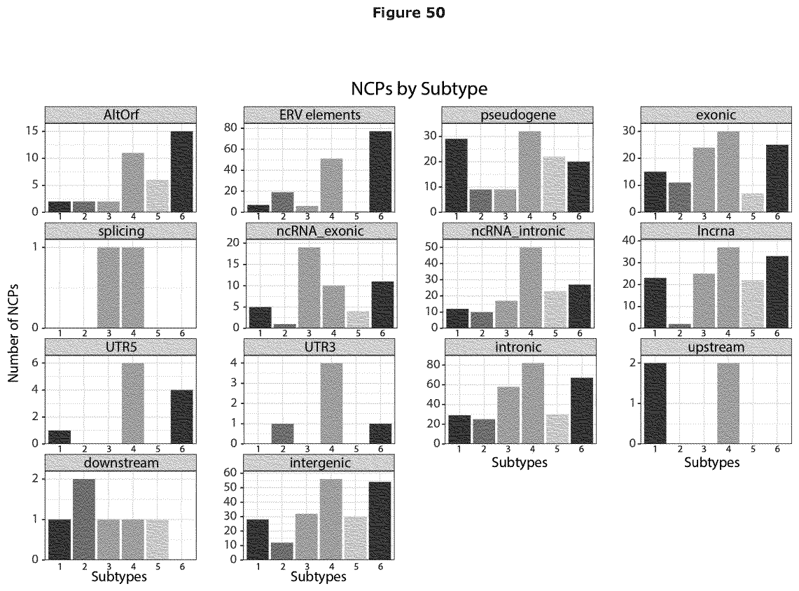

Second, for an unbiased evaluation of non-canonical peptides, the inventors performed proteogenomics by searching MS-data against a peptide database produced by 6-reading frame translation (6RFT) of the entire human genome as previously described^{9,10} (FIG. 44b, FIG. 48-50). Searching against a 6RFT database allows for protein level detection of potentially immunogenic NCPs caused by e.g. frame shift mutations or indels, or mapping to e.g. pseudogenes or endogenous retroviral (ERV) elements. Following the same outlier expression pattern as in CT antigens (FC>3), the inventors identified 670 non-canonical peptides (FDR<1%), with 13% of the corresponding genetic loci supported by more than one peptide (FIG. 48). This analysis indicated large differences between samples, as well as significant differences between subtypes in the number and types of NCPs identified as outliers (FIG. 44b, FIG. 49a, FIG. 50). Interestingly, as in the case of CT-antigens, these complex NCP-antigens were detected in highest numbers in immunologically cold tumours (Subtype 4 and 6). Further, regression analysis indicated no significant effect of TMB on the number of NCPs per sample when adjusted for tumour cell content, TP53 mutations and proliferation probed by MKI67 (MS). Instead, the same multivariate model suggested that the number of NCPs per sample was associated with tumour cell content (P=0.0047) and TP53 mutation (P=0.05). (FIG. 49b).

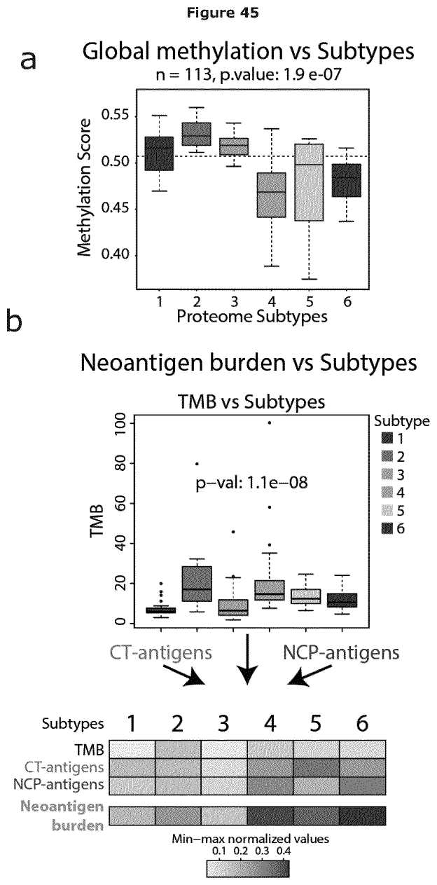

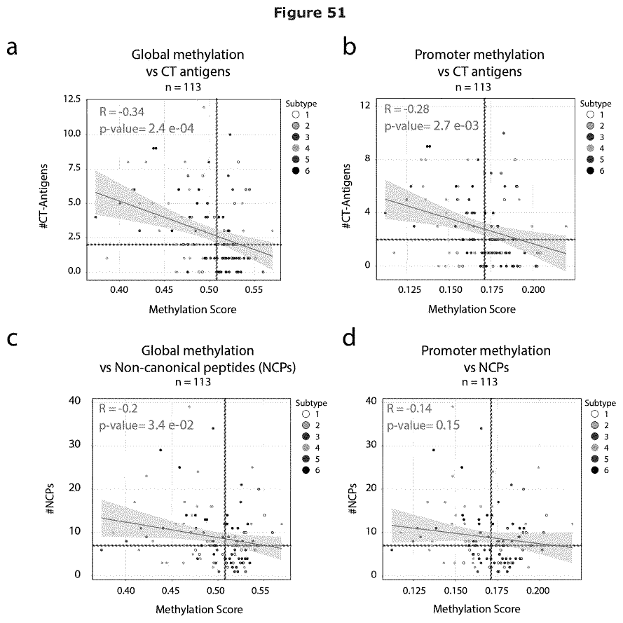

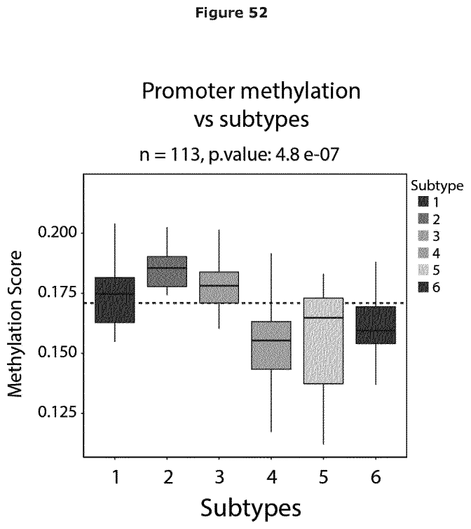

Previous research has shown that global hypomethylation as well as promoter-specific hypomethylation is associated with CT-antigen expression⁴². In inventors' proteome-wide analysis, the number of identified CT-antigens per sample showed a significant negative correlation to both global methylation and promoter methylation, indicating that looser epigenetic control contributes to protein level expression of CT-antigens in NSCLC (FIG. 51a-b). Importantly also the number of identified non-canonical peptides per sample showed negative correlation to global methylation (FIG. 51c-d). Further, the analysis revealed significant differences between subtypes in global methylation (FIG. 45a), as well as promoter methylation (FIG. 52), with the lowest methylation found in Subtypes 4 and 6.

To more comprehensively evaluate the potential for activation of anti-cancer immune response, the inventors evaluated TMB In relation to CT-antigens and NCP-antigens in the NSCLC cohort and summarized these three metrices into a tumour neoantigen burden (TN B) score (FIG. 45b). This analysis indicates that while Subtype 2 has the highest TMB, Subtypes 4, 5 and 6 produce other types of neoantigens that could elicit a strong immune response given efficient presentation and infiltration of immune cells.

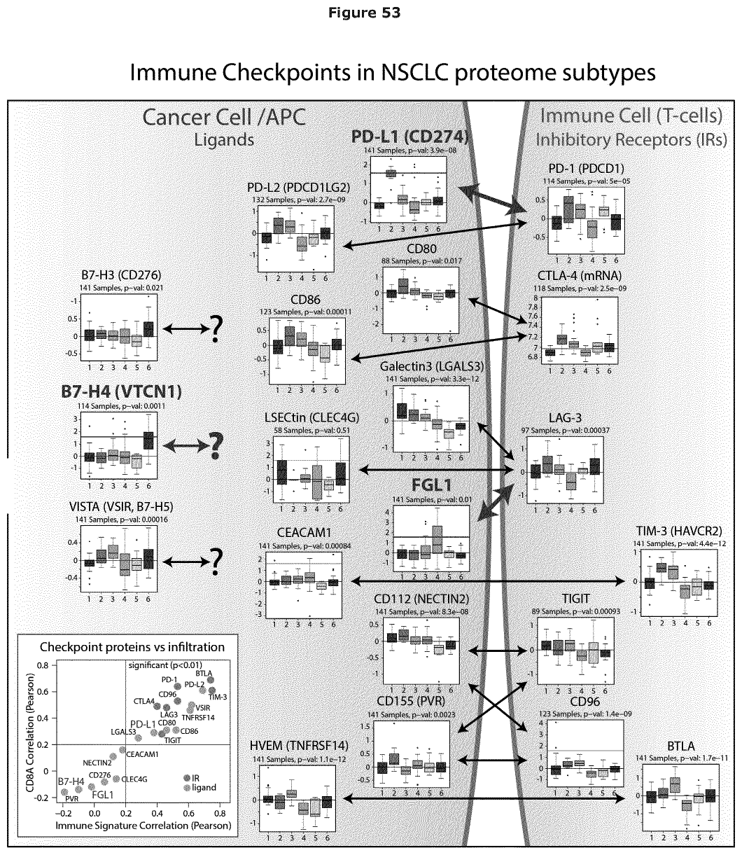

The analysis above indicated differences in immune infiltration and neoantigen burden between the NSCLC subtypes. To further elucidate the picture, the inventors performed a systematic evaluation of immune checkpoints based on previously identified inhibitory receptors (IRs) and their corresponding ligands^{43,44} (FIG. 53). This analysis indicated that the protein levels of IRs in general correlated with infiltration of T-cells. IR ligands, (expressed by cancer cells and APCs) on the contrary, showed more variable patterns, suggesting that different subtypes may use different immune evasion mechanisms. The most striking IR ligand expression was found for PD-L1 In Subtype 2, but intriguingly the analysis also revealed two other subtype specific IR ligands, FGL1 in Subtype 4 and B7-H4 in Subtype 6 (FIG. 53). FGL1 was recently identified as a tumour cell secreted, high affinity ligand to LAG3, causing FGL1-LAG3 mediated suppression of T-cells⁴⁵. Further it was shown that blockade of this Interaction potentiated anti-tumour immunity. B7-H4 acts as an immune checkpoint to prevent autoimmunity⁴⁶, and it has been shown in mouse models that blocking B7-H4 by therapeutic antibodies increases the tumour infiltration of CD8+ T cells, reduces the tumour growth and the formation lung metastases of CT26 mouse models⁴⁷.

Taken together, the immuno phenotype analysis, the neoantigen burden analysis and the checkpoint analysis show that the NSCLC proteome subtypes here identified may have predictive value for different types of checkpoint inhibitors already in clinical use, or investigated in clinical trials.

### 4. Subtype 4 is Characterized by STK11 Inactivation Resulting in Oncogenic mTOR-Signalling and Immune Evasion Through FGL1

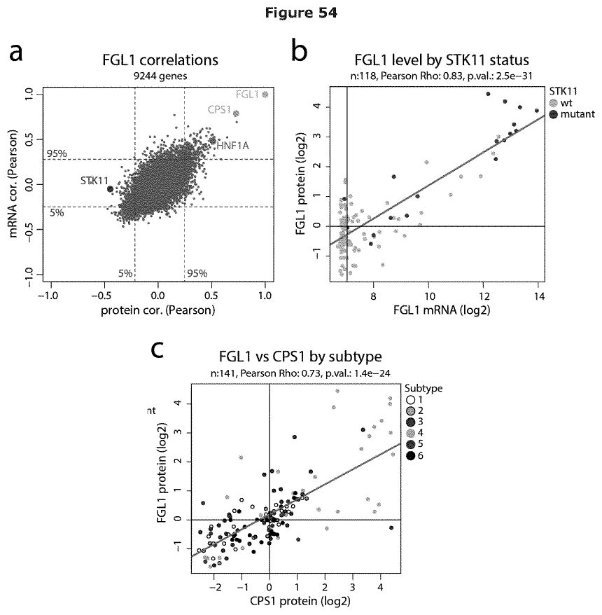

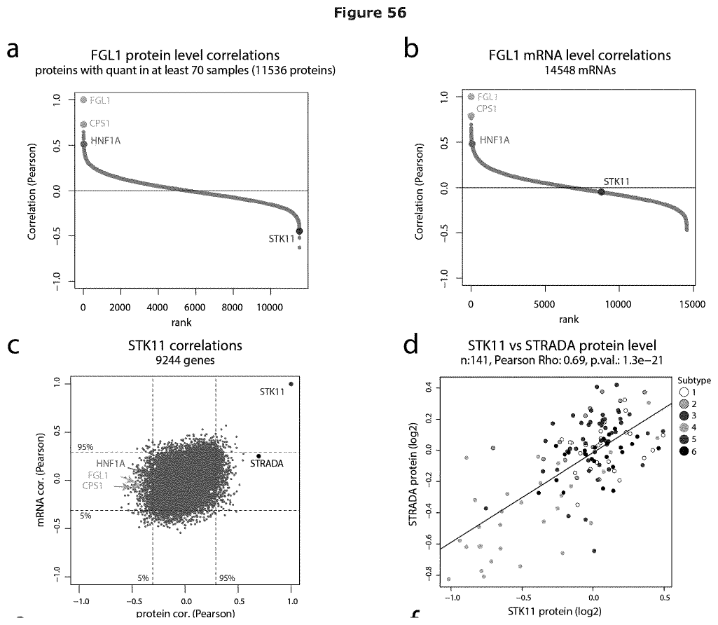

To investigate the mechanism behind FGL1 expression in the immune cold Subtype 4, the inventors performed a correlation analysis to identify FGL1 associated proteins and transcripts (FIG. 54a, FIG. 56a-b). This analysis showed a strong negative protein level correlation between FGL1 and the tumour suppressor STK11 (LKB1) that was also frequently mutated in Subtype 4. Further evaluation revealed a strong coincidence between STK11 mutation and high FGL1 protein and mRNA levels (FIG. 54b). The finding that FGL1 and STK11 only anticorrelate at the protein level, but not at the mRNA level, suggests post-transcriptional regulation of STK11. STK11 forms a functional heterotrimeric complex with STRADα and CAB39 (MO25α)⁴⁸, and in inventors' data a stabilizing effect of this complex was supported as the correlation between STK11 and STRADα was much higher at protein level (0.69) than at the mRNA level (0.25, FIG. 56c). Further, low levels of STK11 and STRADα were found almost exclusively in Subtype 4 samples (FIG. 56d).

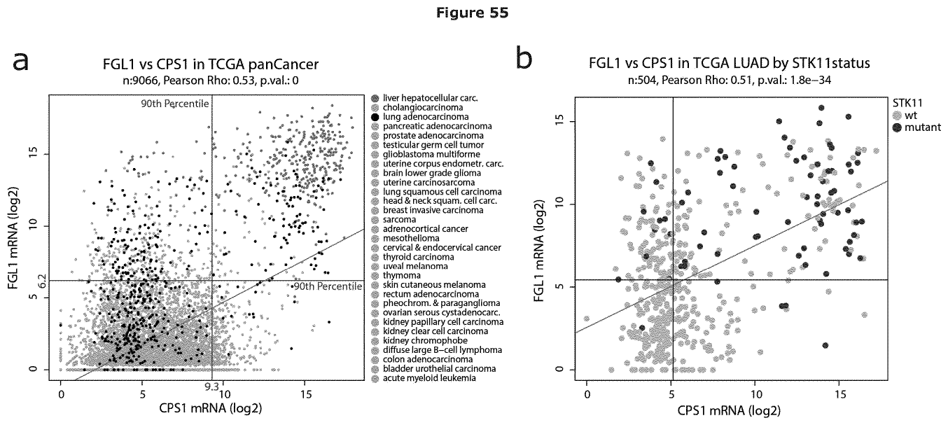

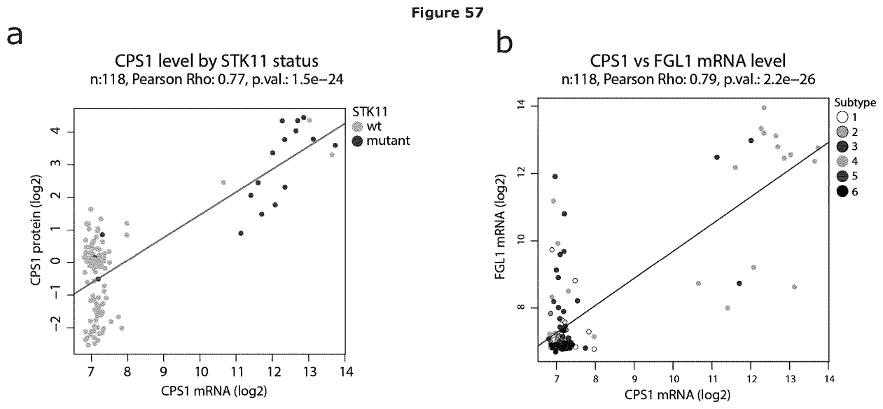

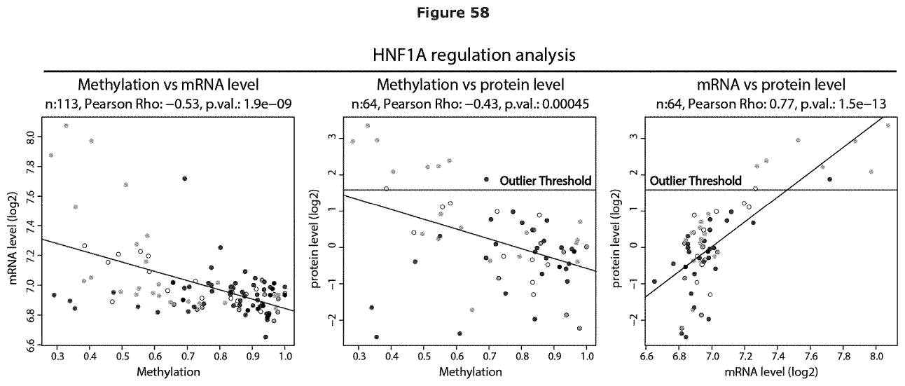

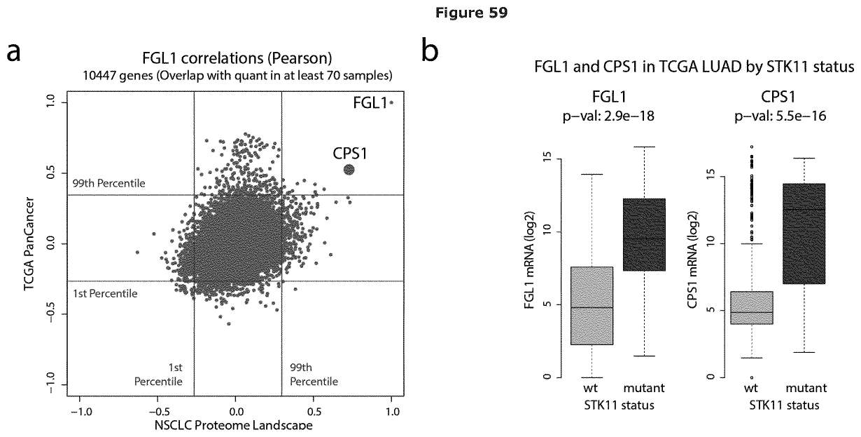

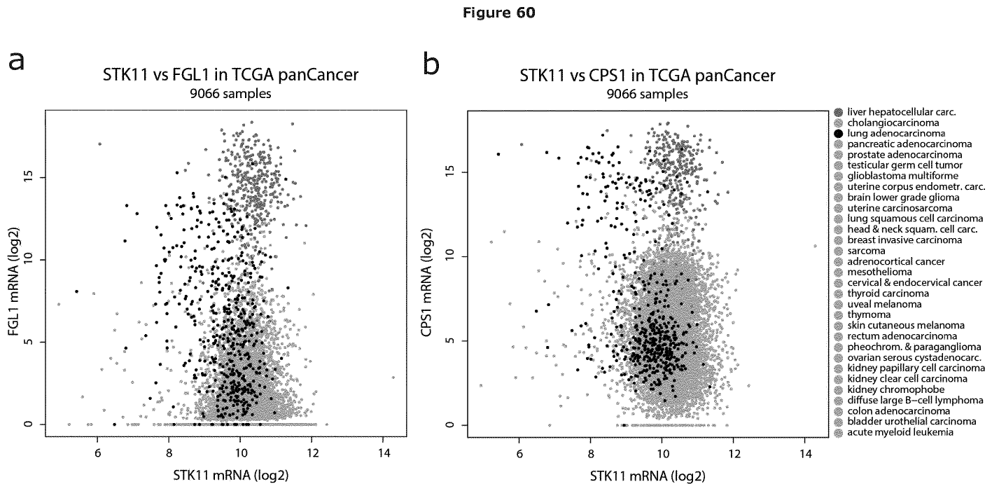

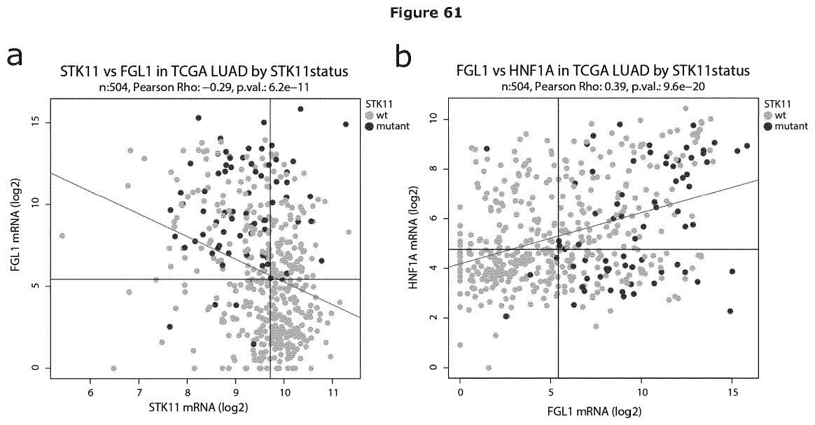

Intriguingly, the protein/mRNA with the highest correlation to FGL1 was found to be CPS1 (FIG. 54a, FIG. 54c and FIG. 57b). CPS1 is a mitochondrial enzyme in the urea cycle previously shown to be upregulated in cancer cells through the AMPK-mTOR signalling pathway after inactivation of STK⁴⁹. This connection is evident also in the current data, as samples with high CPS1 expression at mRNA and protein levels are commonly mutated for STK11 (FIG. 57a). FGL1 and CPS1 are normally only expressed in liver cells^{45,49} and inventors analysis here suggests that STK11 Inactivation results in transcriptional upregulation of both genes. Evaluating the FGL1 mRNA/protein correlation analysis against transcriptions factors as annotated in the animalTF database⁵⁰ indicated the liver specific transcription factor HNF1A as the highest correlating transcription factor (FIG. 54a). Interestingly, as described above, HNF1A was also picked up as a gene potentially regulated by epigenetic mechanisms in NSCLC which is common for tissue/lineage specific genes (FIG. 58). To further investigate the potential link between STK11, FGL1 and CPS1 in cancer the inventors used public domain data generated in The Cancer Genome Atlas project (TCGA). Gene expression data covering 31 different cancer types (PanCancer dataset⁵¹) supported a strong co-expression of FGL1 and CPS1 (FIG. 59a). In particular, high mRNA-levels were evident for hepatocellular carcinoma samples (FIG. 55a), which is in agreement with the documented liver-specific expression of both genes. Importantly, also a subset of lung adenocarcinoma samples showed high gene expression of FGL1 and CPS1, and both genes were significantly higher expressed in STK11 mutated AC samples compared to wild type STK11 cases, supporting that FGL1 and CPS1 transcription is controlled by STK11 dependent signalling (FIG. 55b, FIG. 59b). Multiple samples that were wild type for STK11 also showed high mRNA expression of FGL1 and CPS1 indicating loss of STK11 function by other mechanisms or phenocopying alterations in other regulators of same pathway. In lung adenocarcinoma specifically, Increased FGL1 and CPS1 mRNA levels was associated with reduced STK11 mRNA expression, indicating that transcriptional or epigenetic regulation could contribute to STK11 inactivation (FIG. 60, FIG. 61a). As no protein level data exists for FGL1/CPS1 in the TCGA samples the inventors could not evaluate any potential negative protein level correlation with STK11. Finally, FGL1 mRNA expression significantly correlated to HNF1A mRNA expression in lung adenocarcinoma (FIG. 61b).

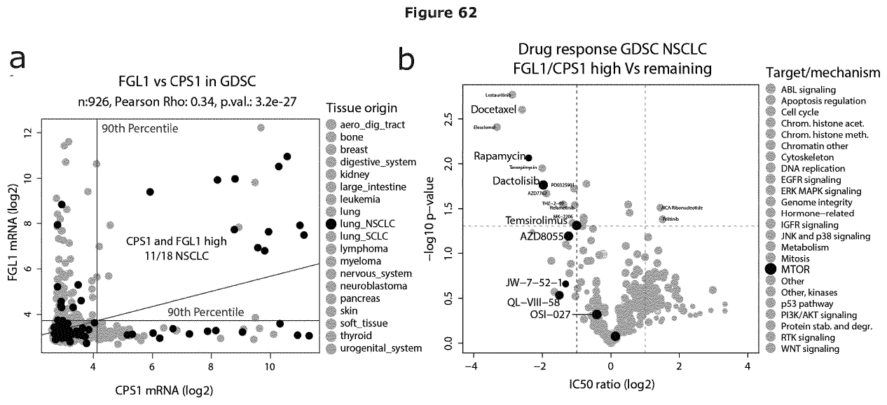

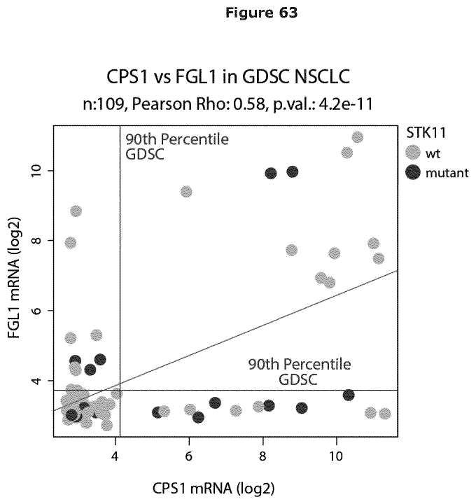

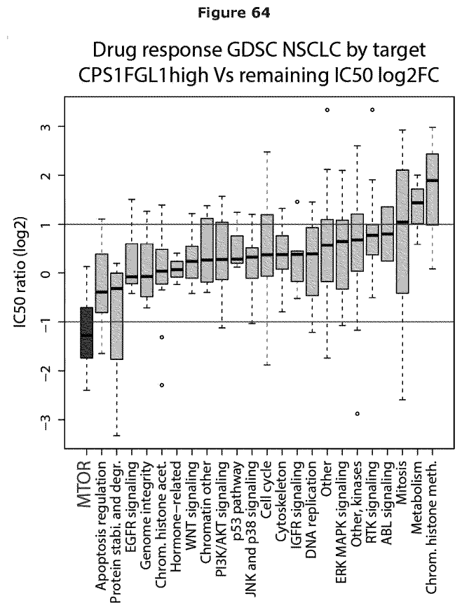

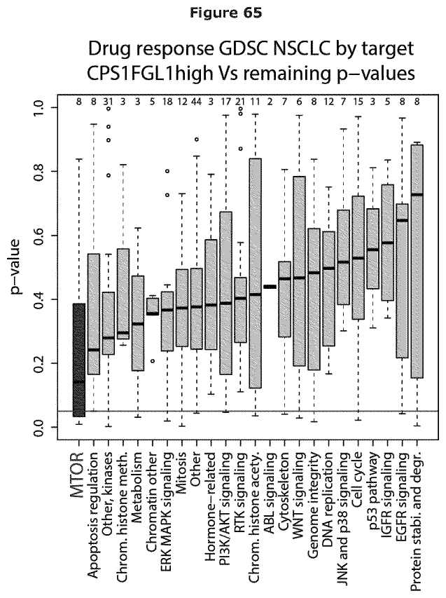

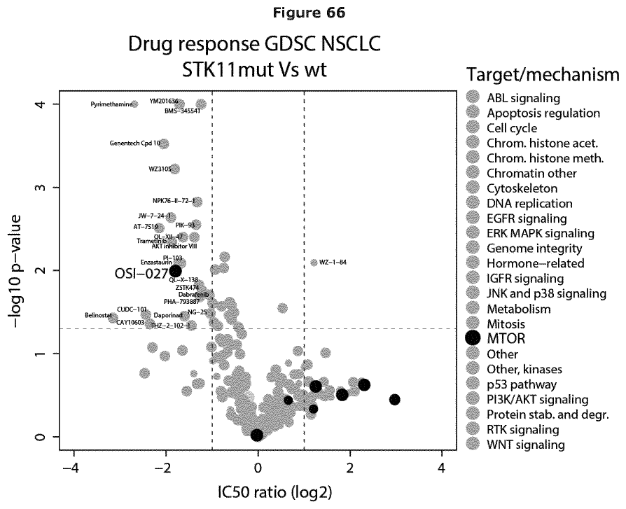

The analyses here performed indicate a distinct lung adenocarcinoma subgroup largely captured by proteome Subtype 4. To evaluate whether this subgroup could be associated with any specific drug sensitivity patterns with potential clinical implications, the inventors used data generated in the Genomics of Drug Sensitivity in Cancer (GDSC) project⁵². The GDSC resource contains drug response measurements for a large number of compounds, as well as gene expression and mutation data for a wide collection of cancer cell lines. Analysis of the mRNA levels of FGL1 versus CPS1 across 926 cell lines again revealed co-expression specifically in a subgroup of NSCLC cell lines (FIG. 62a). Focusing on NSCLC cell lines (n=109), the inventors continued to evaluate differences in drug response between cell lines with high mRNA expression of both FGL1 and CPS1 (n=11 cell lines) and remaining cell lines (n=98) (FIG. 63). This analysis revealed higher sensitivity of FGL1/CPS1 expressing cells to docetaxel, which is a commonly used chemotherapy agent in NSCLC, but strikingly also higher sensitivity to multiple compounds targeting mTOR signalling (FIG. 62b, FIG. 64-65). STK11 inhibits mTOR signalling through activation of AMPK, and in cancer cells with loss of STK11-AMPK activity mTOR becomes an oncogenic driver⁵³. The results indicate that elevated FGL1/CPS1 levels is a solid Indicator of loss of STK11-AMPK signalling, and as such a potential predictor of mTOR addiction in this group of lung adenocarcinoma. Importantly, STK11 mutation alone could not predict sensitivity to mTOR inhibitors, again Indicating alternative STK11 Inactivation mechanism and highlighting the need of phenotype level Information for a more comprehensive understanding of pathway activity (FIG. 66).

Taken together, Inventors' analysis Indicates that Subtype 4 is characterized by inactivation of STK11 resulting in overactivation of mTOR signalling, expression of the liver specific transcription factor HNF1A and transcriptional activation of the two liver specific genes, FGL1 and CPS1, potentially contributing to both immune evasion and cancer growth.

### 5. DDA- and DIA-Based Classification of NSCLC Proteome Subtypes

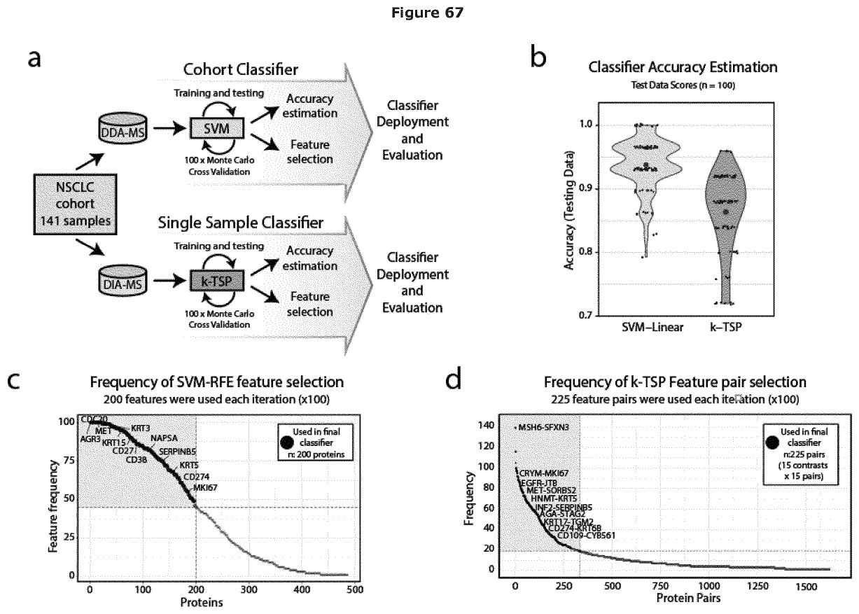

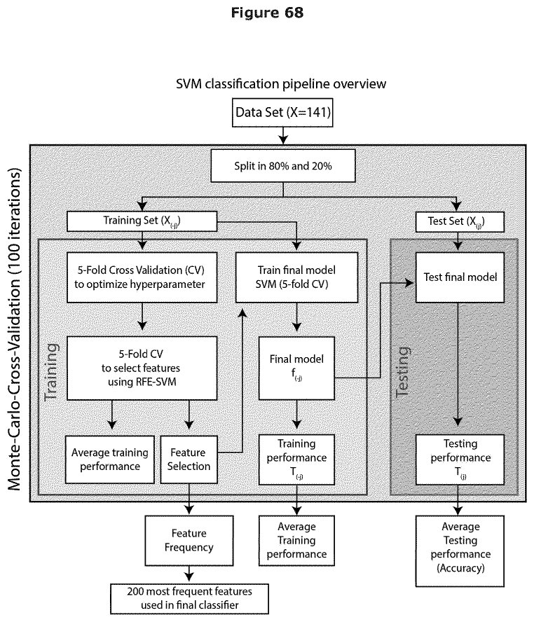

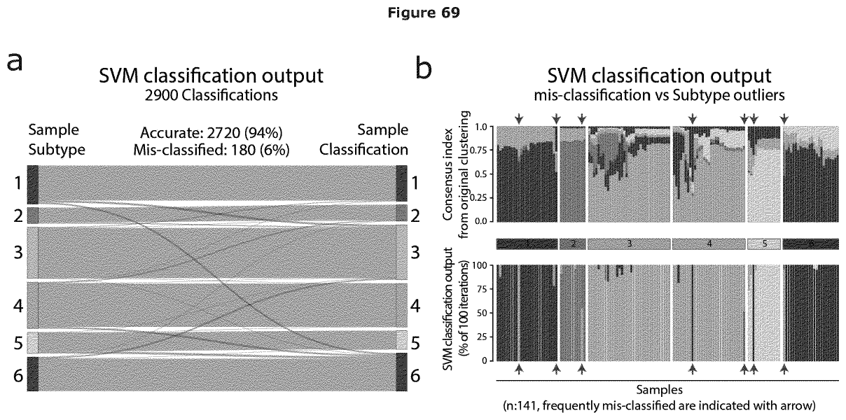

The analysis above indicated clinical value of the NSCLC proteome subtypes here presented. To enable transfer of this knowledge Into a clinical setting, the inventors developed two NSCLC classification pipelines; one support vector machine (SVM)-based for classification of sample cohorts, and one k-Top Scoring Pairs (k-TSP)-based for single sample classification (FIG. 67a). Briefly, the SVM classifier was optimised by Monte Carlo cross validation (100 Iterations), using the TMT-HiRIEF-LC-MS-DDA data of the 141 sample NSCLC cohort described above, where for each iteration the cohort was split Into a training part (80%) and a testing part (20%, FIG. 68). In each iteration, the pipeline reported the classification accuracy based on the test data, as well as the 200 most Important features for the classifier. The accuracy was consistently high (average: 94%, FIG. 67b), and the overlap was large among the Important feature sets (FIG. 5c). Overall misclassifications were sparse (6%, FIG. 69a), and mostly restricted to subtype outliers as evaluated by the consensus Index analysis generated during the original clustering of the 141 samples (FIG. 69b).

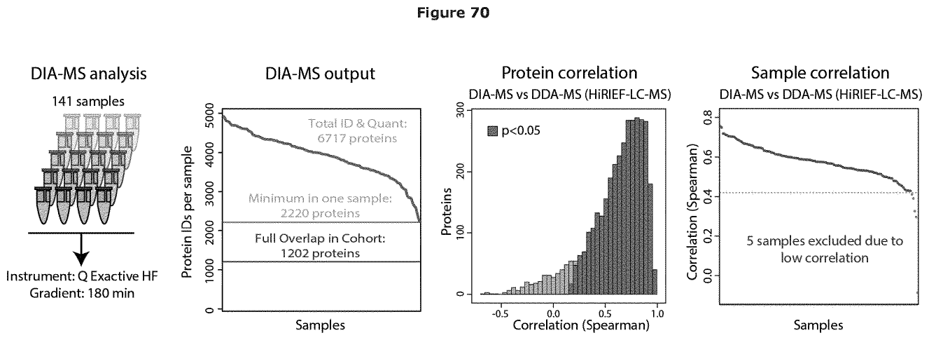

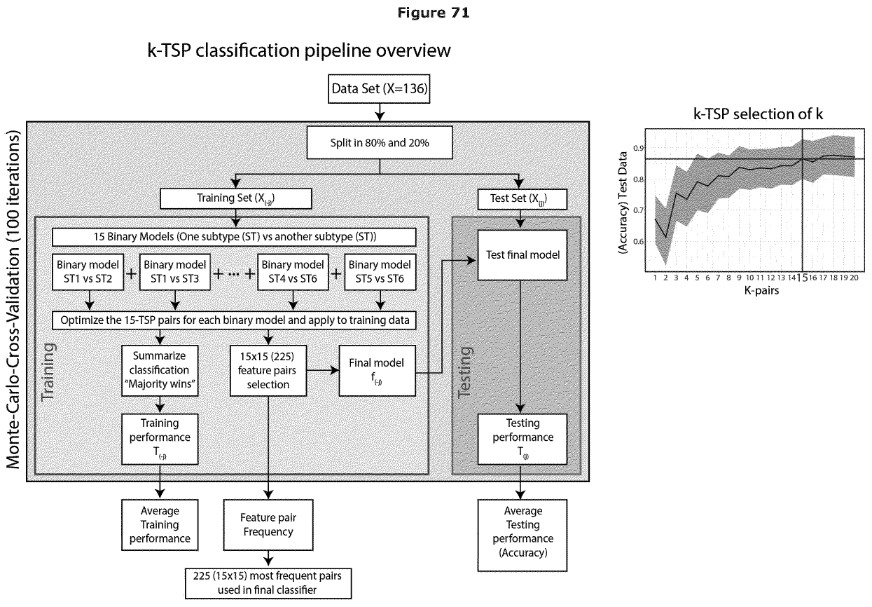

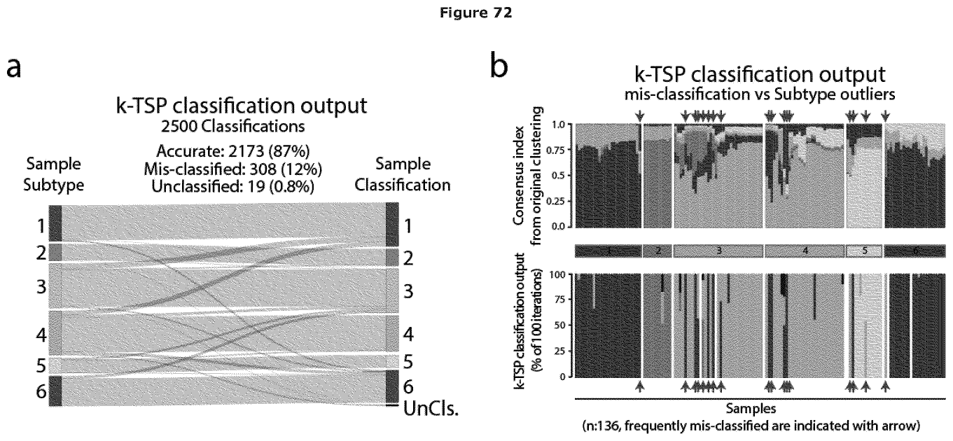

For the k-TSP single sample classifier, the Inventors first re-analysed the NSCLC cohort using label-free, data Independent acquisition (DIA)-based MS analysis (FIG. 70). DIA-MS enables rapid analysis of the proteome in fully complex Individual samples without the need of labelling, simplifying the analytical workflow and Increasing the reproducibility. As expected, due to limited MS time per sample, the proteome coverage of the DIA analysis was less comprehensive than in the DDA data (6717 proteins identified, FDR<1%, FIG. 70). Importantly, the DIA analysis showed overall high correlation to the original DDA data, Indicating that the DIA data would provide the Information needed for NSCLC subtype classification (FIG. 70). The k-TSP classifier uses quantitative information from a set of protein pairs, measured by DIA in a single sample, in order to classify the sample (FIG. 71). The k-TSP classifier was optimised using the same strategy as used for the SVM classifier and resulted in high accuracy (average: 87%, FIG. 67b), as well as a high degree of feature pair redundancy between the iterations (FIG. 67d). Misclassifications were spread out between subtypes but concentrated upon a limited number of samples, again largely overlapping with subtype outliers (FIG. 72).

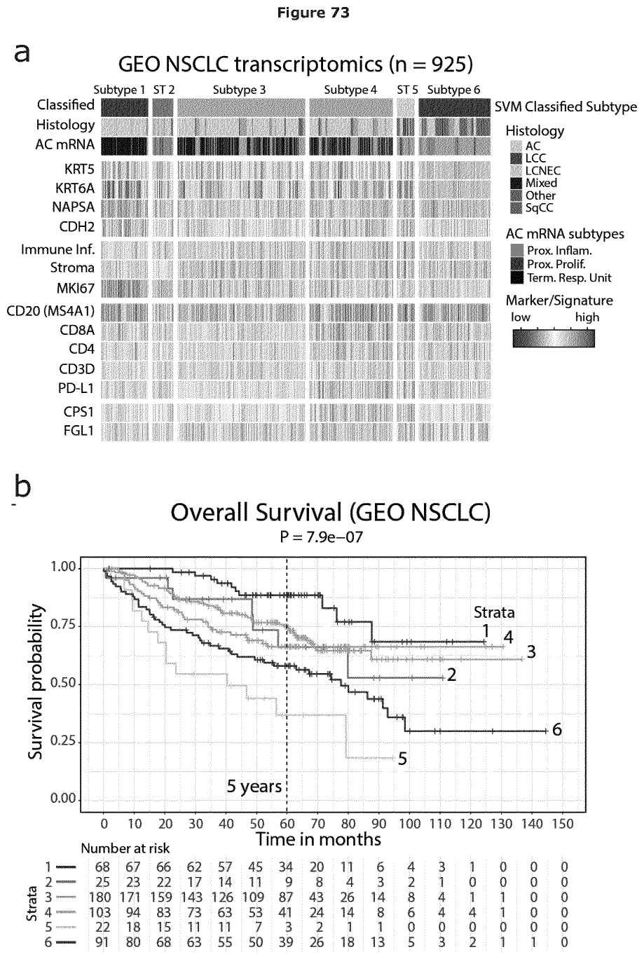

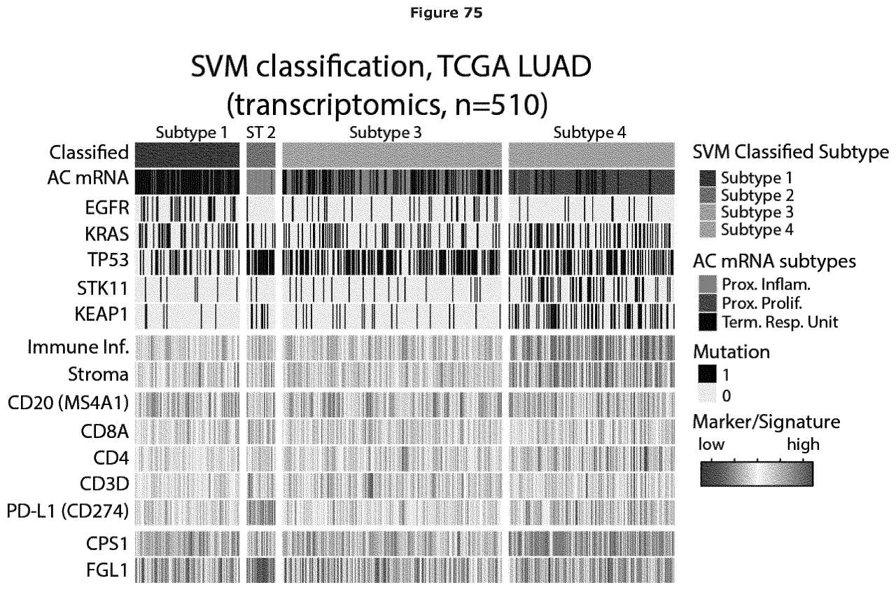

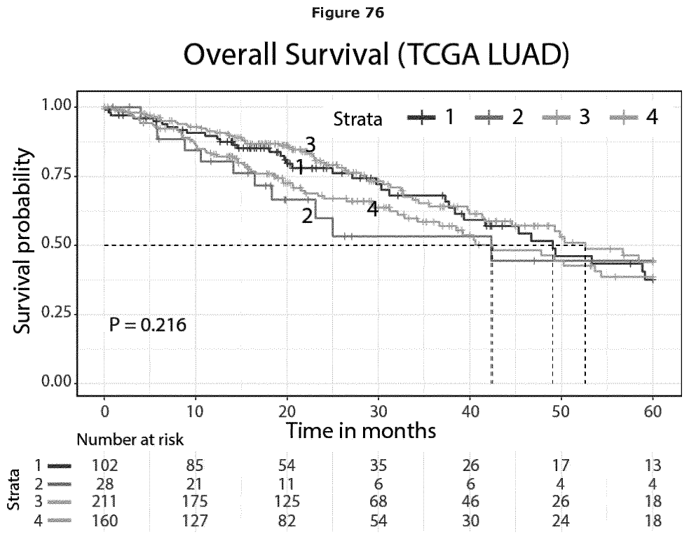

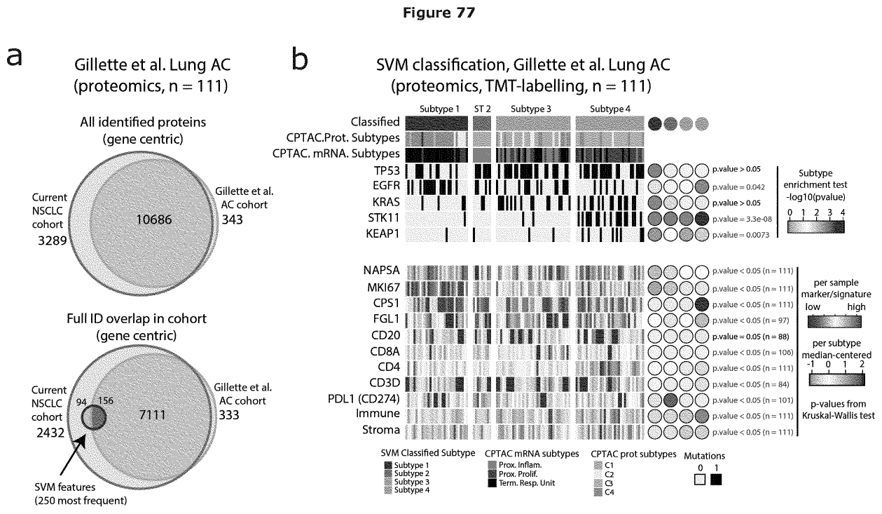

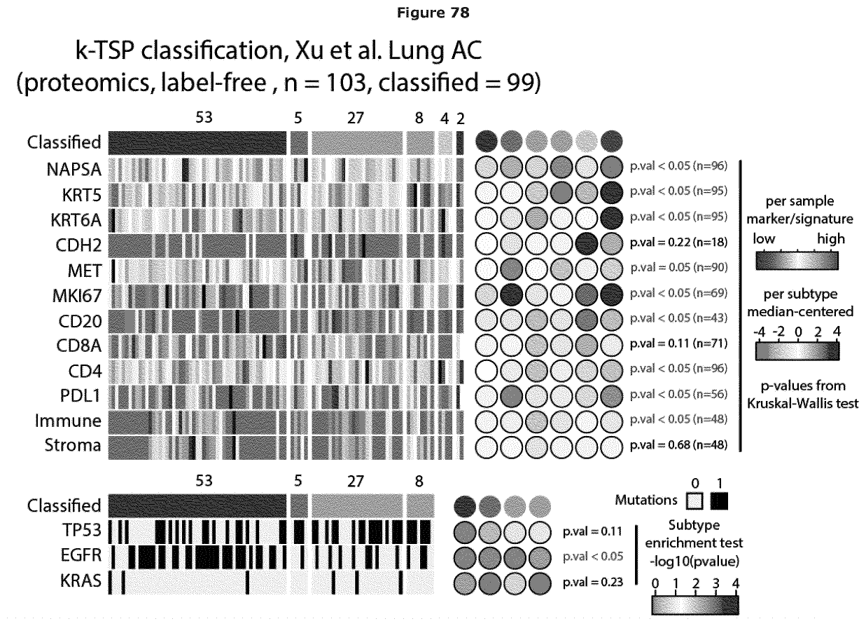

Due to the lack of previous datasets describing the NSCLC proteome, the inventors validated the SVM classifier, as well as the subtypes here identified, using a previously described NSCLC transcriptomics meta-dataset (GEO NSCLC dataset⁵⁴) with mRNA levels as proxy for protein levels. Importantly, the classification of the GEO NSCLC cohort reproduced the six NSCLC proteome subtypes here described with highly similar characteristics in terms of subtype size, signature and marker expression (FIG. 73a). Notably, a subset of AC samples that were classified Into Subtype 6, which is largely a SqCC Subtype, showed expression of SqCC markers (KRT5 and KRT6A), and lacked the AC marker Napsin A (NAPSA). The associated overall survival data indicated differences in prognosis between the classified subtypes, suggesting a predictive value of the NSCLC proteome subtypes (FIG. 73b). Next, the inventors used the TCGA lung AC dataset² (TCGA-LUAD, n=510 samples), again with mRNA levels as proxy for protein levels. As this dataset is restricted to AC, the Inventors re-trained the SVM classifier for the four AC enriched proteome subtypes (Subtypes 1-4). Importantly, the classification of the TCGA cohort reproduced the 4 AC proteome subtypes here described with close to perfectly matching characteristics in terms of subtype size, mutation enrichment pattern, signature and marker expression (FIG. 75). The overall survival data available for the TCGA cohort supported the trends suggested in the GEO cohort analysis, with poor survival in Subtype 4, and better survival in Subtypes 1 and 3 (FIG. 76). This finding is in agreement with previous reports of TLSs (high in Subtype 3) as a positive prognostic factor in lung cancer³⁴ and indicates that adjuvant therapy could be beneficial in Subtype 4. To further validate the proteome subtypes, the inventors analysed a recently published MS-dataset (TMT-labelled) for lung AC (Gillette et al.⁶). Although the analytical depth in this dataset was lower, the Inventors were still able to evaluate 156 of the 250 highest ranking SVM features (FIG. 78). Overall, the classification of this dataset again demonstrated that proteome Subtypes 1-4 were distinct and reproducible between datasets and analytical platforms (FIG. 77b). The k-TSP classifier was evaluated in another recent lung AC MS-dataset (label-free, Xu et al.⁷). In this dataset 209 out of 225 k-TSP feature pairs were identified, and the classification once again produced subtypes with characteristics matching those in the original discovery cohort (FIG. 78).

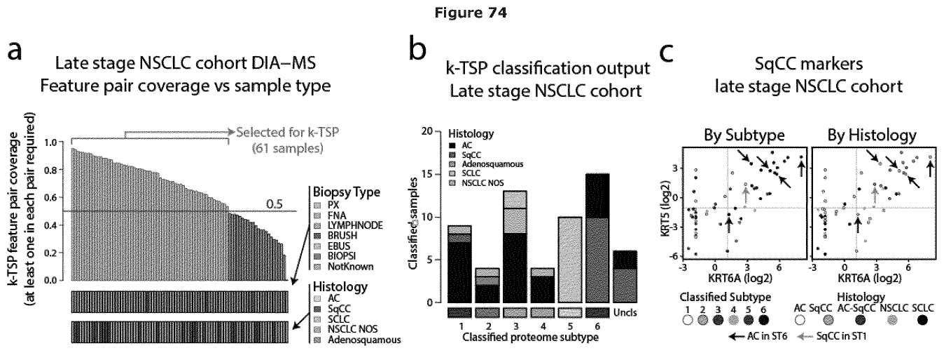

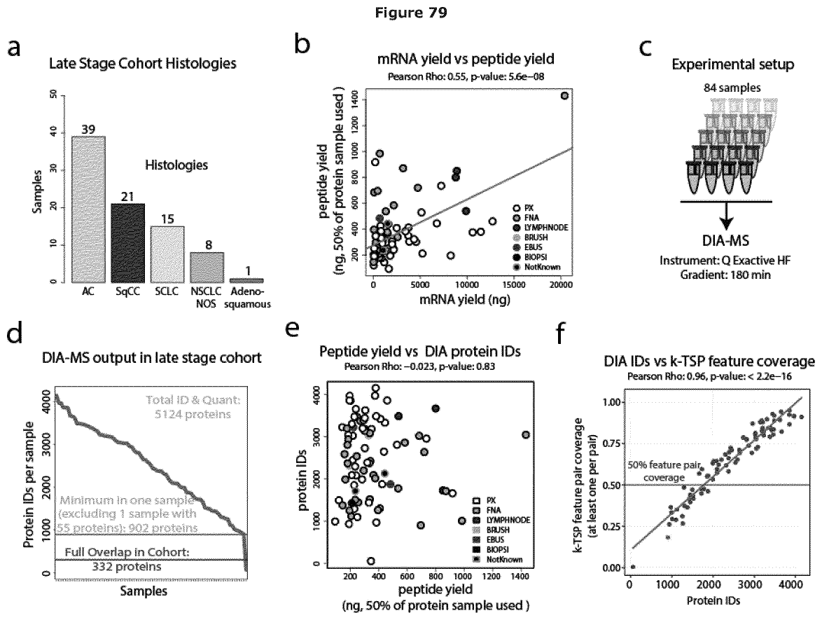

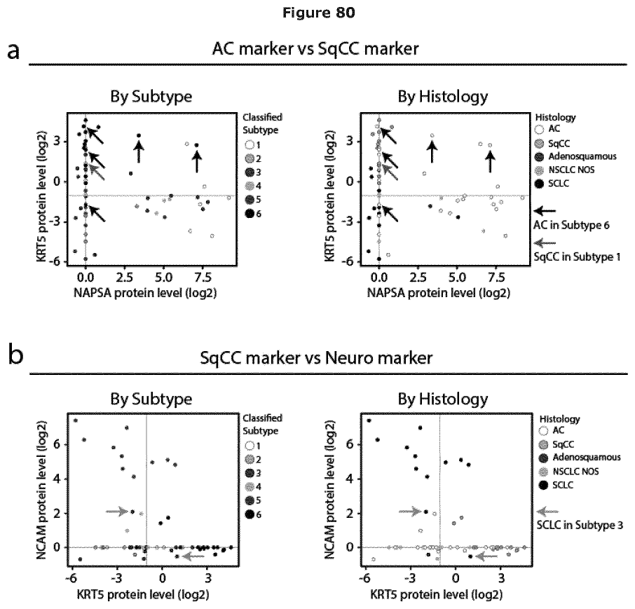

The majority of NSCLCs are diagnosed at late stage when surgery is not an option, and the availability of cancer material for clinical evaluation is restricted to minute biopsies sampled during bronchoscopy or by fine needle aspiration. Ideally, a clinically applicable MS-based diagnostic pipeline should therefore be able to classify lung cancer also based on this type of samples. To evaluate the k-TSP classifier in this setting, the inventors analysed a cohort of late stage NSCLC (84 samples) by label-free DIA-MS (FIG. 79). The total number of identified proteins (5124, FDR<1%) as well as the overlap between samples produced by DIA-MS analysis was lower in the late stage cohort compared to the original early stage cohort (FIG. 79d). This is likely a result of inferior quality in biopsy samples compared to surgical material samples. For accurate k-TSP classification, only samples with at least 50% coverage of the feature pairs were selected for classification (61 samples, FIG. 74a). An evaluation of the Impact of biopsy method on the DIA-MS output (feature coverage) indicated that bronchoscopy biopsies as well as lymph node samples generated better data than fine needle biopsies (FIG. 74a). Importantly, 55/61 samples were successfully classified by the single sample k-TSP classifier with an overall good agreement between histological subgroup as determined by routine clinical diagnostics and classified NSCLC proteome subtype (FIG. 74b). Disagreement was however indicated for a few samples, i.e. five AC samples were classified to Subtype 6, one SqCC sample was classified to Subtype 1 and two SCLC samples were classified to Subtype 3. An evaluation of histological marker proteins using the DIA data however indicated that these samples may be atypical, or even mis-annotated by histology as shown by KRT5/KRT6A levels (FIG. 74c) as well as Napsin A and Neuro-markers (FIG. 80). In summary, this analysis shows that MS based analysis of either early stage surgical material or late stage biopsy material enables accurate classification of NSCLC into the six NSCLC proteome subtypes here described.

### Discussion

Prediction of treatment response as well as optimal combination or sequencing of anti cancer therapies remain as two of the most urgent clinical needs in management of non-small cell lung cancer (NSCLC). To fulfil these needs, more accurate and precise molecular subtyping of the disease is crucial, and this can be achieved by more sophisticated complex biomarkers. The analyses presented here subdivides NSCLC Into six proteome subtypes by in-depth molecular phenotype analysis of tumours, capturing driver pathways, but importantly also new immune phenotypes.

Hitherto, a large number of different immune evasion mechanisms have been described in cancer, but their relation to the level and type of neoantigens produced in different tumours is understudied. Here the Inventors used HiRIEF LC-MS^{9,10} for In-depth proteome analysis and unbiased non-canonical peptide (NCP) discovery to analyse neoantigens in NSCLC. This allowed us to combine tumour mutation burden (TMB) with protein level evidence of complex neoantigens such as CT-antigens and NCP-antigens to provide a sample specific tumour neoantigen burden (TNB) score.

Intriguingly, TNB was highest in the immune-cold Subtype 4 and 6, that also showed common expression of NCP-antigens exemplified by peptides from ERV elements and intronic/intergenic: regions. Such peptides and polypeptides, with longer “non-self” stretches are suggested to be more Immunogenic than SNV-mutation derived neoantigens, which are often too similar to the self-antigen^{39,40}. These findings suggest that expression of highly immunogenic CT- and NCP-antigens may be incompatible with immune infiltration as this would elicit a strong immune response and killing of the cancer cells. Further, non-canonical peptides did not correlate with TMB suggesting that mutations are not the main cause of these types of neoantigens. Instead in inventors' data, both CT-antigens and NCP-antigens are associated with global hypomethylation suggesting looser epigenetic control, in line with previous reports for CT-antigens⁴². The mechanism for the altered methylation in NSCLC however remains to be revealed. From a treatment point of view these findings are also interesting as NCP-antigens are more likely to be widely shared by different tumours and different individuals than SNV-mutation derived neoantigens, which tend to be very patient-specific⁴⁰. This renders non-canonical peptide neoantigens more promising for off-the-shelf immuno-therapy development.

In relation to current checkpoint inhibition targeting PD1/PD-L1, Subtype 2 is characterized by PD-L1 expression, T-cell infiltration, activated interferon gamma signalling, proficient antigen presentation and high TMB. Importantly, patients within this subtype, with potential to response to PD1/PD-L1 checkpoint drugs, could not have been captured by any of these characteristics alone, as for example high TMB or high PD-L1 tumours can be found outside the Subtype 2. Currently used single predictive biomarkers for PD1/PD-L1 checkpoint Inhibitors in NSCLC (PD-L1 IHC or the less established TMB) are insensitive or even un-informative, and complex biomarkers that hold multi-level information are likely to Improve the predictive accuracy⁵⁵. The data presented here indicate that MS-based proteome level subtyping of NSCLC could offer a powerful and competitive method for therapy prediction in the future.

A second wave of checkpoint inhibitors are currently investigated in clinical trials with targets including the Inhibitory T-cell receptors LAG-3, TIM-3 and TIGIT⁴³. LAG-3 is co-expressed with PD-1 in CD4 (+) and CD8 (+) T-cells, and dual targeting of these receptors resulted in a strong synergistic effect and efficient clearance of transplanted tumours⁵⁶. Based on this and other supporting studies, antibody based inhibition of LAG-3 is currently investigated in multiple clinical trials with the majority focusing on combined LAG3 and PD-1/PD-L1 inhibition⁴³. Importantly, FGL1, a protein normally secreted by liver cells was recently shown overexpressed in cancers and identified as a high affinity ligand to LAG-3⁴⁵. Further, FGL1 and LAG-3 Interaction resulted in T-cell suppression while blockade of the interaction potentiated anti-tumour immunity. The analysis reveals that FGL1 is overexpressed in Subtype 4 NSCLC, and that this overexpression depends on inactivation of the tumour suppressor STK11. Interestingly, Subtype 4 is immune cold and secretion of FGL1 could potentially contribute to a systemic inhibition of T-cell activation and of tumour Infiltration by Immune cells. Further, if FGL1 is indeed the major cancer-derived ligand of LAG-3, inventors' data indicate that immune cell infiltration or antra-tumoural CD8 (+) cells would be a poor predictor of response to Inhibitors targeting LAG-3 as neither of these correlates with FGL1 levels. Instead, inventors' analysis suggests that Subtype 4 could function as stratification for checkpoint inhibitors targeting LAG-3, or, if developed, FGL1.

Apart from PD-L1 expression in Subtype 2 and FGL1 expression in Subtype 4, inventors' analysis also Indicates that B7-H4 may contribute to immune evasion in Subtype 6. B7-H4 belongs to the same family as the ligands of PD-1 and CTLA4, and it Inhibits T-cell growth, cytokine secretion and development of cytotoxicity⁵⁷, but so far the target receptor has not been identified. The finding of Subtype 6 specific expression of B7-H4 was supported by a recent TMA-IHC study of checkpoint expression in NSCLC, where expression of B7-H4 as well as B7-H3 was found higher in SqCC than in ACM. Interestingly, like FGL1 also B7-H4 can be secreted as was previously demonstrated in both rheumatoid arthritis⁵⁹ and ovarian carcinoma⁶⁰, however the impact of secreted B7-H4 on the immune response in cancer remains to be shown. The evaluation of T-cell inhibitory receptors (IR) and their ligands vs overall infiltration shows a general correlation between IR levels and T-cell infiltration. Nonetheless, it also shows that there is subtype distinctive expression of specific IR ligands. This underscores the importance of knowing the level of the IR ligand when selecting immunotherapy, as is evident for PD-L1 levels in relation to checkpoint inhibitors targeting PD-1/PD-L1.

For the highly proliferating and relatively immune cold Subtype 5 (LCNEC) inventors' data do not reveal any subtype specific IR ligand expression. The neoantigen burden analysis however Indicates high expression of potentially immunogenic proteins. This raises the question if other, so far unidentified, IR ligands are expressed on the surface of or secreted by these cancer cells. Previous proteogenomics studies of lung AC⁶⁻⁸ were overrepresented for EGFR-driven cancer in never smokers which may have limited the possibility to evaluate different immune subtypes. The inventors show here that Subtype 1 (EGFRmut enriched) has low Neoantigen burden, low Immune infiltration and low levels of all clinically relevant ligands of T-cell inhibitory receptors. These findings are well in line with EGFR mutant NSCLC being refractory to checkpoint inhibitors⁵⁵.

The analyses also show a striking co-expression of FGL1 and CPS1 in a subset of Subtype 4 samples. In analogy to FGL1, CPS1 is normally only expressed in liver cells but overexpressed in cancer cells after STK11 inactivation⁴⁹. This result indicates that Inactivation of STK11 in lung AC may unleash transcriptional programs that are normally only active in liver cells. In relation to this hypothesis, inventors' finding of HNF1A as the transcription factor with the highest correlation to FGL1/CPS1 is interesting. HNF1A is a liver specific transcription factor as shown by us⁶¹ and others⁶², that activates broad liver specific transcriptional programs with the potential to reprogram fibroblasts into hepatocytes⁶³. Further, transfection of HNF1A into human fibroblasts resulted in a dramatic upregulation of multiple genes including FGL1M. No direct link has previously been shown between STK11 inactivation and HNF1A activation, however the mouse equivalent to HNF1A, TCF1 is upregulated and activated by mTORC1-STAT3⁶⁵. The analysis here suggests that reduced HNF1A promoter methylation in STK11 mutated samples contributes to elevated HNF1A mRNA levels, but the mechanism for this epigenetic regulation of HNF1A remains to be further elucidated. Further, analysis of public domain cell line data showed that NSCLC cell lines with mRNA expression of FGL1 and CPS1 were more sensitive to both docetaxel and mTOR inhibition. This result is in agreement with STK11 being an upstream negative regulator of mTOR signalling, as loss of STK11 could confer a cancer cell dependency in mTOR signalling⁵³. The analysis thus indicates that inactivation of STK11 in NSCLC modulates two cancer hallmarks at once by simultaneously Increasing growth rate by loss of mTOR signalling control and promoting immune evasion by expression of FGL1. At the same time, inventors' data point to a potential future combination therapy strategy, where LAG-3/FGL1 checkpoint inhibitors are combined with mTOR inhibitors.

Many crucial questions remain for a more complete understanding of immune evasion and driver pathway activity in NSCLC. The in-depth proteomics data here presented constitutes a valuable resource for Investigation of these and other research questions by providing a resource of molecular phenotype data.

As inventors' analysis demonstrates clinical utility of the proteome subtypes of NSCLC, the inventors continued to develop two methods for classification/subtyping of NSCLC that would be applicable in a clinical setting. The cohort level classifier (SVM-based) Is valuable in a clinical trial setting where multiple samples are collected and analysed together. The single sample classifier (k-TSP) can be used in a routine diagnostic setting for rapid, label-free analysis of individual samples. Both classifiers showed high accuracy and robustness. Importantly, these classifiers rely completely on the quantitative evaluation of discrete panels of biomarkers that the Inventors here define by differential expression analysis as well as during classifier optimisation. Evaluation of the developed SVM and k-TSP classifiers using multiple different external cohorts based on both proteomics and transcriptomics data replicated close to perfectly the characteristics of the six proteome subtypes. This result validates the biological relevance of the subtypes as well as the accuracy of the classifiers.

Further, in a first proof-of-concept analysis the inventors demonstrate that the DIA-MS based single sample k-TSP classifier can be utilized even in late stage NSCLC where very limited sample material is available. Using samples from fine-needle biopsy and bronchoscopy, inventors' classification pipeline classified 55 lung cancer samples into the six proteome subtypes. Importantly, using histology as measurement of classification accuracy this analysis indicated that the classification pipeline produced relevant output. It should be noted that neither the sampling, nor the sample preparation was optimised for MS-based classification, so the inventors predict that there is much room for further improvement and increased quality of the DIA-based classification method.

In summary, the inventors present a first comprehensive proteome analysis of NSCLC, demonstrating the value of high-resolution molecular phenotype analysis as an important component in inventors' quest to understand cancer. Importantly, inventors' analysis indicates for the first time that different immune evasion mechanisms are used by cancer cells depending on the type of neoantigens expressed. immune response towards simpler mutation-derived neoantigens appear to be neutralized locally by PD-L1 as seen in Subtype 2 (high TMB but low non-canonical neoantigens). With complex, more immunogenic neoantigens expressed the cancer cells cannot afford to allow Immune infiltration, and therefore secreted checkpoint ligands like FGL1 are expressed for a systemic inhibition of the immune response as seen in Subtype 4. Further studies are needed to determine how these strong neoantigens push for immune evasion mechanisms that hinder immune cell infiltration, and how to best target these processes.

### Methods

### HiRIEF-LC-MS TMT-DDA Based Analysis of Early Stage NSCLC Cohort

Sample Selection and Preparation.

Resected lung cancer tumour samples from a total of 192 patients with early-stage lung cancer surgically treated at the Skåne University Hospital in Lund, Sweden, were collected, as described in previous studies¹²⁻¹⁴. DNA, RNA and protein from fresh frozen tissue pieces were extracted using the AllPrep Kit (QIAGEN, cat no 80204), as described previously¹². For the current proteomics analysis, 35 samples were excluded due to insufficient protein amount or deviating Protein-RNA or Protein-DNA concentration correlation resulting in 157 samples remaining for protein digestion and further MS analysis. Four volumes (one volume equals the sample volume) of Ice-cold (−20° C.) acetone were added to each protein fraction from the Allprep kit to precipitate the proteins. The tubes were inverted 3 times and Incubated 60 min at −20° C., followed by centrifugation for 10 minutes at 12000 g in a pre-cooled centrifuge at 4° C. The supernatant was discarded, and the pellet was washed once with 100 μl Ice-cold ethanol. The pellet was then dispersed in 100 μl Ice-cold ethanol by ultrasonication (Program: Am 50%, time 10 s, pulse 1.0 s on the Bandelin Sonoplus probe sonicator, from Heco, Norway), centrifuged, and the resulting pellet was air-dried (≈10 min). The pellet was subsequently dissolved in 200 μl reconstitution buffer (4% (w/v) SDS, 25 mM HEPES pH 7.6), and protein concentration was determined using Bio-rad DCC. For each sample, 300 μg (about 150 μl, 2 μg/μl) of reconstituted protein was reduced for 45 min at room temperature (RT) by addition of dithiothreitol (DTT) at a final concentration of 1 mM. Free thiols were subsequently alkylated for 45 min at RT with chloroacetamide at a final concentration of 4 mM.

Proteins were then captured to SP3 (single-pot, solid-phase-enhanced sample-preparation)⁶⁶ beads (GE Healthcare Sera-Mag SpeedBeads™ Carboxyl Magnetic Beads, hydrophobic 65152105050250, hydrophillic 45152105050250) by addition of 15 μl of stock beads solution (10 μg/μl) and addition of acetonitrile with 1% formic acid to obtain a final composition of 50% ACN. The mixture was rotated for 8 minutes at room temperature. To remove the lysis buffer, the tube was placed on a magnetic rack and Incubated for 2 minutes at room temperature. Supernatant was discarded, tubes were removed from the magnetic rack and the bead-attached-proteins were washed twice by addition of 200 μl of 70% ethanol (incubated for 30 seconds on the magnetic stand, followed by supernatant removal). Thereafter, 180 μl of acetonitrile was added and the samples incubated for 15 seconds on the magnetic rack. The supernatant was then discarded, and the beads air-dried for 30 seconds. Proteins were digested by addition of 50 μl of digestion solution (1M Urea/25 mM Hepes) with Lys-C (1:50) and incubated at 37° C. for 16 h, followed by addition of 50 μl of trypsin (1:50) in 25 mM Hepes and Incubation overnight at 37° C. Digested peptides were collected as the supernatant after placing the tube on a magnetic rack. Finally, 50 μl of water was added twice to collect remaining peptides and peptide concentration was measured using Bio-rad DCC. Four out of 157 samples had insufficient peptide amount (<100 μg) for TMT labeling and were excluded. All remaining 153 samples were pre-screened by LC-MS/MS on a QExactive HF using short gradient (60 min) DDA runs to identify outlier samples. Based on analysis of the short gradient data, 10 samples with extensive blood contamination were excluded, resulting in 143 samples remaining for tandem mass tag (TMT) labeling. Subsequent re-analysis of clinical data resulted in the exclusion of two additional samples after MS data generation due to uncertain primary tumour origin. This resulted in a final cohort size of 141 lung cancer samples for subsequent analysis.

Tandem Mass Tag (TMT) Labeling and HiRIEF Pre-Fractionation of Peptides.

A total of 143 samples were TMT labeled. Before labeling, a reference pool was prepared to function as denominator in each TMT set. The pool was made by: peptides from 77 AC samples pooled together to form 1 mg AC sub-pool; the same amount of peptides from 32 SqCC samples were pooled together to form 1 mg SqCC sub-pool; peptides from 22 LCC and 10 LCNEC samples were pooled together to form 1 mg LCC+LCNEC sub-pool; then these 3 mg sub-pools were pooled together to form the final reference pool. 100 μg of peptides from each tumour sample and reference pool was labeled with TMT 10-plex reagent according to the manufacturer's protocol (Thermo Scientific). The 143 tumour samples were distributed across 16 TMT 10-plex sets, with 9 tumours and one reference pool, except in set 16, which had two reference pools. An additional TMT set, nr 17, was designed to include 4 reference pool samples and 6 tumour sample replicates also present on the primary 16 TMT sets. Labeled samples in each TMT set were pooled, cleaned by strata-X-C-cartridges (Phenomenex) and dried in a Speed-Vac.

The TMT labeled peptides, were separated by High Resolution Isoelectric Focusing (HiRIEF) on pH 3.7-4.9 and 3-10 strips (300 μg per strip) as described previously^{9,10}. Peptides were extracted from the strips by a liquid handling robot (Etan digester from GE Healthcare Bio-Sciences AB, which is a modified Gilson liquid handler 215). A polypropylene well former with 72 wells was put onto each strip and 50 μl of MilliQ water was added to each well. After 30 min incubation, the liquid was transferred to a 96 well plate (V-bottom, polypropylene, Greiner 651201), and the extraction was repeated 2 more times with 35% acetonitrile (ACN) and 35% ACN, 0.1% formic acid (FA) in MilliQ water, respectively. The extracted peptides were dried on the 96 well plate in a Speed-Vac.

MS-Based Quantitative Proteomics.

For each LC-MS run of a HiRIEF fraction, the auto sampler (Ultimate 3000 RSLC system, Thermo Scientific Dionex) dispensed 20 μl of 3% ACN, 0.1% FA solvent into the corresponding well of the microtiter plate, mixed by aspirating/dispensing 10 μl ten times, and finally injected 10 μl into a C18 trap desalting column (Acclaim pepmap, C18, 3 μm bead size, 100 Å, 75 μm×20 mm, nanoViper, Thermo Scientific). Peptides were separated using a gradient of mobile phase A (5% DMSO, 0.1% FA) and B (90% ACN, 5% DMSO, 0.1% FA), ranging from 6% to 37% B in 30-90 min (depending on IPG-IEF fraction complexity) with a flow of 250 nl/min. The Q Exactive HF was operated in data dependent acquisition (DDA), selecting top 5 precursors for fragmentation by HCD. The survey scan was performed at 60,000 resolution from 300-1500 m/z, with a max injection time of 100 ms and target of 1×10⁶ ions. For generation of HCD fragmentation spectra, a max ion injection time of 100 ms and AGC of 1×10⁵ were used before fragmentation at 30% normalized collision energy, 30,000 resolution. Precursors were isolated with a width of 2 m/z and put on the exclusion list for 60 s. Single and unassigned charge states were rejected from precursor selection.

Peptide and Protein Identification.

Peptide and protein identification were performed as described previously¹⁰. Briefly, Orbitrap raw MS/MS flies were converted to mzML format using msConvert from the ProteoWizard tool suite. Spectra were then searched using MSGF+(v10072) and Percolator (v2.08), where search results from all HiRIEF fractions of each TMT set were grouped for Percolator target/decoy analysis. All searches were done against the human protein database of Ensembl 92 in a Nextflow pipeline. MSGF+settings Included precursor mass tolerance of 10 ppm, fully tryptic peptides, maximum peptide length of 50 amino acids and a maximum charge of 6. Fixed modifications were TMT-10plex on lysines and peptide N-termini, and carbamidomethylation on cysteine residues. A variable modification was used for oxidation on methionine residues. Quantification of TMT-10plex reporter ions was done using OpenMS project's IsobaricAnalyzer (v2.0). PSMs found at 1% FDR (false discovery rate) were used to infer gene identities.

Protein quantification by TMT 10-plex reporter ions was calculated using TMT PSM ratios to the reference TMT channels and normalized to the sample median. The median PSM TMT reporter ratio from peptides unique to a gene symbol was used for quantification. Protein false discovery rates were calculated using the picked-FDR method using gene symbols as protein groups and limited to 1% FDR.

DIA Based Analysis of NSCLC Cohort

Protein Digestion of Late-Stage NSCLC Cohort

For each tumour, 225 μl of protein extract were obtained using the AllPrep Kit (QIAGEN, cat no 80204). Each sample was reduced for 45 min at room temperature (RT) by addition of dithiothreitol at a final concentration of 10 mM. Free thiols were subsequently alkylated for 30 min at RT with chloroacetamide to give at a final concentration of 40 mM.

Proteins were adhered to the SP3 beads (GE Healthcare P/N 45152105050250 and 65152105050250) by addition of 25 μl of bead stock solution (10 μg/μl) and addition of acetonitrile to obtain a final percentage of 70% ACN. The mixture was incubated for 30 minutes in the rotating rack at RT. The tube was then placed on magnetic rack and incubated for 2 minutes at room temperature, after which the supernatant was discarded. Magnetic beads were then washed by addition of 500 μl of 70% ethanol and incubated for 30 seconds on the magnetic stand. Supernatant was discarded and the wash repeated once. Thereafter, 500 μl of acetonitrile was added and the samples incubated for 15 seconds on the magnetic rack. Supernatant was discarded and the beads air-dried for 30 seconds. Beads were reconstituted in 100 μl of digestion solution (4 M Urea, 25 mM HEPES pH 7.6) with 10 μg Lys-C and Incubated at 37° C. for overnight, followed by addition of 300 μl of trypsin solution (25 mM HEPES pH7.6, 8 μg trypsin) and incubated 8 h at 37° C. Digested peptides were collected as the supernatant after placing the tube on a magnetic rack. Peptide concentration was measured using Bio-rad DCC.

50 μg of peptides from each sample were cleaned by SP3 beads. For that, peptides were dried by SpeedVac, and resuspended in 20 μl water. 10 μl beads were added to each tube and mixed by short vortex. 570 μl acetonitrile was added to each sample to reach 95% ACN composition. The mixture was incubated for 30 minutes in the rotating rack at RT. The tube was then placed on the magnetic rack and incubated for 2 minutes at RT, after which the supernatant was discarded. The magnetic beads were washed by addition of 250 μl of ACN and placed for 30 seconds on the magnetic stand. Supernatant was discarded and the beads air-dried. Tryptic peptides were detached from the beads by addition of 100 μl of 3% ACN, 0.1% FA and transferred to a new tube.

Spectral Library Preparation

A pooled sample containing peptides from 129 different tumour samples from the cohort was combined for spectral library generation. A total of 2 mg pooled peptides was aliquoted in two parts, each one was subjected to the fractionation of peptides, one by HiRIEF and one by High-pH peptide fractionation. For HiRIEF pre-fractionation, peptides were separated by immobilized pH gradient—isoelectric focusing (IPG-IEF) on pH 3-10 strips as described above in “HiRIEF pre-fractionation of peptides”. The extracted peptides were dried in Speed-Vac and dissolved in 3% ACN, 0.1% formic acid, and consolidated to a final of 40 fractions (as described in the HiRIEF fraction scheme file in the PXD dataset). For High-pH pre-fractionation, peptides were fractionated with basic-pH reverse-phase (BPRP) high-performance liquid chromatography (HPLC). Peptides were loaded and separated on a 25 cm C18 packed column (XBridge Peptide BEH C18, 300 Å, 3.5 μm, 2.1 mm×250 mm). 96 fractions were collected from the column and consolidated to a final of 40 fractions.

MS Data Acquisition.

Peptides were separated using an Ultimate 3000 RSLCnano system coupled to a Q Exactive HF (Thermo Fischer Scientific, San Jose, CA, USA). Samples were trapped on an Acclaim PepMap nanotrap column (C18, 3 mm, 100 Å, 75 μm×20 mm, Thermo Scientific), and separated on an Acclaim PepMap RSLC column (C18, 2 μm bead size, 100 Å, 75 μm×50 cm, Thermo Scientific). Peptides were separated using a gradient of mobile phase A (5% DMSO, 0.1% FA) and B (90% ACN, 5% DMSO, 0.1% FA), ranging from 6% to 30% B in 180 min with a flow of 250 nl/min.

To create the spectral library, each of the 80 fractions was analyzed in a data dependent manner (DDA). The method was set for selecting top 10 precursors for fragmentation by HCD. The survey scan was performed at 120,000 resolution from 400-1200 m/z, with a max injection time of 100 ms and target of 1e6 ions. For generation of HCD fragmentation spectra, a max ion injection time of 100 ms and AGC of 2e5 were used before fragmentation at 25% normalized collision energy, 30,000 resolution. Precursors were isolated with a width of 2 m/z and put on the exclusion list for 15 s. Single and unassigned charge states were rejected from precursor selection. For data independent acquisition (DIA) on the individual tumours, data was acquired using a variable window strategy. The survey scan was performed at 120,000 resolution from 400-1200 m/z, with a max injection time of 200 ms and target of 1e6 Ions. For generation of HCD fragmentation spectra, max ion injection time was set as auto and AGC of 2e5 were used before fragmentation at 25% normalized collision energy, 30,000 resolution. The sizes of the precursor Ion selection windows were optimized to have similar density of precursors m/z based on identified peptides from the spectral library. The median size of windows was 18.3 m/z with a range of 15-88 m/z covering the scan range of 400-1200 m/z. Neighbor windows have 2 m/z overlap.

DIA—Peptide and Protein Identification and Quantification.

Spectral library generation as well as peptide and protein identification and quantification were performed on the Spectronaut software package (version 13.10) from Biognosys. For spectral library generation, all 80 MS raw files (40 HiRIEF+40 Hi pH RP fractions) were searched by the integrated search engine Pulsar. Files were searched against ENSEMBL protein database (GRCh38.92.pep.all.fasta). All parameters were set as default and for each peptide, the best 3 to 6 fragments were used. Results were filtered at all the precursor, peptide and protein levels with 1% FDR. Out of 213392 precursors, the peptide library consisted of 160185 peptides representing 11915 protein groups.

For protein identification and quantification, all DIA raw files were analyzed by Spectronaut using the above generated spectral library. All parameters were kept as default for protein Identification. Briefly, runs were recalibrated using IRT standard peptides in a local and non-linear regression. Precursors, peptides and proteins were filtered with FDR 1%. The decoy database was created by mutation method. For quantification, only peptides unique to a protein group were used. Protein groups were defined base on gene symbols to obtain a gene symbol centric quantification. Stripped peptide quantification was defined as the top precursor quantity. Protein group quantification was calculated by the median value of the top 3 most abundant peptides. Quantification was performed at the MS2 level based on the peak area. The quantitative values were filtered using Qvalue for each sample. For an alternative filtering approach, to Impute missing values across samples, the data filtering was set as Qvalue sparse with no-imputing.

MS-Data Deposit

The mass spectrometry proteomics data for DDA and DIA analysis have been deposited to the ProteomeXchange Consortium via the JPOST partner repository with the data set Identifier PXD020191 (DDA) and PXD020548 (DIA).

Panel Sequencing

Library Preparation and Sequencing

An amount of 250 ng genomic DNA of each sample was used for library preparation, which was performed with Twist Biosciences enzymatic library preparation kit (Twist Biosciences) with the following modifications: fragmentation using a 7-minute incubation in fragmentation step, xGen Duplex Seq adapters (3-4 nt unique molecular Identifiers, 0.6 mM, Integrated DNA Technologies) were used for the ligation and xGen Indexing primers (2 mM, with unique dual indices, Integrated DNA Technologies) wer used for PCR amplification (5 cycles). Target enrichment was performed in a multiplex fashion with a library amount of 187.5 ng (8-plex). The libraries were hybridized to a custom designed capture probes panel (Twist Bioscience), xGen Universal Blockers—TS Mix (Integrated DNA Technologies) and COT Human DNA (Life Technologies) for 16 hours. The post-capture PCR was performed with xGen Library Amp Primer (0.5 mM, Integrated DNA Technologies) for 10 cycles. Quality control was performed with the Qubit dsDNA HS assay (Invitrogen) and TapeStation HS D1000 assay (Agilent). Sequencing was done on NovaSeq 6000 (Illumina) using paired-end 150 nt readout, aiming at 30 M read pairs per sample. Demultiplexing was done using Illumina bcl2fastq2 Conversion Software v2.20.

The custom designed panel is a 370-gene panel and has been designed to enable detection of clinically relevant single-nucleotide variants (SNV) and insertion/deletion variants (INDEL), copy-number aberrations (CNA), fusion events (fusions), microsatellite instability (MSI) and to estimate the tumour mutational burden (TMB) in a single assay. The panel also contains selected hotspot variants in 9 genes where there is strong evidence of pharmacogenetic relevance. The panel contains approximately 21,000 baits, covering 1.9 Mb of target. Full coding sequence is captured of 198 genes, hotspot regions of 132 genes, CNVs for 86 genes, intronic sequences for SV detection of 19 genes and full gene-body sequencing of 9 genes.

Sequence Data Analysis

BALSAMIC workflow v4.0.0⁶⁷ was used to analyze each of the FASTQ files. In summary, we first quality controlled FASTQ files using FastQC v0.11.5⁶⁸. Adapter sequences and low-quality bases were trimmed using fastp v0.20.0⁶⁹. Trimmed reads were mapped to the reference genome hg19 using BWA MEM v0.7.15⁷⁰. The resulted SAM files were converted to BAM files and sorted using samtools v1.6^{71,72}. Duplicated reads were marked using Picard tools MarkDuplicate v2.17.0 and promptly quality controlled using CollectHsMetrics, CollectInsertSizeMetrics, and CollectAligntmentSummaryMetrics functionalities. Results of the quality-controlled steps were summarized by MultiQC v1.7⁷³. For each sample, somatic mutations were called using VarDict v2019.06.04⁷⁴ in tumour-only mode and annotated using Ensembl VEP v94.5⁷⁵. Variants recurrently found (more than 10 cases) in the cohort and not previously described as oncogenic were manually reviewed to detect likely artifacts, which were removed from downstream analyses together with variants showing low quality calls. Variants were classified as putative functional versus passengers by using the Interpretation pipeline developed by the Molecular Tumour Board Portal, a clinical decision support tool that evaluates the functional and predictive relevance of genomic alterations⁷⁶. Briefly, the portal classifies a variant as biologically relevant combining up-to-date results from clinical and preclinical studies, bona fide biological assumptions and bioinformatics calculations.

For tumour mutational load calculations, first all low-quality variants were removed via a hard filter of total read depth (DP)>50 and alternative allele depth (AD)>5. Then we followed the procedure demonstrated by Chalmers et al⁷⁷.

Statistical Analysis

All statistical analyses were conducted using R. Correlations and associated p-values (Spearman and Pearson) were calculated with the R functions cor( ) or contest( ). Linear models built with the R function Im( ). Pairwise comparisons were computed by Wilcoxon Signed-Rank Test with the R function wilcox.test( ) or Welch's t-test using t.test( ). For the multiple group comparisons, Kruskal-Wallis test was used with the R function kruskal.test( ) or ANOVA test using anova( ). Enrichment analysis were conducted in R by Hypergeometric test with the R function phyper( ) or fisher.test( ). Where indicated, p-values were corrected for multiple testing using the Benjamini-Hochberg (BH) method⁷⁸ in R. Survival analysis was conducted using Kaplan-Meier estimator from ‘survminer’ and ‘survival’ R packages. For analysis of differential protein levels between samples DEqMS¹⁵ analysis was performed in R.

Gene Expression and DNA-Methylation

Pre-processed Illumina gene expression data for 118 cases was obtained from Karlsson et al.¹² and DNA methylation data was available from previous studies for 113/141 lung cancer tumors in this cohort (GSE60645 and GSE149521)^{13,14}. DNA methylation data processing and filtering were performed as previously described^{13,14}, resulting in a final dataset Interrogating 459790 genomic positions. Methylation probes were annotated using the IlluminaHumanMethylation450kprobe (v2.0.6) R package and promoter regions were defined as TSS+/−500 bp and extracted using the promoters( ) function in the TxDb.Hsapiens.UCSC.hg19.knownGene (v3.2.2) R package. Methylation probes and promoter regions were overlapped using the findOverlaps( ) function in the GenomincRanges R package (v1.34.0), resulting in a total of 72442 methylation probes in the promoter regions of 19327 genes. For each gene, the promoter-overlapping probe with the highest standard deviation was selected and the Pearson correlation between probe methylation beta values and log 2 transformed mRNA levels was derived.

The promoter methylation score for each tumor was calculated as the per sample mean of methylation beta values for promoter-overlapping probes. Similarly, the overall methylation score per sample was derived as the mean of methylation beta values for all probes.

Immunohistochemistry

Sample Collection and Histological Classification Formalin-fixed paraffin embedded (FFPE) samples collected for histology were evaluated with hematoxylin and eosin staining by a certified pathologist with extensive experience in lung pathology (HB). The classification was performed according to the World Health Organization Classification for Lung cancer, employing both 2004⁷⁹ and 2015⁸⁰ editions. Moreover, Tumor Microarrays were constructed from 1.0 mm punches of the FFPE lung cancer blocks described above, using a manual arrayer (Pathology Devices, Inc., Westminster, MD).

Immunohistochemistry for PD-L1, CD3 and CD8

Immunohistochemistry (IHC) for PD-L1 was performed on TMAs with the help of a Ventana Benchmark Ultra (Roche Diagnostics, Switzerland), pre-treating the tissue with Cell Conditioning 1 (cat. 950-124, Roche Diagnostics, Switzerland), incubating the section with the anti-PD-L1 antibody (rabbit monoclonal antibody clone 28-8, dilution 1:100, ab205921, Abcam, UK) and employing an OptiView DAB IHC Detection kit (cat 760-700, Roche Diagnostics, Switzerland). IHC for CD3 and CD8 were done always on TMAs but instead employing a DAKO immunostainer, pre-treating the tissue with Envision FLEX Target retrieval solution High pH (cat K800421-2, DAKO, Denmark) in a PT-Link Module (DAKO, Denmark). Antibodies employed for the reactions were anti-CD3 (polyclonal rabbit antibody, cat A0452, DAKO, Denmark) and anti-CD8 (mouse monoclonal antibody clone C8/144B, cat M7103, DAKO, Denmark).

PD-L1 was evaluated according to the Interpretation guidelines developed for the PD-L1 immunohistochemical test⁸¹ and were evaluated on 53 cases available on the TMAs. Briefly, a minimum of 100 tumour cells were evaluated for each tumour sample (majority between 200 and 400), measuring the percentage of neoplastic cells that showed at least a partial and weak cell membrane positivity (Tumour Proportion Score, TPS). Any cytoplasmic staining was not evaluated; necrotic cells, immune cells and macrophages were not considered in the count. The presence of Internal positive control was assessed on each sample, to assure the reliability of the immunohistochemical reaction.

CD3 and CD8 was evaluated in 90 cases available on the TMAs for immunohistochemical staining and evaluation. The manual annotation of these immunohistochemical markers was performed accordingly to Al-Shibli and collaborators⁸², considering the epithelial and the stromal compartments separated in the evaluation. Briefly, at least 100 nucleated cells were considered for each compartment of the sample and the percentage of positive cells in the membrane was counted. Samples with a percentage of positive cells inferior to 1 were considered negative.

Histology Subtype and Ternary Lymphoid Tissue (TLS) Evaluation on Duster 2 and 3

In order to explore the relationship between PD-L1 protein expression, the histological component and presence of TLSs, 21 cases were selected showing different expression of PD-L1 in the proteomic quantification. The histological classification was performed on hematoxylin and eosin sections, following the WHO classification of tumours of the lung⁸⁰. Focusing on the adenocarcinoma subtyping, the subtype percentages were registered by increments of 5%, according to Travis and collaborators⁸³. A percentage was calculated for each of the 6 major adenocarcinoma subtypes (lepidic, acinar, papillary, micropapillary, solid and invasive mucinous) in each tumour. For squamous carcinomas no further subtyping was performed. The tumour's bulk composition was manually annotated, dividing each tumour into epithelial, stromal and immune compartments and a percentage of necrosis was calculated. For intra-tumoral TLSs, 30 high power fields were considered for counting the number of TLSs.

Integrated Downstream Analysis and Bioinformatics

Consensus Clustering for Determination of NSCLC Proteome Subtypes

Consensus clustering⁸⁴ was used to group samples based on proteins quantified across all samples (input matrix: 9793×141). The following parametrization was applied: clusterAlg=‘hc’, innerLinkage, finalLinkage=‘ward. D2’, distance=“spearman”, pItem=0.8, pFeature=1, reps=1000, maxK=11. The number of clusters (k=6) was determined by the elbow of the relative change in consensus index CDF curve and the empirical assessment of enriched mutations, MSigDB hallmark gene sets and immune/stroma signatures for k=5,6,7. The consensus index for each sample was extracted and normalized to unity as an indication of the sample membership/outlierness to each cluster.

Correlation Network Analysis

Filtering was first performed based on DEqMS analysis (|log 2 ratio|>0.5 and P.adj.<0.01) and quantitative data in at least 70% of samples. Pairwise Pearson correlations were then calculated for the remaining 5257 proteins. The resulting correlation matrix (input matrix: 5257×5257) was used for downstream analysis with Seurat R package⁸⁵. Specifically, PCA dimensionality reduction was performed on standardized correlations and the first 8 principal components were retained according to the elbow of the PCA standard deviation plot. These components were used to project proteins in 2-dimensional UMAP coordinates with n.neighbors=20 and min.dist=0.2 after empirical assessment of the local and global patterns captured in visualizations with different parameters. An Euclidean distance-based, shared nearest neighbor graph was constructed using the same n.neighbors (n=20), and Louvain community detection algorithm⁸⁶ was applied to find distinct protein clusters. The resolution parameter (n_resolution=0.6) was chosen as the maximum value for which every cluster could be assigned to at least one MsigDB hallmark (ClusterProfiler⁸⁷, enrichment adj.p-value<0.05). Cell-type enrichments were assigned with the same p-value significance threshold based on genes with absolute average log 2 fold >0.5, adjusted p-value <0.01) taken from Travaglini et al.⁸⁸. Per subtype networks were visualized after estimating the median of the log 2 ratios for each protein across the respective samples. The heatmap shows the above-estimated ratios averaged per term.

mRNA-Protein Differences

We calculated mRNA—protein Pearson correlations of genes with quantification values in at least 70% of samples (n.genes=8865). Correlations were Fisher z-transformed and differences caused by complex membership, stability—based on ranking in the top (bottom) one third of half-lives for stable (unstable) assignment—and miRNA-targeting were assessed using external experiment data^{23-25.} Two-group and multi-group comparisons were assessed with two-sided t-tests and ANOVA, respectively.

Immune/Stroma Estimation—Immune Gene-Set Scores

Standardized immune and stroma scores were calculated using the ESTIMATE method¹⁷ on the complete proteomics data. Previously defined immune cell markers³² and hallmarks of ‘INTERFERON ALPHA RESPONSE’ and ‘INTERFERON GAMMA RESPONSE’0 from MSigDB⁸⁹ were used as Input for single-sample gene-set enrichment analysis (ssGSEA) in GSVA R package⁹⁰.

TMB—Antigen Presentation Machinery Correlation

To evaluate the relationship between TMB and antigen presentation machinery (APM), a similar analysis to Dou et al.³³ was followed. Specifically, samples were separated into TMB-high/-low cases based on their log 2 TMB values and into APM-high/-low based on their enrichment score in ‘KEGG ANTIGEN_PROCESSING_AND_PRESENTATION’⁹¹. K-means algorithm was used with means of five highest and lowest values of TMB as initial centers for TMB-high and -low groups. We performed a similar analysis based on enrichment scores to define AMP-high/-low samples. For each of the four TMB/APM categories, subtype over representation was evaluated by Hypergeometric test and p-values were corrected for multiple testing.

Cancer and Driver Related Proteins (CDRPs)

CDRPs were defined based on membership in 10 cancer-related signaling pathways as previously described¹⁸, and/or if causally linked to cancer according to the COSMIC cancer gene census effort¹⁹. In total 832 CDRPs were identified and quantified in the current NSCLC cohort. CDRP annotation was performed using previously published information related to protein function as transcription factors, chromatin remodeling factor or transcription factor co-factor according to AnimalTFdb⁵⁰; protein kinase⁹²; protein phosphatase⁹³; ubiquitin E3 ligase⁹⁴; protein subcellular localization according to SubCellBarCode resource (www.subcellbarcode.org)⁹⁵; and annotation as drug target⁹⁶.

Proteogenomics 6RFT Search

The IPAW proteogenomics pipeline for novel peptides was implemented as previously described¹⁰. Specifically, nucleotide sequences for each chromosome (UCSC⁹⁷), hg19-GRCh37) were in silico translated in six-reading frames (6FT) and digested into peptides following trypsin rules (without missed cleavages, no cleaving on N-terminal side of proline residues). Unique peptides with length 8 to 30 amino acids were stored with their chromosome positions after removal of peptide matches to known proteins. Predicted isoelectric points of all 6FT theoretical peptides by PredpI⁹ were used to devise pI-restricted databases with specific pI intervals corresponding to the experimental fractions of IPG strips. Due to both strip manufacturing and strip alignment variations during the process of extraction to 96-well micro-titer plate, the centers of pI intervals may shift slightly run-to-run and were therefore adjusted so that the median value of delta pI (experimental pI minus predicted pI) is equal to 0 for each individual IPG strip (the peptides used to calculate delta pI shift were unique peptides identified with 1% FDR from the standard proteomics search for each TMT set). The pI interval of each pI-restricted database was extended on both sides of the experimental interval with a prediction error margin that corresponds to the 95% confidence interval (0.11 for 3-10, and 0.08 for 3.7-4.9 pH range). Finally, each pI-restricted mini database was appended with Ensembl90⁹⁸ human protein database.

A target-decoy strategy was used to search the peptide spectra. Decoy peptides were generated from the peptides of pI-restricted databases in reversed tryptic manner (i.e., C-terminal residue is maintained, whereas the rest of the target amino acid sequence is reversed). Target and decoy matches to known tryptic peptides were discarded (as well as deamidations of asparagine to aspartic acid and also considering that isoleucine=leucine). The 1% FDR⁹⁹ of 6FT peptides was calculated as the number of decoy 6FT peptides divided by the number of target 6FT peptides above the score threshold. The genomics coordinates were stored as peptide's ID at the six reading-frame translation step. Novel peptides within genomic proximity of 10 kb were grouped and considered to belong to the same locus.

Peptides were further curated by: 1) BLASTP¹⁰⁰. All 6FT peptides were blasted to Ensembl87⁹⁸+Uniprot¹⁰¹+Refseg¹⁰²+GENCODE24¹⁰³ human proteins in order to remove known proteins, 2) SpectrumAI¹⁰. The subset of 6FT peptides with single amino acid substitution identified at 1% FDR were required to fulfill two criteria: First, at least one of the peptide's MS2 spectra should contain Ions flanking both sides of the substituted amino acid; Second, the sum intensity of the supporting flanking MS2 ions should be larger than the median intensity of all fragmentation ions with the exception of a proline residue to the N-terminal side of the substituted amino acid. Novel peptides from the six reading-frame translation (6RFT) search that passed SpectrumAI filter in the majority of TMT sets and lacked a SNPdb match were retained for outlier detection. Assuming that such peptides should be present in one or in a few samples and that the per set quantification depends on the sample composition, ratios to the reference pool were re-centered by the median and log 2 transformed. Outlying peptides were determined by the same threshold used for the cancer-testis antigen analysis (i.e. ratio >3).

Peptides from 6FT search were further annotated with ANNOVAR¹⁰⁴ (genes: RefSeq¹⁰², UCSC⁹⁷, ENSEMBLE⁹⁸, GENCODE¹⁰³ hg19; long non-coding RNAs: LNCipedia v.5.2¹⁰⁵, gencode.v34.long_noncoding_RNAs after liftOver from hg38 to hg19 coordinates, pseudogenes: gencode.v34.2wayconspseudos¹⁰⁶ after liftOver from hg38 to hg19 coordinates), a custom-made script for alternative open reading frame identification, and Uniprot¹⁰¹ protein names (release 03/2020) for transposable elements assignment according to the blastp protein ID. Annotations were prioritized similar to ANNOVAR precedence rules with emphasis on the exon translation complexity (AltOrf—alternative opening reading frame) and the putative origin of the peptides (ERV—endogenous retro-viral elements, pseudogenes): AltOrf, ERV, pseudogene, exonic, splicing, ncRNA_exonic, ncRNA_splicing, ncRNA_intronic, lncrna, UTR5, UTR3, UTR5; UTR3, Intronic, upstream, downstream, upstream; downstream, intergenic.

TMB—6RFT Peptides (NCPs)

Based on prior knowledge about factors that influence tumour mutational burden, the Inventors evaluated the relationship between the number of 6RFT peptides per sample and TMB using Im( ) function in R under the following linear model specification:
6RFT peptides˜TMB+MK167+TP53-mutation+Purity
Where:
6RFT peptides—number of outlying peptides per sample,
TMB—log 2 values of the tumour mutational burden,
MKI67—Proteomics log 2 ratios of KI-67 as a proliferation index,
TP53-mutation—presence/absence of mutation in TP53 gene, and
Purity—ASCAT-estimated sample purity.

Support Vector Machine (SVM) Based Cohort Classifier

For an Initial filtering to remove uninformative proteins (features) and to prevent high-computation time for downstream analysis, the Inventors applied DEqMS¹⁵ as described above (BH adjusted p-value <0.01 and |log 2(ratio)|>0.5, 5872 proteins, Supplementary Table 3). Next, for a balanced first selection of features, for each comparison, the most upregulated and downregulated 200 (100×2) proteins were included, resulting in a list of 1549 proteins after removal of redundant proteins. Support-Vector-Machine with linear kernel was used to build the classifier. Hyperparameter C and the model was optimized using 5-fold Cross-Validation. The algorithm was implemented using scikit-learn library in Python (version 3)¹⁰⁷.

In machine learning with large datasets, a dataset is often split into three parts, for training, validation and testing. However, in this study the inventors were not in a data-rich situation and could therefore not split the data into three parts. Instead the inventors used the Monte-Carlo-Cross-Validation (MCCV) method¹⁰⁸ to provide an unbiased performance estimation and to optimize the model. The whole process (described below) was repeated 100 times to maximize the number of samples included in training and testing. From each iteration, the testing performance (accuracy) and 200 most important features was reported.

First, the inventors partitioned the dataset randomly into two parts; 80% for training and 20% for testing. Testing data was separated before developing the model and it was only used for the testing, while training data was used to select features and to tune the parameters in order to build a model. To select the most important features in each iteration, Support Vector Machine—Recursive Feature Elimination (SVM-RFE) algorithm was applied¹⁰⁹. SVM-RFE selects the features based on how important they are for separating the groups. It starts with all features (1549) and for each step, a number of least important features are eliminated from the feature set. This process is repeated until the specified number of top features (200) are left in the dataset. The algorithm was implemented using scikit-learn library in python (version 3)¹⁰⁷. The model with the 200 most important features were then applied to test data for estimation of the accuracy.

Finally, the overall accuracy was reported as the average accuracy from the 100 MCCV Iterations, and to build the final model and deploy it, the Inventors selected the most frequently used 200 features from the output of MCCV (100 iterations.

Applying SVM Classifier to External Data

As the model was built on normalized-proteomics-data, training and testing data should be in the same scale in order to estimate the evaluation of the model robustly. Therefore, the model was built on Z-score distributed data and the external data (GEO, TCGA, and Gillette et al.) were transformed to Z-score distribution.

k-Top Scoring Pairs (k-TSP) Based Single Sample Classifier

The k-TSP algorithm¹⁰, developed for solving binary classification problems, was here used for development of a diagnostic single-sample classifier Intended for a clinical setting. Such a setting would not allow for HiRIEF-LC-MS/TMT-labelling, and therefore the classifier was trained and applied on label-free DIA-MS data generated as described above. To remove samples with low quality DIA data, sample-wise correlation (Spearman) analysis between the original HiRIEF-LC-MS data and the DIA-MS was performed for overlapping proteins. This analysis revealed five samples with low correlation, possibly due to low amount of available starting material for DIA-MS, and these samples were excluded from downstream analysis.

For an Initial filtering to remove uninformative proteins (features) and to prevent high-computation time for downstream analysis, the inventors applied DEqMS¹⁵ as described above (BH adjusted p-value <0.01 and |log 2(FC)|>0.5). Comparison between differentially abundant 5872 proteins and the 6717 proteins identified in DIA analysis resulted in an overlap of 3028 proteins.

Missing values in DIA data were imputed by filling background level or baseline signals for each proteins, individually. The Inventors assumed that any resulting missing value was due to the lack of protein abundance in the sample. Therefore, the inventors imputed the missing values with background level or baseline signals instead of inferring the missing value based on protein abundance of other samples. The inventors sampled value from a Gaussian distribution N(μ, σ) where μ is halve of the minimum MS1 peak area of the protein abundance and σ is 2 in order to replace missing values with baseline signals for each sample Independently.

Protein-wise correlations (Spearman and Pearson) between HiRIEF-LC-MS and imputed DIA-MS data was computed for these 3028 proteins, and proteins with greater than 0.3 spearman and 0.5 Pearson correlations were included, resulting in a list of 1989 proteins. Next, for each comparison, the most upregulated and downregulated 100 (50×2) proteins were included in subsequent analysis resulting in a list of 757 proteins.

For k-TSP classification, the Inventors modified the ‘switchbox’ R package¹¹¹ for multi-class classification problems. The only parameter to tune is the number of feature pairs (k) used in the k-TSP algorithm (optimized k=15). One-versus-one classifiers were built to classify samples (in total 15 classifiers for the 6 subtypes), and for each classifier the sample was classified Into either of the subtypes. Consequently, each sample is classified 15 times and the final decision is made based on a majority vote. As for the SVM classifier the inventors used the Monte-Carlo-Cross-Validation (MCCV) method¹⁰⁸ to provide an unbiased performance estimation and to optimize the classifier. The whole process (described below) was repeated 100 times to guarantee for all samples to be included in training and testing at least once and for each iteration the testing performance (accuracy) and 225 (15×15) most Important feature pairs was reported.

First, the inventors partitioned the dataset randomly into two parts; 80% for training and 20% for testing. Testing data was separated before developing the model and it was only used for the testing, while training data was used to select feature pairs in order to build a model. In the training data, 15 classifiers (Subtype1 vs. Subtype2, Subtype1 vs. Subtype3, etc.) were built independently, while simultaneously determining the 15 feature pairs for each classifier. Next, the corresponding classifiers were applied to the testing data to estimate the classifier accuracy.

Finally, the overall accuracy was reported as the average accuracy from the 100 MCCV Iterations. To build the final model and deploy it, all feature pairs from the MCCV Iterations were sorted based on frequency and the top 15 most frequent pairs for each of the 15 classifiers were selected resulting in a total of 225 feature pairs (281 marker proteins.

Applying k-TSP Classifier to Independent Late-Stage Cohort Dataset

The k-TSP algorithm does not require any data normalization steps. It only compares the quantitative values of the proteins in each pair and assign samples to subtypes based on rules established during training. Therefore, the inventors can directly apply k-TSP algorithm to new samples. The final classification is based on a majority vote from the 15 classifiers, and in case of a tie in classifications, the sample is labeled as “unclassified” to prevent final ambiguous calls.

### REFERENCES

1 Cancer Genome Atlas Research, N. Comprehensive genomic characterization of squamous cell lung cancers. Nature 489, 519-525, doi:10.1038/nature11404 (2012).
2 Cancer Genome Atlas Research, N. Comprehensive molecular profiling of lung adenocarcinoma. Nature 511, 543-550, doi:10.1038/nature13385 (2014).
3 Perou, C. M. et al. Molecular portraits of human breast tumours. Nature 406, 747-752, doi:10.1038/35021093 (2000).
4 Egeblad, M., Nakasone, E. S. & Werb, Z. Tumors as organs: complex tissues that interface with the entire organism. Dev Cell 18, 884-901, doi:10.1016/j.devcel.2010.05.012 (2010).
5 Stewart, P. A. et al. Proteogenomic landscape of squamous cell lung cancer. Nat Commun 10, 3578, doi:10.1038/s41467-019-11452-x (2019).
6 Gillette, M. A. et al. Proteogenomic Characterization Reveals Therapeutic Vulnerabilities in Lung Adenocarcinoma. Cell 182, 200-225 e235, doi: 10.1016/j.cell.2020.06.013 (2020).
7 Xu, J. Y. et al. Integrative Proteomic Characterization of Human Lung Adenocarcinoma. Cell 182, 245-261 e217, doi:10.1016/j.cell.2020.05.043 (2020).
8 Chen, Y. J. et al. Proteogenomics of Non-smoking Lung Cancer in East Asia Delineates Molecular Signatures of Pathogenesis and Progression. Cell 182, 226-244 e217, doi: 10.1016/j.cell.2020.06.012 (2020).
9 Branca, R. M. et al. HiRIEF LC-MS enables deep proteome coverage and unbiased proteogenomics. Nat Methods 11, 59-62, doi:10.1038/nmeth.2732 (2014).
10 Zhu, Y. et al. Discovery of coding regions in the human genome by integrated proteogenomics analysis workflow. Nat Commun 9, 903, doi: 10.1038/s41467-018-03311-y (2018).
11 Johansson, H. J. et al. Breast cancer quantitative proteome and proteogenomic landscape. Nat Commun 10, 1600, doi:10.1038/s41467-019-09018-y (2019).
12 Karlsson, A. et al. Gene Expression Profiling of Large Cell Lung Cancer Links Transcriptional Phenotypes to the New Histological WHO 2015 Classification. J Thorac Oncol 12, 1257-1267, doi: 10.1016/j.jtho.2017.05.008 (2017).
13 Karlsson, A. et al. Genome-wide DNA methylation analysis of lung carcinoma reveals one neuroendocrine and four adenocarcinoma epitypes associated with patient outcome. Clin Cancer Res 20, 6127-6140, doi:10.1158/1078-0432.CCR-14-1087 (2014).
14 Arbajian, E. et al. Methylation Patterns and Chromatin Accessibility in Neuroendocrine Lung Cancer. Cancers (Basel) 12, doi:10.3390/cancers12082003 (2020).
15 Zhu, Y. et al. DEqMS: A Method for Accurate Variance Estimation in Differential Protein Expression Analysis. Mol Cell Proteomics 19, 1047-1057, doi:10.1074/mcp.TIR119.001646 (2020).
16 McInnes, L., Healy, J. & Melville, J. UMAP: Uniform Manifold Approximation and Projection for Dimension Reduction. arXiv:1802.03426 (2018).<https://ui.adsabs.harvard.edu/abs/2018arXiv180203426M>.
17 Yoshihara, K. et al. Inferring tumour purity and stromal and immune cell admixture from expression data. Nat Commun 4, 2612, doi:10.1038/ncomms3612 (2013).
18 Sanchez-Vega, F. et al. Oncogenic Signaling Pathways in The Cancer Genome Atlas. Cell 173, 321-337 e310, doi:10.1016/j.cell. 2018.03.035 (2018).
19 Futreal, P. A. et al. A census of human cancer genes. Nat Rev Cancer 4, 177-183, doi:10.1038/nrc1299 (2004).
Ma, X. et al. Characterization of the Src-regulated kinome identifies SGK1 as a key mediator of Src-induced transformation. Nat Commun 10, 296, doi:10.1038/s41467-018-08154-1 (2019).
21 Castel, P. et al. PDK1-SGK1 Signaling Sustains AKT-Independent mTORC1 Activation and Confers Resistance to PI3Kalpha Inhibition. Cancer Cell 30, 229-242, doi:10.1016/j.ccell.2016.06.004 (2016).
22 Gao, D. et al. Rictor forms a complex with Cullin-1 to promote SGK1 ubiquitination and destruction. Mol Cell 39, 797-808, doi:10.1016/j.molcel.2010.08.016 (2010).
23 Helwak, A., Kudla, G., Dudnakova, T. & Tollervey, D. Mapping the human miRNA interactome by CLASH reveals frequent noncanonical binding. Cell 153, 654-665, doi:10.1016/j.cell.2013.03.043 (2013).
24 Giurgiu, M. et al. CORUM: the comprehensive resource of mammalian protein complexes-2019. Nucleic Acids Res 47, D559-D563, doi:10.1093/nar/gky973 (2019).
25 Schwanhausser, B. et al. Global quantification of mammalian gene expression control. Nature 473, 337-342, doi:10.1038/nature10098 (2011).
26 Mayr, C., Hemann, M. T. & Bartel, D. P. Disrupting the pairing between let-7 and Hmga2 enhances oncogenic transformation. Science 315, 1576-1579, doi:10.1126/science. 1137999 (2007).
27 Campanero, M. R. & Flemington, E. K. Regulation of E2F through ubiquitin-proteasome-dependent degradation: stabilization by the pRB tumor suppressor protein. Proc Nat Acad Sci USA 94, 2221-2226, doi: 10.1073/pnas.94.6.2221 (1997).
28 Joshi, S., Kumar, S., Ponnusamy, M. P. & Batra, S. K. Hypoxia-induced oxidative stress promotes MUC4 degradation via autophagy to enhance pancreatic cancer cells survival. Oncogene 35, 5882-5892, doi:10.1038/onc.2016.119 (2016).
29 Ikink, G. J., Boer, M., Bakker, E. R. & Hilkens, 1. IRS4 induces mammary tumorigenesis and confers resistance to HER2-targeted therapy through constitutive PI3K/AKT-pathway hyperactivation. Nat Commun 7, 13567, doi:10.1038/ncomms13567 (2016).
30 Liu, J. et al. An Integrative cross-omics analysis of DNA methylation sites of glucose and Insulin homeostasis. Nat Commun 10, 2581, doi:10.1038/s41467-019-10487-4 (2019).
31 Valkovicova, T., Skopkova, M., Stanik, J. & Gasperikova, D. Novel Insights into genetics and clinics of the HNF1A-MODY. Endocr Regul 53, 110-134, doi:10.2478/enr-2019-0013 (2019).
32 Charoentong, P. et al. Pan-cancer Immunogenomic Analyses Reveal Genotype-Immunophenotype Relationships and Predictors of Response to Checkpoint Blockade. Cell Rep 18, 248-262, doi:10.1016/j.celrep.2016.12.019 (2017).
33 Dou, Y. et al. Proteogenomic Characterization of Endometrial Carcinoma. Cell 180, 729-748 e726, doi: 10.1016/j.cell.2020.01.026 (2020).
34 Sautes-Fridman, C., Petitprez, F., Calderaro, J. & Fridman, W. H. Tertiary lymphoid structures in the era of cancer Immunotherapy. Nat Rev Cancer 19, 307-325, doi:10.1038/s41568-019-0144-6 (2019).
35 Cabrita, R. et al. Tertiary lymphoid structures Improve Immunotherapy and survival in melanoma. Nature 577, 561-565, doi:10.1038/s41586-019-1914-8(2020).
36 Attermann, A. S., Bjerregaard, A. M., Saini, S. K., Gronbaek, K. & Hadrup, S. R. Human endogenous retroviruses and their Implication for Immunotherapeutics of cancer. Ann Oncol 29, 2183-2191, doi:10.1093/annonc/mdy413 (2018).
37 Chong, C. et al. Integrated proteogenomic deep sequencing and analytics accurately Identify non-canonical peptides in tumor immunopeptidomes. Nat Commun 11, 1293, doi:10.1038/s41467-020-14968-9 (2020).
38 Laumont, C. M. et al. Noncoding regions are the main source of targetable tumor-specific antigens. Sci Trans Med 10, doi:10.1126/scitranslmed.aau5516 (2018).
39 Ott, P. A. et al. An Immunogenic personal neoantigen vaccine for patients with melanoma. Nature 547, 217-221, doi:10.1038/nature22991 (2017).
40 Smith, C. C. et al. Alternative tumour-specific antigens. Nat Rev Cancer 19, 465-478, doi:10.1038/s41568-019-0162-4 (2019).
41 Almeida, L. G. et al. CTdatabase: a knowledge-base of high-throughput and curated data on cancer-testis antigens. Nucleic Acids Res 37, D816-819, doi:10.1093/nar/gkn673 (2009).
42 Simpson, A. J., Caballero, O. L., Jungbluth, A., Chen, Y. T. & Old, L. J. Cancer/testis antigens, gametogenesis and cancer. Nat Rev Cancer 5, 615-625, doi:10.1038/nrc1669 (2005).
43 Andrews, L. P., Yano, H. & Vignali, D. A. A. Inhibitory receptors and ligands beyond PD-1, PD-L1 and CTLA-4: breakthroughs or backups. Nat Immunol 20, 1425-1434, doi:10.1038/s41590-019-0512-0 (2019).
44 Qin, S. et al. Novel immune checkpoint targets: moving beyond PD-1 and CTLA-4. Mol Cancer 18, 155, doi:10.10.1186/s12943-019-1091-2 (2019).
45 Wang, J. et al. Fibrinogen-like Protein 1 is a Major immune Inhibitory Ligand of LAG-3. Cell 176, 334-347 e312, doi:10.1016/j.cell.2018.11.010 (2019).
46 Wei, J., Loke, P., Zang, X. & Allison, J. P. Tissue-specific expression of B7x protects from CD4 T cell-mediated autoimmunity. J Exp Med 208, 1683-1694, doi:10.1084/jem.20100639 (2011).
47 Jeon, H. et al. Structure and cancer Immunotherapy of the B7 family member B7x. Cell Rep 9, 1089-1098, doi:10.1016/j.celrep.2014.09.053 (2014).
48 Zeqiraj, E., Filippi, B. M., Deak, M., Alessi, D. R. & van Aalten, D. M. Structure of the LKB1-STRAD-M025 complex reveals an allosteric mechanism of kinase activation. Science 326, 1707-1711, doi:10.1126/science.1178377 (2009).
49 Kim, J. et al. CPS1 maintains pyrimidine pools and DNA synthesis in KRAS/LKB1-mutant lung cancer cells. Nature 546, 168-172, doi: 10.1038/nature22359 (2017).
50 Zhang, H. M. et al. AnimalTFDB 2.0: a resource for expression, prediction and functional study of animal transcription factors. Nucleic Adds Res 43, D76-81, doi:10.1093/nar/gku887 (2015).
51 Cancer Genome Atlas Research, N. et al. The Cancer Genome Atlas Pan-Cancer analysis project. Nat Genet 45, 1113-1120, doi:10.1038/ng.2764 (2013).
52 Yang, W. et al. Genomics of Drug Sensitivity in Cancer (GDSC): a resource for therapeutic biomarker discovery in cancer cells. Nucleic Acids Res 41, D955-961, doi:10.1093/nar/gks1111 (2013).
53 Shackelford, D. B. & Shaw, R. J. The LKB1-AMPK pathway: metabolism and growth control in tumour suppression. Nat Rev Cancer 9, 563-575, doi:10.1038/nrc2676 (2009).
54 Lim, S. B., Tan, S. 7., Lim, W. T. & Lim, C. T. A merged lung cancer transcriptome dataset for clinical predictive modeling. Sci Data 5, 180136, doi:10.1038/sdata.2018.136 (2018).
55 Camidge, D. R., Doebele, R. C. & Kerr, K. M. Comparing and contrasting predictive biomarkers for immunotherapy and targeted therapy of NSCLC. Nat Rev Clin Oncol 16, 341-355, doi:10.1038/541571-019-0173-9 (2019).
56 Woo, S. R. et al. immune inhibitory molecules LAG-3 and PD-1 synergistically regulate T-cell function to promote tumoral immune escape. Cancer Res 72, 917-927, doi:10.1158/0008-5472.CAN-11-1620 (2012).
57 Sica, G. L. et al. B7-H4, a molecule of the B7 family, negatively regulates T cell Immunity. Immunity 18, 849-861, doi:10.1016/s1074-7613(03)00152-3 (2003).
58 Parra, E. R. et al. Immunohistochemical and Image Analysis-Based Study Shows That Several immune Checkpoints are Co-expressed in Non-Small Cell Lung Carcinoma Tumors. J Thorac Oncol 13, 779-791, doi:10.1016/j.jtho.2018.03.002 (2018).
59 Azuma, T. et al. Potential role of decoy B7-H4 in the pathogenesis of rheumatoid arthritis: a mouse model informed by clinical data. PLoS Med 6, e1000166, doi:10.1371/journal.pmed.1000166 (2009).
60 Simon, I. et al. B7-h4 is a novel membrane-bound protein and a candidate serum and tissue biomarker for ovarian cancer. Cancer Res 66, 1570-1575, dot:10.1158/0008-5472.CAN-04-3550 (2006).
61 Wei, B. et al. A protein activity assay to measure global transcription factor activity reveals determinants of chromatin accessibility. Nat Biotechnol 36, 521-529, doi:10.1038/nbt.4138 (2018).
62 Courtois, G., Morgan, J. G., Campbell, L. A., Fourel, G. & Crabtree, G. R. Interaction of a liver-specific nuclear factor with the fibrinogen and alpha 1-antitrypsin promoters. Science 238, 688-692, doi:10.1126/science.3499668 (1987).
63 Huang, P. et al. Direct reprogramming of human fibroblasts to functional and expandable hepatocytes. Cell Stem Cell 14, 370-384, doi:10.1016/j.stem.2014.01.003 (2014).
64 Simeonov, K. P. & Uppal, H. Direct reprogramming of human fibroblasts to hepatocyte-like cells by synthetic modified mRNAs. PLoS One 9, e100134, doi:10.1371/journal. pone. 0100134 (2014).
65 Xu, L. et al. The Kinase mTORC1 Promotes the Generation and Suppressive Function of Follicular Regulatory T Cells. Immunity 47, 538-551 e535, doi:10.1016/j.immuni.2017.08.011 (2017).
66 Hughes, C. S. et al. Single-pot, solid-phase-enhanced sample preparation for proteomics experiments. Nat Protoc 14, 68-85, doi:10.1038/s41596-018-0082-x (2019).
67 Foroughi Asl, H. BALSAMIC: A bioinformatic analysis pipeline for somatic mutations in cancer [Online]. Available online at: https://github.com/Clinical-Genomics/BALSAMIC. (2019).
68 Andrews, S. A Quality Control Tool for High Throughput Sequence Data [Online]Available online at: http://www.bioinformatics.babraham.ac.uk/projects/fastqc/. (2010).
69 Chen, S., Zhou, Y., Chen, Y. & Gu, J. fastp: an ultra-fast all-in-one FASTQ preprocessor. Bioinformatics 34, 1884-1890, doi:10.1093/bioinformatics/bty560 (2018).
70 Li, H. & Durbin, R. Fast and accurate short read alignment with Burrows-Wheeler transform. Bioinformatics 25, 1754-1760, doi: 10.1093/bioinformatics/btp324 (2009).
71 Li, H. A statistical framework for SNP calling, mutation discovery, association mapping and population genetical parameter estimation from sequencing data. Bioinformatics 27, 2987-2993, dot:10.1093/bioinformatics/btr509 (2011).
72 Li, H. et al. The Sequence Alignment/Map format and SAMtools. Bioinformatics 25, 2078-2079, doi:10.1093/bioinformatics/btp352 (2009).
73 Broad Institute. Picard toolkit. Broad Institute, GitHub repository (2019).
74 Lai, Z. et al. VarDict: a novel and versatile variant caller for next-generation sequencing in cancer research. Nucleic Acids Res 44, e108, doi:10.1093/nar/gkw227 (2016).
75 McLaren, W. et al. The Ensembl Variant Effect Predictor. Genome Biol 17, 122, doi:10.1186/513059-016-0974-4 (2016).
76 Tamborero, D. et al. Support systems to guide clinical decision-making in precision oncology: The Cancer Core Europe Molecular Tumor Board Portal. Nat Med, doi:10.1038/541591-020-0969-2 (2020).
77 Chalmers, Z. R. et al. Analysis of 100,000 human cancer genomes reveals the landscape of tumor mutational burden. Genome Med 9, 34, doi:10.1186/s13073-017-0424-2 (2017).
78 Benjamini, Y. & Hochberg, Y. CONTROLLING THE FALSE DISCOVERY RATE—A PRACTICAL AND POWERFUL APPROACH TO MULTIPLE TESTING. J. R. Stat. Soc. Ser. B-Stat. Methodol. 57, 289-300 (1995).
79 Travis, W. D., Brambilla, E., Muller-Hermelink, H. K. & Harris, C. C. Pathology and Genetics: Tumours of the Lung, Pleura, Thymus and Heart. (World Health Organization, 2004).
80 WHO Classification of Turnouts of the Lung, Pleura, Thymus and Heart. Fourth edn, Vol. 7 (WHO Press, 2015).
81 Dako, A. (ed Agilent Technologies) (Agilent, United States, 2018).
82 Al-Shibli, K. I. et al. Prognostic effect of epithelial and stromal lymphocyte infiltration in non-small cell lung cancer. Clinical cancer research: an official journal of the American Association for Cancer Research 14, 5220-5227, doi:10.1158/1078-0432.ccr-08-0133 (2008).
83 Travis, W. D. et al. International association for the study of lung cancer/american thoracic society/european respiratory society International multidisciplinary classification of lung adenocarcinoma. Journal of thoracic oncology: official publication of the International Association for the Study of Lung Cancer 6, 244-285, doi:10.1097/JTO.0b013e318206a221 (2011).
84 Wilkerson, M. D. & Hayes, D. N. ConsensusClusterPlus: a class discovery tool with confidence assessments and Item tracking. Bioinformatics 26, 1572-1573, dot:10.1093/bioinformatics/btq170 (2010).
85 Butler, A., Hoffman, P., Smibert, P., Papalexi, E. & Satija, R. Integrating single-cell transcriptomic data across different conditions, technologies, and species. Nat Biotechnol 36, 411-420, doi:10.1038/nbt.4096 (2018).
86 Blondel, V. D., Guillaume, J.-L., Lambiotte, R. & Lefebvre, E. Fast unfolding of communities in large networks. Journal of Statistical Mechanics: Theory and Experiment 2008, P10008, doi: 10.1088/1742-5468/2008/10/p10008 (2008).
87 Yu, G., Wang, L. G., Han, Y. & He, Q. Y. clusterProfiler: an R package for comparing biological themes among gene clusters. OMICS 16, 284-287, doi:10.1089/omi.2011.0118 (2012).
88 Travaglini, K. J. et al. A molecular cell atlas of the human lung from single cell RNA sequencing. bioRxiv, 742320, doi:10.1101/742320 (2019).
89 Liberzon, A. et al. The Molecular Signatures Database (MSigDB) hallmark gene set collection. Cell Syst 1, 417-425, doi:10.1016/j.cels.2015.12.004 (2015).
90 Hanzelmann, S., Castelo, R. & Guinney, J. GSVA: gene set variation analysis for microarray and RNA-seq data. BMC Bioinformatics 14, 7, doi:10.1186/1471-2105-14-7 (2013).
91 Ogata, H. et al. KEGG: Kyoto Encyclopedia of Genes and Genomes. Nucleic Acids Res 27, 29-34, doi:10.1093/nar/27.1.29 (1999).
92 Manning, G., Whyte, D. B., Martinez, R., Hunter, T. & Sudarsanam, S. The protein kinase complement of the human genome. Science 298, 1912-1934, doi:10.1126/science.1075762 (2002).
93 Chen, M. J., Dixon, J. E. & Manning, G. Genomics and evolution of protein phosphatases. Sci Signal 10, doi:10.1126/scisignal.aag1796 (2017).
94 Li, W. et al. Genome-wide and functional annotation of human E3 ubiquitin ligases Identifies MULAN, a mitochondrial E3 that regulates the organelle's dynamics and signaling. PLoS One 3, e1487, doi:10.1371/journal.pone.0001487 (2008).
95 Orre, L. M. et al. SubCellBarCode: Proteome-wide Mapping of Protein Localization and Relocalization. Mol Cell 73, 166-182 e167, doi:10.1016/j.molcel.2018.11.035 (2019).
96 Santos, R. et al. A comprehensive map of molecular drug targets. Nat Rev Drug Discov 16, 19-34, doi:10.1038/nrd.2016.230 (2017).
97 Kent, W. J. et al. The human genome browser at UCSC. Genome Res 12, 996-1006, doi:10.1101/gr.229102 (2002).
98 Yates, A. D. et al. Ensembl 2020. Nucleic Adds Res 48, D682-D688, doi:10.1093/nar/gkz966 (2020).
99 Nesvizhskii, A. I. Proteogenomics: concepts, applications and computational strategies. Nat Methods 11, 1114-1125, doi:10.1038/nmeth.3144 (2014).
100 Kent, W. J. BLAT—the BLAST-like alignment tool. Genome Res 12, 656-664, doi:10.1101/gr.229202 (2002).
101 UniProt, C. UniProt: a worldwide hub of protein knowledge. Nucleic Adds Res 47, D506-D515, doi:10.1093/nar/gky1049 (2019).
102 O'Leary, N. A. et al. Reference sequence (RefSeq) database at NCBI: current status, taxonomic expansion, and functional annotation. Nucleic Adds Res 44, D733-745, doi:10.1093/nar/gkv1189 (2016).
103 Harrow, J. et al. GENCODE: the reference human genome annotation for The ENCODE Project. Genome Res 22, 1760-1774, doi:10.1101/gr.135350.111 (2012).
104 Wang, K., Li, M. & Hakonarson, H. ANNOVAR: functional annotation of genetic variants from high-throughput sequencing data. Nucleic Adds Res 38, e164, doi:10.1093/nar/gkq603 (2010).
105 Volders, P. J. et al. LNCipedia 5: towards a reference set of human long non-coding RNAs. Nucleic Acids Res 47, D135-D139, doi:10.1093/nar/gky1031 (2019).
106 Pei, B. et al. The GENCODE pseudogene resource. Genome Biol 13, R51, doi:10.1186/gb-2012-13-9-r51 (2012).
107 Pedregosa, F. et al. Scikit-learn: Machine learning in Python. Journal of Machine Learning Research 12 (2011).
108 Xu, Q.-s. & Liang, Y.-Z. Monte Carlo cross validation. Chemometrics and Intelligent Laboratory Systems 56, 1-11 (2001).
109 Guyon, I., Weston, I., Barnhill, S. & Vapnik, V. Gene Selection for Cancer Classification using Support Vector Machines. Machine Learning 46, 389-422, doi:10.1023/A:1012487302797 (2002).
110 Tan, A. C., Naiman, D. Q., Xu, L., Winslow, R. L. & Geman, D. Simple decision rules for classifying human cancers from gene expression profiles. Bioinformatics 21, 3896-3904, doi:10.1093/bioinformatics/bti631 (2005).
111 Afsari, B., Fertig, E. J., Geman, D. & Marchionni, L. switchBox: an R package for k-Top Scoring Pairs classifier development. Bioinformatics 31, 273-274, doi:10.1093/bioinformatics/btu622 (2015).

### Example 2: Peptide-Centric Classification Using Support Vector Machine (SVM-Peptide

Here below follows the description of DIA-MS based analysis of lung cancer samples and SVM based classification of cancers by quantitative patterns of peptide features. The method is intended for both label-free quantification and quantification based on spiked-in peptide standards or any other peptide level quantification method.

Data Preparation

DIA-MS (Data-Independent Acquisition) analysis resulted in the identification of 6717 proteins across the 141 samples in the lung cancer cohort. To remove samples with low quality DIA-MS data, sample-wise correlation (Spearman) analysis between the original HiRIEF-LC-MS data (DDA, Data-Dependent Acquisition) and the DIA-MS data was performed for overlapping proteins. This analysis revealed five samples with low correlation, possibly due to low amount of available starting material for DIA-MS, and these samples were excluded from downstream analysis. For an initial filtering to remove uninformative proteins (features) and to prevent high-computation time for downstream analysis, we applied DEqMS¹ to Identify proteins that were differentially abundant between the six subtypes based on the DDA analysis (BH adjusted p-value <0.01 and |log 2(FC)|>0.5). Comparison between differentially abundant 5872 proteins in the DDA analysis and the 6717 proteins identified in DIA analysis resulted in an overlap of 3028 proteins in the 136 cohort samples.

Missing protein level quantifications in the DIA data were imputed by estimating baseline MS1 peak areas for each protein individually. This was performed by sampling values from a Gaussian distribution (N(μ, σ), where μ=(min. MS1 peak area of the samples with quantification)/2, and σ=2) in order to replace missing values with baseline/low values for each sample independently.

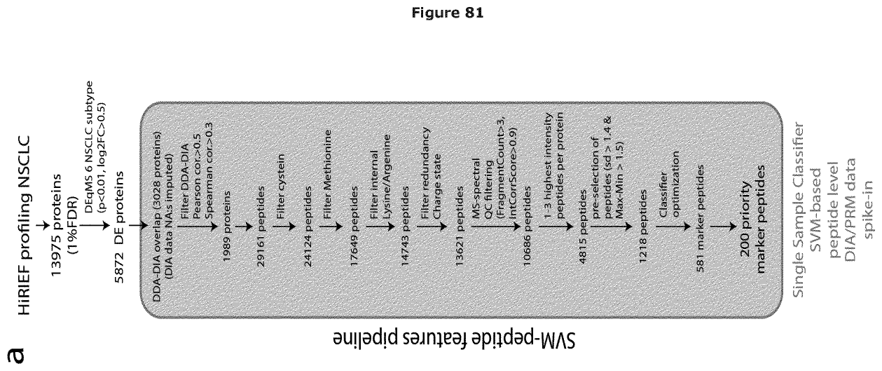

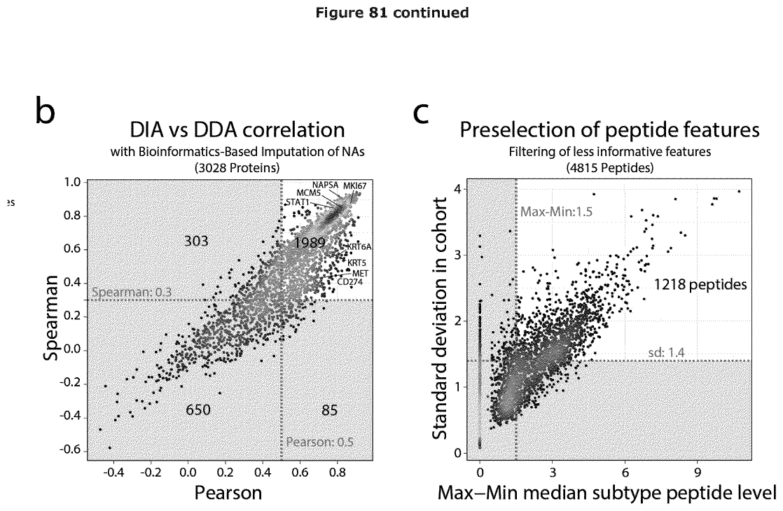

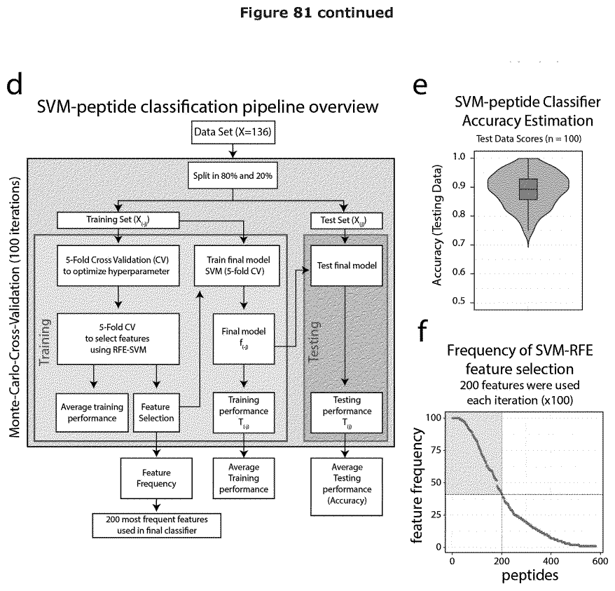

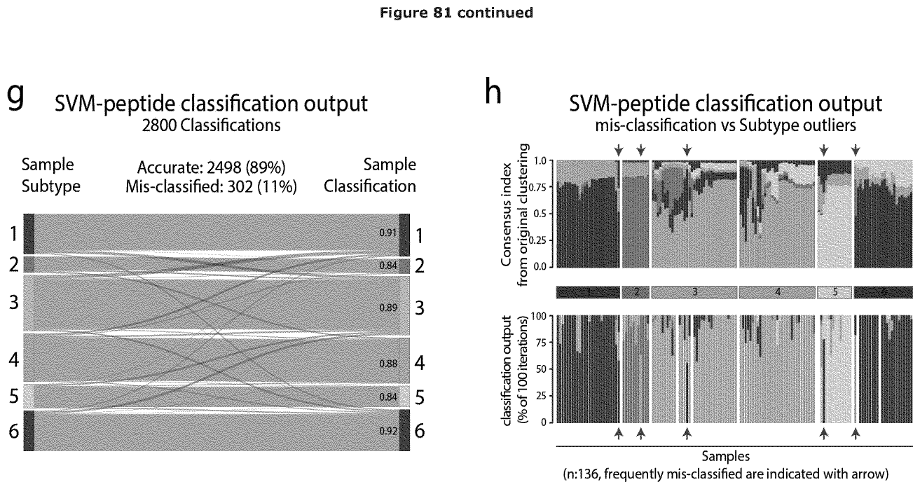

Next, protein-wise correlation between DDA and DIA data (3028 proteins) was used for filtering the data (Spearman>0.3 and Pearson>0.5) resulting in a list of 1989 proteins (FIG. 81a-1b). The corresponding 29161 peptides from the 1989 proteins were further processed and analyzed.

Peptides with Cysteine and Methionine modifications were removed to avoid problems related to disulfide cross-linking and oxidation in future assay development, and peptides containing internal Lysine and Arginine amino acid were removed as these peptides included missed trypsin cleavage sites. Peptides with redundant charge state were subsequently filtered out to avoid replicated non-unique peptide quantifications. For the remaining 13621 peptides, MS-spectral quality filtering was applied (Fragment Count >3 and IntCorrScore >0.9), followed by selection of the 1-3 highest intensity peptides per protein. Peptide quantifications for the remaining 4815 peptides were median normalized by dividing each value with the median of the MS1 quantifications across the 136 samples, and log 2 transformed.

Support Vector Machine (SVM) Based Classifier for Peptide Centric DIA Data

Missing peptide level quantifications in the DIA data were imputed by estimating baseline intensities for each peptide individually. This was performed by sampling values from a Gaussian distribution (N(μ, σ), where μ=(min. MS1 peak intensity of the samples with quantification)/2, and σ=2) in order to replace missing values with baseline/low values for each sample independently. For an initial filtering to remove less informative peptides (features) and to prevent high-computation time for feature selection, we kept peptides with high overall standard deviation (sd>1.4) and large differences between subtypes (maximum—minimum median subtype peptide level >1.5), resulting in a list of 1218 peptides (FIG. 81c).

Support-Vector-Machine (SVM) with linear kernel was used to build the SVM-peptide classifier. In machine learning with large datasets, a dataset is often split into three parts, for training, validation and testing. However, in this study we were not in a data-rich situation and could therefore not split the data into three parts. Instead, we used the Monte-Carlo-Cross-Validation (MCCV) method² to provide an unbiased performance estimation and to optimize the model. The whole process (FIG. 81d) was repeated 100 times to maximize the number of samples included in training and testing. From each Iteration, the testing performance (accuracy) and 200 most important features were reported.

First, we partitioned the dataset randomly into two parts; 80% for training and 20% for testing. Testing data was separated before developing the model and it was only used for the testing, while training data was used to select features and to tune the parameters in order to build a model. Hyperparameter C ranges from 0.001 to 1000 and the model was optimized using 5-fold Cross-Validation to avoid overfitting. To select the most important features in each iteration, Support Vector Machine-Recursive Feature Elimination (SVM-RFE) algorithm was applied³. SVM-RFE selects the features based on how important they are for separating the groups. It starts with all features (1218) and for each step, a number of least important features are eliminated from the feature set. This process is repeated until the specified number of top features (200) are left in the dataset. The algorithm was implemented using scikit-learn library in python (version 3)⁴. The model with the 200 most important features were then applied to test data for estimation of the accuracy.

The SVM peptide classifier achieved high accuracy (average accuracy from the 100 MCCV iterations: 89%, FIG. 81e), as well as a high degree of feature pair redundancy between the Iterations, and to build the final model and deploy it, we selected the most frequently used 200 features (FIG. 81f) from the output of MCCV. Overall, misclassifications of the SVM-peptide classifier were spread out between subtypes but concentrated upon a limited number of samples, largely overlapping with subtype outliers (FIG. 81g-1h).

### REFERENCES FOR EXAMPLE 2

1. Zhu, Y. et al. DEqMS: A Method for Accurate Variance Estimation in Differential Protein Expression Analysis. Mol Cell Proteomics 19, 1047-1057, doi:10.1074/mcp.TIR119.001646 (2020).
2. Xu, Q.-s. & Liang, Y.-Z. Monte Carlo cross validation. Chemometrics and Intelligent Laboratory Systems 56, 1-11 (2001).
3. Guyon, I., Weston, I., Barnhill, S. & Vapnik, V. Gene Selection for Cancer Classification using Support Vector Machines. Machine Learning 46, 389-422, doi:10.1023/A:1012487302797 (2002).
4. Pedregosa, F. et al. Scikit-learn: Machine learning in Python. Journal of Machine Learning Research 12 (2011).

1. A method for determining the prognosis of Non-Small Cell Lung Cancer (NSCLC) in an individual, the method comprising:
(1-a) providing a test sample from the individual;
(1-b) determining a biomarker signature, by measuring in the test sample the presence and/or amount of:
39 or more of the biomarkers defined in Table 1; and/or
11 or more of the biomarkers defined in Table 2; and/or
2 or more of the biomarkers defined in Table 3; and/or
8 or more of the biomarkers defined in Table 4; and/or
137 or more of the biomarkers defined in Table 5; and/or
36 or more of the biomarkers defined in Table 6;
(1-c) classifying the NSCLC in the individual on the basis of Step (1-b));
wherein the prognosis of NSCLC in the individual is determined on the basis of the classification in step (1-c).

2-43. (canceled)

44. The method according to claim 1, wherein Step (1-b) comprises measuring in the test sample the presence and/or amount of all of the biomarkers defined in Table 1 and/or Table 2 and/or Table 3 and/or Table 4 and/or Table 5 and/or Table 6.

45. The method according to claim 1, wherein Step (1-b) comprises measuring in the test sample the presence and/or amount of the biomarkers defined in two or more, or three or more, or four or more, or five or more, or all of Tables 1-6.

46. The method according to claim 1, wherein Step (1-c) comprises comparing the biomarker signature in (1-b) with the corresponding biomarker signature of a control sample.

47. The method according to claim 46, wherein Step (1-c) comprises determining whether the biomarkers of the biomarker signature in (1-b) are present in an elevated amount compared to the biomarkers of the biomarker signature of the control sample.

48. The method according to claim 46, wherein the control sample is a sample derived from normal lung tissue from the individual; or from a healthy individual; or a pool of healthy individuals.

49. The method according to claim 46, wherein the control sample is a sample derived from an individual with confirmed NSCLC; or a pool of NSCLC samples.

50. A method for determining the prognosis of Non-Small Cell Lung Cancer (NSCLC) in an individual, the method comprising:
(2-a) providing a test sample from the individual;
(2-b) determining in the test sample the presence and/or amount of the biomarkers in Table A and/or one or more of Tables B-G;
(2-c) applying a classification algorithm to the information obtained in step (2-b) in order to classify the NSCLC in the individual;
(2-d) classifying the NSCLC in the individual on the basis of Step (2-c), wherein the NSCLC is classified according to the biomarkers defined in Table 1 (as Prognosis Subtype 1) and/or Table 2 (as Prognosis Subtype 2) and/or Table 3 (as Prognosis Subtype 3) and/or Table 4 (as Prognosis Subtype 4) and/or Table 5 (as Prognosis Subtype 5) and/or Table 6 (as Prognosis Subtype 6);
wherein the prognosis of NSCLC in the individual is determined on the basis of the classification in step (2-d).

51. The method of claim 50, wherein the classification algorithm is selected from:
Support Vector Machine-protein (“SVM-protein”);
K-Top Scoring Pairs (“k-TSP”); or
Support Vector Machine-peptide (“SVM-peptide”).

52. The method of claim 51, wherein the classification algorithm is a Support Vector Machine-protein (“SVM-protein”), and wherein Step (2-b) comprises measuring the presence and/or amount of 145 or more of the biomarkers defined in Table B, and/or 60 or more of the biomarkers defined in Table C, optionally wherein all of the biomarkers of Table B and/or Table C are measured.

53. The method of claim 51, wherein the classification algorithm is K-Top Scoring Pairs (“k-TSP”) and wherein Step (2-b) comprises measuring the presence and/or amount of 489 or more of the biomarkers defined in Table D, and/or 67 or more of the biomarkers defined in Table E, optionally wherein all of the biomarkers of Table D and/or Table E are measured.

54. The method of claim 51, wherein the classification algorithm is Support Vector Machine-peptide (“SVM-peptide”) and wherein Step (2-b)) comprises measuring the presence and/or amount of 174 or more of the biomarkers defined in Table F, and/or 60 or more of the biomarkers defined in Table G, optionally wherein all of the biomarkers of Table F and/or Table G are measured.

55. A method for determining the prognosis of Non-Small Cell Lung Cancer (NSCLC) in an individual, the method comprising:
(3-a) providing a test sample from the individual;
(3-b) determining in the test sample the presence and/or amount of the biomarkers defined in Table A and/or one or more of Tables B-G;
(3-c) applying a classification algorithm to the information obtained in step (3-b) in order to classify the NSCLC in the individual, wherein the classification algorithm is selected from:
Support Vector Machine-protein (“SVM-protein”) and the biomarkers defined in Table B or C; or
K-Top Scoring Pairs (“k-TSP”) and the biomarkers defined in Table D or E; or
Support Vector Machine-peptide (“SVM-peptide”) and the biomarkers defined in Table F or G; and
(3-d) classifying the NSCLC in the individual on the basis of Step (3-c);
wherein the prognosis of NSCLC in the individual is determined on the basis of the classification in step (3-d).

56. The method according to claim 55, wherein the NSCLC is classified in Step (3d) according to the biomarkers defined in Table 1 (as Prognosis Subtype 1) and/or Table 2 (as Prognosis Subtype 2) and/or Table 3 (as Prognosis Subtype 3) and/or Table 4 (as Prognosis Subtype 4) and/or Table 5 (as Prognosis Subtype 5) and/or Table 6 (as Prognosis Subtype 6).

57. The method according to claim 50, wherein determining in the test sample the presence and/or amount of the biomarkers defined in Table A comprises measuring 526 or more of the biomarkers defined in Table A.

58. The method according to claim 57, wherein determining in the test sample the presence and/or amount of the biomarkers defined in Table A comprises measuring all of the biomarkers defined in Table A.

59. The method according to claim 50, wherein determining in the test sample the presence and/or amount of the biomarkers defined in Table A comprises determining the presence and/or amount of:
39 or more of the biomarkers defined in Table A(i); and/or
11 or more of the biomarkers defined in Table A(ii); and/or
2 or more of the biomarkers defined in Table A(iii); and/or
8 or more of the biomarkers defined in Table A(iv); and/or
137 or more of the biomarkers defined in Table A(v); and/or
36 or more of the biomarkers defined in Table A(vi).

60. The method according to claim 59, wherein determining in the test sample the presence and/or amount of the biomarkers defined in Table A comprises determining the presence and/or amount of all of the biomarkers defined in Table A(i) and/or Table A(ii) and/or Table A(iii) and/or Table A(iv) and/or Table A(v) and/or Table (vi).

61. The method according to claim 59, wherein determining in the test sample the presence and/or amount of the biomarkers defined in Table A comprises determining the presence and/or amount of all of the biomarkers defined in each of Table A(i) and Table A(ii) and Table A(iii) and Table A(iv) and Table A(v) and Table A(vi).

62. The method according to claim 59, wherein determining in the test sample the presence and/or amount of the biomarkers defined in Table A further comprises determining the presence and/or amount of one or more biomarkers defined in Table A(vii), optionally wherein the presence and/or amount of 335 or more biomarkers defined in Table A(vii) are determined, optionally wherein the presence and/or amount of all of the biomarkers defined in Table A(vii) are determined.

63. The method according to claim 1, wherein Step (I-b), Step (2-b) or Step (3 b) comprises measuring the expression of the protein or peptide biomarker(s) or comprises measuring the expression of a nucleic acid molecule encoding the biomarker(s).

64. The method according to claim 63, wherein the nucleic acid molecule is an mRNA molecule or a cDNA molecule.

65. The method according to claim 1, wherein the step of classifying the individual comprises:
classifying the individual as Prognosis Subtype 1, if the individual has a biomarker signature comprising an elevated amount of the biomarkers defined in Table 1;
classifying the individual as Prognosis Subtype 2 if the individual has a biomarker signature comprising an elevated amount of the biomarkers defined in Table 2;
classifying the individual as Prognosis Subtype 3 if the individual has a biomarker signature comprising an elevated amount of the biomarkers defined in Table 3;
classifying the individual as Prognosis Subtype 4 if the individual has a biomarker signature comprising an elevated amount of the biomarkers defined in Table 4;
classifying the individual as Prognosis Subtype 5 if the individual has a biomarker signature comprising an elevated amount of the biomarkers defined in Table 5; or
classifying the individual as Prognosis Subtype 6 if the individual has a biomarker signature comprising an elevated amount of the biomarkers defined in Table 6.

66. The method according to claim 1, wherein determining the prognosis of NSCLC in the individual comprises determining the probable survival time of the individuals.

67. The method according to claim 1, wherein the test sample comprises one or more lung cancer cells, and is optionally selected from a biopsy; a tissue sample; an organ sample; or a bodily fluid sample.

68. The method according to claim 1, wherein the NSCLC in the individual is at Stage O, I, II, III, or IV NSCLC.

69. The method according to claim 1, wherein the presence and/or amount of the biomarkers in the test sample is determined using Mass Spectrometry.

70. The method according to claim 1, wherein the presence and/or amount of the biomarkers in the test sample is determined using an affinity-based method.

71. The method according to claim 1, wherein the presence and/or amount of the biomarkers in the test sample is determined using a transcriptomics-based method.

72. The method according to claim 1, further comprising after determining the prognosis of NSCLC in the individual, selecting a treatment for the individual on the basis of the prognosis and, optionally, administering the selected treatment to the individual.

73. A method for treating NSCLC in an individual, the method comprising:
determining the prognosis of NSCLC in the individual by the method defined in claim 1; and
selecting a treatment for the individual, on the basis of the prognosis of NSCLC in the individual, and administering the selected treatment to the individual.

74. The method according to claim 73, wherein the treatment is one or more of: chemotherapy, immunotherapy, adoptive cell therapies, gene therapies, cancer vaccines, or oncolytic virus therapies.

75. A method for treating NSCLC in an individual, the method comprising:
determining the prognosis of NSCLC in the individual by the method defined in claim 1; and
selecting a treatment for the individual, on the basis of the classification of the NSCLC in Step (1-c), Step (2-d) or Step (3-d), and administering the selected treatment to the individual.

76. The method according to claim 75, wherein the treatment is one or more of: chemotherapy, immunotherapy, adoptive cell therapies, gene therapies, cancer vaccines, or oncolytic virus therapies.

77. The method according to claim 75, wherein selecting the treatment is also on the basis of the prognosis of the NSCLC in the individuals.

78. The method according to claim 75, wherein the NSCLC is classified as Prognosis Subtype 1 and/or Prognosis Subtype 2 and/or Prognosis Subtype 3 and/or Prognosis Subtype 4 and/or Prognosis Subtype 5 and/or Prognosis Subtype 6.

79. The method according to claim 78, wherein:
the NSCLC is classified as Prognosis Subtype 1 and the treatment is an EGFR targeting therapy; or
the NSCLC is classified as Prognosis Subtype 4 and the treatment is an mTOR pathway targeting therapy.

80. The method according to claim 1, wherein the NSCLC is selected from: adenocarcinoma; squamous cell carcinoma; adenosquamous carcinoma; large cell carcinoma; or large cell neuroendocrine cancer.

81. The method according to claim 1, wherein the individual is selected from: a primate; a rodent; a canine; a feline; an equine; a bovine; or a porcine.
